SEQUENCE LISTING (116 sequences)

SEQ ID NO: 1 - DNA, 68 bp, Artificial Sequence
  Synthetic
tgtctttagtttactcagcatcagctactaacatacctgaacgaagatcttgttctaagacattgtat

SEQ ID NO: 2 - protein, 102 aa, Artificial Sequence
  Synthetic
DTVKVMVVMLAICFLARSDGKPVKKRSVSEIQLMHNLGKHLSSLERVEWLRKKLQDVHNFVVLGASIVHRDGGSQRPPKKEDNVLVESHQKSLGEADKAAVG

SEQ ID NO: 3 - DNA, 306 bp, Artificial Sequence
  Synthetic
gacacagttaaagtaatggttgtcatgcttgcaatttgttttcttgcaagatcagatgggaagcctgttaagaagagatctgtgagtgaaatacagcttatgcataacctgggcaaacacctgagctctctggagagagtggaatggctgcgaaagaagctgcaggatgtgcacaactttgttgttctcggagcttctatagttcacagagatggtggttcccagagacccccaaaaaaggaagacaatgtcctagttgagagccatcaaaaaagtctcggagaagcagataaagctgctgtgggg

SEQ ID NO: 4 - protein, 67 aa, Artificial Sequence
  Synthetic
MERVEWLRKKLQDVHNFVALGAPLAPRDAGSQRPRKKEDNVLVESHEKSLGEADKADVNVLTKAKSQ

SEQ ID NO: 5 - DNA, 290 bp, Artificial Sequence
  Synthetic
aattctgtaagggatccaagaagagatcggtttctgagatccaattgatgcataacttgggtaagcacttgaactctatggaaagagttgaatggttgagaaagaagctgcaggacgttcacaacttcgttgctttgggagctccattggctccaagagacgctggttctcaaagaccaagaaagaaggaagacaacgttttggttgaatctcacgaaaagtctttgggtgaagctgacaaggctgacgttaacgtgttaactaaggctaaatcgcaataaagatcttga

SEQ ID NO: 6 - DNA, 120 bp, Homo sapiens
aggagctccaagggcaacaaaattgtgcacatcctgcagcttcttacgcagccattctactctctccatcgagttcagatgttttcccaggttatgcataagctgtatttcactcacaga

SEQ ID NO: 7 - DNA, 116 bp, Homo sapiens
gagtcatcatccatttcccattttccctctccatccacaagcatatactcaatattcagtcttttttttttttttttttgagacagggtctcactctgtcgcccaggctggagtac

SEQ ID NO: 8 - protein, 115 aa, Homo sapiens
MIPAKDMAKVMIVMLAICFLTKSDGKSVKKRSVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 9 - DNA, 772 bp, Homo sapiens
tgtctttagtttactcagcatcagctactaacatacctgaacgaagatcttgttctaagacattgtatgtgaagatgatacctgcaaaagacatggctaaagttatgattgtcatgttggcaatttgttttcttacaaaatcggatgggaaatctgttaagaagagatctgtgagtgaaatacagcttatgcataacctgggaaaacatctgaactcgatggagagagtagaatggctgcgtaagaagctgcaggatgtgcacaattttgttgcccttggagctcctctagctcccagagatgctggttcccagaggccccgaaaaaaggaagacaatgtcttggttgagagccatgaaaaaagtcttggagaggcagacaaagctgatgtgaatgtattaactaaagctaaatcccagtgaaaatgaaaacagatattgtcagagttctgctctagacagtgtagggcaacaatacatgctgctaattcaaagctctattaagatttccaagtgccaatatttctgatataacaaactacatgtaatccatcactagccatgataactgcaattttaattgattattctgattccacttttattcatttgagttattttaattatcttttctattgtttattctttttaaagtatgttattgcataatttataaaagaataaaattcgacttttaaacctctcttctaccttaaaatgtaaaacaaaaatgtaatgatcataagtctaaataaatgaagtatttctcactc

SEQ ID NO: 10 - DNA, 27 bp, Unknown
  Synthetic
gatcctcattactgggatttagcttta

SEQ ID NO: 11 - DNA, 42 bp, Unknown
  Synthetic
tgaaaaatccctaggcgaggcagacaaggccgatgtgaatgt

SEQ ID NO: 12 - DNA, 263 bp, Artificial Sequence
  Synthetic
tatgtctgtgtccgagattcagttaatgcataaccttggcaaacatttgaactccatggagcgtgtagaatggctgcgtaagaagttgcaggatgtgcacaattttgttgccttaggtgccccattggctcctcgtgatgctggttcccaaagaccacgtaaaaaggaagacaatgtcttagttgagagccatgaaaaatccctaggcgaggcagacaaggccgatgtgaatgtattaactaaagctaaatcccagtaatgag

SEQ ID NO: 13 - DNA, 614 bp, Artificial Sequence
  Synthetic
tttgagtgagaaatacttcatttatttagacttatgatcattacatttttgttttacattttaaggtagaagagaggtttaaaagtcgaattttattcttttataaattatgcaataacatactttaaaaagaataaacaatagaaaagataattaaaataactcaaatgaataaaagtggaatcagaataatcaattaaaattgcagttatcatggctagtgatggattacatgtagtttgttatatcagaaatattggcacttggaaatcttaatagagctttgaattagcagcatgtattgttgccctacactgtctagagcagaactctgacaatatctgttttcattttcactgggatttagctttagttaatacattcacatcagctttgtctgcctctccaagacttttttcatggctctcaaccaagacattgtcttccttttttcggggcctctgggaaccagcatctctgggagctagaggagctccaagggcaacaaaattgtgcacatcctgcagcttcttacgcagccattctactctctccatcgagttcagatgttttcccaggttatgcataagctgtatttcactcacagatctctt

SEQ ID NO: 14 - DNA, 68 bp, Unknown
  Synthetic
atacaatgtcttagaacaagatcttcgttcaggtatgttagtagctgatgctgagtaaactaaagaca

SEQ ID NO: 15 - protein, 115 aa, Homo sapiens; identical to SEQ ID NO: 8

SEQ ID NO: 16 - DNA, 1156 bp, Homo sapiens
aagcttctcgtgaaaaccaacccaattagttagtattgcattctgtgtactatagtttggaatattaaaaatattttaaaatacctccattttgcttatccttttagtgaagatgatacctgcaaaagacatggctaaagttatgattgtcatgttggcaatttgttttcttacaaaatcggatgggaaatctgttaagtaagtactgttttgccttggaattggatttttaatgttgactttatcatttcgaagtggggagctaatgggaagtggccctctctgtttctcttcttcccaggaagagatctgtgagtgaaatacagcttatgcataacctgggaaaacatctgaactcgatggagagagtagaatggctgcgtaagaagctgcaggatgtgcacaattttgttgcccttggagctcctctagctcccagagatgctggttcccagaggccccgaaaaaaggaagacaatgtcttggttgagagccatgaaaaaagtcttggagaggcagacaaagctgatgtgaatgtattaactaaagctaaatcccagtgaaaatgaaaacagatattgtcagagttctgctctagacagtgtagggcaacaatacatgctgctaattcaaagctctattaagatttccaagtgccaatatttctgatataacaaactacatgtaatccatcactagccatgataactgcaattttaattgattattctgattccacttttattcatttgagttattttaattatcttttctattgtttattctttttaaagtatgttattgcataatttataaaagaataaaattgcacttttaaacctctcttctaccttaaaatgtaaaacaaaaatgtaatgatcataagtctaaataaatgaagtatttctcactcattgcaagtatatctttttggttatcactgatacccacatgtttacattgatcatgactaggtagaacaatacaaagtatttttttagtcatgtgtttcacatttggatattttgaacatcaacgttttagtattaccaaagtattaggtttccaaatcttcactagctcaatactgttgtccttttggtttcaggaaaggaaataaaatgctcagcaaaaaaagggggcataaaagtggacc

SEQ ID NO: 17 - DNA, 526 bp, Homo sapiens
gattcattaatccacatagaatttttctcgatggtataattctgtatttgttaaaagtctttgcataagccccttgtcaagccaaatgctgttttccttttagtatccaattatctgaaacttaagaagagtgtgcaccgcccaatgggtgtgtgtatgtgctgctttgaacctatagttgagatccagagaattgggagtgacatcatctgtaacaataaaagagcctctcttggtaagcagaagacctatatataaaagtcaccatttaaggggtctgcagtccaattcatcagttgtctttagtttactcagcatcagctactaacatacctgaacgaagatcttgttctaagacattgtatggtaagtaaacttaaaaattcacttctgaatctcatgagattttgataatcaagttattatttaatgtgtaccatttctacaaataccatgttgtttcttcaaggtaaaatgctaagaagtttgagttatgtttaatataaaatgccacatacaaaaataa

SEQ ID NO: 18 - protein, 115 aa, Mus musculus
MMSANTVAKVMIIMLAVCLLTQTDGKPVRKRAVSEIQLMHNLGKHLASMERMQWLRRKLQDMHNFVSLGVQMAARDGSHQKPTKKEENVLVDGNPKSLGEGDKADVDVLVKSKSQ

SEQ ID NO: 19 - DNA, 714 bp, Mus musculus
ctgcatatgaaactcagacttgaagaactgcagtccagttcatcagctgtctggtttactccagcttactacagcatcagtttgtgcatccccgaaggatcccctttgagagtcattgtatgtaaagatgatgtctgcaaacaccgtggctaaagtgatgatcatcatgctggcagtctgtcttcttacccaaacggatgggaaacccgtgaggaagagagctgtcagtgaaatacagcttatgcacaacctgggcaaacacctggcctccatggagaggatgcaatggctgagaaggaagctgcaagatatgcacaattttgttagtcttggagtccaaatggctgccagagatggcagtcaccagaagcccaccaagaaggaggaaaatgtccttgttgatggcaatccaaaaagtcttggtgagggagacaaagctgatgtggatgtattagttaaatcaaaatctcagtaaatgctgatttattctagacagtgcagggcactgacatatgctgctaccttttcaagcttatgaagatcaccaagtgctaatacttctactgtaatgaaactttggaatttttttgattacatttttgctcatttaaggtctctttcaatgattccatttcaatatgctcttctttttaaagtactactcatttccacttctctccttaaatataaataaagctttaatgctcatgaatc

SEQ ID NO: 20 - DNA, 5198 bp, Homo sapiens
agatctgctatacaatatagtgtttatacctaataatactgcattgtgaacttcaaaatttaagaaggtaggtctcatgttgtgttcttctaaacgacaaggaaactttgagaagtgctggctatgtctattaccttaattgtggtgatggtatcacagggataacatgtatgtccaaactcatcacattgtacacattaaatatgaacagttctttgtatatcaattgaacctcagtaaatttgttttttttttttaaaaagggagtagaaattatccaatctgttcacaaaacatggaattagagagatgtagctggatttttcagccctggaagcttgttgatgtcaaattcccaaagctgtactaattcctattgagcaaacagaaggaaaaagtatccgtcttaacttaaaccttccataagggaacagggcacaacacatggtattttgtccttcccaaatgtgaggaatacattagattgtttgaccctagcttcagtactttagaatgctaagaaaagctgacgttttcaaagcaaaaagctgtgctcagctcttctctgtcacggtgtcatcagtctcttgtgcccacatatcccctaaccctgtcctcctaagtgatttccttctatctaatacctgcaggtccagaacgtgatggttcagataaactatatatcaagtcttggcagctatattactgctttgtgggaaggtcaagtagtcctttgaaaaatttgaaggacacaacaatattactcatgcacacatttagagtttagagaggactgcaagacaaaactccttggcattcacacaataatcttccaaaattgtacccaaatcttacttctttggagtcatcatccatttccccattttccctctccatccacaagcatatactcaatattcagtcttttttttttttttttttgagacagggtctcactctgtcgcccaggctggagtacagtggcatgatcagagttcactgcagcctccatcttccaggtttgggtgatcctcccatctcagcctcctgagtatctgagactacaggtgtgcaccaccacatcctgctgattttttttttaacttttaggagagatgaggtcttgctatgttgcccaggctggtctcaaactcctaaactcaagcaatccccttgccttggcctcccaaagtgctgagattacaagtgtgagccactgcgtctattcagtcttcattgcctgcatgcctgtgcgcatgcagttccctctgtcctttcttaccttctctgatggaaaacacaatcacctgtcaggacagttcagcttttcagtaaaacagcttccaacaactttagggaaattgtcacttcctcttctgtgcttttataagtctttgttcataaccctattataatacctatcacattatattataattatgtgttaaatttcaaggcattgattatgtctcaattatccagtgattaatacagcccctggcagatagtattcaacgcatttttttgagtgaacaaatgagtgaccactgtctgaactatttgctttatttttctgtaaaactagtttaataatgtggttgccttggtatgacaatttattctataaataagtgtaagagaaagaagaacaagaaagtgataattgcatcactcctagaaggctgtcaattacattaaacttacccaaagcgggaaaaatctccaaatcagaatattaagcatggctttataataacaatgcaaaaaatgtgtttataaaaccatttgtgtatgcagaagattgaaactggacctgttcccttacactatatacaaaaattaactcaagatggattaaagatccgaaactataaaaaccctggaagacaacattggcaatactgttatagacataggaatgagcaaagatttcatgaagaagatgccaaaagcaatggcaatgaaagcaaaaattaacaaatgagatataattaaactaaagagcttctgcacagcaaaagaaactatcaacagagtaaagagacaacctacagaatgggagaaaatttttgcaaactatgcatctgacaaaggcctaatatccaacatctataaattaaacaaatttacaagaaacaaacaaccccattaaaaagtgggtaaaggacatgaacagacatttttcaaaataagacatacatgtggccaacaagcgtatggaaaaaagctcaacatcactgatcatcagagaaatgcaaatcaaaaccataacgagccaccatctcacaccagtcagaatggctgttatcaaaaagccaaaaaataacaggtatgggtgaggttgtggagaaaaaggaacatttatacactgttagtgggagtgtaaattagttcaacaattgtggaaaacagtgtggcgatccctcaaagactaaaagcaaaactacaatttgatccagcaatcccattactgggtatatactcaaaggaatataaatcattctataataaagacatatgcatgcgtatgttcattatagcactattcacaatagcaaagacatgcaatcaacctaaatgcccaacagtggtagactggataaagaatacgtggtgcctacataccatggaatactaggcagccataaaaaagaacaagatggagctagaggccactatccttagcaaactaacataggaacagaaaaccaaatatctcatggtctcacttataattgggagctaaaagatgagaacacatggacacaaagaagggaaccacacacactggggcttactgaaggatggagagtggaaggaggcggaggatcaggaaaaataactaatgggcactaggcttaatacctgggtcatgatataattagtacaataaacctccacgacatgagtttacctatataacaggcctgtacatgtacccctgaacttaaaagttaaaaaataaaaatccattagtggctgggtgtggtggctcatgcctgtaatcccagcactttgggagcgagaggtgggaggactgcttgagccaaggacagcctgggcaatttagtacactctgcctccacaaaaatttaaaaattagctggttgtggtgctgtgtgcctatagcccccactactcagaaggctgaggtggaaggactgcttgagccccgaaagttgaggctgccatgtccataccactgcaccactccagtctgagtgacagagtgagatcctatctcaaagaaaaaagaaaaaccatctgtatttccaattcttcttctcaacagagtatagcagaaaaatgacacacaaacatcacatgtagagtttgcatcagaagaggtctttaaaaagatcaagacaacagatttttacagttaaaagagacctcttggttatctagttcaactttcttctatgtatgaatctctatagtatgcctgataaatgctcatctaccttctacttgaatattctcagtgatggagaaatcactatttgagaaggtacttgattccttattttgacaattataatgtccaagttaaacccaatgaacccagcccttccttcttaaaccacagagaatactccttcttccttatggtaaccctataaataattaaacacagctataaagtcatcccgtctttttttcctcagacttttctagtttcattcaactacttctcatgttatagttttcaacattttcaccaaactgattgatatcctcagtgtatgttcccatttgtttctactgcttctaaaatggagcctggagcaacactctaagtgaggttaacccagagtggagaatctcctaggacaggactagtatatatctctcattataaactccatacggttactattcaaatttgtactggctcttacctaaggtagagtcaaaccttgacaatggctagtaggaaaaaggatgacatgaagccaggatgggaccagagctgagagaactgatcccactctggcaccggatctttggtgggaaggcaaagctgtctaaaggagtcctagatatatcaaagacattcagggtgttttgacaggtggaccaggcaaatattgctggaggaatatttgaagtcagacatgtggcagcatcatgcctccaataggttttggagcaggctctcatcatcccttagggaacatgagaataaagacctaacccctaggggaaaatgttgcaaagagcttttgctgtgttttcagatgactttgcaaattaaagtcaagaagactggagttcaaagaagggctgtatttctaggcagaaactgaggtagtaagaatctggtgtcataaagacctttgttgaattccatcttttctgctttccaagcctaagtttcctcatgtaaaaatggggataagcacttaactttagtactttaagtactaactttaaaggactgctgtgaagatctagtgggataatatatgtagttaggcatgcagttagtgcttatcaaatgttattattatagattaagatgcacaaataaaatataagttaaataaattttaaaataatttcacttttgaagcttttaaagtaattatgtactaagagcattttctcaaattattcttaacacttcctttaagaaaagggctatatctgttttttgaaagatgaccagaagtgacatggattggttgaaaatggcttgtaaagtaagcctaacatttatgatttattaccataaaaactgtaccaacagtacggttataacaaatacacttatttttggattttattttcaagtaagataatgactttatcataaacctttgaaatcagtctttttacagtataaattcagattcattaatccacatagaatttttctcgatggtataattctgtatttgttaaaagtctttgcataagccccttgtcaagccaaatgctgttttccttttagtatccaattatctgaaacttaagaagagtgtgcaccgcccaatgggtgtgtgtatgtgctgctttgaacctatagttgagatccagagaattgggagtgacatcatctgtaacaataaaagagcctctcttggtaagcagaagacctatatataaaagtcaccatttaaggggtctgcagtccaattcatcagttgtctttagtttactcagcatcagctactaacatacctgaacgaaga

SEQ ID NO: 21 - protein, 84 aa, Homo sapiens
MACLAGRCLGLTFFEKCWLVPAPGKPGVGGGSVLTRGAPGIQDASRGLWLPVLMKDSASASTLGTGSWVAKPSQRKDWAQLRLS

SEQ ID NO: 22 - DNA, 1675 bp, Homo sapiens
atctgcacgctttctgcgaggcctcagaacttcccagggcccctccctcaaattgtctccatgggaaacttgacccagtggcaagttgcactttggtgatcttggtggtctacacacccgttctgtggagagtcgatttacataagctgtgtatacacacacacacacacacacacacacccctaccccacactgactgtctaccgacaaagaccctatttcctggcaaacggcctcctgaaccctgactttttgtgtacatacttgtaaacacggatttttctgggttttggtttgctttttccttttttccccctgcccctgttctagcttgttcttcttggtttgctttcaacctgcttgatggatgtctgcagagtgctctctaagagtccacctcagtgcctcgtgtgctcagtggtcatgggaaaggagcgaaggaaccatccttggttctcccagcttggttgtgtagcaatccctcagcattgtttttctcagcttcttggcaaaaattaaaacaacaacaacaacaacaacaacaacaacaaacagaaggataaactggcttgcctgtggaccctccctggctctggggccagtcgagagccactgagggacccagcactcagagacacaacacacatgtgtagctgcttctggctgagtgtgtttcctgtcaccaatggcctgtttggctggacgatgcctcggcttgaccttttttgaaaagtgctggttagttcccgcccctggtaaacctggggtaggtgggggttctgtcttaactcgaggggcacctgggatccaggacgcttctagggggctctggctgcccgtgttaatgaaggacagcgcttccgcgagcaccctgggaactgggtcttgggtagcaaagccctcccagagaaaagattgggcacaactaaggctttcctgagcaggaagggggtgaagaccaatcccttcctttggtcctttggtacgcaccccctcagagctgagatggaagacatggctagttcttttcagccttgtggagcctgtcagtcgccatcatacctcgagtgaggcccagctagataatgacttgtccaagatggcacacgtggaaagttgatctgcaccagaacccggatgactgtcaccttgaagcatcctgttctccttctgtgctgtcccaggaagtgtctggcgggcgtgggcagcacagctctacactgtacgattcactagggcatcctgcgagcctcactagccttctggttcatgcctttgacaagcatttttgtgccccctctgcttactgtgacagtcgatgatgaatcttgcgttgccattttctgctgtgggtaactgcgtgcagtgtcttgccttgctttctcttcttactgtcccacagcttggtttcatgttacaaacagaaaagctcgaggctcccaccccgccacatcccaacttcatttccccctcactgtagcccatttccaccccaccacaaagttgccacaggttttctttgtatagaatatttattttgaagctctattttaatagtatttattttagaaagtctactattgtaagagttcttctgtttgtgaagaaaaaaacaagttaaaaactgaatgtactgatttagaaaatatatataaatatatattgttaaatatac

SEQ ID NO: 23 - protein, 115 aa, Felis catus
MMSAKDMVKVMVVMFAICFLAKSDGKPVKKRSVSEIQFMHNLGKHLSSVERVEWLRRKLQDVHNFVALGAPIAHRDGGSQRPRKKEDNVPAENHQKSLGEADKADVDVLIKAKSQ

SEQ ID NO: 24 - DNA, 737 bp, Felis catus
gcacgaggaaagtatcagctgtcaagacacctgaaagatcttgtcacaaccttgtgtgtgaagatgatgtctgcgaaagacatggttaaggtcatggttgtcatgtttgcaatttgctttcttgcaaaatcggatgggaaacctgttaagaagaggtctgtgagtgaaatacagtttatgcataacctgggcaagcatctgagctccgtggagagggtagaatggctgcggaggaaactacaggatgtacacaactttgtcgccctcggagctccaatagctcacagagatggtggttcccagaggccccgaaaaaaggaagacaatgtcccggctgagaaccatcaaaaaagtcttggagaagcagacaaagctgatgtggatgtgttaatcaaagctaaatcccagtgaagacagagcagagcactgctatacacaggatagggcaacaaaattacatgctgctaacattctcaagctttgaagatcaccaaatgccaatatttacgtctaatccatggctagccacgatagctgaaattctaattgattgttttgattctacttttattcatgtaaggcctcttttaattattccatttctgttgtttattctttttaaagtatgttattgcataatttataaaagaataaaattgcactttgtaacctctctcccatcgtacactgcaaaataaaaatttaatgatcataattttaaaaaaaaaaaaaaaaaa

SEQ ID NO: 25 - protein, 115 aa, Rattus norvegicus
MMSASTMAKVMILMLAVCLLTQADGKPVKKRAVSEIQLMHNLGKHLASVERMQWLRKKLQDVHNFVSLGVQMAAREGSYQRPTKKEENVLVDGNSKSLGEGDKADVDVLVKAKSQ

SEQ ID NO: 26 - DNA, 704 bp, Rattus norvegicus
ctgcatatgaaactcaggcttgaagaactgcagtccagttcatcagctgtctggcttactccagcttaatacagggtcactcctgaaggatcctctctgagagtcattgtatgtgaagatgatgtctgcaagcaccatggctaaggtgatgatcctcatgctggcagtttgtctccttacccaggcagatgggaaacccgttaagaagagagctgtcagtgaaatacagcttatgcacaacctgggcaaacacctggcctctgtggagaggatgcaatggctgagaaaaaagctgcaagatgtacacaattttgttagtcttggagtccaaatggctgccagagaaggcagttaccagaggcccaccaagaaggaggaaaatgtccttgttgatggcaattcaaaaagtcttggcgagggggacaaagctgatgtggatgtattagttaaggctaaatctcagtaaatgctgacgtattctagaccgtgctgagcaataacatatgctgctatcctttcaagctccacgaagatcaccaagtgctaattcttctactgtaataaaagtttgaaatttgattccacttttgctcatttaaggtctcttccaatgattccatttcaatatattcttctttttaaagtattacacatttccacttctctccttaaatataaataaagtttaatgatcatgaaccaaa

SEQ ID NO: 27 - protein, 115 aa, Macaca fascicularis
MIPAKDMAKVMIVMLAICFLTKSDGKSVKKRSVSEIQLMHNLGKHLNSMERVEWLRKKLQDVHNFIALGAPLAPRDAGSQRPRKKEDNILVESHEKSLGEADKADVDVLTKAKSQ

SEQ ID NO: 28 - DNA, 606 bp, Macaca fascicularis
tcgtgaaaaccaacccaattagttagtattgcattctgtgtactatagttttgaatattaaaagtattttaaaatacctccattttgcctttccttttagtgaagatgatacctgcaaaagacatggctaaagtaatgattgtcatgttggcaatttgctttcttacaaaatcagatgggaaatctgttaagtaagtactgttttgccttggaattggatttttaatgttgactttatcattttgaagtggggagctaatgggaagtggccctctctgtttctcttcttcccaggaagagatctgtgagtgaaatacagcttatgcataacctgggaaaacatctgaactcgatggagagagtagaatggctgcgtaagaagctgcaggatgtgcacaattttattgcccttggagctcctctagctcccagagatgctggttcccagaggccccgaaaaaaggaagacaatatcttggtagagagccatgaaaaaagtcttggagaggcagacaaagctgatgtggatgtattaactaaagctaaatcccaatgaaaatgaaaatagatatggtcagagttctgctctagacagtgtagggcaac

SEQ ID NO: 29 - DNA, 552 bp, Mus musculus
aaaatataaactcagtattatcgattatgtcaaaaatttctaggtggacacttagtatttgctacatcttcttttgcactttctttgtcagaaccctgctcaattttcatctactatctagttatctgaaactttagaggagtgggcaccaccccatgagggtatgtggctgttctgatcctgtgattgagagccagagaaccaggagtgacatcatccttaacaataaaatactcctcttggtgagcaaaaagcctgcatatgaaactcagacttgaagaactgcagtccagttcatcagctgtctggtttactccagcttactacagcatcagtttgtgcatccccgaaggatcccctttgagagtcattgtatggtaaggaacctctcaactgccctttttaaattctgtgaggtttagaaaattgagctagtttttttaatacataccatctcctaccaatatcatgccatttttaaaaaaattgaaatgttaaaggaaggtgaattttgctaaacagaaaaatgtagaggtaaaatacatttatggt

SEQ ID NO: 30 - protein, 115 aa, Mus musculus; identical to SEQ ID NO: 18

SEQ ID NO: 31 - DNA, 1045 bp, Mus musculus
gctaatcattgcttgctttggtggctatggataacaactactgaggctgtatttacaatactttagaacactgacagtatcttaaaatatctcaaggttgtttcttcttttagtaaagatgatgtctgcaaacaccgtggctaaagtgatgatcatcatgctggcagtctgtcttcttacccaaacggatgggaaacccgtgaggtaagtgctgcagcccattgtgcacagggaagtgtggactcgaggctttgtagtgggttttaacgttgtgggcatggggagctaatggaacggtccgctctttctcttctttctaggaagagagctgtcagtgaaatacagcttatgcacaacctgggcaaacacctggcctccatggagaggatgcaatggctgagaaggaagctgcaagatatgcacaattttgttagtcttggagtccaaatggctgccagagatggcagtcaccagaagcccaccaagaaggaggaaaatgtccttgttgatggcaatccaaaaagtcttggtgagggagacaaagctgatgtggatgtattagttaaatcaaaatctcagtaaatgctgatttattctagacagtgcagggcactgacatatgctgctaccttttcaagcttatgaagatcaccaagtgctaatacttctactgtaatgaaactttggaatttttttgattacatttttgctcatttaaggtctctttcaatgattccatttcaatatgctcttctttttaaagtactactcatttccacttctctccttaaatataaataaagctttaatgctcatgaatcaagtaagcagtgtttcttgttaaaactttgtctcagttggagggttggctcagaagttaaaattgcaaactgtagggctggagagatggttcagcggttaagagcactgaccgctcttccagaggtcctgagtttaattcccagcaaccacatggtggctcacaatcatctgtaatgggatctgatgccctcttctggtgtgtctgaagacagcaacagtgtac

SEQ ID NO: 32 - DNA, 100 bp, Homo sapiens
aaaaatctccaaatcagaatattaagcatggctttataataacaatgcaaaaaatgtgtttataaaaccatttgtgtatgcagaagattgaaactgggac

SEQ ID NO: 33 - protein, 115 aa, Sus scrofa
MMSAKDTVKVMVVMLAICFLARSDGKPIKKRSVSEIQLMHNLGKHLSSLERVEWLRKKLQDVHNFVALGASIVHRDGGSQRPRKKEDNVLVESHQKSLGEADKAAVDVLIKAKPQ

SEQ ID NO: 34 - DNA, 698 bp, Sus scrofa
aattcatcagccttctcgggtttactcaactttgagaaagcatcagctgctaacacacctgaaagaagatcgtgtcctaagacgttgtgtgtgaagatgatgtctgcaaaagacacagttaaagtaatggttgtcatgcttgcaatttgttttcttgcaagatcagatgggaagcctattaagaagagatctgtgagtgaaatacagcttatgcataacctgggcaaacacctgagctctctggagagagtggaatggctgcgaaagaagctgcaggatgtgcacaactttgttgccctcggagcttctatagttcacagagatggtggttcccagagaccccgaaaaaaggaagacaatgtcctggttgagagccatcaaaaaagtctcggagaagcagataaagctgctgtggatgtattaattaaagctaaaccccagtgaaaacacatatgatcagagagcactgctctagacagcataaggcaaacaatatttcatgctgctaatgttttcaatctctattaagattaagtgccaatatttctaatattactaaacttgatgggtaatcattgctagccatgattgctgaaattttaattgatcattttgattctacttttactcatttaagagcttcttttaacaattctatttctattgattctaaataaatgaagtatttcttccttgtt

SEQ ID NO: 35 - protein, 102 aa, Bos taurus
MIHYNPHNNCANVMAVRNILIFNFTFKYNKMMWWWFSAKSCPTLVTPWTVAASSSVHGFFRARILEWVAISFSGESSQSRNQTQVSCIAGEILYQLSYERSL

SEQ ID NO: 36 - DNA, 641 bp, Bos taurus
gttactgcttttcagtgtttttattgaaaactaaaattaaaaatataaggtaaatgaattttaaaatagttttacttttgaagccctttaaaattatgtacagagagccatattctcaaaatatttttaacacttcctttaagaaaagcactatatatatttttttggaacaagatcaggaatgatacagactggtccaatatggcttgtaaagtaagcataatgtaatatgattcattataatcctcataataattgtgccaatgttatggctgtaagaaatatacttattttcaatttcactttcaaatacaataagatgatgtggtggtggtttagtgctaagtcgtgtccaactcttgtgaccccatggactgtagctgccagctcctctgttcatggattcttcagggcaagaatactggagtgggttgccatttccttctctggggagtcttcccaatccaggaatcaaacccaggtctcctgcattgcaggcgagattctttaccaactgagctatgagagaagcctgtgataagatgatgacttagccataaatttttgaaatctgacctttttaaaaaaattttgttgctgttgctcagttgctaattcatgtccaaccgctttacactgca

SEQ ID NO: 37 - DNA, 296 bp, Bos taurus
atactcccttttcttcttattacctcaaccccattcacatatcaggcttagatttcttagagaagtatcatgctggaatttcttttttccagaaggataaaggataaataattctagaacttgacagttgtcattaaagtaacaaccaggtgaattaaataaccccttgatagctcagttggtatagaatcaccttcaatgcaggagaccctggttcaattcctgggttgggacgatccactggataagggataggctacccactccagtattcttggactttccttgtgtctcag

SEQ ID NO: 38 - protein, 115 aa, Homo sapiens; identical to SEQ ID NO: 8

SEQ ID NO: 39 - DNA, 772 bp, Homo sapiens; identical to SEQ ID NO: 9

SEQ ID NO: 40 - protein, 115 aa, Rattus norvegicus; identical to SEQ ID NO: 25

SEQ ID NO: 41 - DNA, 704 bp, Rattus norvegicus; identical to SEQ ID NO: 26

SEQ ID NO: 42 - protein, 115 aa, Rattus norvegicus; identical to SEQ ID NO: 25

SEQ ID NO: 43 - DNA, 973 bp, Rattus norvegicus
gttctatactaaagtatctgtcctataaagatcccacagacccatgaaaagtggcatcagctaatgactgtttccttgggtggttatgaataacagctactgggactgtgtttacacactttagagcactgacagtgtcttaaaatatctctgtctctccttgtagtgaagatgatgtctgcaagcaccatggctaaggtgatgatcctcatgctggcagtttgtctccttacccaggcagatgggaaacccgttaagtaagtgctgcagcccgtcgtcccagggaagtcggacatgaggctctgtaggttttaatgttgtgggcatggggagctaatggagtggtcctctctttctgttctctctaggaagagagctgtcagtgaaatacagcttatgcacaacctgggcaaacacctggcctctgtggagaggatgcaatggctgagaaaaaagctgcaagatgtacacaattttgttagtcttggagtccaaatggctgccagagaaggcagttaccagaggcccaccaagaaggaggaaaatgtccttgttgatggcaattcaaaaagtcttggcgagggggacaaagctgatgtggatgtattagttaaggctaaatctcagtaaatgctgacgtattctagaccgtgctgagcaataacatatgctgctatcctttcaagctccacgaagatcaccagtgctaattcttctactgtaataaaagtttgaaatttgattccacttttgctctttaaggtctcttccaatgattccatttcaatatattcttctttttaaagtattacacatttccacttctctccttaaatataaataaagtttaatgatcatgaaccaaataagcagtgtttcttacttgttaaaacttttgtctcagtgttggagggctggctcagaagttaagagtgcatactgcttcctcaaatgacccgagtttgcttctcgg

SEQ ID NO: 44 - DNA, 709 bp, Rattus norvegicus
gaaagtgcaggcccagtggcaatgtctttaactttgcactcatgaatagaggcaagtggatcttgagttttgggtcagcctgatctacatattgagttctaggccagccagagttcatagtgagacgctatctcaaactgttttaaatataaaatcagtatcatggattacgtcagatttttctaagtggttacttaagcatttgctgcaacttcttttgcagattctttgccagcaccttgctctttttgaatccattatctaagtatctgaaactttagaggagtgggcaccgcccgatgagggtaggtggctgttctgattcctatgattgagaaccagagaaccaggcatgacatcatccttcccaataaaatactcctcttggtgagcaaaaggcctgcatatgaaactcaggcttgaagaactgcagtccagttcatcagctgtctggcttactccagcttaatacagggtcactcctgaaggatcctctctgagagtcattgtatggtaaggaatctctcaattgcccttttaaattccgtgagatttagaaaattgtgctagtttttaatacataccattttctatcaatatgtgccattttttaaaattggaaggtaggggtgaatttgccaaaatggaaggatatggcaataaaataatttagggtaggaaattcggactcatctatgtatatggttaa

SEQ ID NO: 45 - DNA, 523 bp, Gallus gallus
  misc_feature (426)..(430): n is a, c, g, or t
gctaacagaggaactgcgacgaacaacggtttaagccccggaggatatgatgttcacgaaactctttcagcagtggagtttcgcggtgtttcttctgagttattctgtgccctcctacggaagatcagtagaggggatcagccgcagactcaaacgggccgtatcagagcaccagctactgcatgacaagggcaaatcaatccaagacttacgaagaaggatattccttcaaaatttaattgaaggtgtcaacactgcagaaatccgtgcaacttcagaggtctcacctaaccctaagcctgctaccaacacaaagaactaccctgtccgttttggcagtgaagatgagggcagatacctaactcaggagacaaacaaatcacagacctacaaggaacagcccctgaaggtatcagggaagaaaannnnngcaaagcnnnnnnaacgtnnngaacaagagaagaaaaagaggcgagctcgctcagcttggcnnnattctggcatgcatatcatacagttactg

SEQ ID NO: 46 - protein, 175 aa, Mus musculus
MLRRLVQQWSVLVFLLSYSVPSRGRSVEGLGRRLKRAVSEHQLLHDKGKSIQDLRRRFFLHHLIAEIHTAEIRATSEVSPNSKPAPNTKNHPVRFGSDDEGRYLTQETNKVETYKEQPLKTPGKKKKGKPGKRREQEKKKRRTRSAWPSTAASGLLEDPLPHTSRTSLEPSLRTH

SEQ ID NO: 47 - DNA, 1592 bp, Mus musculus
cgccggcccgctcacccctgcctgctccgccggcgcgcgttcctcgggcgccacctctttgcgactcgctcacttctcagcaggttggccccggagcgtgtgaaccttccagggctgggctcacagctactctccaacctgcgccgccccagctgggccgctccgccccgctgccggaacccgccctcgcgccacctggcctcgcatccacgacacgcgcgcctgcaacttgttcaagggcgttgtggaatcaactttccggaagcaaccagcccaccgaaggagggtgcacccagaggcccggtgcgcaggacagctgactcctgaggaacacccgcgtttgaagaggggtttgacctgccccacgacccagagtgctgccgccaagactaattagacattgctatgggagccacagcaacgcgccacgcatccccgacgcctatgtaaaacgcccggttttcgctcttctttcagaggaagctctctgattgctttttcccctctcgggtccctttttgcctgtgcggtttgagagaggcgcagttagaggcgctgattcctacacaagtccccagagccagcgagcggcacgatgctgcggaggctggttcagcagtggagtgtcctggtattcctgctcagctactccgtgccctcccgcgggcgttcggtggaggggcttggccgcaggctcaaacgcgctgtgtctgaacatcagctactgcatgacaagggcaagtccatccaagacttgcgccgccgtttcttcctccaccatctgatcgcggagatccacacagccgaaatcagagctacctcggaggtgtcccccaactccaaacctgctcccaacaccaaaaaccaccccgtgcggtttgggtcagacgatgagggcagatacctaactcaggaaaccaacaaggtggagacgtacaaagaacagccactcaagacacccgggaagaagaagaaaggcaagcctgggaaacgcagagaacaggagaaaaagaagcgaaggactcggtctgcctggccaagcacagctgcgagtggcctgcttgaggaccccctgccccacacctccaggacctcgctggagcccagcttaaggacgcattgaaattttcatcgaagatcttccaaggacacgttacaggattttgtaatagtaaacatatggaaagtattagacatatttattgcctgtacatactgtaaatgcattgggatcaaactgtctccccaggaaactgcacatgggtcatgtgaatatttttcccttttgccaaggctaatccaattattcctgtcactgttaccataatttattttgtcaactgatgtatttatttgtaaatgtatcttggtgctgctgactctgtttttttgtaacataatgcactttaggtatacatatcgagtatgtggatgaatttaacacataaaaggatctctattttgtggttcattttaatgagttctgaaatataattatctagactgatttccttctgtgcatgtaaaaatggcagtattttaaatttgttaaataatgtctaataaaatataatctaattataccatgactc

SEQ ID NO: 48 - DNA, 242 bp, Felis catus
aatctttagcaaagacattcagaggacgtattacaggattctgtaatagtgaacatatggaaagtattagaaatatttattgtctgtaaatactgtaaatgcattggaataaaactgtcttcccattgctctatgaaactgcacattggtcattgtgaatatttttttgccaaggctaatccaattattattatcacatttaccataatttattttgtcaactgatgtatttattttgtaaa

SEQ ID NO: 49 - DNA, 351 bp, Felis catus
atggcttctgtgccccattgcctttgctcggtgctctccgctggggtgtgtaacgtcccttccactgtggcttggacaaacctagaatgtcctccctttctgtccttctatcaattgctccacaatttcaaagcttaatgaaagcaagatggctcagaatattgtctgctttcatacagtgtcctcctatgccctctatacacaccagcattgaataacccgtttctcttcctccaccatcacaccccaacgccaatcctttactcctccactaaagaatttctttttttcgtcaggcttcagaagctagggaccaccttcatcatttgctggaggagtgtatttattccc

SEQ ID NO: 50 - protein, 94 aa, Felis catus
LLSYSVPSCGRSVEELGRRLKRAVSEHQLLHDKGKSIQDLRRRFFLHHLIAEIHTAEIRATSEVSPNSKPAPNTKNHPVRFGSDDEGRYLTQET

SEQ ID NO: 51 - DNA, 282 bp, Felis catus
ctgctgagctactcggtgccctcctgcgggcgctcggtggaggaactcggccgccggctcaaaagagctgtgtctgaacatcagctccttcatgacaaggggaaatctatccaagacttacgacgacgattcttccttcaccacctgattgcagaaatccacacagctgaaatcagagctacctcggaggtttcccccaactccaagcctgctcccaacacaaagaaccacccagtccgatttgggtctgacgatgaaggcagatacctaactcaggaaacc

SEQ ID NO: 52 - protein, 202 aa, Oryctolagus cuniculus [sequence omitted]

SEQ ID NO: 53 - DNA, 650 bp, Oryctolagus cuniculus
cttaagcttatgcagcggagactggttcagcagtggagcgtcgcggtgttcctgctgagctacgcggtgccctcctgcgggcgctcggtggagggtctcagccgccgcctcaaaagagctgtgtctgaacatcagctcctccatgacaaggggaagtccatccaagatttacggcgacgattcttccttcaccatctgatcgcagaaatccacacagctgaaatcagagctacctcggaggtgtcccctaactccaagccctctcccaacacaaagaaccaccccgtccgatttgggtctgatgatgagggcagatacctaactcaggaaactaacaaggtggagacgtacaaagagcagccgctcaagacacctgggaagaaaaagaaaggcaagcccgggaaacgcaaggagcaggaaaagaaaaaacggcgaactcgctctgcctggttagactctggagtgactgggagtgggctagaaggggaccacctgtctgacaccttcacaacgtcgcttggagctcgattcacgtacagcacttctgtggggtttgaaaaaaaaaaaggaaaacaacagaagaacacatcatatgcaactaatgatctcattatttaagagttccctgttacttctttagtctagaccca

SEQ ID NO: 54 - protein, 175 aa, Mus musculus
MLRRLVQQWSVLVFLLSYSVPSRGRSVEGLGRRLKRAVSEHQLLHDKGKSIQDLRRRFFLHHLIAEIHTAEIRATSEVSPNSKPAPNTKNHPVRFGSDDEGRYLTQETNKVETYKEQPLKTPGKKKKGKPGKRREQEKKKRRTRSAWPSTAASGLLEDPLPHTSRPSLEPSLRTH

SEQ ID NO: 55 - DNA, 572 bp, Mus musculus
ccagagccagcgagcggcacgatgctgcggaggctggttcagcagtggagtgtcctggtattcctgctcagctactccgtgccctcccgcgggcgttcggtggaggggcttggccgcaggctcaaacgcgctgtgtctgaacatcagctactgcatgacaagggcaagtccatccaagacttgcgccgccgtttcttcctccaccatctgatcgcggagatccacacagccgaaatcagagctacctcggaggtgtcccccaactccaaacctgctcccaacaccaaaaaccaccccgtgcggtttgggtcagacgatgagggcagatacctaactcaggaaaccaacaaggtggagacgtacaaagaacagccactcaagacacccgggaagaagaagaaaggcaagcctgggaaacgcagagaacaggagaaaaagaagcgaaggactcggtctgcctggccaagcacagctgcgagtggcctgcttgaggaccccctgccccacacctccaggccctcgctggagcccagcttaaggacgcattgaaattttcatcgaagatcttccaa

SEQ ID NO: 56 - protein, 162 aa, Sparus aurata
MCSIVILHHWSLAVFLLCSPVTLDGKPVDALGSRTRRSVSHAQLMHDKGRSLQEFKRRMWLHELLEEVHTADDRPVQSRTQSQTFSGNALHEKPPGATKNIPDRFRLDREGPNLPQETNKALAYKDQPLKVATKRKKKVRLGRRRESDKKRRRARSVTTKEQ

SEQ ID NO: 57 - DNA, 1787 bp, Sparus aurata
gcttctagtgctggactgaacaaccctctcagctctgctctccagctctcgccaccagagaagatgcttcatcgttactcttcatgagattactgcactgagcggagtggcagatctgcaagcggatgggctgctgctgctctttccatctttatctctcttttaattgggacgctggggagaaataaatagctcagcacacactgttgggaggcaaatgaatggaacagactgacgggggatttccccaggctgccttcacaccaatggacctggaaacaagttttgagctggccaaagtttatttggcagaataatgtttcttgtcgttttgtaaatttgtttttgcaacaagatcatcttttaagagcttgcagactgatattttctaggactttcacagtaaacatacataccatatcttaggctcaaggatgtgctctatagtaattcttcatcattggagtctggcggtgttcttgctgtgctccccagtgactcttgatgggaaaccagttgatgcacttggtagcagaacgaggaggtcagtgagccacgcccagctgatgcatgacaagggtcgctccttacaagagttcaagcgtcgtatgtggctgcacgagctgctcgaggaggtgcacacagccgatgatcgacctgtgcagagcagaacccagagccaaacattcagtggcaatgccctgcacgagaagcccccaggggccaccaagaacatccctgacaggttcaggctggacagagaaggtcctaacctgccccaggagaccaacaaggctctggcttataaggaccaaccacttaaagtggccacaaagaggaaaaaaaaggtgaggttaggccgacgtagagagagcgacaagaagaggaggcgggcacggtctgttacaacaaaggaacaatgaatgatgcagtaccacagcaaagagactctgaacagactttaaatggtactgcgctgagacactgacaaaggttcaaccagactgggatctgacagactcgatccatcactgactcagttgcttcacatcacgggcccaggattgtgcttgtataatatttcgctgtttgtattttcaatctcatagttcctgaagttttgtgaattatcatcataacatcttcttcagttcatatttatttggaaaatctgaagacctctgtaatcctgatttacatcttaacaattccatgtgttgtcatctctaaccaaccgtgtgatcaagtagatcataggaggaactatttatttcctgaataatgtcattatgtcaacttcatgaacttgggaggcttagtggtctacttttgttggctccatttgtgttaactatcttggacttacagttacatacaaactgttcaatgaatacatctgttgcatctttttcataatttatttcactttgaaagtgtgtatttattttgtgaatgtatcttggtgctgctgactaaattccgtaatgcactttagttatatcctgtacatgttcttttgtggttgagttttgatttgatgtttctttttgatggtcctgctttcctttcccaccctagattcctaatggacttgtaacttattggatgcttcatcaaacccactctcaccgttttacattatagtttatttttatagaacaagaccgagacaggggctctcagactcatataatgttcttcacttgcactgagcgcatactgttcatgtttccagttaatcaaccattaaaagtgtagtcattagaaacaaaaaaaaaaaaaaaaaa

SEQ ID NO: 58 - protein, 177 aa, Oryctolagus cuniculus
MLRRLVQQWSVAVFLLSYSVPSCGRSVEGPGRRLKRAVSEHQLLHDKGKSIQDLRRRFFLHHLIAEIHTAEIRATSEVSPNSKPAANTKNHAVRFGSDDEGRYLTQETNKVEPYKEQPLKTPGKKKKGKPGKRKEQEKKKRRTRSAWPLSAGAGSGLAGDHLSDISEPEPELDSRRH

SEQ ID NO: 59 - DNA, 534 bp, Oryctolagus cuniculus
atgctgcggaggctggttcagcagtggagcgtggccgtgttcctgctgagctactccgtgccctcctgcgggcgctcggtggaggggccaggccgccgcctcaagagagccgtgtccgaacaccaactcctccatgacaagggcaagtccatccaagacctccggcgccgcttcttcctgcaccacctgatagccgaaatccacaccgccgagatcagagctacctccgaggtctcccccaactccaagcccgccgccaacaccaagaaccacgcggtgcgcttcgggtcggacgatgagggcaggtacctcactcaggaaaccaacaaggtggagccctacaaggagcagcccctcaagacgcctggcaagaaaaagaaaggcaagcccgggaagcgcaaggagcaggagaagaagaagcggcgaactcgctccgcctggccgctgtcagcgggcgcgggcagtgggctcgcgggcgaccacctgtctgacatctccgagccggagcccgagctcgattcacggaggcattga

SEQ ID NO: 60 - protein, 163 aa, Takifugu rubripes
MCSVVMLHQWSLAVFLLCSPVTLDGKPVDAVSSRMRRSVSHAQLMHDKGRSLQEFRRRMWLHKLLEEVHTANEEAPPVQSRTQTQTFSGNSLHEKPPGATKNLPDRFSLDREGTNLPQETNKALAYKDQPLKLATKRKKKARLGRHREADKKRRRARSVAKEP

SEQ ID NO: 61 - DNA, 2272 bp, Takifugu rubripes
ccctcactgtgctccagtgaaaacaccctctcagctctgatctcccgcccggggccaccagagaagatgcttcgccgttgctctctgtgaaatcacagcgagcagggctgctgccaatgggctgctgctgctccttcctcggtcgtctctttttaacctggactctcgggagaaataaatagcgcctcgcttgctgttgggaggcaaattaacggaacagagctacaggggatttccccaggctgcctgcacgccaatggacctggacccgggttttgaaggggacaatttgtttttggcaaaataatattttttcccccccgtttcgcctcgacacgtatcaaatgtgcaacgagatcatctcgatttaagggcttgggaagcagctctgaggaattttcccgtacaaactcgcaccacgtaagtctgatttacttttgtactttaataaactgctccctggtgtcattaatgcagcctgggaggtcccacatatgcagggaacgtctcatttccccagctgcaaacacattaataagccctaactattcattcttaagctccgttcgacagactgaagtgacttctgtctccttttctccagaccgtgggctgaaggatgtgctccgtagtcatgcttcatcagtggagcctggctgtcttcttgctctgctcaccagtgactcttgatgggaaaccagtggacgcagtcagcagcagaatgtgagtttgctaattacgttaattcccagtaatgcaacgtggaacttcaatgtgagcatttgtaacattttcccgccgttttggactcggagagctcggtttacctgtgatgactcagttcaggtacaagcagccttcacagcttcggcgctggcattttcgtgttcagaaacatgtttatgctgccacctactgcactgaagtctcctggtgtgtggccctcaaattaatctgacatcatttataaaactgttccacttataaagtcaacactgtagagtactgtgaaggagaaacccgtgggtgccaataaagtctggcttcagacgggttccagaggaacacgacaggatgcgactgatttatgacaggttctgggaaatgcctgcgtctgctgtcacgtttttctggattatgtctcacagatctctctgtctttcgaccttgtgctttaaggcggaggtcggtgagccacgcccagctgatgcacgacaagggccgctccctgcaggagttcaggcgtcgcatgtggctccacaagctgctggaagaggtccacacggccaacgaagaagccccacctgtgcagagcaggacccagacccaaaccttcagcgggaactccctgcacgagaagcccccgggggccaccaagaacctccctgacaggttcagtttggaccgagagggcacgaacctgccccaggagacgaacaaggctttggcttacaaagaccagccgcttaaactggccaccaagaggaaaaagaaggcgaggttaggtcggcacagagaggctgacaagaagcggaggcgggcgcgctcggtagcaaaggagccttgaaggatgcgctgttgccctggcaaagagactctcagtaaccttcctatggtactgcactaagacactgacacaggcccagccagactgggatctgagactctgcccgacttcatattacgatgccaaaatccttttgtatgataaaatgttgctatgcgtctattttttatacttcctggatttccaggaacacgccatcttcttcttaggttcatatttatttggcttctttaaccccgattcacacccaaacaaacaccatctctacacagccgccaagaggaactatttattctctgacatcaaaattgggacgtcgactggactcgcatacggttcactgaacacatccgtcggcgctgttccgccaataatttatttcactttgaacgtgtgtatttattttgtgaatgtatcacagtgctgctgacaaaattccattgtgcactttaggtatatccagaacatgttcttatgtggtggagttttagatttcttttcgatgttctctcttctcaccaaaagcaaatgtaacttatgggtcgcttccacagacccactctcgccgtttgacatgatagtttatttttgcaggacacgccaaagcagcggctctcagcctcatttaaagttcttcatttgcactgagggcatactgtacatgtttccagttaatccaacattaaaaatgtagccaatagaaac

SEQ ID NO: 62 - DNA, 249 bp, Artificial Sequence
  Synthetic
aattcatcgatatgcggtacctgacccaggaaaccaacaaagttgaaacctacaaagaacagccgctgaaaaccccgggtaaaaaaaagaaaggtaaaccgggtaaacgtaaagaacaggaaaaaaagaaacgtcgtacgcgttctgcttggctggactctggtgttaccggatccggtctggaaggtgaccacctgtctgacacctctaccacaagcctggaactggactctcgtcgtcactaataag

SEQ ID NO: 63 - DNA, 207 bp, Artificial Sequence
  Synthetic
aattcatcgatatgctgctgcacgacaaaggtaaatctatccaagatctgcgtcgccgtttcttcctgcaccacctgatcgctgaaatccacactgcagaaatccgtgctacctctgaagtttctccgaactctaaaccgtctccgaacaccaaaaaccacccggttcgtttcggttctgacgacgaaggtcggtacctgtaataag

SEQ ID NO: 64 - protein, 177 aa, Canis familiaris
MLRRLVQQWGVAVFLLSYSVPSCGRSVEELGRRLKRAVSEHQLLHDKGKSIQDLRRRFFLHHLIAEIHTAEIRATSEVSPNSKPAPNTKNHPVRFGSDDEGRYLTQETNKVETYKEQPLKTPGKKKKGKPGKRKEQEKKKRRTRSAWLNSGVAESGLEGDHPYDISATSLELNLRRH

SEQ ID NO: 65 - DNA, 1166 bp, Canis familiaris
cggcacgaggaagtttttcattcatttaagacttcattttactcttttgaagcaaaaaaaaaaaaattaatttctggaagagactgctgagaagatccccttcgtttggccagaagagaagagaggactgtaaatcaaggaaaaggtcccgcgagcgacaggggacgatgctgcggaggctggttcagcagtggggcgtcgcggtgttcctgctgagctactcggtgccctcctgcgggcgctcggtggaggagctcggccgccggctcaaaagagctgtgtctgaacatcagctccttcatgacaaggggaaatctatccaagacttacggcgacgattcttccttcatcacctgattgcagaaatccacacagcagaaatcagagctacctcggaggtttcccccaactccaagcctgctcccaacacaaagaaccaccccgtccgatttgggtctgatgatgagggcagatacctaactcaggaaaccaacaaggtggagacatacaaggagcagccactgaagacacctggcaagaaaaagaaaggcaaacccggaaaacgcaaggagcaggaaaaaaagaaacgtcgaactcggtctgcctggctaaactctggcgtggctgaaagtgggctagaaggcgaccacccatatgacatctcagcgacatcgctggagctcaatttacggaggcattgaaatttttagcaaagaccttcaaaggacgtattacaggattctgtaatagtgaacatatggaaagtattagaaatatttattgtctgtaaatactgtaaatgcattggaataaaactgtcttccccattgctctatgaaactgcacattggtcattgtgaatatttttttttgccaaggctaatccaattattattatcacatttaccataatttattttgtcaactgatgtatttattttgtaaatgtatcttggtgctgctgaatttctctattttttgtaacataatgcactttaggtatacatatcaagtatgttgataaatgacacaataaagtgtctttattttgtggttgattttaataatgcctaaatataattatccaaactgattttcctctgtgcatgtaaaaatagcagtattttaaatttgtaaagaatgtctaataaaatataatctaaatt

SEQ ID NO: 66 - protein, 175 aa, Mus musculus; identical to SEQ ID NO: 46

SEQ ID NO: 67 - DNA, 971 bp, Mus musculus
ggatcctgcctgcctctttcacatctttattgagtgttgtccggtctgcaagagagattaatctctttcttccccttccttgtcttcacaggacgcattgaaattttcatcgaagatcttccaaggacacgttacaggattttgtaatagtaaacatatggaaagtattagacatatttattgcctgtacatactgtaaatgcattgggatcaaactgtctccccaggaaactgcacatgggtcatgtgaatatttttcccttttgccaaggctaatccaattattcctgtcactgttaccataatttattttgtcaactgatgtatttatttgtaaatgtatcttggtgctgctgactctgtttttttgtaacataatgcactttaggtatacatatcgagtatgtggatgaatttaacacataaaaggatctctattttgtggttcattttaatgagttctgaaatataattatctagactgatttccttctgtgcatgtaaaaatggcagtattttaaatttgttaaataatgtctaataaaatataatctaattataccatgactcacaatgtgaattttattctttaaagtttctaatcagagaaacacaacacaattttttttaattctacttgaatataatcctaaacttaatatgtttatatgggccatctattccaatgtgtccacattagcttttagttaaaaaccacttaaaatgtattctgtccaatatttcagttcaagtcaataaaatggctgaacagcctgaacttcaagttgaaataatggcaacaaagggcaaaaataaataagtagagaagtcagaagaaaaggtcaaagaggttatccagagttaaaaccagagggagaggtggacccaagactttgactctgaataaattttgccaatataaagtttagtttgcaagggcttgtctcattcataacaataatgaaagatct

SEQ ID NO: 68 - protein, 175 aa, Mus musculus; identical to SEQ ID NO: 46

SEQ ID NO: 69 - DNA, 935 bp, Mus musculus
cctaggtctcccattcctatctcccctccctactctaaaagccagctacttgccagttattccttcaaggaagccacgggaaggacctctgctgacaacttgtcttctcccctaacagcaaacgcgctgtgtctgaacatcagctactgcatgacaagggcaagtccatccaagacttgcgccgccgtttcttcctccaccatctgatcgcggagatccacacagccgaaatcagagctacctcggaggtgtcccccaactccaaacctgctcccaacaccaaaaaccaccccgtgcggtttgggtcagacgatgagggcagatacctaactcaggaaaccaacaaggtggagacgtacaaagaacagccactcaagacacccgggaagaagaagaaaggcaagcctgggaaacgcagagaacaggagaaaaagaagcgaaggactcggtctgcctggccaagcacagctgcgagtggcctgcttgaggaccccctgccccacacctccaggacctcgctggagcccagcttaaggtactgttcactgtgttcagcagggctcccctgcctgcctctatcttggtttcagagaccccgattttctgtatcttgatctgtgatttaaaaccagtataggccacaccagtcctagtccttattaccctgctagacaatttaatttccctacatctctctccccaccaacctacctacctacctacctaccttcctttcttcctccctccctccctccctccctccctccctcctttccttccttccttccctcccccttccttcttcacttcctccccatcctctctcctctcttcctccctcctcccttcactccctcctccctccctcctcctccccttcctatcttactcccacacacacagggtcttcctcacagccaagactgactgagctc

SEQ ID NO: 70 - protein, 175 aa, Mus musculus; identical to SEQ ID NO: 46

SEQ ID NO: 71 - DNA, 1869 bp, Mus musculus
aagctttaactgctggcgagcagcaaggcttaggggaggggaggggtgagaagtcctaggagtacccaccccagaagtctgtagagcatcacagcccaggtctgacaaaggtctttgagtgacactagcctcctagagagtcagatgaaattttccagcagtattgtctagaaggcaaacttctttgagtggttcccatgttcatttcagcgtgatctaggacctggggggaaactagtattcacacacacacacacacacacacacacacacacacacacacacacacctctagacccacaatcagtaactcacaactttctcccccaccccccacccccactccctgcccagcagcactgagaagctgaagcaaatgggcactcagtgaatctttatcaggacatgagtaggttatcagaagaaggtgagaaagaaggactctgcgcattccaggtcgtctgtgatcacaaagtgggtcttcttccttgctcacacacccggttctaccacctgctccagacgtggatgtccccaacccttcccaaacagtaagacccaaaactgcctttgctcttctgggtggctaggtttagtccctccccaggagaaggccacaccaggcaggaatgcagtaatgcgagcttgcaacccggggttcctccttgctgtttgtagggacctggactcctggacctgccactcaggcgacagcaggccggttcaggaggaggagagcgtcagatcgcccacgcgggccaggactggggtggggctccgtggctccgggctcccctgtgctccccgcccccgccggcccgctcacccctgcctgctccgccggcgcgcgttcctcgggcgccacctctttgcgactcgctcacttctcagcaggttggccccggagcgtgtgaaccttccagggctgggctcacagctactctccaacctgcgccgccccagctgggccgctccgccccgctgccggaacccgccctcgcgccacctggcctcgcatccacgacacgcgcgcctgcaacttgttcaagggcgttgtggaatcaactttccggaagcaaccagcccaccgaaggaggtagaccgacagctatgtatatatacgcgctctgccgcaagtggctgtgcacccagaggcccggtgcgcaggacagctgactcctgaggaacacccgcgtttgaagaggggtttgacctgccccacgacccagagtgctgccgccaagactaattagacattgctatgggagccacagcaacgcgccacgcatccccgacgcctatgtaaaacgcccggttttcgctcttctttcagaggaagctctctgattgctttttcccctctcgggtccctttttgcctgtgcggtttgagagaggcgcagttagaggcgctgattcctacacaagtaagtgctcaggggctcggagacacttttttgctttctttttcttgttttcttttttcctcctaacttgtgagatgccctcgactttggagtggctgttgggaagccacggggttccaaaagagggagcggggcagtgcgaggaagagatggagagccgggactgaccctcgggttccactcttgcaggtccccagagccagcgagcggcacgatgctgcggaggctggttcagcagtggagtgtcctggtattcctgctcagctactccgtgccctcccgcgggcgttcggtggaggggcttggccgcaggctgtaagtaccggtcttcctcctggggtgaggggtggggaagaccagcgccagctgaggcgggcgatgctgaggaagccatggggtccctgatggtcccaacctgagttggacacagtccacggggtacc

SEQ ID NO: 72 - protein, 177 aa, Homo sapiens
MQRRLVQQWSVAVFLLSYAVPSCGRSVEGLSRRLKRAVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRRH

SEQ ID NO: 73 - DNA, 1025 bp, Homo sapiens
gtcccgagcgcgagcggagacgatgcagcggagactggttcagcagtggagcgtcgcggtgttcctgctgagctacgcggtgccctcctgcgggcgctcggtggagggtctcagccgccgcctcaaaagagctgtgtctgaacatcagctcctccatgacaaggggaagtccatccaagatttacggcgacgattcttccttcaccatctgatcgcagaaatccacacagctgaaatcagagctacctcggaggtgtcccctaactccaagccctctcccaacacaaagaaccaccccgtccgatttgggtctgatgatgagggcagatacctaactcaggaaactaacaaggtggagacgtacaaagagcagccgctcaagacacctgggaagaaaaagaaaggcaagcccgggaaacgcaaggagcaggaaaagaaaaaacggcgaactcgctctgcctggttagactctggagtgactgggagtgggctagaaggggaccacctgtctgacacctccacaacgtcgctggagctcgattcacggaggcattgaaattttcagcagagaccttccaaggacatattgcaggattctgtaatagtgaacatatggaaagtattagaaatatttattgtctgtaaatactgtaaatgcattggaataaaactgtctcccccattgctctatgaaactgcacattggtcattgtgaatatttttttttttgccaaggctaatccaattattattatcacatttaccataatttattttgtccattgatgtatttattttgtaaatgtatcttggtgctgctgaatttctatattttttgtaacataatgcactttagatatacatatcaagtatgttgataaatgacacaatgaagtgtctctattttgtggttgattttaatgaatgcctaaatataattatccaaattgattttccttcgtgcatgtaaaaataacagtattttaaatttgtaaagaatgtctaataaaatataatctaattac

SEQ ID NO: 74 - protein, 258 aa, Leishmania major
MLVSVKPKPEVAGTAHVPLGPTGTNVRGAFPITAASRSAHAMFPSTWYSALKPKAAYYFAKPDTSSWKLSDFELKNTLGTGSFGRVRIAHRKGTEEYYAIKCLRKREIIKMKQQQHVAQEKGILMELCHPFIVNMMCSFQDEKKVYFLLEFVMGGEMFTHLRTAGRFPNDVAKFYHAKLVLAFEYLHSLDVIYRDLKPENLLLDNKGHVKMTDFGFAKKVPDRTFTLCGTPEYLAPEVIQSKGHGKAVDWWTMGVLLY

SEQ ID NO: 75 - DNA, 838 bp, Leishmania major
tttgacacagccgatgtggggtggcatcagctcgggcactgctgattgcatctcactggcgccatgctagtgagtgtgaagccgaaacctgaggtggctggcacagctcacgtaccccttgggcccacggggacgaatgtgcgcggtgcgtttcccatcaccgctgcgtccagatcggcgcatgctatgtttcctagcacgtggtactccgctttaaaacccaaggccgcttattacttcgcaaagccagacacctccagctggaaactctcagatttcgaactgaagaacacgctcggcaccggttccttcggccgcgtgcgcatcgcccaccgcaagggcacggaggagtactacgcgatcaagtgcctgagaaagcgcgagatcatcaagatgaagcagcagcagcacgttgcgcaggaaaaggggatcctaatggagctgtgtcacccgttcatcgtgaacatgatgtgctccttccaggacgagaagaaggtgtactttctgctggagtttgtcatgggcggcgagatgttcacgcacctgcgcactgccgggcggttcccgaatgacgttgcgaagttctaccacgcgaagctggtgcttgcgttcgagtatctgcactcgctggacgtgatctaccgcgatctgaagccggagaatctgctgctggacaacaaggggcatgtgaagatgacggactttgggtttgcgaagaaggtgccggaccggacgttcacgctgtgcgggacaccggagtaccttgcgccggaggtgatccagagcaagggccacgggaaggcggtggactggtggacgatgggcgtgctgctgtacg

SEQ ID NO: 76 - protein, 381 aa, Leishmania major
MLVSVKPKPEVAGTAHVPLGPTGTNVRGAFPITAASRSAHAMFPSTWYSALKPKVACNFAKPDTSSWKLSDFELKNTLGTGSFGRVRIAHRKGTEEYYAIKCLRKREIIKMKQQQHVAQEKGILMELCHPFIVNMMCSFQDEKKVYFLLEFVMGGEMFTHLRTAGRFPNDVAKFYHAELVLAFEYLHSLDVIYRDLKPENLLLDNKGHVKMTDFGFAKKVPDRTFTLCGTPEYLAPEVIQSKGHGKAVDWWTMGVLLYEFIAGYPPFYDDTPFRIYEKILAGRLKFPNWFDGRARDLVKGLLQTDHTKRLGTLKGGPADVKNHPYFHGANWDKLYARYYPAPIPVRVKSPGDTSNFEKYPDSPVDRTPALTSAQQAELKGF

SEQ ID NO: 77 - DNA, 2333 bp, Leishmania major
tctgtgcttgcgcgtctccacgtgcgcgttttcaccgtatatctcctctggggaggggtcagacgtcgtcgcgagacgtcgcggttcctttcgggaagtccccgaaggcaggacgtccgtaccacttgtgaggtgtctgctttgcattgcattccgccgtcgaggcacggctttctctcgaacacgtcattggcattcgaacgggtgtctttgctcgactctttcgtggcgcacccgcaacgcaagcatacgacacaccttcgctagcgaaccgcaagaggacggcttgcagagaccaacgtgtttttcttgatttctgagaccatttcagggctctctctctctctctctctcaacctcttccgtcccccatctctttgacacagccgatgtggggtggcatcagctcgggcactgctgattgcatctcactggcgccatgctagtgagtgtgaagccgaaacctgaggtggctggcacagctcacgtaccccttgggcccacggggacgaatgtgcgcggtgcgtttcccatcaccgctgcgtccagatcggcgcatgctatgtttcctagcacgtggtactccgctttaaaacccaaggtcgcgtgcaacttcgcaaagccagacacctccagctggaaactctcagatttcgaactgaagaacacgctcggcaccggttccttcggccgcgtgcgcatcgcccaccgcaagggcacggaggagtactacgcgatcaagtgcctgagaaagcgcgagatcatcaagatgaagcagcagcagcacgttgcgcaggaaaaggggatcctaatggagctgtgtcacccgttcatcgtgaacatgatgtgctccttccaggacgagaagaaggtgtactttctgctggagtttgtcatgggcggcgagatgttcacgcacctgcgcactgccgggcggttcccgaatgacgttgcgaagttctaccacgcggagctggtgcttgcgttcgagtatctgcactcgctggacgtgatctaccgcgatctgaagccggagaatctgctgctggacaacaaggggcatgtgaagatgacggactttgggtttgcgaagaaggtgccggaccggacgttcacgctgtgcgggacaccggagtaccttgcgccggaggtgatccagagcaagggccacgggaaggcggtggactggtggacgatgggcgtgctgctgtacgaattcattgccggatacccgccgttctacgacgacacgccgttccggatttacgagaagatccttgctgggcggctgaagttcccgaactggtttgacggccgcgcgcgcgacctcgtgaaggggctgctgcagacggaccatacgaagcgcctggggacgctgaaaggcgggccggcggacgtgaagaaccatccgtacttccacggtgcgaactgggacaagctgtacgcacgttactaccccgcgccgatccctgtgcgggtgaagagccctggcgacacgagcaacttcgagaagtacccggacagcccggtcgaccgcacgcctgcgctgacgtcggcgcagcaggccgaattgaaggggttttaactgcatccgctgccttgcgatatgattttcacgtgtgcatctccttatgtgggcatatgtttgtgctcagtggcgaggtttggtgttttgtttttgtagccgctgtagcctgtgcccatctgaaggcgcttcttttgttttttgtttgccttttgggcattgttaccttcccgagcttctgtagtggggaaagatctgtattgtacccgcctactggaagctgtttggaacccctcttgtttcatattgtgcactgggctcaccgaatttctcacgtggactttacacggttgtctattgtcctcctctctgtcttcgatgtagttgtacacaggcgtgcatgtgtatgtgtgtgcgcttgtggccagtcttctcttcgtctccatttttatcgtctctgaatgttcgtttgtgttttgtttccgactggaagttgtgtatgtgtatgtatgtgtgtgctggtggtggtgggtgcagggctgcgcgtgctgctctttttttgtcgcttcgttttgtttctgatatgtcttcaagaagtgaaagaagcacgggggagacacgacagtagaatgagcaccaggtatctgtagctgatgttgtcatgaaccgttggcgtgtcttttctttgcttgtattattctcatcctacatcccacatccccagcctttcgcatgcagccgtgagtacagcacaaactaaacgcgtcgcggcgtctttcttttgtccaagtcgtcgattttgcggctc

SEQ ID NO: 78 - protein, 605 aa, Mucor racemosus
MADFTDSLIKNIGVHSSSPVMTSVNMGQLGEKLRQARTTTLASLSQALSKKPEAAAAAATAPNAVNESTTTPTTMQLPASEKATSQLEINVVEARNLTIADARKADTYCIVHYEGNTTSTLDKVDDGILPSTPLVIKSQVASGAFKAFEIMMSASSPKWMHRVNFDVTAGNKEITVFVYDRGNKLPNGEDRFLGMSSIVPNLVNKKTVELIFPLHGRPDDDQEVTGDVRLQVTFIDPKKANLKPEDFRIVRMIGQGSVGKVYEVIKRDSGRTYAMKVLSKRLLLAENEVDTAFNERNVLVQSLSSPFIANLKYSFQTTNHLFLVMDYFPGGELFDFLERERCLSEKRCQFFAAEIVCAFDNIHARNIVYRNLKPESILLDAHGHIALTDFGLCKQLKNKMDLIQGVPQVITQEYLAPEMVMQKPYGMAADWWSLGVLMFELLTGSPPFHSVEQGELFRQILEAPIKFPAGGCITEEAKDFICQLLERDPAKRLGSHGDVAQVKAHPFFKDLNWDVVYKKQMQLPFVPEVEEQLREEAIAAAAAISIPVTNSKTESTNANVMPVADQSKFKGFSYIREDVMAKKGEHRLGVNPEDEDPEVDFWFRQ

SEQ ID NO: 79 - DNA, 2578 bp, Mucor racemosus
tcgaaatcaaaggtctgctactagtctaataccacaggaagcagatattggtatttgaaactgccacgttattgaaatgcctctgatcgtatgactagctggcccaatgaacattacattggccccaacagccaatcaaagacgtcccaatttaaaggggatgttggcatctaatgttgatcgcagatagacacacctaaaattatgcactgttttgggttacacattgattttaggtaaccacaccatctagaattcaggacatgtagaagccggtatatgagatggaaggtacattgtttacaagtactagcgtcaataaagtatcaaatagattcagtgagtagtctgctatcactctatactagcgagcacagtcaatcggccgataagaaataggaacagaaatatacccccaacatcagtggtcttcaacggaatctcaaacatgaaactgttaaatatgagatggatcttgcctattcttctctcttgctcattcttctcatcatcgcagaatacacatacgtaatatggctgatttcacagattctctcatcaagaacattggcgttcactcatcatctcctgtcatgacatctgtcaatatgggtcaattgggtgaaaagcttcgtcaagctcgtacaacaacacttgcttccttatctcaagctctttcaaagaagcccgaagctgctgctgctgctgccactgcccccaacgctgttaatgaaagtaccaccacacccaccacaatgcaactccctgcttcggaaaaagccactagtcaattggagatcaatgtggttgaagctcgtaatttgaccattgctgatgcgcgcaaagccgacacctactgtattgttcattacgaaggcaacaccacatcaacgcttgataaagtagatgatggcatcttgcccagcacgcctctggtgattaaatctcaagtcgctagcggtgcattcaaggcatttgaaatcatgatgagcgctagttctcccaagtggatgcatcgtgtcaacttgtaagttgctatccagaatatgtcaaaaagggctctgcgctaaccatgttactatagtgatgtaactgctggtaacaaggagatcactgtgtttgtctatgatcgtggtaacaaattgcccaatggtgaagatcgcttcttgggcatgtctagcattgttcccaacttggtcaacaagaagacggtcgagctgatctttcctcttcacggccgtcctgacgatgatcaagaagttactggtgatgtccgtcttcaagttacttttatcgaccctaaaaaggtaattttatatgagtatgattcttgacagctgatgtctgacacttctaaaaccctattcaaggctaatcttaagccagaggatttccgcattgtgcgtatgattggtcaaggctcagtgggtaaggtgtatgaggtgatcaagcgtgattctggccgtacctatgccatgaaggtgctctctaagcgtctcttgctcgccgagaatgaagtcgatactgccttcaacgagcgcaatgtgctggttcagtctctctcaagccctttcattgccaatctcaagtacagtttccaaacaacaaaccatctcttcttggttatggattactttccgggtggcgaattgtttgatttcctggagcgtgagcgttgtttgagcgagaagcgttgccaattctttgctgccgagattgtgtgtgcctttgacaacatccatgctcgcaacattgtctatcgtaacctgaagccagagagcatcttgctggatgcacatggacacattgccttgacagatttcggcttatgcaagcaattgaagaacaagatggatttgattcaaggtgtgcctcaagtcattacacaagaatacctcgcccctgaaatggtaatgcaaaagccctatggcatggctgccgactggtggagtctcggtgttttgatgtttgagctgttgactggatctcctcctttccattctgttgaacaaggtgaattgtttagacaaatcctggaagctcccattaaattccctgctgggggctgcattacagaggaagccaaggatttcatctgccaactgctggagcgtgatcctgccaagcgtctgggctcccatggtgatgttgctcaggtcaaagcacatccattctttaaggatctcaactgggatgtcgtttacaagaagcaaatgcagcttccctttgtgcccgaggtagaagagcagctccgcgaagaagccattgctgctgctgctgccattagcattcctgtgaccaacagcaagaccgagtctaccaatgccaatgtgatgcctgtggctgatcaatccaaattcaagggatttagctatattcgtgaagatgtcatggcaaagaagggcgagcatcgtctgggtgtcaatcctgaggatgaagatcccgaagttgatttctggtttagacagtaaaaatcgtccatctatccttacattttgtacatatatattaatcaagacccccctcctcattcaataaagcacatatttgttcatataccaaaaaaaaaaaaaaaaaa

SEQ ID NO: 80 - protein, 349 aa, Xenopus laevis
GNAATAKKGNEIESVKEFLAKAKEDFLRKWETPPQNTASLDDFDRMKTLGTGSFGRVMLVKHKGAEQYYAMKILDKQKVVKLKQIEHTLNEKRILQAVNFPFLVRLEYSFKDNSNLYMIMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRYGNLKNGVNDIKNHKWFATTDWIAIYQRKVEAPFIPKCRGPGDTSNFDDYEGSFHLSLTEKCAKEFADF

SEQ ID NO: 81 - DNA, 1994 bp, Xenopus laevis
tgggaaacgctgccaccgccaagaaggggaatgaaattgagagcgtgaaagaatttctagccaaagccaaagaagactttttaagaaagtgggaaacgcctcctcagaatactgcaagtctagatgattttgatagaatgaaaacccttggaactggctcatttggaagagttatgttagtgaaacataaaggtgcagagcagtattatgctatgaagatactggataaacagaaggtggtgaagctgaaacaaatagagcatacattaaatgaaaagaggatattgcaggcagtgaactttcctttccttgtcaggttggaatattcctttaaggataactctaatttatatatgattatggaatatgtccctgggggagaaatgttttcacatttaagaaggattggcaggttcagtgagcctcacgcaagattttacgcagctcagatagtgctgacatttgagtacctccattcactagaccttatctacagagatctcaaaccagaaaatcttttaattgaccagcaaggatacattcaggtcacagattttgggtttgccaaaagagtaaagggtagaacttggacattatgtggaacgccagagtatctggcaccagaaattattttaagcaagggttataacaaagcagttgactggtgggcattaggagttttaatatatgaaatggctgctggttatccccctttctttgctgatcaacccattcagatctatgaaaaaattgtgtcaggaaaggtacggtttccttcccacttcagctcagaccttaaagacctgctaaggaacttgctccaggtagatttgaccaaaagatatggcaacttgaagaatggcgttaatgacatcaaaaatcacaaatggtttgctacaacagactggattgcaatttatcagagaaaggtggaagctcccttcataccaaagtgcagagggcctggagataccagtaactttgatgactatgaagggagcttccacctttctctgacagaaaaatgtgcaaaagagtttgccgacttttaggatgtgcatgagaacaagatgacatcagggctcatattggaatctttgcactctttttgcagatggggtgaagctgagaccatcttatgtcacacagttacctagttccttcattccgcaaggttgactaaggtctattttgccatcttccatgtgtgcgtttcgcaccaccctttttatctaggcacaattaatcaagcgatttgtgctgtaacacagaatgctagaccactttccttcgtctcttgttttattttattttttttccttttttgtttacatcttgtatgttttgcccttctcctcacaggtatttttttaagtagtattttacccaactacactccctttattttgaaaatgtattattattagttgggggggcagggggtacgagagagagagagagagagagttctgggaggtgctgcaatgaggaaaatttcagatggaggaaatgttgtttctagtctctgccactccataggtttttgttttacattgtaaaccttatttctgtatggtggaaaatgtttcagctcaaattggaactactggccaatattattttgagtgtttatgaagtaaccataaccacggccaaaatctccaaatggaaataatgttgagaacaaaattcttcctagtgattgaccaaaattggttagaaatcaatttgagtgctacgtttggctagatagaccctgtgccttggatttctattctttaactgcgctgcccctcctggtgtgttccttgctttggcaggacctaaatgttgcgtgaccatactgtacgacacatagcacattgtttggttaaacttgaggatttacagctgaagtctttgaattacataatgctgactttaaaaatatcacgtccttcttggggggggttttaataacattaaacctgctcttcaataaaaaaaaaaaaaaa

SEQ ID NO: 82 - protein, 351 aa, Xenopus laevis
MGNAATTKKGGEIESVKEFLAKAKEDFLKKWESPAQNTANLDQFERMKTLGTGSFGRVMLVRHKENGSHFAMKILDKQKVVKLKQIEHTLNEKRILQAVNFPFLVRLEYSFKDNTNLYMVMEYVAGGEMFSHLRRIGRFSEPHARFYASQIVLTFEYLHALDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVTDIKGHKWFSTTDWIAVYQKKVEAPFIPKCKGPGDTSNFDDYEEEEIRVSITEKCAKEFSDF

SEQ ID NO: 83 - DNA, 2737 bp, Xenopus laevis
cgggaaaggttccgcggcctgcggggttctagaggtgacgagcgggtgctggatgcgtccggtgggaggagagccagcaggcccgtcatatcgcttcccagagatgctgatcgatgtgtaacccagaagtaacggactgatttctcatcgcaggtcttaaacttattggtggggggcgtttgtccagaatttagagggcccgtccgccccccaccatgggcaacgcggctaccacaaagaagggtggcgagattgaaagtgtgaaagaatttctggcaaaagcaaaagaagattttctgaaaaaatgggagagtccagcacagaacacagcaaatctggaccagtttgagcgaatgaaaactcttggcaccgggtcgtttgggcgcgtaatgctggtcaggcacaaagaaaatggcagtcactttgctatgaaaatcttggacaagcagaaggtggtgaagttgaagcagattgagcacacgttgaatgagaaacggatactgcaggctgtgaatttcccattccttgtgcggctggagtattcgtttaaggataacaccaatctgtatatggtcatggagtatgtagccggcggggagatgttctctcatttgcgcagaatcgggcgcttcagtgaacctcatgcacgcttctatgcatcccagattgtattaactttcgaatatctacacgccctggatcttatatacagagacttgaagccagagaatcttctcatagaccagcaggggtatatccaggtgacagattttggatttgcaaagagagtgaaagggaggacctggaccctgtgtgggacaccagaataccttgcgccagaaataatccttagcaagggttataataaggcagtggattggtgggccctaggagtcctgatttatgagatggctgctgggtatcctccatttttcgctgatcagcccattcagatctatgaaaaaatagtatcggggaaggtccggttcccctctcactttagctccgacctgaaggatcttctcagaaatcttttgcaagttgatctgactaagcgttttgggaatttgaagaacggtgtcacagacatcaagggacacaaatggttcagtacaacggactggattgcagtctaccagaaaaaggtggaagctcccttcatccctaaatgtaagggtcctggagacaccagtaattttgacgactatgaagaagaagaaatcagagtttcaatcactgaaaaatgcgctaaagaattctctgacttttaatttgcacccgtaaaggcggagcagtcaccaaacacgcccatcaagcaagagagaccgttggagcaagcagcactgacaggcacacaaccacctcttccctttcacactaccccatcccttctaagcttattggtggtgtggtcagacatggcacccatcctgttttatgctggatctggtggtgtggttctgagacttttgtttctctttctgtgacagatttgaggggccaagaatttatttaaatgtgactggatcctctggggtggagtcagaaccttgggggcagagcctcaaccctcagaatgggcgatgtatggtttctttttttttttttttaaccattttcttcggagcaagccagccactggtttttattgattaatttacatagtatataaacagagaaactgttttaatctaaacattttcctcccccatcaatattctttttcttcatagcctattgtagagggatttttaatttcaatgtgccagttttacccaaaaatctcccttttttgtctttacctgttttttccagcatctttggtggtggtgtttcttgtccttatatctctaagcaaatagccaaccctctgctttgatactaaagttagaaatctggtggttgatcagaaatgcattacataacactctccccgaattcggttttttttttttttttttttttcagattccaagacttttattgaatctaaatacatttaagggtcattgattcttccctcaccaaagtccctgaggtctttgattggacactacatttacagtctgccaaaagcttgtatacagctggtcagttaatccattgcagacttctgttccaccagaaaacaaaggaataatttaagtaacctatagccaccaaatagcttaactgccagctgtagtaagacaaaattctaaagagttgctataggttactcccttccaaccatctatgcctagtctgaaatagattataaattctagcaagttatagccctcaaaacaaagaaaggtgtctaaggaaatatcagacttcaattcaggttctctctcccccattaatgaagtctaagcatctaacatatttcagttcttaggaccaatttctttttatgtgagggaagaagcctcaacctaatgatcacattaaacagaatcaaaaggacgaatgttttaggtgaaagttcttcatggaaggaccaacaagttgtctgcacaaatgcatttggggtatcgaacagttctatgaggagtgaaaaacaaatatgctaagcattggggcataaataataagtgcttcttgggtctggttgtcaaggtgctcaacaagggcagcttgttagatgtcttgagtcccctcatttggtcacctggtgaatagcatgagcaaggtaaccattatttgctgatgttactcctgcaaccgaaatacgtccatccttcgtcatgtagat

SEQ ID NO: 84 - protein, 309 aa, Trypanosoma cruzi
MSGEANYEKPDTSNWKLADFEMGDTLGTGSFGRVRIAKLKGTNDYYAVKCLKKREILKMKQVQHISQEKQILMELSHPFIVNMMCSFQDDRRVYFVLEFVVGGEMFTHLRSAGRFPNDVAKFYHAEIVLAFEYLHSKDIIYRDLKPENLLLDSKGHVKVTDFGFAKKVPERTFTLCGTPEYLAPEVIQSKGHGKAVDWWTMGVLLYEFIAGYPPFYDDTPFRTYEKILSGRFKFPSWFDARARDLVKGLLQTDHTKRNWEKLYARYYPAPIPVKAKSPGDTSNFERYPESQEDRAVPLTATQQAEFIGF

SEQ ID NO: 85 - DNA, 1318 bp, Trypanosoma cruzi
ttttaatgatggaattggcatgcagtttcctctttaattatatcaatatatcttatttttcttttttttttcccgctctttaacgtaattaaaatatctttcttagcgccgtgcttcccccgtttcttagattacttctatgacttcatttccctactttgtttctttttcttatatacatatacatatgtgcatctaagattgggcatttttccttccttataaaagagcccacacagataattttaacagcatttttgaaaaataggtactggcgacttgccaagaaggagacgacagttataaacaacatgtcaggggaggcaaattatgaaaagccggacacctcaaattggaagttagcagactttgagatgggcgacacgttagggacgggttcttttggtcgcgtgcgcattgctaaactgaagggcaccaatgactattatgcggtgaagtgcttgaagaagcgggagattttaaagatgaagcaggtccaacacataagccaagaaaagcaaattttaatggagctttcacacccgttcattgtgaacatgatgtgttcctttcaggatgatcgccgggtgtactttgtgttggagtttgtggtgggcggggagatgtttactcaccttcgctccgccggtcggtttccaaacgatgtagcaaagttctaccacgcggagattgtgcttgccttcgaatacctgcactcgaaggacatcatctaccgtgacctgaagccggagaaccttctgctggacagcaagggccacgtgaaggtgactgactttgggtttgcgaagaaggtgccggaacgtaccttcacgctgtgtgggacaccggagtaccttgcgcccgaggtgattcagagcaagggtcatgggaaggccgtggactggtggacgatgggcgttctgctgtacgaatttattgctgggtatcctccgttttacgacgacacgccgttccgcacgtacgagaagatcctctctgggcgcttcaagtttccgagttggtttgatgcgcgtgcgcgggatcttgtgaaggggcttctgcaaacggaccacacgaagcgtaactgggagaagttgtatgcgcgatactaccctgccccgatccctgtgaaggcaaagagccctggcgacacgagtaactttgagcgttaccctgaaagccaggaagatcgagcggtgccgctaaccgctacgcaacaggcggagtttattgggttctaaacgtttttacttttataaactttttgaatgtacttttttttttttttttaaattctgtgtatatattcttcttattg

SEQ ID NO: 86 - protein, 332 aa, Leishmania major
MITKAEASQWRLSDLEMRETVGTGTFGRVRLVKHKGTGQYAALKILKKQEILRMKQVDHVMAEASLLQEIDHPFIVSMLRGYMDKNRLYILLEYVVGGELFSHLRKAGKFPNDVSKFYCAEVILAFDYLHNKTIVYRDLKPENILLDQDGNIKITDFGFAKRVTERTFTLCGTPEYLAPEIIQSKGHNKAVDWWALGILLYEMLVGYPPFFDDSPMKIYEKILVGKVLFPRWVDSKARDFIKGLLSLDPTKRLGNLPNGTEDIKNHKYFAEVDWNVVLSKKIPAPIPVRQHKEGDTHYFDKYPDSPLNPLRTLTPAQQDCFANFCNGQYTDE

SEQ ID NO: 87 - DNA, 3461 bp, Leishmania major
ttttgtcaccgccttgctctcctacaagcgcccgcagcaggcaaaacagtatatttcgcgtattccaaagatcgagacgcggatggaatactacgtgctctgcagcgactggttcggtgcaggtgccgactgcaagcggagcaacgaccccgatttgctagcgcagctgaaggaccgcgccaaagggaatccagatgcactgcgtcagatcgaagagggctggaacacactgccccacacgtcaggcatcagctttcccaagttcttttagcatcacgtctttacacggaagcgagcagaaatggcagacaacttcctcctccgggtaccacgactatcgactcctctttctgtttttccatctcttcctttgcaaggtgacctcgagtgcatttcgttgctgctaacaaaaaagctctagcgatgcgccggcgacgtgctgtatttgctatatcttcatcacaggagtggcatttctggcctgtggtgcgccgcaacagagacagtttcctgtgtcatgttcatctcccagcagaggttgcgttgcgattctcgttgggcaaggtagggcgccggcatatgcatagggggtgggttgtgcacgtcgtgtgttgttgtcgtccttcgttctcccgtgccatcgaccgggagtgaacgaaaaaaaacgaggcaccaagtggaggtgcaggcttgccgcgttttttttcgttactcttcccaaagcgcacgcgaaccgcgttgctatctctcttagccatcacgtgtcctaggtgtgttgaacaacaagttcgcgaggcttgcatgtggggctttcttttgatcatgcaatcatgtccactgtccaccacacactccagctctcttctatttccgtcctcgtctgtttcgccctctccgtgggcctctggtaccccccaataaagctgtacgaactcctcatgcataggcgaggacgggtgaaacaaaccacgcaaacaggatcgagttaaaaaggcacagctgtacgcctctttcctctccttttctcccccattcgtgttcttgcagctacaaaagactctgcttcatacacattcagtgagggtgaccagctgtcgggcgcacactctgtcagagagagagagagagatccaaattatccgccgttgtctgccgttctcgcgccctggacattgtgttccttttgctcagtcatatccttaccagtcataatagaacaccatgatcaccaaggccgaagcttcccaatggaggctgagcgacttggagatgcgtgagacggtcggcaccggcactttcggccgagtacgcttggtgaagcacaaaggaacaggccagtacgcggccctcaagatcctgaagaagcaggagattctccgaatgaagcaggtcgaccacgtgatggcggaggcaagtcttctccaggagatcgaccacccttttattgtgagcatgctgcggggatacatggacaagaaccggctgtacattctcctcgagtacgtggtaggcggcgagctgttttcgcatctccgcaaggccggcaagttccctaatgatgtctcgaagttctactgcgccgaggtgatcctagcatttgactacctgcacaacaagacgatcgtttaccgtgatctgaagccggagaacattctcctcgaccaggacggcaacataaagatcacggatttcggattcgcgaagcgcgtgacggagcgtacgtttacgctttgtggcacacccgagtacctagcacccgagatcattcagagcaaggggcacaacaaggctgtcgactggtgggcgctcggcatcctgctgtacgaaatgctggttggctatccgccgttcttcgacgacagcccaatgaagatctacgagaagatccttgtgggcaaggtgctgttcccacgatgggtagactcgaaggcccgagacttcatcaagggccttctatccctcgatcccacgaagcgcctcggcaatttgcccaacggcacggaggacatcaagaaccacaagtactttgcggaagtggactggaatgtagtgctgtccaagaagattcccgcgccaatcccagtgcggcaacacaaggagggcgatacccactacttcgacaagtaccccgacagcccactcaaccctttgcgcactttgactccagcacagcaggactgcttcgccaacttctgcaacggccagtatacggacgagtagcatcccgtttctgcctccaaggttatgaagtgcggaggggggggagcgacgagcacgtgtagacctttctcacacgcttagttgtgtttcccgcctcctccttagcggcgttggtgcgcgcgaccgcgacacggatgccgctaagcatctcccctaagccttcgggggaagtcgctttgcctgcacgttcctctgcaaccgccgttgctctgtcttcatgttcagactgtttattttttctttgttttgtgccctttccacgctcgatgcgttttcggcagacgcgcagtggagagggtgtagtgcgtgcgggtcgcacggcgatttctcgccttcacgatgtatgtataggcgtttcagagctcttaagtgtaatgttgacgaacgcgcaagcacaatgtattataaaagacacatacacaaagagaggttattgtggattgtgatctcgaagggttggcgtcggggtcgatggtatttttagttttttgtttttctgcctttccttcgaagtcggctctgattccttttaggcaggcaagtaggcgggatgctgcgttctcaatgatccatgtacgcgcaacgcaaagccagtagagggatcgataccatggctttcgtccgctgatctttacacagtctccctttttgtttctccggcgaccttcccacctttcatgtgtgccccctttttcactttcttttgatgagaaggagctttcctctgagccctctttcacctttcctgtttccctccgctgccgctggatgaaaggtctgtgtgtctgttcgtgatggcaagaggaaaggagcacaagatgcggaaaagggcagagttggatgtgacgggcagtgaaaagacgtgagaggccgctgccgcagcttttgctctcgacgatgtagctgtagccacgttgatggatatcttgtagagcggctatataagtcaaacgctggaggggggggggaactgatggggctgcctctctgtctccacttttggaccttgcttctcctgatttcatccctcctggaatcaacttggaattgacacacatcaacatactcatttgattttaggtgctgcccttctcctgtgtcctctctcctccacaaatgattataaaaaataaggcgtgcctgctgtcatcagaaaaacgaacggaaaaaaggaaatccacatcaatgcacactctcgagactgcgggagtgagttttatgtgtggtcgcgcattagaaca

SEQ ID NO: 88 - protein, 359 aa, Giardia intestinalis
MEDIQVEAYPELSYKDLRHIKNIGSGSFGRVTLAQHIDTGKYYAVKAMSKRKIIALRQVAHINNECSILRVVRSPFIIRQYGTYQDDKNVYIILDFIQGGELFYHLRRYNKFPLQVVKFFAVEILLALGYLHNLGIAYRDLKLENVLVDNTGHIKLADLGFAKRLVEKNADGDTVSQLTFSIVGTPEYLAPEIIRSTGHDMSADWWAFGVLIYEMLTGSPPFFDDNPDMTCKKILGGKITFASGFDKAAKDLIIRLLNPDKTRRLGASINNGTADIMKHAFFSGVNCLGLRKKSGRHPIVPKLTDPADTANYEDYLDENGNFEEDEIDYENLRSDAAELGLAEIPGTQEEKDALFVGFK

SEQ ID NO: 89 - DNA, 1307 bp, Giardia intestinalis
gcacgagctctgctcatggaagatattcaagtagaagcgtatccagagctatcttacaaggatctcaggcatataaagaatattggttcgggatccttcggcagggtcactcttgcacagcacattgacacaggaaagtactacgccgtgaaggcaatgtccaagcggaagattattgcccttcggcaggttgctcacattaacaacgagtgtagtatcttgcgtgtcgtgaggtctccgtttatcattaggcagtatggaacctatcaggacgacaagaatgtatacatcatcctggacttcatccagggcggcgagcttttctatcacctacgcagatataacaaatttccactccaagttgttaagttctttgccgtcgagatcctcctggcattggggtatttacataatctgggcatcgcctatcgtgatctgaagctagagaacgtcctcgttgacaacacaggccatataaagcttgctgatctggggtttgctaagcgcctggtcgagaagaatgcagacggggatacggtgtcccaacttactttcagtatcgtgggcaccccagaatacttggcccccgaaattatccgatcgaccgggcacgatatgtctgcagattggtgggccttcggcgtcctcatatatgagatgctaacagggtctccgcccttcttcgatgacaatcctgatatgacctgtaagaagatccttgggggaaagataacttttgcaagtgggtttgacaaggcagcgaaggacctgattatccgcttactcaaccctgacaaaacaaggagattgggggcatctataaacaacggcacggcagacattatgaagcacgcattcttctcaggggtcaattgtcttggtttgagaaaaaagtcgggaaggcaccccattgtccctaagctcacagaccctgccgacaccgccaattatgaagactatctcgatgagaatggtaactttgaagaggacgagatcgactacgagaatctacggagcgatgcggccgagctgggcctggcagagatacctgggactcaggaagaaaaagacgccctgtttgtgggatttaaataggtgcgtgacccttgacataaaacaaaaccctaccttacttattaatcactccttactccttttgctccctacgttcttttctggccgccttttgtctagtggatcgagagggtccaggctaatattaacagcacttatcatctgatacagatgagcacgctgccttctttgaacatgtaaaccgagactaaaaaaaaaaaaaaaaaaaaaaa

SEQ ID NO: 90 - DNA, 333 bp, Trichinella spiralis
  misc_feature (8)..(8): n is a, c, g, or t
caatttgncgaaatggcggtagnctgatatanaaaaatcacaagtggnttttctagcanccgattggattgccgtgtatcagcggaagatcgaagctccacttgtagcaaagtgtgcgacatccgggcgacacttagcaatgtttgacgaaatacgaagaagaagcgttgcacatttcaccggtggacaaatgtgtcaaagaatttgctgatttttgaatttatagactcgccgtggatttcaaatcttttttgttagtcgtcgtattaaaaacagaaaaatattaataataataataataagacaaagagaggcaaattcattttgcggact

SEQ ID NO: 91 - protein, 351 aa, Homo sapiens
MGNAAAAKKGSEQESVKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVNDIKNHKWFATTDWIAIYQRKVEAPFIPKFKGPGDTSNFDDYEEEEIRVSINEKCGKEFSEF

SEQ ID NO: 92 - DNA, 2685 bp, Homo sapiens
tcgggctgaggttcccgggcgggcgggcgcggagagacgcgggaagcaggggctgggcgggggtcgcggcgccgcagctagcgcagccagcccgagggccgccgccgccgccgcccagcgcgctccggggccgccggccgcagccagcacccgccgcgccgcagctccgggaccggccccggccgccgccgccgcgatgggcaacgccgccgccgccaagaagggcagcgagcaggagagcgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgccaagcgcgtgaagggccgcacttggaccttgtgcggcacccctgagtacctggcccctgagattatcctgagcaaaggctacaacaaggccgtggactggtgggccctgggggttcttatctatgaaatggccgctggctacccgcccttcttcgcagaccagcccatccagatctatgagaagatcgtctctgggaaggtgcgcttcccttcccacttcagctctgacttgaaggacctgctgcggaacctcctgcaggtagatctcaccaagcgctttgggaacctcaagaatggggtcaacgatatcaagaaccacaagtggtttgccacaactgactggattgccatctaccagaggaaggtggaagctcccttcataccaaagtttaaaggccctggggatacgagtaactttgacgactatgaggaagaagaaatccgggtctccatcaatgagaagtgtggcaaggagttttctgagttttaggggcatgcctgtgcccccatgggttttcttttttcttttttcttttttttggtcgggggggtgggagggttggattgaacagccagagggccccagagttccttgcatctaatttcacccccaccccaccctccagggttagggggagcaggaagcccagataatcagagggacagaaacaccagctgctccccctcatccccttcaccctcctgccccctctcccacttttcccttcctctttccccacagccccccagcccctcagccctcccagcccacttctgcctgttttaaacgagtttctcaactccagtcagaccaggtcttgctggtgtatccagggacagggtatggaaagaggggctcacgcttaactccagcccccacccacacccccatcccacccaaccacaggccccacttgctaagggcaaatgaacgaagcgccaaccttcctttcggagtaatcctgcctgggaaggagagatttttagtgacatgttcagtgggttgcttgctagaatttttttaaaaaaacaacaatttaaaatcttatttaagttccaccagtgcctccctccctccttcctctactcccacccctcccatgtccccccattcctcaaatccattttaaagagaagcagactgactttggaaagggaggcgctggggtttgaacctccccgctgctaatctcccctgggcccctccccggggaatcctctctgccaatcctgcgagggtctaggcccctttaggaagcctccgctctctttttccccaacagacctgtcttcacccttgggctttgaaagccagacaaagcagctgcccctctccctgccaaagaggagtcatcccccaaaaagacagagggggagccccaagcccaagtctttcctcccagcagcgtttccccccaactccttaattttattctccgctagattttaacgtccagccttccctcagctgagtggggagggcatccctgcaaaagggaacagaagaggccaagtccccccaagccacggcccggggttcaaggctagagctgctggggaggggctgcctgttttactcacccaccagcttccgcctcccccatcctgggcgcccctcctccagcttagctgtcagctgtccatcacctctcccccactttctcatttgtgcttttttctctcgtaatagaaaagtggggagccgctggggagccaccccattcatccccgtatttccccctctcataacttctccccatcccaggaggagttctcaggcctggggtggggccccgggtgggtgcgggggcgattcaacctgtgtgctgcgaaggacgagacttcctcttgaacagtgtgctgttgtaaacatatttgaaaactattaccaataaagttttgtttaaaaaaaaaaaaaaaaaa

SEQ ID NO: 93 - protein, 7 aa, Mus musculus
MASSSND

SEQ ID NO: 94 - DNA, 912 bp, Mus musculus
ccaagagggtcttcccaggcagctttgccttttgcccagtcaggccaacagaagtttgaagggttctatctgcccctaccctgcacccattagtctgcaggttgagtttctcttcctgttcccaccctatcactccctggctccctctacaggcagggctcccccccaggactggcagccaaactgctgcagcagatcttatgaggcttccgagccaccgtaatgctagtgccctgagaaagactgagtgatggcttccagctccaacgatggtgaggctgagccctgttgttatagcaacttcctagacactgtctgccccttaagtcagggaggggctgtgatgggggctgaggggagcaggtggagagaaggagtctgtggacccttaagtatccctggaaatgcctccattcctaaaatcctgggctagaaaacctgaggaagaagaacagggggatgaatggtccatttcagaccaaagcagatggtctacccggagtagggctccagctgggacaggggatggagtagtcccacccagctttgggattcagaccagaagaaagataggtccaccttagacctgggtacagtagagtttgcttgataccctgaaccctgcctggagatagagggaacctagtgttgggggggtccccctcaaactgggggtatagtgcagtacactcttgcctgctgctgtgaccccatatctgaatccaaggtgatttccatgtcttgccgtggcactggctttcctagaggtgtctggagttttcgtagcacatcaaatccagagagtggaggccaatagcaccctttcctttcaaatgagagacatgggctctaccggctagcagcttaggctatcctctctgtccacttcctgttggtggcaggcgtccatgg

SEQ ID NO: 95 - protein, 31 aa, Homo sapiens
MASNSSDVKEFLAKAKEDFLKKWESPAQNTA

SEQ ID NO: 96 - DNA, 120 bp, Homo sapiens
ggtgccctgagaacaggactgagtgatggcttccaactccagcgatgtgaaagaattcttagccaaagccaaagaagattttcttaaaaaatgggaaagtcccgctcagaacacagccca

SEQ ID NO: 97 - protein, 472 aa, Blumeria graminis
MPTFGFLKKRKKVSLGHRDLDSPASTLSQPASPITPSTSRGLGEPIDTTSSKITSPGQKLGSNITKDHRGTRTTVEDLPSMTDVSHLQQAYRNLSQGSTASKSNLLTIHNLINPPQHDGAGQSKVMPEKHMNNKTERPVQGKAAVAQVRQTKGKYSLSDFEILRTLGTGSFGRVHLVQSKHNQRFYAVKVLKKQQVVKMKQVEHTNDERSMLQEVKHPFLITLWGTFQDSKNLYMVMDFVEGGELFSLLRKSQRFPNPVAKFYAAEVTLALEYLHKKDIIYRDLKPENLLLDRHGHLKITDFGFAKKVTDITWTLCGTPDYLAPEVVSSKGYNKSVDWRWSLGILIFEMLCGYTPFWDNGSPMKIYENILKGRVKYPPYIHPDAQDLIQRLITADLTKRLGNLHGGAEGIKSHQWFAEVTWERLAKKDIDAPYVPPVKAGSGDASQFDKYPEETERYGQTGPDEHGSLFENF

SEQ ID NO: 98 - DNA, 1573 bp, Blumeria graminis
atgccgacatttggatttttgaagaagcggaaaaaggtgtcattgggtcacagggaccttgattctcctgcttcgacacttagtcaacctgctagtccgattaccccatctacctcccgaggtctcggtgaacccattgatacaacttcatcgaaaatcacgtcgcctggccaaaaactcggttctaacatcacaaaagatcatcggggaacacgaactactgtggaggatttaccttcgatgacagacgtatcacatctccaacaagcttaccgaaacttatcacagggatctacagcaagtaaaagcaaccttctaaccattcataacctcatcaatcccccgcagcacgacggggcaggtcagtcaaaagtcatgccagaaaaacacatgaacaataaaaccgaacgcccagttcagggcaaagcagcagtggctcaagtcagacagactaaggggaagtactcgttgagcgattttgaaatattgcgtactcttgggacaggtagtttcggtagggttcatctggttcaatcaaagcataatcaaaggttttacgccgtgaaagtattgaaaaagcagcaagtagtaaaaatgaagcaagtagagcatacaaatgacgaacgaagtatgttacaagaagtcaagcatcctttcctgataactttatggggaactttccaagattcaaaaaatctatatatggttatggatttcgtcgaaggtggcgaactcttttctctattgcgaaaatcgcaggtataataacaaccctcagattctatggaagtatctgctaattttccaagcgattcccaaatccagtggccaagttttacgccgctgaagtcacattagctctcgaatacctgcacaaaaaagacattatttatcgagatttgaagccggagaatttacttctcgatcgtcacggacatctaaagattactgattttgggtttgcaaagaaggtaactgatatcacatggacactctgtggtacaccagattacttagcgcctgaagtggtttcgagtaaaggctataataaatcagtcgattggtgagtcccactttaagaaacaaaaacgctccgtctaatatatacatgaggtggtcgctaggcatattaatattcgaaatgctttgtggctatacgcccttctgggataatggctcaccaatgaaaatttatgaaaatattctcaagggtcgcgtcaagtaccctccatatattcacccagatgcacaagatctcattcaacgacttataactgccgatcttaccaagcggttaggaaatctacatggtggtgctgaaggaattaaaagccaccagtggtttgctgaagtaacctgggaaagattagcgaaaaaagacatagatgcaccatatgtgccaccggtcaaggcagggtctggtgatgcaagtcagtttgacaaatatcctgaagagacggaacggtacgggcagacaggaccagacgaagttttgttccccgtaaatcacaacagcttatatttgggtttactgacaaagcttagacacggaagcttgtttgaaaacttctga

SEQ ID NO: 99 - protein, 360 aa, Homo sapiens
MAAPAAATAMGNAPAKKDTEQEESVNEFLAKARGDFLYRWGNPAQNTASSDQFERLRTLGMGSFGRVMLVRHQETGGHYAMKILNKQKVVKMKQVEHILNEKRILQAIDFPFLVKLQFSFKDNSYLYLVMEYVPGGEMFSRLQRVGRFSEPHACFYAAQVVLAVQYLHSLDLIHRDLKPENLLIDQQGYLQVTDFGFAKRVKGRTWTLCGTPEYLAPEIILSKGYNKAVDWWALGVLIYEMAVGFPPFYADQPIQIYEKIVSGRVRFPSKLSSDLKDLLRSLLQVDLTKRFGNLRNGVGDIKNHKWFATTSWIAIYEKKVEAPFIPKYTGPGDASNFDDYEEEELRISINEKCAKEFSEF

SEQ ID NO: 100 - DNA, 1635 bp, Homo sapiens
ctcccgtagtcgtagttcccgtagtatggcggcccctgccgccgccaccgccatgggcaacgcccccgccaagaaggacaccgagcaggaggagagcgtgaacgagttcctagccaaagccagaggagatttcctctacagatggggaaaccccgctcaaaacaccgccagctcggatcagttcgaacggctcaggacgctgggcatgggctccttcgggcgggtgatgctggtgaggcaccaggagaccggcggccactacgccatgaagatcctcaacaagcagaaggtggtgaagatgaagcaggtcgagcacatactgaacgagaagcgcatcctgcaggcgatcgactttccgttcctcgtcaagctccagttctcctttaaggacaactcctacctgtacctggtgatggagtacgtgccgggtggggagatgttctcccgcctacagcgcgtcggaaggtttagcgagccccatgcctgtttctatgccgcccaggtcgtcctggccgtccagtacctacactcgctcgacctcatccaccgcgacctgaagcccgagaatctcctcatcgaccagcagggctacctgcaggtgacggacttcggtttcgccaagcgcgtgaagggccgcacttggaccttgtgcgggaccccagagtacctggcccccgagatcatcctgagcaaaggctacaacaaggccgtggactggtgggccctaggggtgctcatctatgagatggccgtgggcttcccacccttctacgccgaccagcccatccagatctacgagaagatcgtctctgggagggtgcggtttccctccaaactcagctctgacctcaaggatctgctgcggagcctgctgcaggtggacctcaccaagcgcttcggaaacctcaggaacggggttggcgacatcaagaaccacaagtggttcgccacaaccagctggatcgccatctatgagaagaaggtggaagctcccttcatcccgaagtacacaggccctggggatgccagtaactttgacgactacgaggaggaagagctccggatctccatcaatgagaagtgtgccaaggagttttctgagttttaggggtgtgcttgtgcccctgtggttttctttcctttttgtttttggtggtttgggggatgggagggttggattgaacagccagagggccccagagttccttgtatctaatttcatcctcacccacctccagggttgggggagcaggaagcccagatatttggaggaacagaaacaccagctgctccctcacccccccccatgccttcctggtccctctgtgcttctctctttctcctccacaggtcccccttgccccagcccccttctgcctgttttaaacgagtttctcagctctattcaggccaggtcttgctgttgtatcaagggacacggtgtggaaagaggggctcaaacttaactccagccctgaacaggcaccacttactaagagaggatgaatgaaaagcacacctaccctttggcgtaatcctgcctgggaaggagagaggtttagtgccatgttcagtgggctgtttgctagaataaaaaattaaaacaaaaaacaattaaaatcttatttaagttcc

SEQ ID NO: 101 - protein, 337 aa, Ascaris suum
MENREQEEIEPCVSITIDPNNNKLNVDDFDRICTIGTGSFGRVYLVQHRASEQYFALKKMAIREVVSMRQTEHVHSEKRLLSRLSHPFIVKMYCASWDKYNLYMLFEYLAGGELFSYLRASRTFSNSMARFYAAEIVCALQYLHSKNIAYRDLKPENLMLNKEGHLKMTDFGFAKEVIDRTWTMCGTPEYLAPEVIGNKGHDTAVDWWSLGVLIYEMMIGIPPFRGKTLDEIYEKIILGKLRFTRSFDLFAKDLVKKLLQVDRTQRLGNQKDGAADVMNHKWFTDIDWDDVQNMKLTPPIIPTLYSNGDTGNFDSYDECSDDEIAAPQHELELFEDW

SEQ ID NO: 102 - DNA, 1336 bp, Ascaris suum
ctgcagtctgtgacattccgccgagagggtttaattacccaagtttgagcgcgacaccggagtggtttaagccttaaagaggcaaattcgccgatagttttacgagcgatattttacgctatcatatcggtgaattgtaattctcggcaatggagaatcgagaacaagaagaaattgaaccatgtgtttcaatcactatcgatccaaacaataacaaacttaacgtcgatgattttgatcgtatttgcactatcggaacgggatcgtttggtcgagtatatcttgtgcagcatcgtgcttctgagcaatattttgcgcttaagaaaatggccattcgagaggtggtctcgatgcgtcaaaccgagcatgtccactccgagaagagactgctgtcgcgtctttcccatcccttcatcgttaaaatgtattgcgcttcgtgggacaaatacaatctctacatgctattcgagtatctagcaggtggagagctgttctcatacttgcgtgcctcgcgaactttctcgaactcaatggctcgtttctacgcagctgaaattgtctgcgcacttcaatacttacactcgaaaaatatcgcttatcgtgatttgaaaccggaaaatttaatgctaaataaagagggacatctcaaaatgactgatttcggtttcgcaaaagaagttattgacagaacatggacaatgtgtggtactccggagtacttagcacctgaggtgatcggcaataaaggacacgatacagcggtcgattggtggtcattgggtgttctcatctatgagatgatgatcggtataccaccgtttcgtggtaaaactcttgacgagatctacgaaaaaatcattttgggcaaacttcgcttcactcgctcgtttgatttatttgctaaggatcttgtgaagaagttgctacaagtggatcgtacacaacgattagggaatcagaaggatggtgcagctgatgtaatgaatcataaatggttcaccgatatcgattgggatgatgtacaaaatatgaagcttacccctccaatcattccgacattatattcaaacggagatacgggcaatttcgattcttatgatgaatgcagcgatgatgaaattgcagcacctcaacatgagctcgaactattcgaagactggtgatacattgtatatgtgctgtgtgtgcattatgtatgtatatatatccactccctgtcgcactcattaaccatcttttagaaattatgttctcatcacattgtgttcttatttacaccaacaaataacatgcgcatgcgcatgttcgagtactgctaaaaattttgtgtagaccc

SEQ ID NO: 103 - protein, 334 aa, Rattus norvegicus
EFLSKAKEDFLRKWENPPPSNAGLEDFERKKTLGTGSFGRVMLVKHKATEQYYAMKILDKQKVVKLKQIEHTLNEKRILQAVEFPFLVGLEYSFKDNSNLYMVMEYVPGGEMFSHLRRIGRFSEPHARFYAAQIVLTFEYLHSLDLIYRDLKPENLLIDHQGYIQVTDFGFAKRVKGRTWTFCGTPEYLAPEIILSKGYNKAVDWWALGVLIYEMAAGYPPFFADQPIQIYEKIVAGKVRFPSHFSSDLKDLLRNLLQVDLTKRFGNLKDGVNDIKNHKWFATTEWIAIYPRKVEAPFIPKFKGFGDTSNFDDYEEEEIRVRITEKCGKEFSEF

SEQ ID NO: 104 - DNA, 1002 bp, Rattus norvegicus
gaattcctatccaaagccaaagaagactttctgaggaaatgggagaaccctcccccgagtaatgctgggcttgaggattttgaaaggaaaaaaaccttgggaacgggttccttcggaagagtcatgctggtaaagcataaggccactgagcagtactacgccatgaagatcttagacaagcagaaggtcgttaagctaaagcaaatagagcacactctgaatgagaagagaatcctgcaggcagtggagttcccgttccttgttgggctggagtattcttttaaggataattctaatttatacatggttatggaatacgttcctgggggggaaatgttttcacatctaagaagaattggaaggttcagtgaaccccatgctcgtttctatgcagctcagatcgtgctaacatttgagtacctccattccctcgacctcatctacagagatctcaagccggaaaacctcttaattgaccaccagggttacatccaggtcacagattttgggttcgccaagagagtcaagggcaggacttggacattctgtggcaccccagagtacctggccccagagatcatcctcagcaagggttacaataaggcagtggactggtgggcattgggtgtactgatctacgagatggctgctggctaccccccgttctttgctgaccagccaattcagatttatgagaagattgttgccggaaaggtccgcttcccgtcgcacttcagttccgatctcaaggaccttctgcggaatctgctgcaggtggatctcaccaagcgctttggaaaccttaaggacggggttaatgacatcaaaaaccacaagtggtttgctactaccgaatggatcgcgatttacccgagaaaggttgaggctcctttcataccaaaattcaaaggctttggcgatacatctaacttcgatgactatgaagaagaagagatccgagtccgtataacagaaaaatgtggaaaggagttttctgaattt

SEQ ID NO: 105 - protein, 207 aa, Homo sapiens
EFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKVGASPALPFP

SEQ ID NO: 106 - DNA, 936 bp, Homo sapiens
gaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgccaagcgcgtgaagggccgcacttggaccttgtgcggcacccctgagtacctggcccctgagattatcctgagcaaagtaggagcctccccagccctccccttcccctgaggccggctctgctctcctgctctcgcctcctcctcaccctgtgcccccccatcttgctccagggctacaacaaggccgtggactggtgggccctgggggttcttatctatgaaatggccgctggctacccgcccttcttcgcagaccagcccatccagatctatgagaagatcgtctctgggaaggtgaggtccggatgtgggacacagccctggaagaaacagaccgttccctgctcacccatcctattccctggggagccctgcttgttgtcagaataatctagaagttccttaaaaaaaaaaaaaaaaa

SEQ ID NO: 107 - DNA, 377 bp, Aplysia californica
tacgcactggaaaataattcagctgtcaaatatctgttactatcgtgttacagccaaaagctcgtttaaattgtcatttttcaaatcattttaggattttgaaattgtttttattcattttgagttcaggaagaatgtaatgaaatatcactacccggaacgaccgtgtttcttgcattgattcggtaataaacatttctcagttctgagagaagaggtgttagtattgtttgtggctttcgtgttgcgtgcgtggactaggattcttatcgggattacacttctgtttttcaaactaacgggattatctgctcagcacctgtttgtgcagtcaaacactatcagattggaggtgcagtagcagttacactcaggat

SEQ ID NO: 108 - protein, 67 aa, Aplysia californica
MADIIHKLFGQKHGKHSDQGAKSSDGEGYTKQQHEFFKEFLARAKEEFQNKWDHPPASTSCLDDFDR

SEQ ID NO: 109 - DNA, 343 bp, Aplysia californica
ctgcagcaacaacaactgctacaacaacaacgtgttcaaacgtttcggaggattattcactaccgacacggaggaagcaactgcgccgttgattggatttgaaccccgaacctttcagaatcggggtggttgagcgaccgaaatggctgatattattcacaagttgttcggtcagaaacatggaaagcattcggatcagggagccaagtcgtctgatggagaaggctacaccaaacagcagcacgagttcttcaaagaattcttggccagagccaaagaggaatttcagaacaaatgggatcacccaccagcaagcacatcatgcttagacgacttcgacaga

SEQ ID NO: 110 - protein, 351 aa, Mus musculus [sequence omitted]

SEQ ID NO: 111 - DNA, 2292 bp, Mus musculus
cttgggctgaggctcccccgcgggcgggcgcagagagacgcgggaagcaggggctgggcgggggtcgtggcgccgcagccagcgcagccagccccaggggccgccgcctccgctgcccagcgcgctccggggccgccggccaccttagcacccgccgcgtcgcagctccgggactggccccggccgcgacgccgccgcgatgggcaacgccgccgccgccaagaagggcagcgagcaggagagcgtgaaagagttcctagccaaagccaaggaagatttcctgaaaaaatgggagaccccttctcagaatacagcccagttggatcagtttgatagaatcaagacccttggcaccggctcctttgggcgagtgatgctggtgaagcacaaggagagtgggaaccactacgccatgaagatcttagacaagcagaaggtggtgaagctaaagcagatcgagcacactctgaatgagaagcgcatcctgcaggccgtcaacttcccgttcctggtcaaacttgaattctccttcaaggacaactcaaacctgtacatggtcatggagtatgtagctggtggcgagatgttctcccacctacggcggattggaaggttcagcgagccccatgcccgtttctacgcggcgcagatcgtcctgacctttgagtatctgcactccctggacctcatctaccgggacctgaagcccgagaatcttctcatcgaccagcagggctatattcaggtgacagacttcggttttgccaagcgtgtgaaaggccgtacttggaccttgtgtgggacccctgagtacttggcccccgagattatcctgagcaaaggctacaacaaggctgtggactggtgggctctcggagtcctcatctacgagatggctgctggttacccacccttcttcgctgaccagcctatccagatctatgagaaaatcgtctctgggaaggtgcggttcccatcccacttcagctctgacttgaaggacctgctgcggaaccttctgcaggtggatctcaccaagcgctttgggaacctcaagaacggggtcaatgacatcaagaaccacaagtggtttgccacgactgactggattgccatctatcagagaaaggtggaagctcccttcataccaaagtttaaaggccctggggacacgagtaactttgacgactatgaggaggaagagatccgggtctccatcaatgagaagtgtggcaaggagtttactgagttttaggggtgtgcttgtgccccttgggttctctttcattttttctttttctttctattttttttccggttgggggtgggagggttggatcgaacagccagagggccctagagttccatgcatctaatttaacatccactccacacccccagggttaaggagagcaggaaagcgctccagatactggggaaggggcaacatcagctgctccccctctcccttcttctccacccttccctgcctgttttcaatgaatttcttagctccagccatacccaatcttgctggtgtatccaggggcagggtacggaaagagggccccaaattcagcctccttcccgaccctagcactggatactaaggatgaacgaacagtaacgccaaccttcccttccatgcagccctacctggaaagggagattttatgacctgtacagagggctgcttgccagtggggtttttttttttttttcatttaaattaagttccaccagtgcctcccaccctccaaactgtcccaccctccccaaacaccctcctcactccctaaatccattctgatgagacccgggtagccaactgaccctgtcaaggaaggaactgggcttggaatctcgccctgagctgctagcctcccggcccccctttccagtggtctcatgccaattgtcctgtgcatcagcccccttaagaagcctcccccatcctgggcgcctcgcttctagcttagctgtcagctgtccatcacctcttgccgtgcgtccccactcactgcaaccccaagtctgattgtgctttttctctcaatagaaaggtggggagctgctggggaaattaccccatttatccctgtgtttatccctctcgtaacttctcccaaaaaggaggagctctcaggcctgggtgggggccccgggtggacgagggggtcgttcaacctgtgtgcttcgaaggatgagacttcctcttgaacagtgtgctgttgtaaacatatttgaaaactattaccaataaagttttgttttaaaaaaaaaaaaaaaaaa

SEQ ID NO: 112 - protein, 115 aa, Homo sapiens; identical to SEQ ID NO: 8

SEQ ID NO: 113 - protein, 175 aa, Homo sapiens
MQRRLVQQWSVAVFLLSYAVPSCGRSVEGLSRRLKRAVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSR

SEQ ID NO: 114 - protein, 10 aa, Artificial Sequence
  Synthetic
AVSEHQLLHX

SEQ ID NO: 115 - protein, 10 aa, Artificial Sequence
  Synthetic
SVSEIQLMNL

SEQ ID NO: 116 - protein, 21 aa, Artificial Sequence
  Synthetic
VAPSDSIQAEEWYFGKITRRE

(19) United States
(12) Patent Application Publication
(10) Pub. No.: US 2006/0241067 A1
(43) Pub. Date: Oct. 26, 2006

(54) Methods For inhibiting angiogenesis, cell migration, cell adhesion, and cell survival

(76) Inventors: Judith A. Varner, Encinitas, CA (US); Manjiri Bakre, Singapore (SG); Hui Jin, Ann Arbor, MI (US)

Correspondence Address: Medlen & Carroll, 101 Howard Street, Suite 350, San Francisco, CA, 94105, US

(21) Appl. No.: 10/518,181

(22) PCT Filed: Jun. 25, 2003
(86) PCT No.: PCT/US03/20041

§ 371 Date: Sep. 9, 2005

Related U.S. Application Data
(60) Provisional application No. 60/391,484, filed on Jun. 25, 2002.

(51) Int. Cl. A61K 48/00

(52) U.S. Cl. 514/44; 424/93.21

(57) ABSTRACT

The invention relates to methods for detecting and inhibiting angiogenesis, cell migration, cell adhesion, and/or cell survival in endothelial and non-endothelial cells as well as in normal and tumor cells. The invention further relates to methods for screening test compounds for their ability to inhibit angiogenesis, cell migration, cell adhesion, and/or cell survival.

This invention was supported in part by grants CA71619, CA83133, AR47347, DK60588 from the National Institutes of Health. Accordingly, the United States government may have rights in this invention.

### FIELD OF THE INVENTION

The invention relates to methods for detecting and inhibiting angiogenesis, cell migration, cell adhesion, and/or cell survival in endothelial and non-endothelial cells as well as in normal and tumor cells. The invention further relates to methods for screening test compounds for their ability to inhibit angiogenesis, cell migration, cell adhesion, and/or cell survival.

### BACKGROUND OF THE INVENTION

The movement of cells in vivo controls embryonic development, angiogenesis, tumor metastasis, the immune system response and numerous other normal and abnormal physiological events. Multiple cell surface receptors and signal transduction pathways regulate cell migration.

Angiogenesis, for example, is essential in the female reproduction system and during development and wound repair. However, inappropriate angiogenesis can have severe consequences. Indeed, the proliferation of new blood vessels from pre-existing capillaries plays a key role in diseases, such as the pathological development of solid tumor cancers, solid tumor metastases, angiofibromas, skin cancer, retrolental fibroplasia, Kaposi's sarcoma, childhood hemangiomas, diabetic retinopathy, neovascular glaucoma, age related macular degeneration, psoriasis, gingivitis, rheumatoid arthritis, osteoarthritis, ulcerative colitis, Crohn's disease, inflammatory bowel disease, and capillary proliferation in atherosclerotic plaques. Because these serious diseases afflict several million people in the United States each year, considerable scientific effort has been directed toward gaining an understanding of the mechanisms regulating angiogenesis and toward developing therapies for such diseases.

With respect to cancer, over six hundred thousand new cases of lung, colon, breast and prostate cancer will be diagnosed in the United States each year, accounting for 75% of new solid tumor cancers and 77% of solid tumor cancer deaths. Although advances in therapy and in our understanding of cancer causes and risk factors have lead to improved outcomes overall, most cancers still have low five year survival rates. Despite these advances in primary tumor management, 50% of patients will ultimately die of their disease largely due to side effects of current therapies or to the metastatic spread of tumors to numerous or inoperable sites through the tumor associated vasculature. It is now known that the growth and spread of solid tumor cancer depends on the development of a tumor-associated vasculature by a process known as angiogenesis.

One of the most significant consequences of tumor angiogenesis is the invasion of tumor cells into the vasculature. Thus, vascularization permits the survival and growth of primary tumors, as well as the metastatic spread of cancer. Metastases arise from tumor cells which enter the tumor's own vasculature to be carried to local and distant sites where they create new tumors. Tumors have typically established a vasculature and metastasized to local and distant sites by the time that primary tumors are detectable.

Current treatments for cancer rely mainly on treatments which are not selective for the disease but which have deleterious effects on other organs of the body. For example, chemotherapeutics reagents or radiation have serious side effects because they kill or impair all proliferating cells in the body, including healthy cells. Side effects are unpleasant and often create health problems that themselves increase patient mortality.

Angiogenesis also plays a major role in the progression and exacerbation of a number of inflammatory diseases. Psoriasis, a disease which afflicts 2 million Americans, is characterized by significant angiogenesis. In rheumatoid arthritis and possibly osteoarthritis, the influx of lymphocytes into joints induces blood vessels of the joint synovial lining to undergo angiogenesis; this angiogenesis appears to permit a greater influx of leukocytes and the destruction of cartilage and bone. Angiogenesis may also play a role in chronic inflammatory diseases such as ulcerative colitis and Crohn's disease. In addition, the growth of capillaries into atherosclerotic plaques is a serious problem; the rupture and hemorrhage of vascularized plaques is thought to cause coronary thrombosis. To date, however, no effective therapies exist for these diseases.

Angiogenesis is also a factor in many ophthalmic disorders which can lead to blindness. In age-related macular degeneration (ARMD), a disorder afflicting 25% of otherwise healthy individuals over the age of 60, and in diabetic retinopathy, a condition prevalent among both juvenile and late onset diabetics, angiogenesis is induced by hypoxic conditions on the choroid or the retina, respectively. Hypoxia induces an increase in the secretion of growth factors including vascular endothelial growth factor (VEGF). VEGF expression in the eye may induce the migration and proliferation of endothelial cells into regions of the eye where they are not ordinarily found. Vascularization in ocular tissue has adverse effects on vision. New blood vessels on the cornea can induce corneal scarring, whereas new blood vessels on the retina can induce retinal detachment, and angiogenic vessels in the choroid may leak vision-obscuring fluids; these events often lead to blindness.

For other pathological conditions associated with abnormal angiogenesis such as diabetic retinopathy, there are no effective treatments short of retinal transplants. However, even if retinal transplantation is performed, the new retina would be subject to the same conditions that resulted in the original retinopathy.

Furthermore, there exist several pathological conditions in which undesirable cell migration, cell adhesion and/or cell survival are implicated. While agents which prevent angiogenesis, cell migration, cell adhesion, and/or cell survival are currently being tested, there remains a need to identify the molecular interactions involved in these phenomena when they attain undesirable levels in certain pathological conditions, and to develop methods and compositions for diagnosing and specifically treating such pathologies.

### SUMMARY OF THE INVENTION

The invention relates to methods for detecting and inhibiting angiogenesis, cell migration, cell adhesion, and/or cell survival in endothelial and non-endothelial cells as well as in normal and tumor cells. The invention further relates to methods for screening test compounds for their ability to inhibit angiogenesis, cell migration, cell adhesion, and/or cell survival.

In particular, the invention provides a method for reducing at least one of cell migration, cell survival, cell adhesion, and angiogenesis, comprising: a) providing: i) at least one cell; and ii) at least one nucleotide sequence encoding a protein comprising a protein kinase A catalytic subunit; and b) expressing said nucleotide sequence in said at least one cell such that at least one of migration of said cell, survival of said cell, adhesion by said cell, and angiogenesis by said cell is reduced. In one embodiment, the method further comprises step c) detecting a reduction in at least one of migration of said cell, survival of said cell, adhesion by said cell, and angiogenesis by said cell. In another embodiment, the cell is chosen from endothelial cell, vascular smooth muscle cell, monocyte cell, macrophage cell, benign tumor cell, malignant tumor cell, fibroblast cell, B cell, T cell, myocyte cell, megakaryocyte cell, eosinophil cell, neurite cell, and synoviocyte cell. In a further embodiment, the cell is an endothelial cell. In yet another embodiment, expression of said nucleotide sequence in said endothelial cell results in reduced angiogenesis by said endothelial cell. In an alternative embodiment, the cell is in a tissue, and said tissue is in a subject, such as a human. In another embodiment, the cell is an endothelial cell, and said tissue comprises at least one of ocular tissue, skin tissue, bone tissue, and synovial tissue. In another embodiment, the tissue comprises a tumor, such as a malignant tumor, and preferably the malignant tumor is metastatic. In a further embodiment, the subject has a pathological condition associated with angiogenesis in said tissue. In another embodiment, the subject has a pathological condition chosen from angiogenesis, restenosis, atherosclerosis, cancer, tumor metastasis, fibrosis, hemangioma, lymphoma, leukemia, psoriasis, arthritis, autoimmune disease, diabetes, amyotrophic lateral sclerosis, graft rejection, retinopathy, macular degeneration, and retinal tearing. Alternatively, the pathological condition is fibrosis and said tissue is chosen from heart, lung, and liver. In a futher embodiment, the pathological condition is an autoimmune disease chosen from Lupus, Crohn's disease, and multiple sclerosis.

The invention also provides a method for reducing at least one of cell migration, cell survival, cell adhesion, and angiogenesis, comprising: a) providing: i) at least one cell; and ii) at least one polypeptide sequence comprising a sequence chosen from at least one of AVSEHQLLHS/D (SEQ D NO:114) and SVSEIQLMNL (SEQ ID NO:115); and b) treating said at least one cell with said polypeptide sequence such that at least one of migration of said cell, survival of said cell, adhesion by said cell, and angiogenesis by said cell is reduced. In one embodiment, the method further comprises step c) detecting a reduction in at least one of migration of said cell, survival of said cell, adhesion by said cell, and angiogenesis by said cell.

Also provided is a method for reducing at least one of cell migration, cell survival, cell adhesion, and angiogenesis, comprising: a) providing: i) at least one cell that is not an endothelial cell; and ii) at least one antibody specific for an integrin expressed by said at least one cell; and b) treating said at least one cell with said at least one antibody such that at least one of migration of said cell, adhesion by said cell, and survival of said cell is reduced. In one embodiment, the method further comprises step c) detecting a reduction in at least one of migration of said cell, adhesion by said cell, and survival of said cell. In an alternative embodiment, the antibody reduces specific binding of said integrin to at least one ligand of said integrin. In a further embodiment, the integrin is chosen from alpha v beta 1, alpha v beta 3, alpha v beta 5, alpha v beta 6, alpha v beta 8, alpha 1 beta 1, alpha 2 beta 1, alpha 3 beta 1, alpha 4 beta 1, alpha 5 beta 1, alpha 6 beta 1, alpha 7 beta 1, alpha 8 beta 1, alpha 9 beta 1, alpha 10 beta 1, alpha 6 beta 4, alpha 4 beta 7, alpha M beta 2, alpha L beta 2, and alpha X beta 2.

The invention additionally provides a method for reducing at least one of endothelial cell migration, endothelial cell survival, endothelial cell adhesion, and angiogenesis by an endothelial cell, comprising: a) providing: i) at least one endothelial cell; and ii) at least one antibody specific for an integrin expressed by said at least one endothelial cell; and b) treating said at least one endothelial cell with said at least one antibody such that at least one of endothelial cell migration, adhesion by said endothelial cell, endothelial cell survival, and angiogenesis by said endothelial cell is reduced. In a further embodiment, the method comprises step c) detecting a reduction in at least one of migration of said cell, survival of said cell, adhesion by said cell, and angiogenesis by said cell. In another embodiment, the antibody reduces specific binding of said integrin to at least one ligand of said integrin. In one embodiment, the integrin is not alpha 5 beta 1. In a further embodiment, the integrin is chosen from alpha v beta 1, alpha v beta 3, alpha v beta 5, alpha v beta 6, alpha v beta 8, alpha 1 beta 1, alpha 2 beta 1, alpha 3 beta 1, alpha 4 beta 1, alpha 6 beta 1, alpha 7 beta 1, alpha 8 beta 1, alpha 9 beta 1, alpha 10 beta 1, alpha 6 beta 4, alpha 4 beta 7, alpha M beta 2, alpha L beta 2, alpha X beta 2.

Also provided is a method for reducing at least one of cell migration, cell survival, cell adhesion, and angiogenesis, comprising: a) providing: i) at least one cell; and ii) at least one agent chosen from pertussis toxin, cholera toxin, G alpha i minigene, dominant negative G alpha i, dominant negative G alpha 12/13, constitutively active G alpha s, anti-CD47 antibody, dominant positive Rho (RhoV14), dominant negative Src, and active Csk; and b) treating said at least one cell with said at least one agent such that at least one of cell migration, cell survival, cell adhesions, and angiogenesis by said cell is reduced. In one embodiment, the method further comprises step c) detecting a reduction in at least one of migration of said cell, survival of said cell, adhesion by said cell, and angiogenesis by said cell.

Also provided by the invention is a method for reducing at least one of cell migration, cell survival, cell adhesion, and angiogenesis, comprising: a) providing: i) at least one cell; and ii) at least one Src inhibitor; and b) treating said at least one cell with said at least one Src inhibitor such that at least one of cell migration, cell survival, cell adhesion, and angiogenesis by said cell is reduced. In one embodiment, the method further comprises step c) detecting a reduction in at least one of migration of said cell, survival of said cell, adhesion by said cell, and angiogenesis by said cell.

In particular, the present invention provides a method of inhibiting cell migration, comprising: providing: a cell; and a nucleotide sequence encoding a protein kinase A catalytic subunit; and expressing the nucleotide sequence in the cell such that migration of the cell is inhibited. In some of these embodiments, the cell is selected from the group of endothelial cell, vascular smooth muscle cell, monocyte cell, macrophage cell, benign tumor cell, malignant tumor cell, fibroblast cell, B cell, T cell, myocyte cell, megakaryocyte cell, eosinophil cell, neurite cell, and synoviocyte cell. In further embodiments, the cell is in a tissue, and the tissue is in a subject (e.g., a human). In some embodiments of the present invention, the subject has angiogenesis in the tissue, alternatively, in other embodiments, the subject is suspected of being capable of developing angiogenesis in the tissue.

In some embodiments, the present invention provides methods of treating endothelial cells (e.g., vascular cells, smooth muscle cells, and the like), and the tissues, include, but are not limited to, ocular tissue (e.g., the ocular tissue is selected from retina, macula, cornea, choroids, and vitreous humor), skin tissue, bone tissue, or synovial tissue.

In some embodiments, the treated tissue comprises a tumor (e.g., metastatic, begin, malignant). In preferred embodiments, malignant tumors, include, but are not limited to, carcinoma, sarcoma, glioblastoma, astrocytoma, neuroblastoma, and retinoblastoma). In still further embodiments, the malignant tumor is metastatic. In yet other embodiments, malignant tumors contemplated for treatment with the methods and compositions of the present invention include, but are not limited to, gastric cancer, head cancer, neck cancer, lung cancer, breast cancer, prostate cancer, cervical cancer, pancreatic cancer, colon cancer, ovarian cancer, stomach cancer, esophagus cancer, mouth cancer, tongue cancer, gum cancer, skin cancer, muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, gall bladder cancer, ocular cancer, joint cancer, glioblastoma, mesothelioma, lymphoma, leukemia, melanoma, squamous cell carcinoma, osteosarcoma, and Kaposi's sarcoma.

In still some other embodiments, the subject has a pathological condition associated with angiogenesis in the tissue. The present invention is not intended to be limited to treating any particular tissue in a subject. For example, the present invention provides methods and compositions for treating tissues, including, but not limited to, ocular tissue (e.g., retina, macula, cornea, choroids, and vitreous humor), skin tissue, bone tissue, or synovial tissue.

In yet other embodiments, the subject has a pathological condition selected from the group of angiogenesis, restenosis, atherosclerosis, cancer, tumor metastasis, fibrosis (e.g., hear, liver, lung, and the like), hemangioma, lymphoma, leukemia, psoriasis, arthritis, autoimmune disease, diabetes, amyotrophic lateral sclerosis, graft rejection, retinopathy, macular degeneration, and retinal tearing.

In some embodiments, the subject has an autoimmune disease, such as, Lupus, Crohn's disease, and multiple sclerosis, and the like. In other embodiments, the subject has a pathological condition, including, but not limited to, restenosis, atherosclerosis, fibrosis, hemangioma, lymphoma (e.g., B cell lymphoma), leukemia (e.g., B cell leukemia), psoriasis, arthritis, amyotrophic lateral sclerosis, graft rejection, retinopathy, macular degeneration, and retinal tearing, rheumatoid arthritis and osteoarthritis, and the like). The present invention specifically contemplates therapeutic methods and compositions for treating any of the aforementioned disease/conditions, as well as other related disease/conditions within the scope of the present invention in any particular cells or tissues where these conditions/disease occur.

In still further embodiments, the present invention provides methods of inhibiting cell migration, comprising: providing: a cell; and a polypeptide sequence comprising the 10 amino acids at the N-terminal end of a peptide selected from the group of parathyroid hormone and parathyroid hormone related peptide; and treating the cell with the polypeptide sequence such that migration of the cell is inhibited. In one embodiment, the 10 amino acids at the N-terminal end of parathyroid hormone related peptide is AVSEHQLLHS/D (i.e., wherein the amino acid at position 10 is chosen from S and D) (SEQ ID NO:114). In another embodiment, the 10 amino acids at the N-terminal end of parathyroid hormone is SVSEIQLMNL (SEQ ID NO:115).

Alternatively, the present invention also provides methods of inhibiting cell migration, comprising: providing: an endothelial cell; and an antibody specific for an integrin (e.g., alpha v beta 1, alpha v beta 3, alpha v beta 5, alpha v beta 6, alpha v beta 8, alpha 1 beta 1, alpha 2 beta 1, alpha 3 beta 1, alpha 4 beta 1, alpha 6 beta 1, alpha 7 beta 1, alpha 8 beta 1, alpha 9 beta 1, alpha 10 beta 1, alpha 6 beta 4, alpha 4 beta 7, alpha M beta 2, alpha L beta 2, alpha X beta 2, and alpha IIb beta 3, and the like); and treating the endothelial cell with the antibody such that migration of the endothelial cell is inhibited. In some embodiments, the antibody inhibits specific binding of the integrin to a ligand of the integrin.

Still further embodiments of the present invention provide methods of inhibiting cell migration, comprising: providing: a cell, wherein the cell is not an endothelial cell; and an antibody specific for an integrin (e.g., alpha v beta 1, alpha v beta 3, alpha v beta 5, alpha v beta 6, alpha v beta 8, alpha 1 beta 1, alpha 2 beta 1, alpha 3 beta 1, alpha 4 beta 1, alpha 5 beta 1, alpha 6 beta 1, alpha 7 beta 1, alpha 8 beta 1, alpha 9 beta 1, alpha 10 beta 1, alpha 6 beta 4, alpha 4 beta 7, alpha M beta 2, alpha L beta 2, alpha X beta 2, and alpha IIb beta 3, and the like); and treating the cell with the antibody such that migration of the cell is inhibited. In some embodiments, the antibody inhibits specific binding of the integrin to a ligand of the integrin.

The present invention is not limited, however, to providing antibodies that bind to integrins alpha v beta 1, alpha v beta 3, alpha v beta 5, alpha v beta 6, alpha v beta 8, alpha 1 beta 1, alpha 2 beta 1, alpha 3 beta 1, alpha 4 beta 1, alpha 5 beta 1, alpha 6 beta 1, alpha 7 beta 1, alpha 8 beta 1, alpha 9 beta 1, alpha 10 beta 1, alpha 6 beta 4, alpha 4 beta 7, alpha M beta 2, alpha L beta 2, alpha X beta 2, and alpha IIb beta 3. Indeed, the compositions and methods of the present invention find use with antibodies that bind to a number of other integrins known to those skilled in the art.

Some embodiments of the present invention provide methods of inhibiting cell migration, comprising: providing: a cell; and an agent (e.g., pertussis toxin, cholera toxin, G alpha i minigene, dominant negative G alpha i, dominant negative G alpha 12/13, constitutively active G alpha s, anti-CD47 antibody, dominant positive Rho (RhoV14), dominant negative Src, and active Csk, and the like); and treating the cell with the agent such that migration of the cell is inhibited.

Still further embodiments of the present invention provide, methods of inhibiting cell survival, comprising: providing: a cell; and a nucleotide sequence encoding a protein kinase A catalytic subunit; and expressing the nucleotide sequence in the cell such that survival of the cell is inhibited. In some of these embodiments, the cell is an endothelial cell, vascular smooth muscle cell, monocyte cell, macrophage cell, benign tumor cell, malignant tumor cell, fibroblast cell, B cell, T cell, myocyte cell, megakaryocyte cell, eosinophil cell, neurite cell, and synoviocyte cell, and the like. In some embodiments, the cell is a tissue. In some further embodiments, the tissue sis a subject. In still some other embodiments, the tissue comprises a tumor (e.g., carcinoma, sarcoma, glioblastoma, astrocytoma, neuroblastoma, and retinoblastoma, gastric cancer, head cancer, neck cancer, lung cancer, breast cancer, prostate cancer, cervical cancer, pancreatic cancer, colon cancer, ovarian cancer, stomach cancer, esophagus cancer, mouth cancer, tongue cancer, gum cancer, skin cancer, muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, gall bladder cancer, ocular cancer, joint cancer, glioblastoma, mesothelioma, lymphoma, leukemia, melanoma, squamous cell carcinoma, osteosarcoma, and Kaposi's sarcoma, etc.).

In still some other embodiments, the subject has a pathological condition associated with angiogenesis in the tissue (e.g., ocular tissue (e.g., retina, macula, cornea, choroids, and vitreous humor) skin tissue, bone tissue, or synovial tissue).

In alternative embodiments of the present invention, the subject has a pathological condition, including, but not limited to, angiogenesis (e.g., endothelia cell and vascular smooth muscle cell), restenosis (e.g., a vascular smooth muscle cell), atherosclerosis (e.g., vascular smooth muscle cell, monocyte cell and macrophage cell), cancer, tumor metastasis, fibrosis (e.g., heart, lung, and liver), hemangioma (e.g., endothelial cell), lymphoma (e.g., B cell), leukemia (e.g., B cell), psoriasis (e.g., endothelial cell), arthritis (e.g., endothelial cell, synoviocyte cell, and fibroblast cell), autoimmune disease (e.g., Lupus, Crohn's disease, and multiple sclerosis), diabetes, amyotrophic lateral sclerosis (e.g., B cell), graft rejection (e.g., B cell), retinopathy, macular degeneration, and retinal tearing.

Other embodiments of the present invention provide methods of inhibiting cell survival, comprising: providing: a cell; and a polypeptide sequence comprising the 10 amino acids at the N-terminal end of a peptide (e.g., parathyroid hormone and parathyroid hormone related peptide); and treating the cell with the polypeptide sequence such that survival of the cell is inhibited.

Still other embodiments of the present invention provide methods of inhibiting cell survival, comprising: providing: an cell (e.g., an endothelial cell); and an antibody specific for an integrin (e.g., alpha v beta 1, alpha v beta 3, alpha v beta 5, alpha v beta 6, alpha v beta 8, alpha 1 beta 1, alpha 2 beta 1, alpha 3 beta 1, alpha 4 beta 1, alpha 6 beta 1, alpha 7 beta 1, alpha 8 beta 1, alpha 9 beta 1, alpha 10 beta 1, alpha 6 beta 4, alpha 4 beta 7, alpha M beta 2, alpha L beta 2, alpha X beta 2, and alpha IIb beta 3, and the like); and treating the endothelial cell with the antibody such that survival of the endothelial cell is inhibited.

Additional embodiments provide methods of inhibiting cell survival, comprising: providing: a cell (e.g., endothelial cell); and an agent (e.g., pertussis toxin, cholera toxin, G alpha i minigene, dominant negative G alpha i, dominant negative G alpha 12/13, constitutively active G alpha s, anti-CD47 antibody, dominant positive Rho (RhoV14), dominant negative Src, and active Csk, and the like); and treating the cell with the agent such that survival of the cell is inhibited.

Yet other additional embodiments of the present invention provide methods of inhibiting angiogenesis in a tissue, comprising: providing: a tissue; and a nucleotide sequence encoding a protein kinase A catalytic subunit; and expressing the nucleotide sequence in the cell such that angiogenesis in the tissue is inhibited.

Methods of inhibiting angiogenesis, comprising: providing: a tissue; and a polypeptide sequence comprising the 10 amino acids at the N-terminal end of a peptide, such as, parathyroid hormone and parathyroid hormone related peptide; and treating the tissue with the polypeptide sequence such that angiogenesis in the tissue is inhibited.

In some embodiments, the present invention provides methods of inhibiting angiogenesis, comprising: providing: a tissue; and an antibody specific for an integrin (e.g., alpha v beta 1, alpha v beta 3, alpha v beta 5, alpha v beta 6, alpha v beta 8, alpha 1 beta 1, alpha 2 beta 1, alpha 3 beta 1, alpha 4 beta 1, alpha 6 beta 1, alpha 7 beta 1, alpha 8 beta 1, alpha 9 beta 1, alpha 10 beta 1, alpha 6 beta 4, alpha 4 beta 7, alpha M beta 2, alpha L beta 2, alpha X beta 2, and alpha IIb beta 3, and the like); and treating the endothelial cell with the antibody such that survival of the endothelial cell is inhibited.

Additionally, the present invention provides methods of inhibiting angiogenesis, comprising: providing: a tissue; and an agent (e.g., pertussis toxin, cholera toxin, G alpha i minigene, dominant negative G alpha i, dominant negative G alpha 12/13, constitutively active G alpha s, anti-CD47 antibody, dominant positive Rho (RhoV14), dominant negative Src, and active Csk, and the like); and treating the tissue with the agent such that angiogenesis in the tissue is inhibited.

The present invention also provides methods for inhibiting angiogenesis, and/or cell migration in a tissue, comprising: providing: a tissue; an agent that activates protein kinase A; treating the tissue with the agent under conditions such that protein kinase A is activated. In some of these embodiments, the agent activates the protein kinase A by inhibiting integrin ligation (e.g., α5β1 integrin). In still some other embodiments, the agent is an anti-integrin α5β1 antibody. In some other embodiments, the agent is a cell permeable cAMP (e.g., dibutyryl cAMP). In yet other embodiments, agent comprises a peptide (e.g., catalytic subunit of protein kinase A, PTH, PTHrP).

It is understood that any of the methods of the present invention may optionally comprise an additional step of observing the effects of the agent(s) of interest on cells/tissues.

In still further embodiments, the present invention provides methods of inhibiting cell migration, comprising: providing: cells; an agent that activates protein kinase A and inhibits integrin α5β1 ligation; and treating the cells with the agent under conditions such that protein kinase A is activated. Further embodiments, of the present invention provide methods of inhibiting angiogenesis in a subject, comprising: providing: a subject comprising a tissue; and an agent that activates protein kinase A; and administering the agent to the subject under conditions such that protein kinase A is activated.

In some embodiments, the present invention provides therapeutic methods of treating malignant tumors, including, but not limited to, lung cancer, breast cancer, prostate cancer, cervical cancer, pancreatic cancer, colon cancer, ovarian cancer, stomach cancer, esophagus cancer, mouth cancer, tongue cancer, gum cancer, skin cancer, muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer, joint cancer, glioblastoma, lymphoma, leukemia, osteosarcoma, Kaposi's sarcoma, and the like.

Still further embodiments provide methods for screening a test compound, comprising: providing: cells; a test compound suspected of activating protein kinase A; and treating the cells with the test compound to produce treated cells; detecting activation of protein kinase A in the cells; and identifying the test compound as inhibiting angiogenesis and/or cell migration.

In some embodiments, the present invention further provides kits for inhibiting angiogenesis and/or cell migration in a tissue, comprising: providing: a tissue; an agent that activates protein kinase A; and instructions for using the kit for inhibiting angiogenesis and/or cell migration in the tissue.

In yet another embodiment, the invention provides a method for reducing at least one of cell migration, cell survival, cell adhesion, and angiogenesis, comprising: a) providing: i) at least one cell; and ii) at least one Src inhibitor; and b) treating said at least one cell with said at least one Src inhibitor such that at least one of cell migration, cell survival, adhesion by said cell, and angiogenesis by said cell is reduced. In one embodiment, the method further comprises step c) detecting a reduction in at least one of migration of said cell, survival of said cell, adhesion by said cell, and angiogenesis by said cell. In a further embodiment, the Src inhibitor is chosen from 4-Amino-1-tert-butyl-3-(1′-naphthyl)pyrazolo[3,4-d]pyrimidine (PP1), 4-Amino-5-(4-chlorophenyl)-7-(t-butyl)pyrazolo[3,4-d]pyrimidine (PP2), (Z)-3-[4-(Dimethylamino)benzylidenyl]indolin-2-one, a-Cyano-(3,4-dihydroxy)cinnamoyl-(3′,4′-dihydroxyphenyl)ketone, 5-Amino-[(N-2,5-dihydroxybenzyl)-N′-2-hydroxybenzyl]salicylic Acid, 2′,4′,3,4-Tetrahydroxychalcone, 3-Hydroxy-1-methoxyanthraquinone-2-aldehyde, 5-Amino-[(N-2,5-dihydroxybenzyl)-N′-2-hydroxybenzyl]salicylic Acid, (Z)-5-Bromo-3-(4,5,6,7-tetrahydro-1H-indol-2-ylmethylene)-1,3-dihydroindol-2-one, 2′,4′3,4-Tetrahydoxychalcone, 3-Hydroxy-1-methoxyanthraquinone-2-aldehyde, 4-(4′-Phenoxyanilino)-6,7-dimethoxyquinazoline, Herbimycin A, Streptomyces sp., Lavendustin A, Radicicol, Diheterospora chlamydosporia, and p60v-src 137-157 Inhibitor Peptide (VAPSDSIQAEEWYFGKITRRE) (SEQ ID NO:116).

### DESCRIPTION OF THE DRAWINGS

The following figures form part of the specification and are included to further demonstrate certain aspects and embodiments of the present invention. The present invention is not intended to be limited however to the embodiments specifically recited in these figures.

FIG. 1. Unligated integrins suppress cell migration. (A) Immunolocalization of integrin αvβ3, integrin α5β1, fibronectin and major histocompatibility complex antigen in proliferating human umbilical vein endothelial cells (“HUVECs”) cultured on vitronectin coated culture plates for 4 hours. Arrowheads indicate localization of proteins in focal adhesions. (B) Immunoblotting for fibronectin expression in lysates of endothelial cell (HUVECs) and TE24 epidermal carcinoma cells. Endothelial cell migration (C) adhesion (D) on vitronectin in the presence of basal endothelial culture medium or 25 μg/ml anti-α5β1, anti-αvβ3 and anti-α2β1 antibodies. (E) Endothelial cell migration on collagen in the presence of medium, anti-α5β1, anti-αvβ3, or anti-α2β1 antibodies. (F) Rhodamine-phalloidin staining of filamentous actin in endothelial cells attached to vitronectin in the absence (medium) or presence of anti-integrin α5β1 or α2β1 antibodies. Arrowheads indicate actin filaments. Asterisks indicate results that are significantly different from control treatments (P<0.05) as determined by Student's t-test.

FIG. 2. PKA is negatively regulated by integrin ligation. (A) Time course of PKA activation in endothelial cells placed in suspension (suspended) or maintained in monolayers (attached). (B) Time course of PKA activation in endothelial cells maintained in suspension for 30 minutes (t=−30), then retained in suspension (suspended) or allowed to re-attach to vitronectin coated culture plates (re-attached). (C) PKA activity in endothelial cells treated with or without dibutyryl cAMP. (D) ECs were maintained in suspension for 60 minutes (sus) or allowed to attach for 60 minutes to vitronectin, fibronectin, or collagen-coated culture plates in the absence (med) or presence of antibody antagonists of integrins α5β1, α2β1 and αvβ3.

FIG. 3. Unligated integrins suppress cell migration in a PKA dependent manner. (A-B) Migration of endothelial cells (A) transfected with a control transgene, N1GFP (black bars), or co-transfected with pcDNA 3.1 V5/his-dnPKA and N1GFP (gray bars), on vitronectin in the presence of 25 μg/ml anti-α5β1, anti-α2β1 or anti-αvβ3 antibodies. Transfected cells were identified by green fluorescence. (B) Migration of endothelial cells transfected with pcDNA 3.1 V5/his-dnPKA and N1GFP (stippled bars) or N1GFP alone (black bars) on vitronectin in the presence of 25 μg/ml anti-αvβ3, anti-αvβ5, anti-α2β1 or anti-αvβ3 in combination with anti-αvβ5. Transfected cells were identified by green fluorescence. (C) PKA activity cells in transfected with dnPKA or GFP and treated with 250 μM dibuytyrl cAMP for 30 minutes. (D) Expression of dnPKA and GFP transgenes in endothelial cells and COS cells was detected by Western blotting for the V5 tag and GFP. (E) Endothelial cells transfected with N1GFP or with pcDNA 3.1 V5/his-dnPKA and N1GFP attached to vitronectin-coated coverslips for 60 minutes were stained with rhodamine-phalloidin. Transfected cells were identified by green fluorescence. Arrowheads indicate stress fibers. Asterisks indicate results that are significantly different from control treatments (P<0.05) as determined by Student's t-test.

FIG. 4. Activated PKA directly inhibits cell migration See also online supplementary movies FIG. 4. Mov.1 (−cAMP) and FIG. 4. Mov.2 (+cAMP). (A) Freeze frame selections of time lapse video microscopy of cell migration without (left panel) and with (right panel) activation of PKA with dibutyryl cAMP, shown at 20 minute intervals. Arrowheads mark the location of nuclei at time 0. (B) Average distance traveled by nuclei over 40 minutes. (C) Spindle shaped cells (upper left, arrowhead) and membrane ruffling (upper right, arrowhead) are observed in untreated cells (−cAMP) while flattened cells and no ruffling are observed in cAMP treated cells (+cAMP) 40 minutes after cAMP addition. (D) Average cell polarity before and after cAMP treatment. (E) Rhodamine phalloidin and anti-vinculin staining before and after cAMP treatment. (F) Average number of focal adhesions+/−S.E.M. per cell (n=20) before and after cAMP treatment. Asterisks indicate results that are significantly different from control treatments (P<0.05) as determined by Student's t-test.

FIG. 5. Activated PKA inhibits cell migration and stimulates cell adhesion. (A) Migration on vitronectin of endothelial cells transfected with pcDNA 3.1 V5/his-dnPKA and N1GFP (gray bars) or N1GFP (black bars) vectors in the absence or presence of 250 μM dibutyryl cAMP. (B) Migration of endothelial cells transfected with pcDNA 3.1 V5/his-PKA catalytic subunit and N1GFP or N1GFP vectors alone on vitronectin. (C) Expression of transgenes in endothelial cells was detected by Western blotting for the V5tag on PKAcat and for GFP. (D) Attachment to vitronectin of endothelial cells treated with cAMP or basal culture medium. (E) Attachment to vitronectin of endothelial cells transfected with pcDNA 3.1 V5/his-PKA catalytic subunit and N1GFP (PKACat) or N1GFP (GFP) vectors. (F) Endothelial cells treated with (+cAMP) or without dibutyryl cAMP (−cAMP) were allowed to attach to vitronectin-coated coverslips for 60 minutes and were stained with rhodamine-phalloidin. Endothelial cell transfected with N1GFP (GFP) or pcDNA 3.1 V5/his-PKAcat and N1GFP (PKAcat) were allowed to attach to vitronectin-coated coverslips for 60 minutes and were stained with rhodamine-phalloidin. Transfected cells were identified by green fluorescence. Arrowheads indicate well-formed stress fibers. Asterisks indicate results that are significantly different from control treatments (P<0.05) as determined by Student's t-test.

FIG. 6. Unligated integrins activate the small GTPase Rho. (A) Affinity precipitated RhoGTP and total Rho levels were measured in endothelial cells attached to vitronectin for 0-90 minutes in the presence of culture medium. The ratio of active Rho (RhoGTP) to total Rho was determined and graphed versus time. (B) Affinity precipitated RhoGTP and total Rho levels were measured in endothelial cells attached to vitronectin for 0-30 minutes in the presence of culture medium, anti-α5β1 and anti-α2β1. The ratio of active Rho (RhoGTP) to total Rho was determined and graphed versus time. (C) Rhodamine-Phalloidin staining for F-actin in V14Rho, N19Rho or N1GFP (GFP) transfected endothelial cells attached to vitronectin for one hour in the absence or presence of anti-integrin α5β1 or α2β1. Transfected cells were identified by green fluorescence. Arrowheads indicate stress fibers. (D) Expression of GFP and mutated Rho proteins were detected by Western blotting of detergent extracts of transfected cells with anti-GFP or -HA tag antibodies. (E) The migration of endothelial cells transfected with N1GFP (GFP) or with HA-tagged pcDNA 3.1 expression vectors encoding N19Rho or V14Rho with N1GFP on vitronectin in the presence or absence of anti-α5β1. Transfected cells were identified by green fluorescence. (F) Anti-vinculin staining to detect focal adhesions in GFP and V14Rho transfected cells. (Left) Micrographs of GFO and V14Rho transfected cells. Focal adhesions are indicated by arrowheads. (Right) Average number of focal adhesions per cell (n=20). Asterisks indicate results that are significantly different from control treatments (P<0.05) as determined by Student's t-test.

FIG. 7. PKA activates Rho, thereby inhibiting stress fiber formation and cell migration. (A) Affinity precipitated RhoGTP and total Rho levels were measured in endothelial cells treated with medium or 250 μM dibutyryl cAMP that were allowed to attached to vitronectin for 0 or 15 minutes. The ratio of active Rho (RhoGTP) to total Rho was determined. (B) Affinity precipitated RhoGTP and total Rho levels were measured in endothelial cells transfected with N1GFP (GFP) or PKAcat (PKAcat) that were allowed to attached to vitronectin for 0 or 15 minutes. The ratio of active Rho (RhoGTP) to total Rho was determined. (C) Migration on vitronectin of GFP or N19Rho transfected cells that were treated with medium or 250 μM dibutyryl cAMP or co-transfected with PKAcat. Transfected cells were identified by green fluorescence. (D) Phalloidin staining for F-actin in endothelial cells transfected with N1GFP (GFP), PKAcat (PKAcat) or N19Rho and PKAcat (N19Rho+PKAcat) after one hour on vitronectin. Transfected cells were identified by green fluorescence. Arrowheads indicate stress fibers. (E) Immunolocalization of Rho A in cAMP treated and untreated cells. Arrowheads indicate plasma membrane. Asterisks indicate results that are significantly different from control treatments (P<0.05) as determined by Student's t-test.

FIG. 8. Unligated integrins inhibit the small GTPase Rac. (A) Affinity precipitated RacGTP and total Rac levels were measured in endothelial cells attached to vitronectin for 0-30 minutes in the presence of culture medium anti-αβ1 and anti-α2β1. The ratio of active Rac (RacGTP) to total Rac was determined and graphed versus time. (B) Affinity precipitated RacGTP and total Rac levels were measured in endothelial cells treated with 25 μg/ml anti-α2β1 or anti-α5β1 and allowed to attached to vitronectin for 30 minutes. The ratio of active Rac (RacGTP) to total Rac was determined. (C) The migration of HUVECs cells transfected with N1GFP (GFP) or with HA-tagged pcDNA 3.1 expression vectors encoding N17Rac or V12Rac with N1GFP on vitronectin in the presence or absence of anti-α5β1. Transfected cells were identified by green fluorescence. (D) Rhodamine-Phalloidin staining of pcDNA 3.1 V12Rac and N1GFP (RacV12) or N1GFP (GFP) transfected endothelial cells attached to vitronectin in the absence or presence of anti-integrin α5β1 or α2β1. Transfected cells were identified by green fluorescence. Arrowheads indicate stress fibers. (E) Expression levels of GFP and mutated Rac proteins was detected by Western blotting of detergent extracts of transfected cells with anti-GFP or -HA tag antibodies. (F) Cell migration on vitronectin of endothelial cells transfected with GFP or with GFP and mutationally inactive (dnPAK) or constitutively active p21 kinase (dpPAK). (G) Cell migration on vitronectin of endothelial cells transfected with GFP (black bars) or (gray bars) with GFP and constitutively active p21 kinase (dpPAK) in the absence or presence of cAMP, anti-α5β1 or anti-α2β1. Asterisks indicate results that are significantly different from control treatments (P<0.05) as determined by Student's t-test.

FIG. 9. PKA inhibits Rac, thereby inhibiting cell migration. (A) Affinity precipitated RacGTP and total Rac levels were measured in endothelial cells treated with medium or 250 μM dibutyryl cAMP or transfected with N1GFP or PKAcat and allowed to attached to vitronectin for 30 minutes. The ratio of active Rac (RhoGTP) to total Rac was determined. (B) Migration on vitronectin of GFP or V12Rac transfected endothelial cells that were treated with medium or 250 μM dibutyryl cAMP or co-transfected with PKAcat. Transfected cells were identified by green fluorescence. (C) Rhodamine-Phalloidin staining for F-actin in endothelial cells transfected with N1GFP (GFP), PKAcat (PKAcat), V12Rac and PKAcat (V12Rac+PKAcat) and N17Rac (N17Rac). Transfected cells were identified by green fluorescence. Arrowheads indicate stress fibers. (D) Affinity precipitated RacGTP and total Rac levels were measured in endothelial cells transfected with N1GFP or V14Rho and allowed to attached to vitronectin for 30 minutes. The ratio of active Rac (RacGTP) to total Rac was determined. Asterisks indicate statistically significant results (P<0.05) as determined by Student's t-test.

FIG. 10. Fibronectin and integrin α5β1 support endothelial cell survival. (A-C) HUVECs were maintained in suspension (SUS) or on fibronectin (Fn) or poly-L-lysine (PLL)-coated plates. (A) The percentage of annexin V positive cells on PLL or FN coated dishes was determined at regular intervals from 0-8 h. (B) Cell lysates prepared after 4 h of attachment were immunoblotted to detect intact (116 kDa) and cleaved PARP (85 kDa). The ratio of intact to cleaved PARP was determined by densitometry. (C) Soluble DNA extracted from cells attached to PLL or fibronectin was electrophoresed on 1.6% agarose gels. Relative DNA cleavage was determined by densitometry. (D-F) HUVECs were plated on fibronectin, anti-α6β1 or control antibody-coated plates. (D) The percentage of annexin V positive cells was determined from 0-8 h. (E) Cell lysates were immunoblotted to detect intact and cleaved PARP. (F) DNA fragmentation was evaluated as in C.

FIG. 11. Integrin α5β1 supports endothelial cell survival during angiogenesis in vivo. (A) CAMs were stimulated with bFGF or saline and then treated for 24 h with saline, anti-α5β1, and control antibodies. CAMs were then injected with 50 μl FITC-Annexin V, harvested 2 h later and analyzed directly by confocal microscopy. (B) Green pixels (annexin V+) present per optical section were quantified. (C) CAMs treated as in A were cryosectioned and immunostained with anti-cleaved caspase 3 (green) and anti-vWF (red). Cleaved caspase 3 positive blood vessels are yellow (arrows). (D) CAMs treated as in A were cryosectioned and immunostained with anti-VWF (red) and for DNA fragmentation (TUNEL staining, green). Arrows indicate blood vessels. Yellow structures are apoptotic blood vessels. (E) Soluble DNA isolated from CAMs treated as in A was electrophoresed on 1.6% agarose gels. Molecular weight markers are 1 kb DNA ladders. Relative DNA cleavage was determined by densitometry. (F) Individual cells isolated from CAMs treated as in A were stained with FITC-Annexin V.

FIG. 12. Unligated integrin α5β1 regulates endothelial cell survival. HUVECs were plated on poly-L-lysine, fibronectin (A-B) or vitronectin (C-D) coated culture plates in culture medium (med) or culture medium containing anti-α5β1, anti-αvβ3, anti-α2β1 antibodies. After 1 h, cell attachment was determined (B, D). After 24 h, the percentage of FITC-Annexin V (A, C) positive cells was determined. (E) HUVECs plated on vitronectin-coated plates in the presence of function blocking anti-α5β1 or control antibodies were collected at regular intervals from 0-8 h and PARP cleavage assessed by Western blotting. Relative PARP cleavage levels were determined by densitometry.

FIG. 13. Unligated integrin α5β1 induces caspase 3 and 8, but not 9, activation. HUVECs were plated on (A) fibronectin, (B) vitronectin or (A-B) poly-L-lysine (PLL) coated culture plates in the presence of anti-α5β1 antibodies and 50 μM z-DEVD-fmk (caspase-3) or z-IETD-fmk (caspase-8) inhibitors or vehicle control (0.33% DMSO) for 24 h. The percentage of annexin V positive cells was then determined. (C-D) Caspase 3 and 8 activities were determined in HUVECs plated on vitronectin (VN), or poly-L-lysine (PLL) coated plates in the presence of culture medium, anti-α5β1 or control antibodies. (E) Cell lysates were immunoblotted with anti-caspase 3 and anti-cleaved caspase 3 antibodies. Relative caspase 3 cleavage was determined by densitometry. (F) Cell lysates were immunoblotted with anti-caspase 9 and anti-cleaved caspase 9 antibodies.

FIG. 14: Integrin antagonists induce caspase 3- and 8-dependent apoptosis in vivo. (A-D) CAMs stimulated with saline or bFGF were treated with 2.5% DMSO (vehicle control), anti-α5β1, or anti-α5β1 with 500 μM caspase 3 (A-B) or caspase 8 (C-D) inhibitors. (A, C) Blood vessels branchpoints were quantified after 48 h. (B, D) Caspase 3 and 8 cleavage was evaluated by Western blotting with (B) anti-cleaved caspase 3 or (D) anti-cleaved caspase 8 antibodies and anti-actin antibodies. (E) Cryosections of CAMs treated as in A-D were immunostained for VWF expression (red) and to detect DNA fragmentation (TUNEL staining, green). Arrows indicate blood vessels. Apoptotic vessels appear yellow.

FIG. 15. Unligated α5β1 mediated death is PKA-dependent. (A) PKA activity was measured in HUVECs attached to poly-L-lysine, fibronectin or vitronectin in the presence or absence of integrin antagonists. (B) HUVECs were plated on vitronectin or poly-L-lysine (PLL) coated culture plates in the presence or absence of anti-α5β1 or anti-αvβ3 antibodies, a selective PKA inhibitor (1 μM HA1004), or anti-integrin antibodies in combination with 1 μM HA1004. After 24 h, the percentage of FITC-Annexin positive cells was determined. C. Cell lysates from B were immunoblotted with anti-caspase 3 and anti-cleaved caspase 3 antibodies. Relative caspase 3 cleavage was determined by densitometry.

FIG. 16. PKA negatively regulates cell survival. (A-B) HUVECs transfected with GFP (−) or a mutationally inactive PKA (dnPKA, +) were plated on (A) fibronectin, (B) vitronectin, or poly-L-lysine (PLL) coated plates in the absence or presence of anti-α5β1, anti-αvβ3 or anti-α2β1. After 24 h, the percentage of Annexin V positive cells was determined. (C) HUVECs treated with culture medium or dibutyryl cAMP (250 μM) and HUVECs transfected with GFP or the catalytic subunit of PKA (PKAcat) were plated on vitronectin (Vn), or poly-L-lysine (PLL) coated plates. After 24 h, the percentage of Annexin V positive cells was determined. (D) Expression of transgenes was detected by Western blotting cell lysates with anti-GFP or anti-V5.

FIG. 17. PKA inhibits angiogenesis by inducing apoptosis. (A) CAMs stimulated with bFGF were transfected 24 h later by placing 4 μg pcDNA/V5/His dnPKA or N1-GFP expression plasmid on CAMs. CAMs were treated on the same day with saline or anti-α5β1 antibodies and were harvested 48 h later. Blood vessel branch points were quantified. (B) Cryosections of CAMS from A were immunostained with anti-VWF (red) and were stained to detect fragmented DNA by the TUNEL method (green). Arrows indicate blood vessels. Apoptotic blood vessels appear yellow. (C) Western blots of lysates prepared from CAMs treated as in A were immunoblotted with anti-cleaved caspase 3 and anti-cleaved caspase 8 as well as anti-actin as a loading control. (D) CAMs stimulated with bFGF were treated with saline or 250 μM cAMP or were transfected by placing 4 μg pcDNA/V5/His PKAcat or N1-GFP expression plasmid on stimulated CAMs. Blood vessel branch points were quantified 48 hours later. (E) Cryosections of CAMS from D were immunostained with anti-VWF (red) and were stained to detect fragmented DNA by the TUNEL method (green). Arrows indicate blood vessels. Apoptotic blood vessels appear yellow. (F) Detergent lysates prepared from freshly excised CAMs from D were immunoblotted for expression of cleaved caspase 3 and actin as a loading control. (G) Cryosections of CAMS from A and D were immunostained with anti-pentaHis (red) to detect expression of His-tagged transgenes in the transfected CAM tissue. Sections were counterstained with DAPI. Arrows indicate blood vessels.

FIG. 18. PTHrP inhibits angiogenesis in vivo and endothelial cell migration in vitro. (a-b) bFGF or saline stimulated CAMs stimulated were treated with (a-b) PTHrP (1-173), calcitonin and calcitonin gene related peptide (CGRP). (a) Blood vessel branch points+/−S.E.M. above background. (b) Cryosections of CAMs were immunostained with an anti-αvβ3 (filled bars) or an anti-VWF antibody (open bars) and were stained with hematoxylin and eosin. Left panel: quantification of immunoreactive vessels per 200× microscopic field. Right panel: Representative photographs for each treatment. (c) bFGF stimulated CAMs were treated with saline, PTHrP (1-173) or PTHrP with PTHrP function blocking or control antibodies. Blood vessels branch points+/−S.E.M. above background were quantified (left panel) and representative CAMs (right panel) were photographed. (d) Mean fluorescence intensity in lysates of growth factor-depleted matrigel plugs containing bFGF and PTHrP or bFGF and calcitonin. (e-g) Mice bearing PTHrP negative DU145 tumors were treated with saline, PTHrP or a scrambled control peptide. (e) Net tumor volume after treatment. Average tumor volume is indicated by a solid line. (f) Cryosections of tumors were stained with hematoxylin and eosin. (g) Cryosections were immunostained stained for expression of vascular antigen CD31 (red) and DNA (blue). Immunoreactive vessels were quantified (left) and photographed at 200× magnification (right). “*” indicates P<0.05 as determined by Student's t-test.

FIG. 19. Inhibition of angiogenesis and tumor growth by PTHrP gene delivery. (a, b) Chicken embryos bearing CAMs stimulated with bFGF or VEGF were injected with adenoviruses expressing Green Fluorescent Protein (GFP) or full length PTHrP. (a) Blood vessels branch points+/−S.E.M. above background were quantified (left panel). Cryosections of treated CAMs were immunostained with anti-PTHrP antibodies (right panel). Arrow indicates PTHrP positive blood vessel. (b) Cryosections of CAMs from (a) were immunostained with anti-αvβ3 antibodies. Immunoreactive vessels per microscopic field were quantified (left panel) and photographed (right panel). (c) Mean fluorescence intensity in lysates of bFGF supplemented growth factor-depleted matrigel plugs containing PTHrP- or GFP-expressing adenoviruses. (d-e) CAMs bearing DU145 prostate carcinoma tumors were injected with adenoviruses expressing Green Fluorescent Protein (GFP) or full length PTHrP. (d) Tumors weight (left panel) and appearance at 10× magnification (right panel) after seven days. (e) Cryosections of tumors from (d) were immunostained with anti-αvβ3 antibodies and immunoreactive vessels per 200× microscopic field were quantified (left panel) and photographed (right panel). “*” indicates P<0.05 as determined by Student's t-test.

FIG. 20: The N-terminal 34 amino acids of PTHrP inhibit endothelial cell migration in vitro and angiogenesis in vivo. (a-b) Endothelial cell migration (a) and attachment (b) to vitronectin in the presence of buffer, anti-αvβ3, PTHrP, calcitonin or CGRP. (c-d) Endothelial cell migration (c) and attachment (d) to vitronectin in the presence of buffer, anti-αvβ3, PTHrP 1-141, 1-86 and 1-34, as well as calcitonin. (f) bFGF stimulated CAMs were treated with saline, PTHrP fragments 1-141, 1-86 and 1-34 and calcitonin. Blood vessels branch points were determined. “*” indicates P<0.05 as determined by Student's t-test.

FIG. 21. PTHrP amino acids 1-10 are sufficient to inhibit endothelial cell migration in vitro and angiogenesis in vivo. (a) Endothelial cell migration on vitronectin in the presence of buffer, anti-αvβ3, PTHrP 1-34, 1-10, 15-34 and scrambled 1-10, as well as calcitonin. (b) bFGF stimulated CAMs were treated with saline, PTHrP 1-10, scrambled 1-10 and 1-34. Blood vessels branch points were determined. (c) Mean fluorescence intensity in lysates of growth factor-depleted matrigel plugs containing purified bFGF and saline, PTHrP 1-10 or scrambled PTHrP 1-10. (d) CAMs stimulated by bFGF (open squares), VEGF (open triangles), IL-8 (solid diamonds) or TNFα (solid circles) treated with saline or PTHrP 1-34. Blood vessels branch points were counted and percent inhibition of saline control was determined. “*” indicates P<0.05 as determined by Student's t-test.

FIG. 22. PTHrP inhibits migration by blocking Rac activation in a PKA dependent manner. (a) PKA activity in endothelial cells in the presence of culture medium (filled circle), PTHrP (filled diamond) or dibutyryl cAMP (open square). Specific activity is expressed as percent of total PKA that can be directly activated by cAMP in cell lysates. (b) Endothelial cell migration on vitronectin in the presence of culture medium, anti-αvβ3, PTHrP (1-34), the protein kinase A inhibitor, H89, and PTHrP combined with H89. (c) The effect of a mutationally inactive PKA (dnPKA) on cell migration in the presence of PTHrP, a scrambled peptide control and cAMP. (d) The effect of cAMP and transient transfection with the PKA catalytic subunit (PKAcat) on endothelial cell migration. (e) Western blots of lysates of GFP, dnPKA, and PKAcat transfected cells incubated with anti-V5 tag and anti-GFP antibodies. (f) Rac activity in endothelial cells treated with culture medium or PTHrP and in cells transfected with activated PKA or GFP. (g) Migration of endothelial cells expressing mutationally active Rac (V12 Rac, +) or GFP (−) in the presence of culture medium, PTHrP or cAMP. (h) Migration on vitronectin of endothelial cells transfected with PKAcat, V12 Rac+PKA cat, GFP or V12 Rac+GFP. (i) Quantification of annexin V positive endothelial cells cultured on vitronectin substrates in the presence of PTHrP. (j) Quantification of annexin V positive endothelial cells transfected with GFP (filled bars) or dnPKA (open bars) and treated with culture medium, PTHrP or scrambled control peptide. (k) Quantification of annexin V positive endothelial cells transfected with GFP or PKAcat and treated with culture medium or cAMP. “*” indicates P<0.05 as determined by Student's t-test.

FIG. 23. PTHrP mediated inhibition of angiogenesis is protein kinase A dependent. (a) Saline or bFGF stimulated CAMs treated with saline, PTHrP 1-34, H89, or PTHrP+H89. (b) bFGF stimulated CAMs were transfected with pcDNA/V5 tagged GFP DNA or pcDNA/V5 tagged dnPKA DNA and treated with PTHrP or saline. (c) bFGF, VEGF or saline stimulated CAMs were treated with saline (−) or dibutyryl cAMP (+). (d) bFGF stimulated CAMs were treated with saline or transfected with pcDNAV5 tagged PKAcat or pcDNAV5 tagged GFP DNA. (a-d) Blood vessels branch points were quantified. “*” indicates P<0.05 as determined by Student's t-test. (e) Cryosections of saline- or bFGF-stimulated CAMS treated with CGRP or PTHrP (see FIG. 18a-b) were immunostained to detect fragmented DNA as a marker of apoptosis (green) and VWF (red). Digitally merged images reveal VWF positive apoptotic vessels (yellow) indicated by arrows. (f) Cryosections of bFGF stimulated CAMs transfected with PKAcat DNA or treated with saline or cAMP were immunostained to detect fragmented DNA as a marker of apoptosis (green) and VWF (red). Digitally merged images reveal VWF positive apoptotic vessels (yellow). Arrows indicate VWF positive vessels.

### DEFINITIONS

To facilitate an understanding of the present invention, a number of terms and phrases are defined below.

The term “biologically active,” as used herein, refers to a molecule (e.g., peptide, nucleic acid sequence, carbohydrate molecule, organic or inorganic molecule, etc.) having structural, regulatory, and/or biochemical functions.

The term “agonist,” as used herein, refers to a molecule which, when interacting with a biologically active molecule, causes a change (e.g., enhancement) in the biologically active molecule, which modulates the activity of the biologically active molecule. Agonists may include proteins, nucleic acids, carbohydrates, or any other molecules which bind or interact with biologically active molecules. For example, agonists can alter the activity of gene transcription by interacting with RNA polymerase directly or through a transcription factor.

The terms “antagonist,” or “inhibitor,” as used herein, refer to a molecule that, when interacting with a biologically active molecule, blocks or modulates the biological activity of the biologically active molecule. Antagonists and inhibitors may include proteins, nucleic acids, carbohydrates, or any other molecules that bind or interact with biologically active molecules. Inhibitors and antagonists can affect the biology of entire cells, organs, or organisms (e.g., an inhibitor that slows tumor growth). In a preferred embodiment, an antagonist acts as a competitive inhibitor, or as a noncompetitive inhibitor of ligand binding.

As used herein, the term “tissue exhibiting angiogenesis” refers to a tissue in which new blood vessels are developing from pre-existing blood vessels.

The term “ligand” as used herein in reference to a ligand for an integrin (e.g. α5β1) receptor refers to a molecule, or portion thereof, to which that integrin specifically binds, thereby initiating a specific biological response (e.g., angiogenesis, cell migration, cell adhesion, cell survival, etc.) and/or the transduction of a signal in a cell. Integrin ligands may be present on the cell surface and/or present in the extracellular matrix (ECM).

The terms “specific binding,” “binding specificity,” and grammatical equivalents thereof when made in reference to the binding of a first molecule (such as a polypeptide, glycoprotein, nucleic acid sequence, etc.) to a second molecule (such as a polypeptide, glycoprotein, nucleic acid sequence, etc.) refer to the preferential interaction between the first molecule with the second molecule as compared to the interaction between the second molecule with a third molecule. Specific binding is a relative term that does not require absolute specificity of binding; in other words, the term “specific binding” does not require that the second molecule interact with the first molecule in the absence of an interaction between the second molecule and the third molecule. Rather, it is sufficient that the level of interaction between the first molecule and the second molecule is higher than the level of interaction between the second molecule with the third molecule. “Specific binding” of a first molecule with a second molecule also means that the interaction between the first molecule and the second molecule is dependent upon the presence of a particular structure on or within the first molecule; in other words the second molecule is recognizing and binding to a specific structure on or within the first molecule rather than to polypeptides, glycoproteins, or nucleic acids in general. For example, if a second molecule is specific for structure “A” that is on or within a first molecule, the presence of a third molecule containing structure A will reduce the amount of the second molecule which is bound to the first molecule.

For example, the term “specific binding” as used herein in reference to the binding of an agent to either an integrin (e.g., α5β1) or an integrin ligand means that the interaction is dependent upon the presence of a particular structure on the integrin or its ligand, respectively. For example, if an agent is specific for epitope “A”, the presence of a protein containing epitope A (or free, unlabelled A) in a reaction containing labelled “A” and the agent will reduce the amount of labelled A bound to the agent.

The term “capable of binding” when made in reference to the interaction between a first molecule (such as a polypeptide, glycoprotein, nucleic acid sequence, etc.) and a second molecule (such as a polypeptide, glycoprotein, nucleic acid sequence, etc.) means that the first molecule binds to the second molecule in the presence of suitable concentrations of salts, and suitable temperature, and pH. The conditions for binding molecules may be determined using routine and/or commercially available methods.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand. As used herein, the terms “nucleic acid molecule encoding,” “DNA sequence encoding,” “DNA encoding,” “RNA sequence encoding,” and “RNA encoding” refer to the order or sequence of deoxyribonucleotides or ribonucleotides along a strand of deoxyribonucleic acid or ribonucleic acid. The order of these deoxyribonucleotides or ribonucleotides determines the order of amino acids along the polypeptide (protein) chain translated from the mRNA. The DNA or RNA sequence thus codes for the amino acid sequence.

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ non-translated sequences. The sequences that are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “5′-A-C-T-3′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids.

As used herein, the term “Tm” is used in reference to the “melting temperature.” The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41(% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tm.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution of 5×SSPE (43.8 g/l NaCl, 6.9 g/l NaH₂PO₄—H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1× SSPE, 1.0% SDS at 42° C. when a probe of about 500 nucleotides in length is employed. In another embodiment, high stringency conditions comprise conditions equivalent to binding or hybridization at 68° C. in a solution containing 5×SSPE, 1% SDS, 5× Denhardt's reagent and 100 μg/ml denatured salmon sperm DNA followed by washing in a solution containing 0.1×SSPE, and 0.1% SDS at 68° C. when a probe of about 100 to about 1000 nucleotides in length is employed.

“Medium stringency conditions” when used in reference to nucleic acid hybridization comprise conditions equivalent to binding or hybridization at 42EC in a solution of 5×SSPE (43.8 g/l NaCl, 6.9 g/l NaH₂PO₄—H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42EC when a probe of about 500 nucleotides in length is employed.

“Low stringency conditions” comprise conditions equivalent to binding or hybridization at 42EC in a solution of 5×SSPE (43.8 g/l NaCl, 6.9 g/l NaH2PO4 H2O and 1.85 g/l EDTA, pH adjusted to 7.4 with NaOH), 0.1% SDS, 5× Denhardt's reagent (50× Denhardt's contains per 500 ml: 5 g Ficoll (Type 400, Pharamcia), 5 g BSA (Fraction V; Sigma)) and 100 Fg/ml denatured salmon sperm DNA followed by washing in a solution comprising 5×SSPE, 0.1% SDS at 42EC when a probe of about 500 nucleotides in length is employed.

The term “equivalent” when made in reference to a hybridization condition as it relates to a hybridization condition of interest means that the hybridization condition and the hybridization condition of interest result in hybridization of nucleic acid sequences which have the same range of percent (%) homology. For example, if a hybridization condition of interest results in hybridization of a first nucleic acid sequence with other nucleic acid sequences that have from 85% to 95% homology to the first nucleic acid sequence, then another hybridization condition is said to be equivalent to the hybridization condition of interest if this other hybridization condition also results in hybridization of the first nucleic acid sequence with the other nucleic acid sequences that have from 85% to 95% homology to the first nucleic acid sequence.

As used herein, the term “antigen” is used in reference to any substance that is capable of reacting with an antibody. It is intended that this term encompass any antigen and “immunogen” (i.e., a substance which induces the formation of antibodies). Thus, in an immunogenic reaction, antibodies are produced in response to the presence of an antigen or portion of an antigen.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

As used herein, the term “antigen binding protein” refers to proteins which bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Köhler and Milstein (Köhler and Milstein, Nature, 256:495-497 (1975)), as well as the trioma technique, the human B-cell hybridoma technique (See e.g., Kozbor et al., Immunol. Today, 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)).

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al., Science, 246:1275-1281 (1989)) to allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

Genes encoding antigen binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.) etc.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of a cell by experimental manipulations and may include gene sequences found in that cell so long as the introduced gene does not reside in the same location as does the naturally-occurring gene.

As used herein, the term “exogenous gene” refers to a gene that is not naturally present in a host organism or cell, or is artificially introduced into a host organism or cell.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “gene transfer system” refers to any means of delivering a composition comprising a nucleic acid sequence to a cell or tissue. For example, gene transfer systems include, but are not limited to vectors (e.g., retroviral, adenoviral, adeno-associated viral, and other nucleic acid-based delivery systems), microinjection of naked nucleic acid, and polymer-based delivery systems (e.g. liposome-based and metallic particle-based systems). As used herein, the term “viral gene transfer system” refers to gene transfer systems comprising viral elements (e.g., intact viruses and modified viruses) to facilitate delivery of the sample to a desired cell or tissue. As used herein, the term “adenovirus gene transfer system” refers to gene transfer systems comprising intact or altered viruses belonging to the family Adenoviridae.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein, the terms “instructions for administering said therapeutic compound to a subject,” “instructions for administering a therapeutic compound to a subject,” and equivalents, includes instructions for using the compositions contained in the kit for the treatment of conditions characterized by undesirable angiogenesis, cell migration, cell adhesion, cell survival in a cell or tissue. In some embodiments, the instructions further comprise a statement of the recommended or usual dosages of the compositions contained within the kit pursuant to 21 CFR §201 et seq. Additional information concerning labeling and instruction requirements applicable to the methods and compositions of the present are available at the Internet web page of the U.S. FDA.

The terms “protein of interest,” “peptide of interest,” “nucleotide sequence of interest,” and “molecule of interest” refer to any peptide sequence, nucleotide sequence, and molecule, respectively, the manipulation of which may be deemed desirable for any reason, by one of ordinary skill in the art. In one embodiment, the protein of interest refers to a protein encoded by a nucleic acid sequence of interest.

As used herein, the term “native” (or wild type) when used in reference to a protein, refers to proteins encoded by partially homologous nucleic acids so that the amino acid sequence of the proteins varies.

The term “reverse Northern blot” as used herein refers to the analysis of DNA by electrophoresis of DNA on agarose gels to fractionate the DNA on the basis of size followed by transfer of the fractionated DNA from the gel to a solid support, such as nitrocellulose or a nylon membrane. The immobilized DNA is then probed with a labeled oligo-ribonucleotide probe or RNA probe to detect DNA species complementary to the ribo probe used.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables).

As used herein, the term “subject” refers to organisms to be treated by the methods of the present invention. In one embodiment, the subject is a mammal. In one embodiment, the mammal includes, without limitation, human and non-human animals such simians, rodents, ovines, bovines, ruminants, lagomorphs, porcines, caprines, equines, canines, felines, aves, etc. Preferred non-human animals are members of the Order Rodentia (e.g., mouse and rat). Thus, the compounds of the invention may be administered by human health professionals as well as veterinarians. In the context of the invention, the term “subject” generally refers to an individual who will receive or who has received treatment (e.g., administration of antagonists and/or agonists of integrins).

The term “diagnosed,” as used herein, refers to the to recognition of a disease by its signs and symptoms (e.g., undesirable angiogenesis), genetic analysis, pathological analysis, or histological analysis, and the like.

As used herein, the term “purified” or “to purify” refers, to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. For example, an “isolated polynucleotide” is therefore a substantially purified polynucleotide.

The term “apoptosis” means non-necrotic cell death that takes place in metazoan animal cells following activation of an intrinsic cell suicide program. Apoptosis is a normal process in the development and homeostasis of metazoan animals. Apoptosis involves characteristic morphological and biochemical changes, including cell shrinkage, zeiosis, or blebbing, of the plasma membrane, and nuclear collapse and fragmentation of the nuclear chromatin, at intranucleosomal sites, due to activation of an endogenous nuclease. During apoptosis, cells undergo various changes that result in the eventual lysis of the cell into apoptotic bodies which are then typically phagocytosed by other cells. One of skill in the art appreciates that reducing the level of apoptosis results in increased cell survival, without necessarily (although it may include) increasing cell proliferation. Accordingly, as used herein, the terms “increase apoptosis” and “reduce survival” are equivalent. Aslo, as used herein, the terms “reduce apoptosis” and “increase survival” are equivalent. Apoptosis may be determined using methods known in the art. For example, apoptosis may be determined by measuring the cells' display of increased annexin-V binding to phosphatidylserine in plasma membranes, an early indicator of apoptosis, by live microscopy, or cell sorting analysis (FACS) for the transfection indicator green fluorescent protein and annexin-V. Also, cell death may confirmed by nuclear staining with Hoechst 33342.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

The term “polymorphic locus” is a locus present in a population that shows variation between members of the population (i.e., the most common allele has a frequency of less than 0.95). In contrast, a “monomorphic locus” is a genetic locus at little or no variations seen between members of the population (generally taken to be a locus at which the most common allele exceeds a frequency of 0.95 in the gene pool of the population).

As used herein, the term “genetic variation information” or “genetic variant information” refers to the presence or absence of one or more variant nucleic acid sequences (e.g., polymorphism or mutations) in a given allele of a particular gene (e.g., the PTH gene, PThrP gene and/or the gene encoding the catalytic subunit of PKA).

As used herein, the term “detection assay” refers to an assay for detecting the presence of absence of variant nucleic acid sequences (e.g., polymorphism or mutations) in a given allele of a particular gene (e.g., e.g., the PThrP gene and/or the catalytic subunit of PKA). Examples of suitable detection assays include, but are not limited to, those described in greater detail infra as well as being known in the art.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (D. L. Kacian et al., Proc. Natl. Acad. Sci. USA 69:3038 (1972)). Other nucleic acid will not be replicated by this amplification enzyme. Similarly, in the case of T7 RNA polymerase, this amplification enzyme has a stringent specificity for its own promoters (Chamberlin et al., Nature 228:227 (1970)). In the case of T4 DNA ligase, the enzyme will not ligate the two oligonucleotides or polynucleotides, where there is a mismatch between the oligonucleotide or polynucleotide substrate and the template at the ligation junction (D. Y. Wu and R. B. Wallace, Genomics 4:560 (1989)). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press (1989)).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” refers to a nucleic acid sequence or structure to be detected or characterized. Thus, the “target” is sought to be sorted out from other nucleic acid sequences. A “segment” is defined as a region of nucleic acid within the target sequence.

As used herein, the term “polymerase chain reaction” (“PCR”) refers to the method of K. B. Mullis U.S. Pat. Nos. 4,683,195, 4,683,202, and 4,965,188, hereby incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

As used herein, the term “recombinant DNA molecule” as used herein refers to a DNA molecule that is comprised of segments of DNA joined together by means of molecular biological techniques.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. In some embodiments, the isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein, a “portion of a chromosome” refers to a discrete section of the chromosome. Chromosomes are divided into sites or sections by cytogeneticists as follows: the short (relative to the centromere) arm of a chromosome is termed the “p” arm; the long arm is termed the “q” arm. Each arm is then divided into 2 regions termed region 1 and region 2 (region 1 is closest to the centromere). Each region is further divided into bands. The bands may be further divided into sub-bands. For example, the 11p15.5 portion of human chromosome 11 is the portion located on chromosome 11 (11) on the short arm (p) in the first region (1) in the 5th band (5) in sub-band 5 (0.5). A portion of a chromosome may be “altered;” for instance the entire portion may be absent due to a deletion or may be rearranged (e.g., inversions, translocations, expanded or contracted due to changes in repeat regions). In the case of a deletion, an attempt to hybridize (i.e., specifically bind) a probe homologous to a particular portion of a chromosome could result in a negative result (i.e., the probe could not bind to the sample containing genetic material suspected of containing the missing portion of the chromosome). Thus, hybridization of a probe homologous to a particular portion of a chromosome may be used to detect alterations in a portion of a chromosome.

The term “sequences associated with a chromosome” means preparations of chromosomes (e.g., spreads of metaphase chromosomes), nucleic acid extracted from a sample containing chromosomal DNA (e.g., preparations of genomic DNA); the RNA that is produced by transcription of genes located on a chromosome (e.g., hnRNA and mRNA), and cDNA copies of the RNA transcribed from the DNA located on a chromosome. Sequences associated with a chromosome may be detected by numerous techniques including probing of Southern and Northern blots and in situ hybridization to RNA, DNA, or metaphase chromosomes with probes containing sequences homologous to the nucleic acids in the above listed preparations.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein the term “coding region” when used in reference to structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets, which specify stop codons (i.e., TAA, TAG, TGA).

The term “recombinant DNA molecule” as used herein refers to a DNA molecule that is comprised of segments of DNA joined together by means of molecular biological techniques.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 (1989)).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 (1989)).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabelled antibodies.

The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis (See, Examples for protocols for performing Northern blot analysis). Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA of interest (e.g., the PThrP gene and/or the catalytic subunit of PKA) is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 (1973)), has been modified by several groups to optimize conditions for particular types of cells. The art is well aware of these numerous modifications.

A “composition comprising a given polynucleotide sequence” as used herein refers broadly to any composition containing the given polynucleotide sequence. The composition may comprise an aqueous solution. Compositions comprising polynucleotide sequences encoding e.g., the PThrP gene and/or the catalytic subunit of PKA or fragments thereof may be employed as hybridization probes. In this case, encoding polynucleotide sequences are typically employed in an aqueous solution containing salts (e.g., NaCl), detergents (e.g., SDS), and other components (e.g., Denhardt's solution, dry milk, salmon sperm DNA, etc.).

As used herein, the term “response,” when used in reference to an assay, refers to the generation of a detectable signal (e.g., accumulation of reporter protein, increase in ion concentration, accumulation of a detectable chemical product).

As used herein, the term “membrane receptor protein” refers to membrane spanning proteins that bind a ligand (e.g., a hormone or neurotransmitter). As is known in the art, protein phosphorylation is a common regulatory mechanism used by cells to selectively modify proteins carrying regulatory signals from outside the cell to the nucleus. The proteins that execute these biochemical modifications are a group of enzymes known as protein kinases. They may further be defined by the substrate residue that they target for phosphorylation. One group of protein kinases is the tyrosine kinases (TKs), which selectively phosphorylate a target protein on its tyrosine residues. Some tyrosine kinases are membrane-bound receptors (RTKs), and, upon activation by a ligand, can autophosphorylate as well as modify substrates. The initiation of sequential phosphorylation by ligand stimulation is a paradigm that underlies the action of such effectors as, for example, epidermal growth factor (EGF), insulin, platelet-derived growth factor (PDGF), and fibroblast growth factor (FGF). The receptors for these ligands are tyrosine kinases and provide the interface between the binding of a ligand (hormone, growth factor) to a target cell and the transmission of a signal into the cell by the activation of one or more biochemical pathways. Ligand binding to a receptor tyrosine kinase activates its intrinsic enzymatic activity. Tyrosine kinases can also be cytoplasmic, non-receptor-type enzymes and act as a downstream component of a signal transduction pathway.

As used herein, the term “signal transduction protein” refers to proteins that are activated or otherwise affected by ligand binding to a membrane or cytostolic receptor protein or some other stimulus. Examples of signal transduction protein include adenyl cyclase, phospholipase C, and G-proteins. Many membrane receptor proteins are coupled to G-proteins (i.e., G-protein coupled receptors (GPCRs); for a review, see Neer, 1995, Cell 80:249-257 (1995)). Typically, GPCRs contain seven transmembrane domains. Putative GPCRs can be identified on the basis of sequence homology to known GPCRs.

GPCRs mediate signal transduction across a cell membrane upon the binding of a ligand to an extracellular portion of a GPCR. The intracellular portion of a GPCR interacts with a G-protein to modulate signal transduction from outside to inside a cell. A GPCR is therefore said to be “coupled” to a G-protein. G-proteins are composed of three polypeptide subunits: an α subunit, which binds and hydrolyses GTP, and a dimeric βγ subunit. In the basal, inactive state, the G-protein exists as a heterotrimer of the α and βγ subunits. When the G-protein is inactive, guanosine diphosphate (GDP) is associated with the α subunit of the G-protein. When a GPCR is bound and activated by a ligand, the GPCR binds to the G-protein heterotrimer and decreases the affinity of the Gα subunit for GDP. In its active state, the G subunit exchanges GDP for guanine triphosphate (GTP) and active Gα subunit disassociates from both the receptor and the dimeric βγ subunit. The disassociated, active Gα subunit transduces signals to effectors that are “downstream” in the G-protein signaling pathway within the cell. Eventually, the G-protein's endogenous GTPase activity returns active G subunit to its inactive state, in which it is associated with GDP and the dimeric βγ subunit.

Numerous members of the heterotrimeric G-protein family have been cloned, including more than 20 genes encoding various Gα subunits. The various G subunits have been categorized into four families, on the basis of amino acid sequences and functional homology. These four families are termed Gαₛ, Gαᵢ, Gα_{q}, and Gα₁₂. Functionally, these four families differ with respect to the intracellular signaling pathways that they activate and the GPCR to which they couple.

For example, certain GPCRs normally couple with Gαₛ and, through Gαₛ, these GPCRs stimulate adenylyl cyclase activity. Other GPCRs normally couple with GGα_{q}, and through GGα_{q}, these GPCRs can activate phospholipase C (PLC), such as the β isoform of phospholipase C (i.e., PLCβ, Stermweis and Smrcka, Trends in Biochem. Sci. 17:502-506 (1992)).

As used herein, the term “nucleic acid binding protein” refers to proteins that bind to nucleic acid, and in particular to proteins that cause increased (i.e., activators or transcription factors) or decreased (i.e., inhibitors) transcription from a gene.

As used herein, the term “ion channel protein” refers to proteins that control the ingress or egress of ions across cell membranes. Examples of ion channel proteins include, but are not limited to, the Na⁺—K⁺ ATPase pump, the Ca²⁺ pump, and the K⁺ leak channel.

As used herein, the term “protein kinase” refers to proteins that catalyze the addition of a phosphate group from a nucleoside triphosphate to an amino acid side chain in a protein. Kinases comprise the largest known enzyme superfamily and vary widely in their target proteins. Kinases may be categorized as protein tyrosine kinases (PTKs), which phosphorylate tyrosine residues, and protein serine/threonine kinases (STKs), which phosphorylate serine and/or threonine residues. Some kinases have dual specificity for both serine/threonine and tyrosine residues. Almost all kinases contain a conserved 250-300 amino acid catalytic domain. This domain can be further divided into 11 subdomains. N-terminal subdomains I-IV fold into a two-lobed structure that binds and orients the ATP donor molecule, and subdomain V spans the two lobes. C-terminal subdomains VI-XI bind the protein substrate and transfer the gamma phosphate from ATP to the hydroxyl group of a serine, threonine, or tyrosine residue. Each of the 11 subdomains contains specific catalytic residues or amino acid motifs characteristic of that subdomain. For example, subdomain I contains an 8-amino acid glycine-rich ATP binding consensus motif, subdomain II contains a critical lysine residue required for maximal catalytic activity, and subdomains VI through IX comprise the highly conserved catalytic core. STKs and PTKs also contain distinct sequence motifs in subdomains VI and VIII, which may confer hydroxyamino acid specificity. Some STKs and PTKs possess structural characteristics of both families. In addition, kinases may also be classified by additional amino acid sequences, generally between 5 and 100 residues, which either flank or occur within the kinase domain.

Non-transmembrane PTKs form signaling complexes with the cytosolic domains of plasma membrane receptors. Receptors that signal through non-transmembrane PTKs include cytokine, hormone, and antigen-specific lymphocytic receptors. Many PTKs were first identified as oncogene products in cancer cells in which PTK activation was no longer subject to normal cellular controls. In fact, about one third of the known oncogenes encode PTKs. Furthermore, cellular transformation (oncogenesis) is often accompanied by increased tyrosine phosphorylation activity (See, e.g., Carbonneau, H. and Tonks, Annu. Rev. Cell Biol. 8:463-93 (1992)). Regulation of PTK activity may therefore be an important strategy in controlling some types of cancer.

As used herein, the term “reporter gene” refers to a gene encoding a protein that may be assayed. Examples of reporter genes include, but are not limited to, luciferase (See, e.g., deWet et al., Mol. Cell. Biol. 7:725 (1987) and U.S. Pat Nos. 6,074,859; 5,976,796; 5,674,713; and 5,618,682; all of which are incorporated herein by reference), green fluorescent protein (e.g., GenBank Accession Number U43284; a number of GFP variants are commercially available from CLONTECH Laboratories, Palo Alto, Calif.), chloramphenicol acetyltransferase, β-galactosidase, alkaline phosphatase, and horse radish peroxidase.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the term “entering” as in “entering said genetic variation information into said computer” refers to transferring information to a “computer readable medium.” Information may be transferred by any suitable method, including but not limited to, manually (e.g., by typing into a computer) or automated (e.g., transferred from another “computer readable medium” via a “processor”).

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “computer implemented method” refers to a method utilizing a “CPU” and “computer readable medium.”

The terms “neoplasm” and “tumor” refer to a tissue growth which is characterized, in part, by increased angiogenesis and/or increased cell proliferation. Neoplasms may be benign and are exemplified, but not limited to, a hemangioma, glioma, teratoma, and the like. Neoplasms may alternatively be malignant, for example, a carcinoma, sarcoma, glioblastoma, astrocytoma, neuroblastoma, retinoblastoma, and the like.

The terms “malignant neoplasm” and “malignant tumor” refer to a neoplasm which contains at least one cancer cell. A “cancer cell” refers to a cell undergoing early, intermediate or advanced stages of multi-step neoplastic progression as previously described (H. C. Pitot (1978) in “Fundamentals of Oncology,” Marcel Dekker Ed.), New York pp 15-28). The features of early, intermediate and advanced stages of neoplastic progression have been described using microscopy. Cancer cells at each of the three stages of neoplastic progression generally have abnormal karyotypes, including translocations, inversion, deletions, isochromosomes, monosomies, and extra chromosomes. A cell in the early stages of malignant progression is referred to as “hyperplastic cell” and is characterized by dividing without control and/or at a greater rate than a normal cell of the same cell type in the same tissue. Proliferation may be slow or rapid but continues unabated. A cell in the intermediate stages of neoplastic progression is referred to as a “dysplastic cell.” A dysplastic cell resembles an immature epithelial cell, is generally spatially disorganized within the tissue and loses its specialized structures and functions. During the intermediate stages of neoplastic progression, an increasing percentage of the epithelium becomes composed of dysplastic cells. “Hyperplastic” and “dysplastic” cells are referred to as “pre-neoplastic” cells. In the advanced stages of neoplastic progression a dysplastic cell become a “neoplastic” cell. Neoplastic cells are typically invasive i.e., they either invade adjacent tissues, or are shed from the primary site and circulate through the blood and lymph to other locations in the body where they initiate one or more secondary cancers, i.e., “metastases.” Thus, the term “cancer” is used herein to refer to a malignant neoplasm, which may or may not be metastatic. Malignant neoplasms that can be diagnosed using a method of the invention include, for example, carcinomas such as lung cancer, breast cancer, prostate cancer, cervical cancer, pancreatic cancer, colon cancer, ovarian cancer; stomach cancer, esophagus cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, lymphoma, and leukemia. Malignant neoplasms are further exemplified by sarcomas (such as osteosarcoma and Kaposi's sarcoma). The invention expressly contemplates within its scope any malignant neoplasm so long as the neoplasm is characterized, at least in part, by angiogenesis associated with alpha 4 beta 1 expression by the newly forming blood vessels.

The terms “disease” and “pathological condition” are used interchangeably to refer to a state, signs, and/or symptoms that are associated with any impairment, interruption, cessation, or disorder of the normal state of a living animal or of any of its organs or tissues that interrupts or modifies the performance of normal functions, and may be a response to environmental factors (such as malnutrition, industrial hazards, or climate), to specific infective agents (such as worms, bacteria, or viruses), to inherent defect of the organism (such as various genetic anomalies, or to combinations of these and other factors. The term “disease” includes responses to injuries, especially if such responses are excessive, produce symptoms that excessively interfere with normal activities of an individual, and/or the tissue does not heal normally (where excessive is characterized as the degree of interference, or the length of the interference).

The term “cell” refers to a single cell as well as to a population of (i.e., more than one) cells. The population may be a pure population comprising one cell type. Alternatively, the population may comprise more than one cell type. In the present invention, there is no limit on the number of cell types that a cell population may comprise. The terms “cells,” “at least one cell,” and “population of cells” refer to two or more cells. In one embodiment, the cell is exemplified by, but not limited to, endothelial cell, vascular smooth muscle cell, monocyte cell, macrophage cell, benign tumor cell, malignant tumor cell, fibroblast cell, B cell, T cell, myocyte cell, megakaryocyte cell, eosinophil cell, neurite cell, and synoviocyte cell. In another embodiment, the cell is exemplified by B cell, stromal cell of lymph organ such as spleen, fibroblast cell such as embryo fibroblasts (EFs), including mouse embryo fibroblasts (MEFs), macrophage cell such as stromal macrophage cell, dendritic cell, neuron cell, plasma cell, lymphoid cell, lymphoblastoid cell, myeloid cell, Reed-Sternber (HRS) cell of Hodgkin's lymphomas, epithelial cell such as breast cell, gastric cell, lung cell, prostate cell, cervical cell, pancreatic cell, colon cell, rectal cell, ovarian cell, stomach cell, esophagus cell, mouth cell, tongue cell, gum cell, skin cell, muscle cell, heart cell, liver cell, bronchial cell, cartilage cell, bone cell, testis cell, kidney cell, endometrium cell, uterus cell, bladder cell, gastrointestinal tract cell, thyroid cell, brain cell, gall bladder cell, gastrointestinal tract cell, and ocular cell (such as cell of the cornea, cell of uvea, cell of the choroids, cell of the macula, vitreous humor cell, etc.).

As used in this specification and the appended claims, the singular forms “a,” “an” and “the” includes both singular and plural references unless the content clearly dictates otherwise.

As used herein, the term “or” when used in the expression “A or B,” where A and B refer to a composition, disease, product, etc., means one, or the other, or both.

The term “on” when in reference to the location of a first article with respect to a second article means that the first article is on top and/or into the second article, including, for example, where the first article permeates into the second article after initially being placed on it.

As used herein, the term “comprising” when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the term “comprising” when placed before the recitation of steps in a method does not (although it may) require sequential performance of the listed steps, unless the content clearly dictates otherwise. For example, a method comprising steps a, b, and c encompasses, a method of performing steps in the order of steps a, c, and b, the order of steps c, b, and a, and the order of steps c, a, and b, etc.

Unless otherwise indicated, all numbers expressing quantities of ingredients, properties such as molecular weight, reaction conditions, and so forth as used in the specification and claims are to be understood as being modified in all instances by the term “about.” Accordingly, unless indicated to the contrary, the numerical parameters in the specification and claims are approximations that may vary depending upon the desired properties sought to be obtained by the present invention. At the very least, and without limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the invention are approximation, the numerical values in the specific examples are reported as precisely as possible. Any numerical value, however, inherently contains standard deviations that necessarily result from the errors found in the numerical value's testing measurements.

The term “not” when preceding, and made in reference to, any particularly named molecule (such as the catalytic subunit of protein kinase, etc.), phenomenon (such as biological activity, biochemical activity, etc.) and/or composition (such as endothelial cells) means that only the particularly named molecule, phenomenon, and/or composition is excluded. For example, the term “integrin is not alpha 5 beta 1” means any integrin (such as alpha v beta 1, alpha v beta 3, alpha v beta 5, alpha v beta 6, alpha v beta 8, alpha 1 beta 1, alpha 2 beta 1, alpha 3 beta 1, alpha 4 beta 1, alpha 6 beta 1, alpha 7 beta 1, alpha 8 beta 1, alpha 9 beta 1, alpha 10 beta 1, alpha 6 beta 4, alpha 4 beta 7, alpha M beta 2, alpha L beta 2, alpha X beta 2, and alpha IIb beta 3) other than alpha 5 beta 1 integrin.

The terms “modulate,” “alter” as used herein refers to a change in the biological activity of a biologically active molecule, or to a change in the activity of a biological process (e.g., angiogenesis, cell migration, cell adhesion, cell survival (contra, cell apoptosis)), and the like). Modulation can be an increase or a decrease in activity, a change in binding characteristics, or any other change in the biological, functional, or immunological properties of biologically active molecules. In particular, the terms “modulate,” “alter” and grammatical equivalents as used herein in reference to the level of any substance and/or phenomenon refers to an increase and/or decrease in the quantity of the substance and/or phenomenon, regardless of whether the quantity is determined objectively, and/or subjectively.

The terms “increase,” “elevate,” “raise,” and grammatical equivalents when in reference to the level of a substance and/or phenomenon in a first sample relative to a second sample, mean that the quantity of the substance and/or phenomenon in the first sample is higher than in the second sample by any amount that is statistically significant using any art-accepted statistical method of analysis. In one embodiment, the increase may be determined subjectively, for example when a patient refers to their subjective perception of disease symptoms, such as pain, clarity of vision, etc. In another embodiment, the quantity of the substance and/or phenomenon in the first sample is at least 10% greater than the quantity of the same substance and/or phenomenon in a second sample. In another embodiment, the quantity of the substance and/or phenomenon in the first sample is at least 25% greater than the quantity of the same substance and/or phenomenon in a second sample. In yet another embodiment, the quantity of the substance and/or phenomenon in the first sample is at least 50% greater than the quantity of the same substance and/or phenomenon in a second sample. In a further embodiment, the quantity of the substance and/or phenomenon in the first sample is at least 75% greater than the quantity of the same substance and/or phenomenon in a second sample. In yet another embodiment, the quantity of the substance and/or phenomenon in the first sample is at least 90% greater than the quantity of the same substance and/or phenomenon in a second sample.

The terms “reduce,” “inhibit,” “diminish,” “suppress,” “decrease,” “block,” and grammatical equivalents when in reference to the level of a substance (such as a protein, nucleic acid sequence, etc.) and/or phenomenon (such as cell migration, cell survival, angiogenesis, enzyme activity such as protein kinase activity, etc.) in a first sample relative to a second sample, mean that the quantity of substance and/or phenomenon in the first sample is lower than in the second sample by any amount that is statistically significant using any art-accepted statistical method of analysis. The first and second samples need not, although they may, be from different sources. For example, a second sample may be the same as the first sample with the exception that the second sample has been manipulated (e.g., temperature change) or treated in the presence or absence of compounds as compared to the first sample. In one embodiment, the reduction may be determined subjectively, for example when a patient refers to their subjective perception of disease symptoms, such as pain, clarity of vision, etc. In another embodiment, the quantity of the substance and/or phenomenon in the first sample is at least 10% lower than the quantity of the same substance and/or phenomenon in a second sample. In another embodiment, the quantity of the substance and/or phenomenon in the first sample is at least 25% lower than the quantity of the same substance and/or phenomenon in a second sample. In yet another embodiment, the quantity of the substance and/or phenomenon in the first sample is at least 50% lower than the quantity of the same substance and/or phenomenon in a second sample. In a further embodiment, the quantity of the substance and/or phenomenon in the first sample is at least 75% lower than the quantity of the same substance and/or phenomenon in a second sample. In yet another embodiment, the quantity of the substance and/or phenomenon in the first sample is at least 90% lower than the quantity of the same substance and/or phenomenon in a second sample.

For example, in one embodiment, as used herein, the term “inhibiting angiogenesis,” “diminishing angiogenesis,” “reducing angiogenesis,” and grammatical equivalents thereof refer to reducing the level of angiogenesis in a tissue to a quantity which is preferably 10% less than, more preferably 50% less than, yet more preferably 75% than, even more preferably 90% less than, the quantity in a corresponding control tissue, and most preferably is at the same level which is observed in a control tissue. A reduced level of angiogenesis need not, although it may, mean an absolute absence of angiogenesis. The invention does not require, and is not limited to, methods which wholly eliminate angiogenesis.

Using another example, in another embodiment, the terms “reducing the level of expression of an integrin of interest,” or “diminishing expression of an integrin of interest or expression of a ligand of an integrin of interest,” and grammatical equivalents thereof, as used herein refer to reducing the level of an integrin of interest or of a ligand of an integrin of interest to a quantity which is preferably 20% less than the quantity in a corresponding control tissue, more preferably is 50% less than the quantity in a corresponding control tissue, yet more preferably is 90% less than the quantity in a corresponding control tissue, and most preferably is at the background level of, or is undetectable by any method, such as a Western blot analysis of the integrin or of the ligand of the integrin, by immunofluorescence for detection of integrin or of the ligand of the integrin, by reverse transcription polymerase chain (RT-PCR) reaction for detection of the integrin or of the ligand of the integrin mRNA, and/or by in situ hybridization for detection of integrin or of the ligand of the integrin mRNA. When a background level, or undetectable level, of the integrin of interest, or of the ligand of the integrin of interest peptide or mRNA is measured, this may indicate that integrin or ligand of the integrin is not expressed. A reduced level of integrin or ligand of the integrin need not, although it may, mean an absolute absence of expression of integrin or of the ligand of the integrin. The invention does not require, and is not limited to, antisense integrin of any particular integrin of interest or to the ligand of any particular integrin of interest.

In yet another example, in another embodiment, the terms “inhibit the specific binding” and “reduce the specific binding” when made in reference to the effect of an agent on the specific binding of an integrin with a corresponding integrin ligand means that the agent reduces the level of specific binding of the integrin with its ligand to a quantity which is preferably 10% less than, more preferably 50% less than, yet more preferably 75% than, even more preferably 90% less than, the quantity of specific binding in a corresponding control sample, and most preferably is at the same level which is observed in a control sample, as detected by (for example) an Enzyme Linked Immunosorbent Assay (ELISA). A reduced level of specific binding need not, although it may, mean an absolute absence of specific binding. The invention does not require, and is not limited to, methods which wholly eliminate specific binding of an integrin with its ligand.

Reference herein to any specifically named polypeptide sequence (such as SEQ ID Nos:114 and) or protein (such as “protein kinase A,” “parathyroid hormone,” “parathyroid hormone related protein,” etc.) refers to a polypeptide and protein, respectively, having at least one of the biological activities of the specifically named protein, wherein the biological activity is detectably by any method. For example, in one embodiment, exemplary polynucleotides encoding PTH include SEQ ID NOs: 1-44 and 112) listed in Table 1 shown below. Exemplary polynucleotides encoding PTHrP include SEQ ID NOs: 45-73 and 113 listed in Table 2 shown below. Exemplary polynucleotides encoding at least a portion of a catalytically active PKA subunit and amino acid sequences encoded thereby (e.g., SEQ ID NOs: 74-111) are shown below in Table 3.

In addition, reference herein to any specifically named polypeptide sequence (such as SEQ ID Nos:114, 115) or protein (such as “protein kinase A,” “parathyroid hormone,” “parathyroid hormone related protein,” etc.) includes within its scope fragments, fusion proteins, and variants of the specifically named polypeptide sequence and protein. The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, up to the entire amino acid sequence minus one amino acid residue. In one embodiment, a fragment spans the portion of the polypeptide required for intermolecular binding of the compositions (claimed in the present invention) with its various ligands and/or substrates. The term a “variant” of a protein as used herein is defined as an amino acid sequence which differs by insertion, deletion, and/or conservative substitution of one or more amino acids from the protein. The term “conservative substitution” of an amino acid refers to the replacement of that amino acid with another amino acid which has a similar hydrophobicity, polarity, and/or structure. For example, the following aliphatic amino acids with neutral side chains may be conservatively substituted one for the other: glycine, alanine, valine, leucine, isoleucine, serine, and threonine. Aromatic amino acids with neutral side chains which may be conservatively substituted one for the other include phenylalanine, tyrosine, and tryptophan. Cysteine and methionine are sulphur-containing amino acids which may be conservatively substituted one for the other. Also, asparagine may be conservatively substituted for glutamine, and vice versa, since both amino acids are amides of dicarboxylic amino acids. In addition, aspartic acid (aspartate) may be conservatively substituted for glutamic acid (glutamate) as both are acidic, charged (hydrophilic) amino acids. Also, lysine, arginine, and histidine may be conservatively substituted one for the other since each is a basic, charged (hydrophilic) amino acid. Guidance in determining which and how many amino acid residues may be substituted, inserted or deleted without abolishing biological and/or immunological activity may be found using computer programs well known in the art, for example, DNAStar™ software. In one embodiment, the sequence of the variant has at least 95% identity with the sequence of the protein in issue. In another embodiment, the sequence of the variant has at least 90% identity with the sequence of the protein in issue. In yet another embodiment, the sequence of the variant has at least 85% identity with the sequence of the protein in issue. In a further embodiment, the sequence of the variant has at least 80% identity with the sequence of the protein in issue. In yet another embodiment, the sequence of the variant has at least 75% identity with the sequence of the protein in issue. In another embodiment, the sequence of the variant has at least 70% identity with the sequence of the protein in issue. In another embodiment embodiment, the sequence of the variant has at least 65% identity with the sequence of the protein in issue.

Reference herein to any specifically named nucleotide sequence (such as a sequence encoding protein kinase A, a sequence encoding parathyroid hormone, AVSEHQLLHS/D, SVSEIQLMNL, etc.) includes within its scope fragments, homologs, and sequences that hybridize under stingnet condition to the specifically named nucleotide sequence. The fragment may range in size from an exemplary 10, 20, 50, 100 contiguous nucleotide residues to the entire nucleic acid sequence minus one nucleic acid residue. Thus, a nucleic acid sequence comprising “at least a portion of” a nucleotide sequence comprises from ten (10) contiguous nucleotide residues of the nucleotide sequence to the entire nucleotide sequence. The term “homolog” of a specifically named nucleotide sequence refers to an oligonucleotide sequence which exhibits greater than or equal to 50% identity to the oligonucleotide sequence in issue when sequences having a length of 100 bp or larger are compared. Alternatively, a homolog of a specifically named nucleotide sequence is defined as an oligonucleotide sequence that has at least 95% identity with the sequence of the nucleotide sequence in issue. In another embodiment, the sequence of the homolog has at least 90% identity with the sequence of the nucleotide sequence in issue. In yet another embodiment, the sequence of the homolog has at least 85% identity with the sequence of the nucleotide sequence in issue. In a further embodiment, the sequence of the homolog has at least 80% identity with the sequence of the nucleotide sequence in issue. In yet another embodiment, the sequence of the homolog has at least 75% identity with the sequence of the nucleotide sequence in issue. In another embodiment, the sequence of the homolog has at least 70% identity with the sequence of the nucleotide sequence in issue. In another embodiment embodiment, the sequence of the homolog has at least 65% identity with the sequence of the nucleotide sequence in issue.

The terms nucleotide sequence “comprising a particular nucleic acid sequence” and protein “comprising a particular amino acid sequence” and equivalents of these tersm, refer to any nucleotide sequence of interst and to any protein of interest that contains the particularly named nucleic acid sequence (such the particularly named AVSEHQLLHS/D and SVSEIQLMNL sequences) and the particularly named amino acid sequence (such as the particularly named protein kinase A catalytic subunit), respectively. The invention does not limit on the source (e.g., cell type, tissue, animal etc.), nature (e.g. synthetic, recombinant, purified from cell extract, etc.), and/or sequence of the nucleotide sequence of interst and/or protein of interst. In one embodiment, the nucleotide sequence of interst and protein of interst include coding sequences of structural genes (e.g., probe genes, reporter genes, selection marker genes, oncogenes, drug resistance genes, growth factors, etc.).

Exemplary “probe” genes sequences (i.e., sequence useful in the detection, identification and isolation of particular polypeptide sequence) encode ligand-binding systems useful for the isolation of polypeptides such as the staphylococcal protein A and its derivative ZZ (which binds to human polyclonal IgG), histidine tails (which bind to Ni²⁺), biotin (which binds to streptavidin), maltose-binding protein (MBP) (which binds to amylose), glutathione S-transferase (which binds to glutathione), etc. Exemplary “reporter” gene sequences (i.e. sequences that encodes a molecule such as RNA, polypeptide, etc., that is detectable in enzyme-based histochemical assays, fluorescent, radioactive, and luminescent systems, etc.) include green fluorescent protein gene, E. coli-galactosidase gene, human placental alkaline phosphatase gene, and chloramphenicol acetyltransferase gene.

In another embodiment, the nucleotide sequence “comprising a particular nucleic acid sequence” encodes a “fusion protein,” i.e., two or more polypeptides that are “operably linked” i.e., wherein the linkage of nucleic acid sequences and/or amino acid sequences is such that the linked sequences perform their intended function. For example, operably linking a promoter sequence to a nucleotide sequence of interest refers to linking the promoter sequence and the nucleotide sequence of interest in a manner such that the promoter sequence is capable of directing the transcription of the nucleotide sequence of interest and/or the synthesis of a polypeptide encoded by the nucleotide sequence of interest. The term also refers to the linkage of amino acid sequences in such a manner so that a functional protein is produced. Exemplary sequences that may be linked to the invention's sequence include those for adenosine deaminase (ADA) gene (GenBank Accession No. M13792); alpha-1-antitrypsin gene (GenBank Accession No. M11465); beta chain of hemoglobin gene (GenBank Accession No. NM_{—}000518); receptor for low density lipoprotein gene (GenBank Accession No. D16494); lysosomal glucocerebrosidase gene (GenBank Accession No. K02920); hypoxanthine-guanine phosphoribosyltransferase (HPRT) gene (GenBank Accession No. M26434, J00205, M27558, M27559, M27560, M27561, M29753, M29754, M29755, M29756, M29757); lysosomal arylsulfatase A (ARSA) gene (GenBank Accession No. NM_{—}000487); ornithine transcarbamylase (OTC) gene (GenBank Accession No. NM_{—}000531); phenylalanine hydroxylase (PAH) gene (GenBank Accession No. NM_{—}000277); purine nucleoside phosphorylase (NP) gene (GenBank Accession No. NM_{—}000270); the dystrophin gene (GenBank Accession Nos. M18533, M17154, and M18026); the utrophin (also called the dystrophin related protein) gene (GenBank Accession No. NM_{—}007124); and the human cystic fibrosis transmembrane conductance regulator (CFTR) gene (GenBank Accession No. M28668).

In a further embodiment, nucleotide sequences of interst also include non-coding regulatory sequences which do not encode an mRNA or protein product, (e.g., promoter sequence, polyadenylation sequence, termination sequence, enhancer sequence, etc.). Exemplary “promoters” include, without limitation, single, double and triple promoters. Double promoters are exemplified, but not limited to, vaRNA I-tRNA, vaRNA I-CMV, vaRNA I-RSV, vaRNA I-SV40, vaRNA I-PEPCK, vaRNA I-MT, vaRNA I-SR, vaRNA I-P450 family, vaRNA I-GAL7, T₇-vaRNA I, T₃-vaRNA, vaRNA I-SP6, vaRNA I-K11, and vaRNA I-heat shock protein double promoters, while triple promoters are exemplified, but not limited to, the CMV-T7-vaRNA I triple promoter.

In addition, the invention's molecules may be linked to a glycopeptide, polysaccharide, lipopeptide, glycolipid, lipid, steroid, etc.

### GENERAL DESCRIPTION OF THE INVENTION

The invention relates to methods for detecting and inhibiting angiogenesis, cell migration, cell adhesion, and/or cell survival in endothelial and non-endothelial cells as well as in normal and tumor cells. The invention further relates to methods for screening test compounds for their ability to inhibit angiogenesis, cell migration, cell adhesion, and/or cell survival. The compositions and methods of the present invention are useful in, for example, in diagnosing and inhibiting pathological conditions associated with angiogenesis, cell migration, cell adhesion, and/or cell survival. Cell migration and invasion is positively regulated by a number of important ligand/receptor interactions between growth factors and their receptors, and integrins and extracellular matrix ligands and proteases. Generally, cell migration is regulated by growth factor receptor and integrin stimulated signaling pathways that activate small GTPases (e.g., Rho, Rac, and cdc42), kinases (e.g., Src and FAK), cytoskeletal proteins (e.g., vinculin and paxillin), and MAP kinases 3. Provisional extracellular matrices are secreted by actively proliferating and migrating cells, while basement membrane type ECM proteins support tissue homeostasis and quiescence. Proteases convert basement membranes components such as laminin and collagen to provisional ECM proteins by exposing cryptic integrin binding sites. In contrast to basement membrane components, provisional ECM components preferentially activate the migratory phenotype. However, the mechanisms regulating these different cellular responses to the two types of ECM components remain unclear. Growth factors stimulate, while integrins support, cell movement along extracellular membrane proteins. While growth factors are required to elicit new blood vessel growth, adhesion to provisional ECM proteins such as fibronectin, vitronectin, and fibrinogen is required for endothelial cell survival, proliferation and motility during new blood vessel growth. (See e.g., J. E. Meredith Jr., et al., Mol. Biol. Cell, 4:953-961 (1993); P. C. Brooks et al., Cell, 79:1157-1164 (1994); N. Boudreau et al., Science, 267:891-893(1995); S. Stromblad et al., J. Clin. Invest., 98:426-433 (1996); R. A. F. Clark et al. Am. J. Path. 148:1407-1421 (1996); and M. Friedlander et al., Science, 270:1500-1502 (1995)).

Importantly, cell migration is mediated in large part by the integrins, a family of over twenty different αβ heterodimeric combinations, which bind to extracellular matrix (ECM) proteins or cell surface immunoglobulin family molecules through short peptide sequences in the ligands. Integrins regulate not only cell adhesion and migration on the ECM proteins found in intercellular spaces and basement membranes, but also signal transduction that promotes motility (e.g., angiogenesis), cell survival (e.g., entry into apoptosis), and gene expression. For instance, some embodiments of the present invention contemplate that unoccupied (unligated) integrins (e.g., unligated integrin α5β1 in tumor cells) dominantly suppress cell survival in attached cells. Thus, in preferred embodiments, the overexpression of certain integrins (e.g., α5β1) suppresses tumor cell proliferation in vitro and tumorigenesis in vivo.

Still further embodiments of the present invention contemplate that ligation of certain integrins promotes endothelial cell motility and angiogenesis. Angiogenesis requires activation of quiescent endothelial cells by growth factors, degradation of basement membrane and disassociation from the supporting vascular smooth muscle, followed by subsequent proliferation, survival and migration of endothelial cells and eventual differentiation of these cells into lumen-bearing structures that may also be lined with vascular smooth muscle cells.

Undesirable (e.g., pathological) angiogenesis, such as occurs in solid tumor cancer, results from an overbalance of stimulatory factors and an insufficiency of inhibitory factors. (See e.g., P. Carmeliet and R. K. Jain, R. K. Angiogenesis in cancer and disease. Nature. 407:249-257 (2000)). Certain embodiments of the present invention provide methods and compositions to inhibit angiogenesis by blocking one or more of these discrete processes.

In some embodiments, the present invention contemplates that ligation of the fibronectin receptor, integrin α5β1, promotes endothelial cell migration and angiogenesis. In contrast, endothelial cell migration, angiogenesis and tumor growth are inhibited by antagonists (e.g., small molecules, antibodies, dominant negative mutations, and the like) of integrin α5β1 and antagonists of fibronectin. (See e.g., S. Kim et al., Am. J. Path., 156:1345-1362 (2000)). Other integrins that positively regulate vascular development and angiogenesis include integrins αβv3 and αβv5. (See e.g., R. A. F. Clark et al., Am. J. Path., 148:1407-1421 (1996); M. Friedlander et al., Science, 270:1500-1502 (1995); W. Arap et al., Science, 279:377-380 (1997); P. C. Brooks et al., Science, 264:569-571 (1994); C. P. Carron et al., Cancer Research, 58:1930-1955 (1998); M. Christofidou-Solomidou et al., Am. J. Path., 151:975-983 (1997); C. J. Drake et al., J. Cell Science, 108:2655-266 (1995); M. Friedlander et al., Proc. Natl. Acad. Sci. U.S.A., 93:9764-9769 (1996); J. A. Varner et al., Angiogenesis, 3:53-61 (1999); and D. A. Sipkins et al., Nature Medicine, 4:623-626 (1998)). The present invention provides antagonists of these, and other, integrins that inhibit angiogenesis and tumor growth by causing endothelial cells in tumors, but not in normal tissues, to undergo apoptosis. Thus, in preferred embodiments, certain unligated integrins negatively regulate angiogenesis.

Further embodiments of the present invention provide methods and compositions that inhibit integrin ligation by activating protein kinase A (“PKA”) even in cells adherent to the matrix though other integrins. In turn, it is contemplated, although the present invention is not limited to any particular mechanism and an understanding of the mechanism is not necessary to practice the present invention, that activated PKA hyperactivates the small GTPase Rho subsequently suppressing stress fiber formation and ultimately cell migration and angiogenesis. Furthermore, the present invention further contemplates that PKA inhibits Rac and PAK activation, thus preventing lamellipodia formation. Thus, preferred embodiments provide methods and compositions that interference with normal actin dynamics and that eliminate cell polarity and suppresses migration of tumor cells and endothelial cells. In vitro activation of PKA by integrin antagonists, by cell permeable cAMP, or by overexpression of the catalytic subunit of PKA, and the like, inhibits cell migration. Indeed, the present invention shows that in vivo activation of PKA inhibits angiogenesis. PKA is a critical regulator of invasive cell behavior during tissue remodeling, such as occurs during angiogenesis and metastasis.

In still another embodiment, the present invention provides methods and compositions comprising PTH and/or PTHrP hormone to activate PKA and phospholipase C. Although an understanding of the mechanism is not necessary to practice the present invention and the present invention is not so limited, it is contemplated that extracellular PTHrP and PTH activate a cell surface G protein coupled receptor (GPCR) that in turn signals the activation of protein kinase A and phospholipase C. (See e.g. A. B. Abou-Samra et al., Proc. Natl. Acad. Sci. U.S.A., 89:2732-2736 (1992); and S. Hoare et al., J. Biol. Chem., 276:7741-7753 (2001)). This receptor is expressed on many cells including chondrocytes, vascular smooth muscle cells, and endothelial cells. (M. Iwamoto, J. Biol. Chem., 269:17245-17251 (1994); S. Maeda et al., Endocrinology, 140:1815-1825 (1999); and B. Jiang et al., J. Cardiovascular Pharmacol., S142:1444 (1998)). Many of the physiological functions of PTHrP are mediated by activation of PKA.

PTH and the closely related PTHrP hormone are important and well-described peptide hormones produced by tumor cells and normal cells that regulate serum calcium levels, vascular tone and bone formation. Undesirable PTH and/or PTHrP levels in a subject can cause sever deleterious effects, for example, mice lacking PTHrP die shortly after birth and suffer from skeletal abnormalities that include shortened bones and increased bone vascularization. (See e.g., A. C. Karaplis et al., Genes. Dev., 8:277-289 (1994); N. Amizuka et al., J. Cell Biol., 126:1611-1623 (1994) and B. Lanske et al., J. Clin. Invest., 104:399-407 (1999)).

Naturally occurring isoforms of PTHrP extend from amino acids 1-139, 1-141, and 1-173, which are found only in humans. (J. M. Moseley and T. J. Martin, Parathyroid Hormone-Related Protein: Physiological Actions, pp. 363-376, in Principles of Bone Biology, Academic Press, Inc., (1996)).

Although PTHrP is produced by many tumor cells, little is known about its effects on tumor vasculature in contrast, PTHrP is known to cause elevated serum calcium levels in cancer patients (e.g., humoral hypercalcemia of malignancy (HHM)) and to promote local bone metastases. (See e.g., G. A. Nickols et al., Endocrinology, 125:834-841 (1989); T. A. Guise et al., J. of Clin. Invest., 98:1544-1549 (1996); and T. Yoneda, European J. Cancer, 34:240-5 (1998)). HHM results from PTHrP stimulation of osteoblasts to express RANKL, a factor that induces osteoclast differentiation from macrophages, subsequent degradation of mineralized bone and eventual serum calcium elevation. (R. J. Thomas et al., Endocrinology, 140:4451-4458 (1990)). Local PTHrP mediated bone degradation may contribute to tumor cell colonization of bone to form metastases. Recent studies indicate that PTHrP also acts in an intracrine fashion to stimulate tumor cell proliferation. (A. Guijal et al., Canc. Res., 61:2282-8888 (2001)).

Recent studies show that PTHrP may promote tumor progression, but also may negatively regulate bone vascularization. Certain embodiments of the present invention are directed to determining the role PTHrP plays in the physiological regulation of angiogenesis. The present invention shows that the angiostatic effects of PTHrP require activation of PKA by its G protein coupled receptor. Once activated, PKA suppresses Rac activation, which in turn inhibits endothelial cell migration and angiogenesis. Thus, in one embodiment, the present invention provides methods and compositions for the localized administration of PTHrP or activation of PKA to treat (e.g., inhibit) diseases characterized by undesirable angiogenesis.

Data herein shows one embodiment where PKA is activated by inhibition of integrin ligation. In particular, data herein shows the time course of PKA activation in endothelial cells placed in suspension or maintained in monolayers: the time course of PKA activation in endothelial cells maintained in suspension for 30 min (t=30), then maintained in suspension or allowed attached to vitronectin coated culture plates; the time course of PKA activation in endothelial cell monolayers treated with or without 250 μM dibutyryl camp; the activity in endothelial cells maintained in suspension for 60 min (sus) or allowed to attach for 60 min to vitronectin, fibronectin, or collagen-coated culture plates in the absence (med) or presence of antibody antagonists of integrins α5β1, α2β1, and αvβ3.

Data herein also shows one embodiment where unligated integrin α5β1 suppresses endothelial and tumor cell migration on, but not cell attachment to, provisional ECM proteins. In particular, data herein shows endothelial cell adhesion (left) and migration (right) on vitronectin in the presence of medium, anti-α5β1, anti-αvβ3, and anti-α2β1 antibodies. Data herein shows endothelial cell adhesion (left) and migration (right) on Del 1 in the presence of medium, anti-α5β1, anti-αvβ3, and anti-α2β1 antibodies; endothelial cell migration on collagen and laminin in the presence of medium, anti-α5β1, anti-αvβ3, anti-α2β1, or anti-a6b1 antibodies; Rhodamine-Phalloidin staining for F-actin in endothelial cells attached to vitronectin in the absence (medium) or presence of anti-integrin α5β1 or α2β1.

Additional data herein also shows an embodiment of the role of PKA in integrin regulation of endothelial cell migration. For example, data herein shows activity in endothelial cells transfected with expression constructs encoding GFP or mutationally inactive PKA (dnPKA) and treated with cAMP or placed in suspension. Furhter data herein shows expression of dnPKA and GFP transgenes in endothelial cells was detected by Western blotting for V5 tag and GFP. Transfection efficiency of HUVECs by electroporation is 70-80%; expressed proteins are detected by immunoblotting of cell lysates for GFP and V5tag on dnPKA; migration of N1GFP (black bars) and pcDNA 3.1 V5/his-dnPKA and N1GFP (gray bars) transfected endothelial cells on vitronectin in the presence of 25 μg/ml anti-α5β1, anti-α2β1 or anti-αvβ3 antibodies. Expression of dnPKA and GFP transgenes in endothelial cells was detected by Western blotting for V5 tag and GFP. Data herein also shows migration of endothelial cells transfected with pcDNA 3.1 V5/his-dnPKA and N1GFP (stippled bars) or N1GFP alone (black bars) on vitronectin in the presence of 25 μg/ml anti-αvβ3, anti-avb5, anti-α2β1 or anti-αvβ3 in combination with anti-avb5; migration of endothelial cells transfected with pcDNA 3.1 V5/his-PKA catalytic subunit (PKAcat) and N1GFP or N1GFP (GFP) vectors alone on vitronectin (VN), fibronectin (FN) and collagen (COLL); and expression of transgenes in endothelial cells was detected by Western blotting for the V5tag on PKAcat and for GFP. Data herein also shows migration of endothelial cells transfected with pcDNA 3.1 V5/his-dnPKA and N1GFP or N1GFP (black bars) vectors alone on vitronectin (VN) in the presence or absence of 250 μM dibutyryl cAMP. The inventors' data also shows Phalloidin staining for F-actin in pcDNA 3.1 V5/his-dnPKA and N1GFP (dnPKA) or N1GFP (GFP) transfected endothelial cells attached to vitronectin in the absence or presence of anti-integrin α5β1 or α2β1. Phalloidin staining for F-actin in pcDNA 3.1 V5/his-PKAcat and N1GFP transfected cells and in cAMP treated endothelial cells. Transfected cells were identified by green fluorescence.

Data herein shows an embodiment of the role of Rho in the suppression of cell migration by unligated integrins. For example, the data shows affinity precipitated RhoGTP and total Rho levels measured in endothelial cells allowed to attach to vitronectin for 0-90 min. The ratio of active Rho (RhoGTP) to total Rho was determined and graphed versus time. Data herein also shows affinity precipitated RhoGTP and total Rho levels measured in endothelial cells attached to vitronectin for 0-30 min in the presence of culture medium, anti-α5β1 and anti-α2β1. The ratio of active Rho (RhoGTP) to total Rho was determined and graphed versus time. The data also shows the migration of endothelial cells transfected with N1GFP (GFP) or with HA-tagged pcDNA 3.1 expression vectors encoding N19Rho or V14Rho with N1GFP on vitronectin in the presence or absence of anti-α5β1. Transfected cells were identified by green fluorescence. Data herein shows expression of GFP and mutated Rho proteins were detected by Western blotting of detergent extracts of transfected cells with anti-GFP or -HA tag antibodies. The invention's data shows Phalloidin staining for F-actin in pcDNA 3.1 N19Rho and N1GFP or N1GFP (GFP) transfected endothelial cells attached to vitronectin in the absence or presence of anti-integrin α5β1 or α2β1. Phalloidin staining for F-actin in pcDNA 3.1 V14Rho and N1GFP transfected cells. Transfected cells were identified by green fluorescence.

Data herein further show that an embodiment of the role of Rac in the suppression of cell migration by unligated integrins. In particular, the data shows affinity precipitated RacGTP and total Rac levels measured in endothelial cells allowed to attach to vitronectin for 0-90 min. The ratio of active Rac (RacGTP) to total Rac was determined and graphed versus time. Data also shows affinity precipitated RacGTP and total Rac levels measured in endothelial cells attached to vitronectin for 0-30 min in the presence of culture medium anti-α5β1 and anti-α2β1. The ratio of active Rac (RacGTP) to total Rac was determined and graphed versus time. The data additionally shows the migration of HUVECs cells transfected with N1GFP (GFP) or with HA-tagged pcDNA 3.1 expression vectors encoding N17Rac or V12Rac with N1GFP on vitronectin in the presence or absence of anti-α5β1. Transfected cells were identified by green fluorescence. The data also shows the expression levels of GFP and mutated Rac proteins were detected by Western blotting of detergent extracts of transfected cells with anti-GFP or -HA tag antibodies. Exemplary data shows Phalloidin staining for F-actin in pcDNA 3.1 V12Rac and N1GFP or N1GFP (GFP) transfected endothelial cells attached to vitronectin in the absence or presence of anti-integrin α5β1 or α2β1. Phalloidin staining for F-actin in pcDNA 3.1 N17Rac and N1GFP transfected cells. Transfected cells were identified by green fluorescence. The invention's data shows cell migration on vitronectin of endothelial cells transfected with GFP or with GFP and constitutively active p21 kinase (dpPAK) in the absence or presence of cAMP, anti-α5β1 or anti-α2β1. The data also shows cell migration on vitronectin of endothelial cells transfected with GFP or with GFP and mutationally inactive (dnPAK) or constitutively active p21 kinase (dpPAK).

Data herein also show one embodiment where PKA negatively regulates migration and angiogenesis: role in hyperactivation of Rho and inhibition of Rac activation. For example, the data shows affinity precipitated RhoGTP and total Rho levels measured in endothelial cells treated with medium or 250 μM dibutyryl cAMP or transfected with N1GFP or PKAcat that allowed to attached to vitronectin for 15 min. The ratio of active Rho (RhoGTP) to total Rho was determined. The data shows migration on vitronectin of GFP or N19Rho transfected endothelial cells treated with medium or 250 μM dibutyryl cAMP or co-transfected with PKAcat. The data further shows affinity precipitated RacGTP and total Rac levels measured in endothelial cells treated with medium or 250 μM dibutyryl cAMP or transfected with N1GFP or PKAcat that allowed to attached to vitronectin for 15 min. The ratio of active Rac (RhoGTP) to total Rac was determined. The data also shows migration on vitronectin of GFP or V12Rac transfected endothelial cells treated with medium or 250 μM dibutyryl cAMP or co-transfected with PKAcat. The data shows CAMs stimulated with bFGF were transfected 24 hrs later by placing 4 μg pcDNA/V5/His dnPKA or N1-GFP expression plasmid on CAMs. CAMs were treated on the same day with saline or anti-α5β1 antibodies. CAMs were harvested 48 hrs later. Blood vessel branch points were quantified. Data herein also shows CAMs stimulated with bFGF, treated with saline or 250 μM cAMP or transfected by placing 4 μg pcDNA/V5/His PKAcat or N1-GFP expression plasmid on stimulated CAMs.

Data herein shows one embodiment where PTHrP inhibits angiogenesis in vivo and endothelial cell migration in vitro. For instance, the data shows CAMs stimulated with saline or bFGF and treated 24 hrs later with (a-b) 1 μM PTHrP (1-173), calcitonin and calcitonin gene related peptide (CGRP). Further data herein shows blood vessel branch points+/−S.E.M. above background. The data also shows cryosections of CAMs were immunostained with LM609, an anti-αvβ3 antibody, and immunoreactive vessels per 100× microscopic field were quantified and photographed for each treatment. The data additionally shows CAMs stimulated with bFGF and treated 24 hrs later with saline, 1 μM PTHrP (1-173) or 1 μM PTHrP in combination with 25 μg/ml PTHrP function blocking antibody 8B12 or control antibody. Blood vessels branch points+/−S.E.M. above background and representative CAMs photographed at 10×. Data herein shows growth factor-depleted matrigel containing purified bFGF (1 μg/ml) and 1 μM PTHrP or calcitonin was injected subcutaneously into 10 nude mice each. After three days, mice were injected intravenously with the endothelial cell binding FITC-Griffonia (Bandeiraea) simplicifolia lectin and fluorescence intensity in lysates of matrigel plugs was determined. The data show that mice bearing 30 mm³ PTHrP negative DU145 tumors were treated for ten days once daily with saline, PTHrP or a scrambled control peptide (1 μM final concentration). In particular, the data shows the average increase in tumor volume+/−S.E.M. over the treatment period. Also shown are cryosections immunostained for expression of vascular antigen CD31 and DNA. Immunoreactive vessels were quantified and photographed. P<0.05 was determined by Student's t-test.

The data herein shows one embodiment where angiogenesis and tumor growth is inhibited by PTHrP gene delivery. For example, the data shows chicken embryos bearing CAMs stimulated with bFGF or VEGF were injected with adenoviruses expressing Green Fluorescent Protein (GFP) or full length PTHrP. In particular, the data shows blood vessels branch points+/−S.E.M. above background. Cryosections of treated CAMs were immunostained with anti-PTHrP antibodie. PTHrP positive blood vessels were observed. The data shows cryosections of bFGF stimulated CAMs were immunostained with anti-αvβ3 antibodies. Immunoreactive vessels per 100× microscopic field were quantified and photographed for each treatment. The data shows growth factor-depleted matrigel containing purified bFGF (1 μg/ml) and PTHrP or GFP expressing adenoviruses was injected subcutaneously into 10 nude mice each. After three days, mice were injected intravenously with the endothelial cell binding FITC-Griffonia (Bandeiraea) simplicifolia lectin and fluorescence intensity in lysates of matrigel plugs was determined. The data shows chicken embryos with CAMs bearing 50 mg fragments of DU145 prostate carcinoma tumors (expressing low levels of endogenous PTHrP) were injected with adenoviruses expressing Green Fluorescent Protein (GFP) or full length PTHrP. In particular, the data shows tumors were excised, weighed and photographed at 10× magnification after seven days of growth. The data also shows cryosections of tumors from samples immunostained with anti-αvβ3 antibodies. Immunoreactive vessels per 100× microscopic field were quantified and photographed for each treatment, and P<0.05 was determined by Student's t-test.

Data herein also shows one embodiment where the N-terminal 34 amino acids of PTHrP inhibit endothelial cell migration in vitro and angiogenesis in vivo. For example, the data shows that endothelial cell migration and attachment to vitronectin in the presence of buffer, 25 μg/ml anti-αvβ3, 1 μM PTHrP, 1 μM calcitonin or 1 μM CGRP was determined. The effect of migration buffer, 25 μg/ml anti-αvβ3, 1 μM PTHrP 1-141, 1-86 and 1-34, as well as 1 μM calcitonin on endothelial cell migration or attachment to vitronectin was determined. The data shows CAMs stimulated with bFGF and treated 24 hrs later with saline, 1 μM PTHrP fragments 1-141, 1-86 and 1-34 and calcitonin. Blood vessels branch points were counted 48 hrs later at 30×. P<0.05 was determined by Student's t-test.

Data herein also shows one embodiment where PTHrP amino acids 1-10 are sufficient to inhibit endothelial cell migration in vitro and angiogenesis in vivo. For instance, the data shows the effect of migration buffer, 25 μg/ml anti-αvβ3, 1 μM PTHrP 1-34, 1-10, 15-34 and scrambled 1-10, as well as 1 μM calcitonin on endothelial cell migration vitronectin was determined. Also shown is that CAMs stimulated with bFGF and treated 24 hrs later with saline, 1 μM PTHrP 1-10, scrambled 1-10 and 1-34. Blood vessels branch points were counted 48 hrs later at 30×. The data shows growth factor-depleted matrigel containing purified bFGF (1 μg/ml) and saline, 1 μM PTHrP 1-10 or 1 μM PTHrP scrambled 1-10 was injected subcutaneously into 10 nude mice each. After three days, mice were injected intravenously with the endothelial cell binding FITC-Griffonia (Bandeiraea) simplicifolia lectin and fluorescence intensity in lysates of matrigel plugs was determined. The data shows CAMs stimulated by bFGF, VEGF, IL-8 or TNFa were treated with saline and a range of concentrations of PTHrP 1-34 extending from 0.001-10 μM. Blood vessels branch points were counted at 30×. Percent inhibition of saline control was determined. P<0.05 was determined by Student's t-test.

The data shows one embodiment where PTHrP inhibits migration by blocking Rac activation in a PKA dependent manner. For example, the data shows protein kinase A activity in endothelial cells stimulated by culture medium, 10 μM PTHrP or 250 μM dibutyryl cAMP determined using a colorimetric assay kit. Specific activity is expressed as percent of total PKA that can be directly activated by cAMP in cell lysates. PKA values for 0-30 minute exposure times were determined. The data shows endothelial cell migration on vitronectin was evaluated in the presence of culture medium, anti-αvβ3, PTHrP (1-34), the protein kinase A inhibitor, H89, and PTHrP combined with H89. The data shows the effect of a mutationally inactive form of PKA (dnPKA) on the inhibition of cell migration on vitronectin by PTHrP, a scrambled peptide control and cAMP was determined. The data shows the effect of PKA activation by cAMP or by transient transfection with the PKA catalytic subunit (PKAcat) on endothelial cell migration on vitronectin was determined. The data also shows Western blots of lysates of GFP, dnPKA, and PKAcat transfected cells incubated in anti-V5 tag and anti-GFP antibodies to detect expression of the transgenes. The data shows Rac activity measured by a PAK affinity pulldown assay in endothelial cells treated with culture medium or PTHrP and in cells transfected with activated PKA or GFP. The data further shows endothelial cells expressing mutationally active Rac (V12 Rac, +) or GFP (−)treated with culture medium, PTHrP or cAMP prior to evaluating cell migration on vitronectin. The data additionally shows endothelial cells transfected with PKAcat, V12 Rac+PKA cat, GFP or V12 Rac+GFP. Forty-eight hrs later, cell migration on vitronectin was evaluated.

The data shows one embodiment where PTHrP mediated inhibition of angiogenesis is protein kinase A dependent. In particular, the data shows CAMs stimulated with bFGF or saline and treated 24 hrs later with saline, 1 μM PTHrP 1-34, 480 nM H89, or 1 μM PTHrP+480 nM H89. The data shows CAMs stimulated with bFGF or saline and treated 24 hrs later with 4 μg pcDNA/V5 tagged GFP DNA, or PTHrP+4 μg pcDNA/V5 tagged GFP DNA, 4 μg pcDNA/V5 tagged dnPKA DNA, or PTHrP+4 μg pcDNA/V5 tagged dnPKA DNA. The data shows CAMs stimulated with bFGF, VEGF or saline (PBS) and treated 24 hrs later with saline (−) or 250 μM dibutyryl cAMP (+). The data shows CAMs stimulated with bFGF or saline and treated 24 hrs later with saline, 4 μg pcDNAV5 tagged PKAcat or 4 μg pcDNAV5 tagged GFP DNA. Blood vessels branch points were counted 48 hrs later at 30×. P<0.05 was determined by Student's t-test.

The data show one embodiment where fibronectin and integrin α5β1 support endothelial cell survival. The data shows serum-starved HUVECs maintained in suspension (SUS) or on fibronectin (Fn), poly-L-lysine (PLL) or collagen (COL)-coated plates. In particular, the data shows the percentage of Annexin V positive cells determined from 0-8 hrs. Cell lysates prepared after 4 hrs of attachment were immunoblotted to detect intact (116 kDa) and cleaved PARP (85 kDa). The ratio of intact to cleaved PARP was determined by densitometry. Soluble DNA extracted from cells attached to PLL, Fn or COL was electrophoresed on 1.6% agarose gels. Relative DNA cleavage was determined by densitometry. The data show HUVECs plated on fibronectin, anti-α5β1 or control antibody-coated plates. In particular, the data shows the percentage of Annexin V positive cells was determined from 0-8 hrs. Data herein shows cell lysates immunoblotted to detect intact and cleaved PARP. DNA fragmentation was evaluated as disclosed elsewhere herein.

Data herein also shows one embodiment where that integrin α5β1 supports endothelial cell survival during angiogenesis in vivo. The data shows bFGF stimulated CAMs treated for 24 hrs with saline, anti-α5β1, anti-αvβ3 and control antibodies. CAMs were then injected with 50 μl FITC-Annexin V, harvested 2 hrs later and analyzed directly by confocal microscopy. Green pixels present per optical section were quantified. Treated CAMs were cryosectioned and immunostained with anti-cleaved caspase 3 (green) and anti-vWF (red). Cleaved caspase 3 positive blood vessels were yellow. The data shows individual cells isolated from CAMs treated as discussed above were stained with FITC-Annexin V. Soluble DNA isolated from CAMs treated as discussed was electrophoresed on 1.6% agarose gels. Molecular weight markers were 1 kb DNA ladders. The data shows relative DNA cleavage determined by densitometry.

The data also shows one embodiment where unligated integrin α5β1 regulates endothelial cell survival. Serum starved or serum cultured HUVECs were plated on poly-L-lysine, fibronectin, vitronectin, or collagen coated culture plates in the absence or presence of anti-α5β1, anti-αvβ3, anti-α2β1 antibodies. After 1 hr, cell attachment was determined. After 24 hrs, the percentage of FITC-Annexin V positive cells was determined. HUVECs plated on vitronectin coated plates in the presence of function blocking anti-α5β1 or control antibodies were collected at regular intervals from 0-8 hrs and PARP cleavage assessed by Western blotting. Relative PARP cleavage levels were determined by densitometry.

Data herein also shows one embodiment where unligated integrin α5β1 induces caspase 3 and 8, but not 9, activation. HUVECs were plated on fibronectin, vitronectin or poly-L-lysine (PLL) coated culture plates in the presence of anti-α5β1 antibodies and 50 μM z-DEVD-fmk (caspase-3) or z-IETD-fmk (caspase-8) inhibitors or vehicle control (0.33% DMSO) for 24 hrs. The percentage of Annexin V positive cells was determined. Caspase 3 and 8 activities were determined in HUVECs plated on vitronectin (VN), or poly-L-lysine (PLL) coated plates in the presence of culture medium, anti-α5β1 or control antibodies for 1 hr. Cell lysates were immunoblotted with anti-caspase 3 and anti-cleaved caspase 3 antibodies. Relative caspase 3 cleavage was determined by densitometry. Cell lysates were immunoblotted with anti-caspase 9 and anti-cleaved caspase 9 antibodies.

Data herein additionally shows one embodiment where integrin antagonists induce caspase 3- and 8-dependent apoptosis in vivo. CAMs stimulated with saline or bFGF were treated with 2.5% DMSO (vehicle control), anti-α5β1, or anti-α5β1 with 500 μM caspase 3 or caspase 8 inhibitors. CAMs stimulated with saline or bFGF were treated with saline containing 2.5% DMSO, anti-αvβ3, or anti-αvβ3 with 500 μM caspase 3 or caspase 8 inhibitors. Blood vessel branch points were counted after 48 hrs.

Data herein also shows one embodiment where cell death induced by unligated α5β1 is blocked by inhibitors of PKA. In particular, PKA activity was measured in HUVECs attached to poly-L-lysine, fibronectin or vitronectin in the presence or absence of integrin antagonists. HUVECs were plated on vitronectin or poly-L-lysine (PLL) coated culture plates in the presence or absence of anti-α5β1 or anti-αvβ3 antibodies, a selective PKA inhibitor (1 μM HA1004), or anti-integrin antibodies in combination with 1 μM HA1004. After 24 hrs, the percentage of FITC-Annexin positive cells was determined. Cell lysates were immunoblotted with anti-caspase 3 and anti-cleaved caspase 3 antibodies. Relative caspase 3 cleavage was determined by densitometry.

Data herein shows one embodiment where PKA negatively regulates integrin mediated cell survival. For instance, HUVECs transfected with GFP (−) or a mutationally inactive PKA (dnPKA, +) were plated on fibronectin, vitronectin, collagen, or poly-L-lysine (PLL) coated plates in the absence or presence of anti-α5β1, anti-αvβ3, or anti-α2β1. Serum cultured cells were plated on fibronectin and vitronectin while serum-starved cells were plated on collagen. After 24 hrs, the percentage of Annexin V positive cells was determined. HUVECs treated with culture medium or dibutyryl cAMP (250 μM) and HUVECs transfected with GFP or the catalytic subunit of PKA (PKAcat) were plated on vitronectin (Vn), or poly-L-lysine (PLL) coated plates. After 24 hrs, the percentage of Annexin positive cells was determined. Expression of transgenes was detected by Western blotting cell lysates with anti-GFP or anti-V5.

Data herein shows one embodiment where PKA inhibits angiogenesis by inducing apoptosis. CAMs stimulated with bFGF were transfected 24 hrs later by placing 4 μg pcDNA/V5/His dnPKA or N1-GFP expression plasmid on CAMs. CAMs were treated on the same day with saline or anti-α5β1 antibodies. CAMs were harvested 48 hrs later. Blood vessel branch points were quantified. CAMs stimulated with bFGF were treated with saline or 250 μM cAMP or were transfected by placing 4 μg pcDNA/V5/His PKAcat or N1-GFP expression plasmid on stimulated CAMs. Detergent lysates prepared from freshly excised CAMs were immunoblotted for expression of caspase 3 and cleaved caspase 3. Caspase 3 cleavage was quantified by densitometry.

### DETAILED DESCRIPTION OF THE INVENTION

The invention relates to methods for detecting and inhibiting angiogenesis, cell migration, cell adhesion, and/or cell survival in endothelial and non-endothelial cells as well as in normal and tumor cells. The invention further relates to methods for screening test compounds for their ability to inhibit angiogenesis, cell migration, cell adhesion, and/or cell survival.

Exemplary compositions and methods of the present invention are described in more detail in the following sections: I. Inhibiting angiogenesis, cell migration, and/or cell adhesion; II. Inducing apoptosis; III. Screening compounds; IV. Exemplary agents; and VI. Pharmaceutical formulations, administration routes, and dosing considerations.

The following exemplarily embodiments primarily focus on methods and compositions for inhibition integrin α5β1 ligation and for activating PKA. The present invention is not, however, intended to be limited to providing therapeutic compositions and methods for modulating undesirable modulating angiogenesis, cell migration, cell adhesion, and/or cell survival as practiced in the following specific embodiments. Indeed, one skilled in the art will recognize the broad applicability of the compositions and methods described in the present invention, including the specific example and embodiments presented, for the modulating undesirable cellular migration, modulating angiogenesis, cell migration, cell adhesion, and/or cell survival more generally.

I. Inhibiting Angiogenesis, Cell Migration, and/or Cell Adhesion

“Angiogenesis,” or neovascularization, is the process by which new blood vessels develop from pre-existing vessels. (See e.g., J. A. Varner et al, Angiogen., 3(1):53-60 (1999); Mousa et al., Angiogen. Stim. Inhib., 35:42-44 (2000); Kim et al., Amer. J. Path. 156:1345-1362 (2000); Kim et al, J. Biol. Chem., 275:33920-33928 (2000); and Kumar et al., Angiogenesis: From Molecular to Integrative Pharm. 169-180 (2000)). Endothelial cells from pre-existing blood vessels or from circulating endothelial stem cells (Takahashi et al, Nat. Med., 5:434-438 (1995); and Isner et al., J. Clin. Invest., 103:1231-1236 (1999)) become activated to migrate, proliferate, and differentiate into structures with lumens, forming new blood vessels, in response to growth factor or hormonal cues or hypoxic or ischemic conditions. During ischemia, such as occurs in cancer, the need to increase oxygenation and delivery of nutrients apparently induces the secretion of angiogenic factors by the affected tissue; these factors stimulate new blood vessel formation. The level of angiogenesis may be determined using methods well known in the art, including, without limitation, counting the number of blood vessels and/or the number of blood vessel branch points as disclosed herein. An alternative assay involves an in vitro cell adhesion assay that shows whether a compound inhibits the ability of integrin alpha 4 beta 1-expressing cells (e.g. M21 melanoma cells) to adhere to VCAM or fibronectin. Another in vitro assay contemplated includes the tubular cord formation assay which shows growth of new blood vessels at the cellular level (D. S. Grant et al., Cell, 58: 933-943 (1989)). Art-accepted in vivo assays are also known, and involve the use of various test animals such as chickens, rats, mice, rabbits and the like. These in vivo assays include the chicken chorioallantoic membrane (CAM) assay which may be used to show anti-angiogenic activity in both normal and neoplastic tissues (D. H. Ausprunk, Amer. J. Path., 79, No. 3: 597-610 (1975) and L. Ossonowski and E. Reich, Cancer Res., 30: 2300-2309 (1980)). Other in vivo assays include the mouse metastasis assay, which shows the ability of a compound to reduce the rate of growth of transplanted tumors in certain mice, or to inhibit the formation of tumors or preneoplastic cells in mice which are predisposed to cancer or which express chemically-induced cancer (M. J. Humphries et al., Science, 233: 467-470 (1986) and M. J. Humphries et al., J. Clin. Invest., 81: 782-790 (1988)).

The term “migration” as used herein refers to the translocation of a cell across one or more components of the extracellular matrix (e.g., fibronectin, collagens I-XVIII, laminin, vitronectin, fibrinogen, osteopontin, Del 1, tenascin, von Willebrands's factor, etc.), and/or along the surface of another cell (e.g., another endothelial cell, fibroblast cell, stromal cell, tumor cell, etc.). Methods for determining cell migration are known in the art, and are also disclosed herein.

The term “adhesion” when made in reference to a cell refers to binding of the cell to one or more molecules, such as to components of the extracellular matrix (e.g., fibronectin, collagens I-XVIII, laminin, vitronectin, fibrinogen, osteopontin, Del 1, tenascin, von Willebrands's factor, etc.), to one or more ligand which is expressed on the cell surface (e.g., VCAM, ICAM, LI-CAM, VE-cadherin, integrin alpha 2, integrin alpha 3, etc.) and/or to another cell (e.g., endothelial cell, fibroblast cell, stromal cell, tumor cell, epithelial cell, etc.). Such measurement may be accomplished using methods described herein and known in the art.

A. Unligated Integrins and Activation of Protein Kinase A

Preferred embodiments of the present invention show that integrin ligation transduces signals that promote cell survival, proliferation and migration, however, unligated integrins transduce signals that negatively regulate cell proliferation and survival (e.g., activation of PKA). To thus examine the roles of integrins in the regulation of PKA activity in normal cells (e.g., endothelial cells) and tumor cells (e.g., lymphoma, leukemia, and carcinomas, and the like), certain embodiments measured PKA levels in attached and suspended cells as a function of time. The present invention shows that integrin ligation suppresses PKA. Conversely, the present invention also shows that inhibition of integrin ligation rapidly activates PKA, and that cell detachment from the ECM also rapidly activates PKA. In some of these embodiments, high levels of PKA activity were observed as little as 2 min after cell detachment and were maintained for up to six hours after detachment. In contrast, cell re-attachment rapidly suppresses PKA activity, with little PKA activity detectable after one hour of attachment. In some embodiments, exposure to cell permeable cAMP similarly activates PKA in normal (e.g., endothelial cells) and cancerous cells attached to ECM proteins.

Although the present invention is not limited to any particular mechanism and an understanding of the mechanism is not necessary to practice the present invention, the present invention contemplates that PKA is activated by the binding of a cAMP molecule to each regulatory subunit, leading to the dissociation of the (activated) catalytic subunit. Cyclic AMP in turn is produced by adenylyl cyclases, which are regulated by stimulatory and inhibitory heterotrimeric G proteins. For example, one Gαi-linked receptor associated with integrins is CD47. Since CD47 regulates integrin αvβ3 function, some embodiments of the present invention contemplate that integrin α5β1 regulates αvβ3 function by modulating the activity of CD47. Moreover, still further embodiments of the present invention contemplate that inhibition of integrin α5β1 blocks CD47 function, leading to the activation of PKA and inhibition of αv specific cell migration.

In some preferred embodiments, the present invention provides methods and compositions for inhibition of integrin ligation comprising administering anti-integrin antibodies to a subject. In some of these embodiments, these antibodies selectively block cell attachment to ECM proteins thus activating PKA. For example, inhibition of cell attachment to vitronectin by integrin αvβ3 antagonists, cell attachment to fibronectin by integrin α5β1 antagonists, or cell attachment to collagen by integrin α2β1 antagonists activates PKA. However, inhibition of integrin α5β1 function in endothelial cells attached to vitronectin also stimulates PKA activity even though cell attachment to this provisional ECM protein is not affected by α5β1 inhibition. In contrast, inhibition of α5β1 does not activate PKA in cells attached to collagen. Integrin α5β1 is a receptor for fibronectin, a provisional matrix protein secreted by proliferating endothelial cells in vitro and during angiogenesis in vivo. These results suggest that integrin α5β1 regulates PKA activation in the context of cell attachment to provisional matrix proteins. Ligation of integrin α5β1 is required to suppress PKA activation in cells attached to provisional matrix proteins, but not in cells attached to non-provisional matrix proteins. Together, these results also indicate that integrin ligation suppresses PKA activation.

In still other embodiments, antagonists of α5β1 not only activate PKA but also inhibit endothelial cell migration on vitronectin without impacting cellular attachment to vitronectin. In contrast, integrin αvβ3 antagonists block migration and adhesion to vitronectin, while antibodies against integrin α5β1 have no effect on cell attachment or migration on vitronectin. The present invention also contemplates that antagonists of α5β1 block cell migration but not adhesion on other provisional matrix protein ligands for αᵥβ₃ such as Del-1 (See, K. Penta et al., J. Biol. Chem., 274:11101-11109 (1999)) and proteolyzed collagen (See, B. P. Elicieri and D. A. Cheresh, J. Clin. Invest., 103:1227-1230 (1999)). In contrast, integrin α5β1 antagonists do not block endothelial cell migration on non-provisional matrix proteins such as collagen or laminin. Importantly, the ability of α5β1 to regulate cell migration on provisional matrix proteins is not restricted to endothelial cells. Indeed, the present invention shows that integrin α5β1 regulates migration of most cells, including normal endothelial cells, fibroblasts, as well as breast, prostate, melanoma and other tumor cells on provisional matrix proteins.

Cell migration requires the formation of stress fibers and lamellipodia. (See e.g. J. Padmanabhan et al., J. Neurobiol., 39:407-422 (1999)). Thus, some embodiments of the present invention are directed to determining the effects of integrin antagonists on stress fiber formation in endothelial cells. Endothelial cells attached to vitronectin in culture medium or in the presence of anti-integrin α2β1 antibody antagonists rapidly spread, polarize and make stress fibers. In contrast, endothelial cells attached to vitronectin in the presence of α5β1 antagonists exhibit a pancake-like morphology. These cells lack stress fibers, as well as pseudopodia, and thus exhibit no polarization. These results suggest that integrin α5β1 indirectly regulates the ability of integrin αvβ3 to promote the actin assembly events required for cell migration.

Since integrin α5β1 antagonists activate PKA but inhibit cell migration on vitronectin, still further embodiments of the present invention tested the role of PKA in cell migration by evaluating the effects of mutationally inactive and active forms of PKA on cell migration. PKA activation induced by inhibition of cell attachment (e.g., suspension culture) or by cell-permeable cAMP is completely suppressed by expression of a mutationally inactive PKA construct (dnPKA), a regulatory subunit that fails to release the catalytic subunit upon exposure to cAMP25. Seventy to eighty percent of cells were routinely transfected in the studies, as monitored by quantifying the number of GFP co-transfected cells. In preferred embodiments, protein expression of dnPKA in transfected endothelial cells was verified by Western blotting. In yet other embodiments, mutationally inactive PKA overcame the inhibition of migration on vitronectin induced by α5β1 antagonists in endothelial cells and tumor cells. Similar effects were seen for migration on proteolyzed collagen, another provisional matrix protein. Therefore, the present invention shows that PKA mediates the indirect inhibition of cell migration on vitronectin by α5β1 antagonists.

Still further studies show that when more than one integrin mediates cell migration on a single ECM substrate, inhibition of one of these integrins can activate PKA and thereby partially suppress cell migration. For example, integrin αvβ3 antibodies partially inhibit cell migration on vitronectin. In some embodiments, the present invention contemplates that this inhibition can be reversed by dnPKA. As integrins αvβ3 and αvβ5 both contribute to cell migration on vitronectin it is possible that PKA activated by antagonized αvβ5 partially suppresses cell migration mediated by αvβ5. Therefore, further studies are directed to determining whether inhibition of cell migration on vitronectin by αvβ5 antibody antagonists could also be reversed by dnPKA. Indeed, in some embodiments, the expression of dnPKA did reverse the inhibition of cell migration induced by αvβ5 antagonists. However, dnPKA could not suppress the combined inhibitory effects of αvβ3 and αvβ3 antibodies on cell migration as endothelial cells cannot attach to the substratum in the presence of inhibitors of both αv integrins. Similarly, inhibition of cell migration on collagen by integrin α2β1 antagonists is not be reversed by dnPKA, as α2β1 is the only receptor for collagen on endothelial cells. The present invention contemplates that unligated or antagonized integrins activate PKA and that activated PKA inhibits cell migration without inhibiting cell attachment. The present invention further contemplates that PKA activation by integrin antagonists suppresses cell migration even in ECM attached cells.

Present studies indicate that inhibition of integrin ligation activates PKA and blocks cell migration. Furthermore, inhibition of PKA permits cell migration in the presence of certain integrin antagonists. These results show that direct activation of PKA by cell permeable cAMP or by expression of activated PKA inhibits cell migration. In fact, preferred embodiments of the present invention show that expression of the catalytic subunit of PKA in endothelial cells blocks cell migration on fibronectin, vitronectin, and collagen. In some embodiments, expression of the transgene was verified by Western blotting. Similarly, the present invention also shows that exposure of cells to cAMP inhibits endothelial cell migration. In yet other embodiments, the present invention contemplates that tumor cell migration on fibronectin, vitronectin, or collagen is inhibited by expression of the catalytic subunit of PKA and by cell permeable cAMP. Accordingly, the present invention shows that the effects of cAMP on cell migration are PKA-dependent, as expression of dnPKA prevents the inhibitory effects of cAMP on endothelial cell migration.

As cell migration requires stress fiber and lamellipodia formation, as well as cell polarization, the present invention further examined the role of PKA in these processes. In one embodiment, endothelial cells transfected with GFP reporter vectors formed stress fibers and were polarized on vitronectin in the presence of control antibodies, but were flat, round and without stress fibers in anti-α5β1 treated cells. In contrast, cells expressing dnPKA and treated with anti-α5β1 re-acquired the ability to form stress fibers and polarize. Cells expressing the active PKA catalytic subunit or treated with cell permeable cAMP were round and exhibited few stress fibers. Accordingly, preferred embodiments of the present invention provide compositions and methods for activating PKA comprising administering integrin antagonists, or inducing the overexpression of active PKA or cAMP in a subject such that stress fiber formation and/or cellular polarization is suppressed.

The small GTPase Rho regulates stress fiber formation. (See e.g., J Padmanabhan et al., J. Neurobiol., 39:407-422 (1999); and X. D. Ren et al., EMBO J., 18:578-585 (1999)). The present invention contemplates that cellular detachment from the ECM activates Rho while adhesion to the ECM briefly suppresses Rho activity. This brief Rho suppression is required for the formation of stress fibers. Therefore, additional embodiments of the present invention are directed to determining the effect of anti-α5β1 antagonists (e.g., antibodies) on the activity of Rho in endothelial cells attached to vitronectin. Endothelial cells briefly suppress Rho after 15 min of cell attachment to vitronectin. However, in some embodiments, in the presence of inhibitors of α5β1, the brief suppression of Rho activity is eliminated. Rho activity remains higher for several hrs in anti-α5β1 treated cells than in control treated cells. Rho hyperstimulation plays an important role in α5β1 antagonists inhibiting cell migration on vitronectin by. In still further embodiments, the inhibition of cell migration mediated by α5β1 antagonists (e.g., antibodies) is nearly completely reversed by expression of mutationally inactive Rho (e.g., N19Rho) in endothelial cells and in tumor cells. In contrast, in other embodiments, expression of constitutively active Rho (e.g., V14Rho) suppresses migration of both endothelial cells and tumor cells. The expression of both transgenes is demonstrated by Western blotting the lysates from endothelial cells and tumor cells. In preferred embodiments, the expression of constitutively active Rho inhibits stress fiber formation, while expression of mutationally inactive Rho restores stress fibers and polarization to cells treated with integrin α5β1 antagonists. Interestingly, other recent studies show that high levels of active Rho suppress stress fiber formation and cell spreading in undifferentiated embryonic mesenchymal cells. (See e.g., B. Beqaj et al., J. Cell Biol., 156:893-903 (2002)). Rho activity declines as smooth muscle cells differentiate from mesenchymal cells; however, expression of constitutively active Rho suppresses cell spreading, stress fiber formation and differentiation. Although the present invention is not limited to any particular mechanism and an understanding of the mechanism is not necessary to practice the present invention, the present invention contemplates that the hyperactivation of Rho inhibits cell migration by inhibiting stress fiber formation. Accordingly, preferred embodiments of the present invention provide compositions and methods for hyperactivating Rho in a subject.

As cell motility is also regulated by the small GTPase Rac (See, W. B. Kiosses et al., Nat. Cell Biol., 3:316-320 (2001); and L. Kjoller and A. Hall, J. Cell Biol., 152:1145-1157 (2001)), still other embodiments provide studies of the effects of integrin antagonists (e.g., α5β1) on Rac activation. Rac normally is rapidly activated upon cell attachment to the ECM. For example, endothelial cells activate Rac after as little as 30 min of attachment to vitronectin. In contrast, in the presence of integrin antagonists (e.g., α5β1), Rac activation is inhibited. In the present invention, in some embodiments, inhibition of endothelial or tumor cell motility by integrin antagonists is reversed by expression of constitutively active Rae (V12Rac). In still some other embodiments, mutationally inactive Rac blocks cell migration. Expression of transgenes was verified by Western blotting. Indeed, the present invention further shows that expression of mutationally inactive Rac (N17Rac), but not active Rac (V12Rac), inhibits stress fiber formation. Expression of mutationally active Rac restores stress fibers and polarization to cells treated with integrin α5β1 antagonists. These findings indicate that integrin antagonists (e.g., α5β1) inhibit Rac, thereby blocking cell migration at least in part by inhibiting stress fiber formation and polarization.

The p21 activated kinase (PAK) is a substrate of Rac that regulates endothelial cell migration. (See e.g., W. B. Kiosses et al., J. Cell Biol., 147:831-843 (1999)). In fact, constitutively active PAK can partially reverse the inhibition of cell migration on vitronectin induced by integrin (e.g., α5β1) antagonists, while mutationally inactive PAK suppresses cell migration. Taken together, these results thus indicate that α5β1 antagonists inactivate Rac and PAK while hyperactivating Rho, thereby inhibiting cell migration.

Since, in preferred embodiments α5β1 antagonists activate PKA and hyperactivate Rho, the present invention contemplates that it is possible that once PKA is activated it activates Rho, thus blocking normal (e.g., endothelial) and tumor (e.g., lymphoma) cell migration. The present invention shows that activating PKA in normal (e.g., endothelial) cells by cAMP, overexpression of PKAcat, and the like, hyperactivates Rho. Indeed, in one preferred embodiment, expression of mutationally inactive Rho (e.g., N19Rho) reverses the inhibitory effects of cAMP and PKA catalytic subunit on cell migration. The present invention contemplates that, in certain embodiments, PKA suppresses cell migration in part by hyperactivating Rho thus preventing stress fiber formation.

The present invention shows that integrin (e.g., α5β1) antagonists activate PKA, inhibit Rac activation, and block cell migration, thus in some embodiments the present invention contemplates that PKA inhibition of cell migration is Rac-dependent. In preferred embodiments, activation of PKA by either cell permeable cAMP or by overexpression of the catalytic subunit of PKA also inhibits Rac. In still further preferred embodiments, expression of constitutively active Rac reverses the effects of PKA activation on cell migration.

Data presented herein demonstrates that PKA regulates endothelial cell migration in vivo during angiogenesis. In one study, chick chorioallantoic membranes (CAM) were stimulated with bFGF and transfected with GFP or mutationally inactive PKA (dnPKA) expression plasmids. CAMs were then incubated in the presence and absence of anti-α5β1 antibodies. Transgene expression was verified by immunoblotting of CAM lysates and by immunohistochemical staining of cryosections of transfected CAMs. While α5β1 antagonists significantly block angiogenesis, the mutationally inactive PKA partially reverses this inhibition. Similar results were observed with pharmacological PKA inhibitors. (See, S. Kim et al., J. Biol. Chem., 275:33920-33928 (2000)). Since PKA plays a role in the negative regulation of angiogenesis by integrin antagonists, the present invention further investigated whether direct activation of PKA also inhibits angiogenesis. In one embodiment, expression of the PKA catalytic subunit during angiogenesis in vivo completely suppresses angiogenesis (P=0.0005), as does exposure of CAMs to cAMP. VEGF stimulated angiogenesis is also inhibited by activation of PKA. Thus, the present invention provides compositions and methods to activate PKA in a subject by integrin antagonists, cAMP, or overexpression of the catalytic subunit of PKA, and the like, thus inhibiting angiogenesis.

Although the present invention is not limited to any particular mechanism and an understanding of the mechanism is not necessary to practice the present invention, the present invention contemplates that taken together, the numerous studies and embodiments of the present invention described herein show that unligated integrins suppress migration of attached cells (e.g., normal cells and tumor cells) by activating PKA. PKA subsequently hyperactivates Rho and inhibits Rac activation, thereby preventing cell migration by inhibiting cell stress fiber formation and/or cellular polarization. Preferred embodiments of the present invention show that integrin receptors for provisional matrix proteins regulate each other and that PKA activation plays an important role in this process. Other preferred embodiments show that PKA plays an important role in the negative regulation of cell migration both in vitro and in vivo.

B. Parathyroid Hormone and Parathyroid Related Hormone and Portions Thereof

In some preferred embodiments of the present invention, parathyroid hormone (“PTH”) and/or closely related parathyroid related hormone (“PTHrP”) are administered to a subject in an effective amount to activate PKA and to thus inhibit angiogenesis and/or cell migration. In particularly preferred embodiments, an effective amount of PTHrP is administered to a subject to inhibit (e.g., retard) undesirable angiogenesis and/or cell migration. In some other preferred embodiments, the present invention provides methods and compositions to prophylacticly administer an effective amount of PTH and/or PTHrP to prevent undesirable angiogenesis, cell migration, cell adhesion, and/or cell survival to a subject at risk for developing or progressing with a disease characterized by undesirable modulating angiogenesis, cell migration, cell adhesion, and/or cell survival (e.g., cancer). The following non-limiting examples provide further details concerning specific therapeutic embodiments of the present invention contemplated herein.

To evaluate the role of PTHrP in blood vessel development, the inventors studied the effects of PTHrP on angiogenesis in the chick chorioallantoic membrane (CAM). In one study, CAMs from 10 day old chick embryos were stimulated with basic fibroblast growth factor (bFGF) in the presence or absence of 1 μM PTHrP (1-173) or two other peptide hormones, calcitonin (a calcium regulating peptide hormone (See, R. D. Bukoski et al., Semin. Nephrol., 15:536-549 (1995)) and calcitonin gene related peptide (CGRP, a vasodilatory peptide hormone (R. D. Bukoski et al., supra). The present invention shows that PTHrP inhibited angiogenesis whether analyzed by enumerating blood vessel branchpoints or by analyzing density of vessels positive for integrin αvβ3 (Gasparini, et al.) or von Willebrand factor (VWF). PTHrP, as well as CGRP and calcitonin, had no effect on the number, size or composition of pre-existing vessels, as determined by macroscopic evaluation of vessels in saline-stimulated CAMs or by examining the number and nature of vascular smooth muscle actin positive vessels in saline- and bFGF-stimulated CAMs. These studies indicate that PTHrP selectively prevents the formation of new microvessels in certain embodiments of the present invention. The half-maximal inhibitory dose of PTHrP was 0.01 μM. This angiogenesis inhibition could be blocked by addition of an anti-PTHrP function blocking antibody (8B12), directed against amino acids 1-34. (See, R. Terkeltaub et al., Arthritis and Rheumatism, 41:2152-64 (1998)). In other embodiments, PTHrP also blocked angiogenesis in the adult mouse, which was stimulated by subcutaneous injection of growth factor-depleted matrigel containing bFGF and 1 μM PTHrP or calcitonin (P<0.001). These studies indicate that PTHrP functions as an angiogenesis inhibitor.

The present invention further contemplates that PTHrP also inhibits tumor angiogenesis and growth. For example, nude mice bearing 30 mm3 DU145 prostate carcinoma tumors (that did not express PTHrP) were treated with daily intravenous injections of PTHrP (final serum concentration 1 μM). PTHrP, but not a scrambled control peptide, suppressed tumor growth and tumor angiogenesis. These studies indicate that PTHrP inhibits tumor angiogenesis as well as growth factor-induced angiogenesis.

Additional embodiments of the present invention provide studies to determine if gene delivery of PTHrP is a useful strategy for the therapeutic inhibition of angiogenesis in vivo, that that end, angiogenesis was stimulated in chick CAMs with either bFGF or vascular endothelial growth factor (VEGF). CAMs were then transduced by injecting adenoviruses expressing Green Fluorescent Protein (GFP) or full length PTHrP (1-173) according to R. Terkeltaub et al., into the chick embryo circulation. (See, R. Terkeltaub et al., Arthritis and Rheumatism 41:2152-64 (1998)). Virally expressed PTHrP, but not GFP, inhibited angiogenesis stimulated by bFGF (P<0.001) or VEGF (P=0.01) as shown in by quantifying blood vessel branchpoints or integrin αvβ3 immunoreactive vessels. PTHrP expression was detected in blood vessels (arrows) in CAMs transduced with PTHrP but not in blood vessels transduced with GFP, using an antibody directed against PTHrP C-terminal amino acids 109-14128. In additional embodiments, virally expressed PTHrP also inhibited angiogenesis in the adult mouse. In this case, angiogenesis was stimulated by subcutaneous injection of growth factor-depleted matrigel containing bFGF and GFP- or PTHrP-expressing adenoviruses. Viral delivery of PTHrP inhibited murine angiogenesis. Virally induced expression of PTHrP, but not GFP, in blood vessels also inhibited tumor growth on the CAM, showing that locally delivered PTHrP suppresses tumor angiogenesis. In preferred embodiments, PTHrP treated tumors are smaller, obviously necrotic and associated with fewer integrin αvβ3 positive blood vessels than GFP treated tumors. Thus, the present invention provides methods of virally expressing PTHrP to inhibit angiogenesis.

In still further embodiments, the present invention provides studies to evaluate the role of PTHrP in endothelial functions in vitro. For example, the present invention provides studies that tested the effects of PTHrP (1-173) and other peptide hormones on endothelial cell migration on different extracellular matrix substrates, such as, vitronectin, collagen, and fibronectin. The present invention shows that PTHrP significantly inhibits endothelial cell migration on vitronectin, collagen, and fibronectin, yet had no effect on cell attachment to these matrix proteins. In a preferred embodiment, 50% inhibition of cell migration was achieved with PTHrP in the range of 1-10 μM. Thus, some embodiments of the present invention contemplate the therapeutic administration of an effective amount (dose) of PTHrP hormone to a subject that inhibits endothelial cell migration, but that does not inhibit integrin ligation.

PTHrP is composed of several domains with distinct physiological properties. (See e.g., L. Jin et al., J. Biol. Chem., 275:27238-27244 (2000)). In a preferred embodiment, the present invention identified the domain(s) of PTHrP responsible for the anti-migratory and anti-angiogenic by evaluating the effects of various fragments of the peptide hormone in migration and angiogenesis assays. In preferred embodiments, these studies included analyzing PTHrP (1-173), fragments containing the N-terminus extending from amino acids 1-141, 1-86, and 1-34 such that the invention shows effective amounts of PTHrP inhibits cell migration on ECM proteins, such as vitronectin, but does not inhibit cell attachment. In other embodiments, fragments lacking the N-terminus, such PTHrP 107-138, were not able to inhibit cell migration. In vivo, PTHrP 1-141, 1-86, and 1-34 potently inhibited angiogenesis stimulated by bFGF. As the first 34 amino acids contain the angiogenesis inhibition properties, further studies were performed to identify the critical residues responsible for angiogenesis inhibition.

In particular, studies show that the first six amino acids of PTH are required for activation of PTH/PTHrP receptor signaling while the last fifteen are required for high affinity binding to the receptor. (See e.g., L. Jin et al., J. Biol. Chem., 275:27238-27244 (2000)). Five out of the first ten amino acids in PTH and PTHrP are identical and structure predictions indicate these regions have similar conformations. (See e.g., M. Shimizu et al., J. Biol. Chem., 275:21836-218343 (2000)). Therefore, to further delineate the active angiogenesis and cell migration inhibition sites on PTHrP, preferred embodiments of the present invention compared the activities of fragments of PTHrP extending from amino acids 1-10, 1-34, and 15-34 to a scrambled 1-10 peptide. While amino acids 1-34 and 1-10 were similarly effective in inhibiting cell migration on vitronectin, amino acids 15-34 and a scrambled version of 1-10 were unable to inhibit cell migration. The N-terminal PTHrP fragment 1-10 inhibited cell migration in a dose-dependent manner, with a maximum of 50% inhibition at a concentration of 10 μM. PTHrP 1-10 and 1-34 were also effective in inhibiting angiogenesis in CAM assays, while scrambled 1-10 was not. PTHrP 1-10 is slightly less effective at inhibiting angiogenesis than 1-34, however, PTHrP 1-10 was also effective in inhibiting angiogenesis in a mouse matrigel model of angiogenesis while scrambled PTHrP 1-10 peptide was not. These studies indicate that, in some particularly preferred embodiments, the first ten PTHrP residues are important for activation of endothelial cell PTHrP receptor in vitro and in vivo.

The present invention contemplates that angiogenesis is inducible by many naturally occurring growth factors such as vascular endothelial growth factor (VEGF), basic fibroblast growth factor (FGF), tumor necrosis factor (TNFα), and interleukin 8 (IL-8). Thus, in some embodiments, to determine if PTHrP is a general inhibitor of angiogenesis, a range of concentrations of PTHrP 1-34 extending from 0.001-10 μM on angiogenesis stimulated in the CAM by bFGF, VEGF, IL-8 or TNFα was tested. Angiogenesis induced by each growth factor was inhibited by PTHrP (1-34), with complete inhibition occurring at 1 μM. The IC₅₀ for each growth factor was 0.001 μM or lower. Thus, in preferred embodiments, PTHrP is a potent general angiogenesis inhibitor.

Although an understanding of the mechanism is not necessary to practice the present invention and the present invention is not so limited, certain embodiments of the present invention evaluated the effects of PTHrP on endothelial cell signal transduction to establish the mechanism by which PTHrP inhibits cell migration in vitro and angiogenesis in vivo. The present invention contemplates that like PTH, PTHrP interacts with the parathyroid hormone (PTH) PTH1 receptor (a multiple membrane spanning G protein coupled cell surface receptor that activates protein kinase A (See, A. B. Abou-Samra et al., Proc. Natl. Acad. Sci. U.S.A., 89:2732-2736 (1992); and S. Hoare et al., J. Biol. Chem., 276:7741-7753 (2001)) and that is expressed on endothelial cells. (See, B. Jiang et al., J. Cardiovascular Pharmacol., S142-1444 (1998)). The present invention shows that PTHrP 1-34 as well as a cell permeable form of cAMP (dibutyryl cAMP), rapidly stimulate PKA activity in endothelial cells. in some embodiments, PKA activation was detected in as little as 2 min, with maximal activity at 15 min after stimulation with both cAMP and PTHrP. Still other embodiments of the present invention are directed to determining whether the anti-migratory properties of PTHrP result from signals transduced through protein kinase A. For example, the present invention evaluated the effects of PTHrP 1-34 on cell migration in the presence and absence of the protein kinase A inhibitor, N-(2-(p-bromocinnamylamino)ethyl)-5-isoquinoline sulfonamide (H89). The PKA inhibitor (48 nM) blocked the anti-migratory properties of PTHrP. Furthermore, expression of a mutationally inactive form of PKA (dnPKA), which blocks PKA activation (See e.g. A. Howe and R. J. Juliano, Nature Cell Biol., 2:593-600 (2000)) suppresses the PTHrP- and cAMP-mediated inhibition of endothelial cell migration. In fact, in some embodiments, directed activation of PKA by either cAMP or by transient transfection with the PKA catalytic subunit inhibits endothelial cell migration on ECM substrates. Expression of both transgenes was detected by Western blotting of lysates of transfected cells. These results indicate that PTHrP inhibition of migration is PKA dependent and that activation of PKA blocks endothelial cell migration.

The present invention contemplates the small GTPase Rac plays an essential role in regulating cell motility by influencing actin assembly and lamellipodia extension. (See e.g., A. J. Ridley et al., Cell, 70:401-410 (1992)). Cell adhesion as well as growth factor stimulation upregulate Rac activity in endothelial cells. (L. S. Price et al., Mol. Biol. Cell, 9:1863-1871 (1998)). The present invention shows that activation of Rac is blocked by PTHrP, expression of activated PKA or by cAMP. Overexpression of mutationally active Rac (V12 Rac) overcomes the PTHrP, cAMP or PKA catalytic subunit mediated inhibition of cell motility. Thus, in preferred embodiments, PTHrP activation of PKA inhibits cell migration by inhibiting Rac activation.

The present invention contemplates that activation of PKA in vivo inhibits angiogenesis. Accordingly, some preferred embodiments of the present invention are directed to evaluating the role of PKA in the inhibition of angiogenesis by PTHrP. In still other preferred embodiments, the present invention shows that either pharmacological or genetic inhibition of PKA reverses PTHrP inhibition of angiogenesis. For example, H89, a selective PKA inhibitor, blocks the suppression of angiogenesis induced by PTHrP 1-34. Likewise, expression of mutationally inactive PKA (dnPKA) also reverses the inhibitory effects of PTHrP. Furthermore, in yet other embodiments, activation of PKA by cAMP or by expression of the catalytic subunit of PKA in the CAM potently inhibits angiogenesis. Preferred embodiments of the present invention thus show that PTHrP inhibits angiogenesis by activating PKA.

As mentioned above, several recent studies show that Rac activity is required for angiogenesis. (See e.g., O. Dormond et al., Nature Med., 7:1041-1047 (2001)). Taken together, in some embodiments, the present invention contemplates that these data show that activation of PKA in endothelial cells in vivo induces Rac inactivation and inhibition of cell migration in vivo. Moreover, the present invention further contemplates that these studies data also show that activation of PKA by hormonal, pharmacological, or genetic means provides potent methods for inhibiting angiogenesis in normal (e.g., endothelial) and tumor (e.g., lymphoma) cells.

PTHrP is a peptide hormone with well-described effects on vascular tone. Although PTHrP regulates vascular tone by relaxing vascular smooth muscle cells, the present invention shows that these properties have no role in the regulation of angiogenesis by PTHrP. For example, studies of smooth muscle actin positive vessels in growth factor-stimulated and unstimulated tissues show that the number, diameter and quality of smooth muscle actin positive vessels does not change during the course of the angiogenesis assays performed in our studies. Also, treatment of tissues undergoing angiogenesis with either PTHrP or CGRP, two vasodilatory hormones, has no effect on the number, size or diameter of smooth muscle actin positive vessels in these assays. The effects of PTHrP on angiogenesis are specific as neither CGRP nor calcitonin, two peptide hormones with related functions that are evolutionarily well conserved, inhibit angiogenesis.

Further, the present invention contemplates that PTHrP impacts tissue growth in multiple ways. In cancer, for example, PTHrP simultaneously inhibits angiogenesis by acting directly on endothelial cells (as described above), while stimulating tumor cells to produce angiogenesis stimulators. In bone, PTHrP inhibits angiogenesis, while stimulating chondrocyte proliferation. As an anti-angiogenic agent, the present invention contemplates that PTHrP is therapeutically useful if delivered at relatively high local concentrations. Preferred embodiments of the present invention provide adenovirally expressed PTHrP for the local administration of PTHrP as an effective strategy to inhibit angiogenesis and tumor growth.

C. Expression and Synthesis of Peptides of Interest

In some embodiments, the present invention employs PTH, PTHrP and/or PKA catalytic subunits and portions thereof. In one embodiment, these peptides and portions thereof are expressed using encoding polynucleotide sequences. Exemplary polynucleotides encoding at least a portion of parathyroid hormone (PTH) and amino acid sequences encoded thereby (e.g., SEQ ID NOs: 1-44 and 112) contemplated for use in the methods of the present invention are listed in Table 1 shown below.

TABLE 1
Parathyroid Hormone Accession Nos.

A08526 (SEQ ID NO. 1)
A08534 (SEQ ID NOS. 2-3)
M26143 (SEQ ID NOS. 4-5)
E01147 (SEQ ID NO. 6)
D10292 (SEQ ID NO. 7)
NM_000315 (SEQ ID NOS. 8-9)
I83610 (SEQ ID NO. 10)
I83607 (SEQ ID NO. 11)
E04335 (SEQ ID NO. 12)
A08533 (SEQ ID NO. 13)
A08527 (SEQ ID NO. 14)
J00301 (SEQ ID NOS. 15-16)
J00300 (SEQ ID NO. 17)
NM_020623 (SEQ ID NOS. 18-19)
AF346654 (SEQ ID NO. 20)
AF251060 (SEQ ID NOS. 21-22)
AF309967 (SEQ ID NOS. 23-24)
NM_017044 (SEQ ID NOS. 25-26)
AF130257 (SEQ ID NOS. 27-28)
AH007117 (SEQ ID NOS. 29-31)
D10291 (SEQ ID NO. 32)
X05722 (SEQ ID NOS. 33-34)
X12515 (SEQ ID NOS. 35-36)
X12516 (SEQ ID NO. 37)
V00597 (SEQ ID NOS. 38-39)
X05721 (SEQ ID NO. 40-41)
K01268 (SEQ ID NO. 42-43)
K01267 (SEQ ID NO. 44)
NP_000306 (SEQ ID NO: 112)

In a preferred embodiment, the PTH is exemplified by GenBank accession no. NP_{—}000306 (SEQ ID NO: 112).

Exemplary polynucleotides encoding at least a portion of parathyroid hormone related protein (PTHrP) and amino acid sequences encoded thereby (e.g., SEQ ID NOs: 45-73 and 113) contemplated for use in the methods of the present invention are listed in Table 2 shown below.

TABLE 2
Parathyroid hormone-related protein Accession Nos:

BM489067 (SEQ ID NO: 45)
NM_008970 (SEQ ID NOS: 46-47)
AY052416 (SEQ ID NO: 48)
AY052415 (SEQ ID NO: 49)
AY052414 (SEQ ID NOS: 50-51)
AF219973 (SEQ ID NOS: 52-53)
AJ278119 (SEQ ID NOS: 54-55)
AF197904 (SEQ ID NOS: 56-57)
AF300703 (SEQ ID NOS: 58-59)
AJ249391 (SEQ ID NOS 60-61)
E02539 (SEQ ID NO: 62)
E02538 (SEQ ID NO: 63)
U15593 (SEQ ID NOS: 64-65)
M60057 (SEQ ID NOS: 66-67)
M60056 (SEQ ID NOS: 68-69)
M60058 (SEQ ID NOS: 70-71)
M17183 (SEQ ID NOS: 72-73)
NP_002811 (SEQ ID NO: 113)

In a preferred embodiment, the PTHrP is exemplified by GenBank accession no. NP_{—}002811 (SEQ ID NO: 113).

Likewise, exemplary polynucleotides encoding at least a portion of a catalytically active PKA subunit and amino acid sequences encoded thereby (e.g., SEQ ID NOs: 74-111) contemplated for use in the methods of the present invention are shown below in Table 3.

TABLE 3
PKA Accession Nos.

AJ297954 (SEQ ID NOS: 74-75)
AJ297564 (SEQ ID NOS: 76-77)
AJ431364 (SEQ ID NOS: 78-79)
AJ413219 (SEQ ID NOS: 80-81)
AJ413218 (SEQ ID NOS: 82-83)
AY055783 (SEQ ID NOS: 84-85)
U43906 (SEQ ID NOS: 86-87)
AF181097 (SEQ ID NOS: 88-89)
BG353008 (SEQ ID NO: 90)
NM_002730 (SEQ ID NOS: 91-92)
AF224719 (SEQ ID NOS: 93-94)
AF224718 (SEQ ID NOS: 95-96)
AJ243654 (SEQ ID NOS: 97-98)
M34182 (SEQ ID NOS: 99-100)
X69806 (SEQ ID NOS: 101-102)
X53261 (SEQ ID NOS: 103-104)
M80335 (SEQ ID NOS: 105-106)
M84336 (SEQ ID NO: 107)
M84335 (SEQ ID NOS: 108-109)
NM_008854.3 (SEQ ID NOS: 110-111)

In a preferred embodiment, the PKA catalytic subunit is a murine PKA catalytic subunit alpha as exemplified by GenBank accession no. NM_{—}008854.3 (SEQ ID NOS: 110-111). In a more preferred embodiment, the PKA catalytic subunit is a human PKA catalytic alpha sequence that has about 91% identity to the mouse sequence. Human sequences are preferred when administered to human subjectes in order to reduce immune responses.

Other embodiments of the present invention provide fragments, fusion proteins or functional equivalents of these proteins. In still other embodiment of the present invention, nucleic acid sequences corresponding to PTH, PTHrP and/or PKA catalytic subunit variants, homologs, and mutants may be used to generate recombinant DNA molecules that direct the expression of the PTH, PTHrP and/or PKA catalytic subunit variants, homologs, and mutants in appropriate host cells. In some embodiments of the present invention, the polypeptide may be a naturally purified product, in other embodiments it may be a product of chemical synthetic procedures, and in still other embodiments it may be produced by recombinant techniques using a prokaryotic or eukaryotic host (e.g., by bacterial, yeast, higher plant, insect and mammalian cells in culture). In some embodiments, depending upon the host employed in a recombinant production procedure, the polypeptide of the present invention may be glycosylated or may be non-glycosylated. In other embodiments, the polypeptides of the invention may also include an initial methionine amino acid residue.

In one embodiment of the present invention, due to the inherent degeneracy of the genetic code, DNA sequences other than the recited polynucleotide sequences that encode substantially the same or a functionally equivalent amino acid sequence, may be used to clone and express PTH, PTHrP and/or PKA catalytic subunit polypeptides. In general, such polynucleotide sequences hybridize to SEQ ID NOs: 1-115 (and to any other sequence referred to herein) under conditions of high to medium stringency as described above. As will be understood by those of skill in the art, it may be advantageous to produce PTH, PTHrP and/or PKA catalytic subunit-encoding nucleotide sequences possessing non-naturally occurring codons. Therefore, in some preferred embodiments, codons preferred by a particular prokaryotic or eukaryotic host (Murray et al., Nucl. Acids Res., 17 (1989)) are selected, for example, to increase the rate of PTHrP expression or to produce recombinant RNA transcripts having desirable properties, such as a longer half-life, than transcripts produced from naturally occurring sequence.

1. Vectors for Production of Polypeptides of Interest

The polynucleotides of the present invention may be employed for producing polypeptides by recombinant techniques. Thus, for example, the polynucleotide may be included in any one of a variety of expression vectors for expressing a polypeptide. In some embodiments of the present invention, vectors include, but are not limited to, chromosomal, nonchromosomal and synthetic DNA sequences (e.g., derivatives of SV40, bacterial plasmids, phage DNA; baculovirus, yeast plasmids, vectors derived from combinations of plasmids and phage DNA, and viral DNA such as vaccinia, adenovirus, fowl pox virus, and pseudorabies). It is contemplated that any vector may be used as long as it is replicable and viable in the host.

In particular, some embodiments of the present invention provide recombinant constructs comprising one or more of the sequences as broadly described above (e.g., SEQ ID NOs: 1-115). In some embodiments of the present invention, the constructs comprise a vector, such as a plasmid or viral vector, into which a sequence of the invention has been inserted, in a forward or reverse orientation. In still other embodiments, a heterologous structural sequence (e.g., SEQ ID NO:1) is assembled in appropriate phase with translation initiation and termination sequences. In preferred embodiments of the present invention, the appropriate DNA sequence is inserted into the vector using any of a variety of procedures. In general, the DNA sequence is inserted into an appropriate restriction endonuclease site(s) by procedures known in the art.

Large numbers of suitable vectors are known to those of skill in the art, and are commercially available. Such vectors include, but are not limited to, the following vectors: 1) Bacterial—pQE70, pQE60, pQE-9 (Qiagen), pBS, pD10, phagescript, psiX174, pbluescript SK, pBSKS, pNH8A, pNH16a, pNH18A, pNH46A (Stratagene); ptrc99a, pKK223-3, pKK233-3, pDR540, pRIT5 (Pharmacia); 2) Eukaryotic—pWLNEO, pSV2CAT, pOG44, PXT1, pSG (Stratagene) pSVK3, pBPV, pMSG, pSVL (Pharmacia); and 3) Baculovirus—pPbac and pMbac (Stratagene). Any other plasmid or vector may be used as long as they are replicable and viable in the host. In some preferred embodiments of the present invention, mammalian expression vectors comprise an origin of replication, a suitable promoter and enhancer, and also any necessary ribosome binding sites, polyadenylation sites, splice donor and acceptor sites, transcriptional termination sequences, and 5′ flanking non-transcribed sequences. In other embodiments, DNA sequences derived from the SV40 splice, and polyadenylation sites may be used to provide the required non-transcribed genetic elements.

In certain embodiments of the present invention, the DNA sequence in the expression vector is operatively linked to an appropriate expression control sequence(s) (promoter) to direct mRNA synthesis. Promoters useful in the present invention include, but are not limited to, the LTR or SV40 promoter, the E. coli lac or trp, the phage lambda P_{L} and P_{R}, T3 and T7 promoters, and the cytomegalovirus (CMV) immediate early, herpes simplex virus (HSV) thymidine kinase, and mouse metallothionein-I promoters and other promoters known to control expression of gene in prokaryotic or eukaryotic cells or their viruses. In other embodiments of the present invention, recombinant expression vectors include origins of replication and selectable markers permitting transformation of the host cell (e.g., dihydrofolate reductase or neomycin resistance for eukaryotic cell culture, or tetracycline or ampicillin resistance in E. coli).

In some embodiments of the present invention, transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Enhancers useful in the present invention include, but are not limited to, the SV40 enhancer on the late side of the replication origin bp 100 to 270, a cytomegalovirus early promoter enhancer, the polyoma enhancer on the late side of the replication origin, and adenovirus enhancers.

In other embodiments, the expression vector also contains a ribosome binding site for translation initiation and a transcription terminator. In still other embodiments of the present invention, the vector may also include appropriate sequences for amplifying expression.

2. Expression of Polypeptides of Interest

The constructs in host cells can be used in a conventional manner to produce the gene product encoded by the recombinant sequence. In some embodiments, introduction of the construct into the host cell can be accomplished by calcium phosphate transfection, DEAE-Dextran mediated transfection, or electroporation (See e.g., Davis et al., Basic Methods in Molecular Biology, (1986)). Alternatively, in some embodiments of the present invention, the polypeptides of the invention can be synthetically produced by conventional peptide synthesizers.

Still other embodiments of the present invention provide mutant or variant forms (i.e., muteins) of the polypeptides of interest (e.g., PTH, PTHrP, and/or catalytically active subunits of PKA). It is possible to modify the structure of a peptide having an activity of, for example, PTHrP, for such purposes as enhancing therapeutic or prophylactic efficacy, or stability (e.g., ex vivo shelf life, and/or resistance to proteolytic degradation in vivo). Such modified peptides are considered functional equivalents of peptides having an activity of the subject proteins as defined herein. A modified peptide can be produced in which the amino acid sequence has been altered, such as by amino acid substitution, deletion, or addition.

Moreover, as described above, variant forms (e.g., mutants or polymorphic sequences) of the subject proteins are also contemplated as being equivalent to those peptides and DNA molecules that are set forth in more detail. For example, as described above, the present invention encompasses mutant and variant proteins that contain conservative or non-conservative amino acid substitutions.

In a preferred embodiment of the present invention, combinatorial libraries are produced by way of a degenerate library of genes encoding a library of polypeptides which each include at least a portion of potential therapeutic protein sequence. For example, a mixture of synthetic oligonucleotides can be enzymatically ligated into gene sequences such that the degenerate set of potential sequences are expressible as individual polypeptides, or alternatively, as a set of larger fusion proteins (e.g., for phage display) containing the set of sequences therein.

There are many ways by which a library of potential PTH, PTHrP, and/or catalytically active PKA subunit homologs and variants can be generated from a degenerate oligonucleotide sequence. In some embodiments, chemical synthesis of a degenerate gene sequence is carried out in an automatic DNA synthesizer, and the synthetic genes are ligated into an appropriate gene for expression. The purpose of a degenerate set of genes is to provide, in one mixture, all of the sequences encoding the desired set of potential therapeutic polypeptide sequences. The synthesis of degenerate oligonucleotides is well known in the art. (See e.g., Narang, Tetrahedron Lett., 39:39 (1983); Itakura et al., Recombinant DNA, in Walton (ed.), Proceedings of the 3rd Cleveland Symposium on Macromolecules, Elsevier, Amsterdam, pp 273-289 (1981); Itakura et al., Annu. Rev. Biochem., 53:323 (1984); Itakura et al., Science 198:1056 (1984); and Ike et al., Nucl. Acid Res., 11:477 (1983)). Such techniques have been employed in the directed evolution of other proteins (See e.g., Scott et al., Science 249:386 (1980); Roberts et al., Proc. Natl. Acad. Sci. USA, 89:2429 (1992); Devlin et al., Science 249: 404 (1990); Cwirla et al., Proc. Natl. Acad. Sci. USA, 87: 6378 (1990); as well as U.S. Pat. Nos. 5,223,409; 5,198,346; and 5,096,815 each of which is incorporated herein by reference).

A wide range of techniques are known in the art for screening gene products of combinatorial libraries made by point mutations, and for screening cDNA libraries for gene products having a certain property. Such techniques will be generally adaptable for rapid screening of the gene libraries generated by the combinatorial mutagenesis or recombination of homologs or variants. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

3. Chemical Synthesis of Polypeptides of Interest

In an alternate embodiment of the invention, the coding sequence of the polypeptides of interest are synthesized, whole or in part, using chemical methods well known in the art (See e.g., Caruthers et al., Nucl. Acids Res. Symp. Ser., 7:215 (1980); Crea and Horn, Nucl. Acids Res., 9:2331 (1980); Matteucci and Caruthers, Tetrahedron Lett., 21:719 (1980); and Chow and Kempe, Nucl. Acids Res., 9:2807 (1981)). In other embodiments of the present invention, the protein itself is produced using chemical methods to synthesize either an entire amino acid sequence or a portion thereof. For example, peptides can be synthesized by solid phase techniques, cleaved from the resin, and purified by preparative high performance liquid chromatography (See e.g., Creighton, Proteins Structures And Molecular Principles, W H Freeman and Co, New York N.Y. (1983)). In other embodiments of the present invention, the composition of the synthetic peptides is confirmed by amino acid analysis or sequencing (See e.g., Creighton, supra).

Direct peptide synthesis can be performed using various solid-phase techniques (Roberge et al., Science 269:202 (1995)) and automated synthesis may be achieved, for example, using ABI 431A Peptide Synthesizer (Perkin Elmer) in accordance with the instructions provided by the manufacturer. Additionally, the amino acid sequence of PTH, PTHrP, or PKA catalytically active subunit, or any part thereof, may be altered during direct synthesis and/or combined using chemical methods with other sequences to produce a variant polypeptide.

The invention's methods are unseful in, for example, gene therapy to alter PTH, PTHrP, and/or catalytically active PKA subunit expression, production, or function. As described above, the present invention provides the exemplary human PTH, PTHrP, and/or catalytically active PKA subunit genes and methods of obtaining the genes from other species are known in the art. Thus, the methods described below are generally applicable across many species. In some embodiments, it is contemplated that the gene therapy is performed by providing a subject with a wild-type allele of therapeutic protein/polypeptide (e.g., PTHrP). Subjects in need of such therapy are identified by the methods described above.

Viral vectors commonly used for in vivo or ex vivo targeting and therapy procedures are DNA-based vectors and retroviral vectors. Methods for constructing and using viral vectors are known in the art (See e.g., Miller and Rosman, BioTech., 7:980-990 (1992)). Preferably, the viral vectors are replication defective, that is, they are unable to replicate autonomously in the target cell. In general, the genome of the replication defective viral vectors that are used within the scope of the present invention lack at least one region that is necessary for the replication of the virus in the infected cell. These regions can either be eliminated (in whole or in part), or be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (i.e., on the isolated DNA) or in situ, using the techniques of genetic manipulation or by treatment with mutagenic agents.

In a preferred embodiment, the vector is an adenovirus vector. Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to type 2 or type 5 human adenoviruses (Ad 2 or Ad 5), or adenoviruses of animal origin (See e.g. WO 94/26914). Those adenoviruses of animal origin that can be used within the scope of the present invention include adenoviruses of canine, bovine, murine (e.g., Mav1, Beard et al., Virol., 75-81 (1990)), ovine, porcine, avian, and simian (e.g., SAV) origin. Preferably, the adenovirus of animal origin is a canine adenovirus, more preferably a CAV2 adenovirus. (See e.g. Manhattan or A26/61 strain (ATCC VR-800)). Replication defective adenoviral vectors may be modified, in particular in the E3 region (e.g., WO 95/02697), the E2 region (e.g., WO 94/28938), the E4 region (e.g., WO 94/28152, WO 94/12649 and WO 95/02697), or in any of the late genes L1-L5. The replication defective recombinant adenoviruses according to the invention can be prepared by any technique known to the person skilled in the art (See e.g., Levrero et al., Gene 101:195 (1991); EP 185 573; and Graham, EMBO J., 3:2917 (1984)). Additionally, the use of vectors derived from the AAVs for transferring genes in vitro and in vivo has been described (See e.g., WO 91/18088; WO 93/09239; U.S. Pat. No. 4,797,368; U.S. Pat. No., 5,139,941; and EP 488 528, all of which are herein incorporated by reference).

Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931).

It is also possible to introduce the vector in vivo as a naked DNA plasmid. Methods for formulating and administering naked DNA to mammalian muscle tissue are disclosed in U.S. Pat. Nos. 5,580,859; and 5,589,466 both of which are herein incorporated by reference.

D. Detecting Angiogenesis

Angiognesis in a tissue may be detected by detecting the presence of integrin α5β1 polypeptide using Western blot analysis or immunofluorescence. Alternatively, angiognesis in a tissue may be detected by detecting the presence of the exemplary integrin α5β1 mRNA using reverse transcription polymerase chain (RT-PCR), or in situ hybridization. These methods are well within the ordinary skill in the art.

In one embodiment, detection of the of the exemplary integrin α5β1 employ an agent (e.g., polypeptide and/or mRNA) that can be detectably labeled. A moiety useful for labeling a polypeptide and/or mRNA agent can be a radionuclide, a paramagnetic material, an X-ray attenuating material, a fluorescent, chemiluminescent or luminescent molecule, a molecule such as biotin, or a molecule that can be visualized upon reaction with a particular reagent, for example, a substrate for an enzyme or an epitope for an antibody. The moiety can be linked to the polypeptide and/or mRNA agent using well known methods, which are selected, in part, based on the chemical nature of the agent and the moiety. For example, where the moiety is an amino acid sequence such as a hexahistidine (His6) sequence, and the agent is a peptide, the His6 sequence can be synthesized as part of the peptide, and the His6-labeled agent can be identified by the binding of a nickel ion reagent to the His6 moiety.

Methods for chemically linking a moiety to several types of agent also can be utilized. For example, methods for conjugating polysaccharides to peptides are exemplified by, but not limited to coupling via alpha- or epsilon-amino groups to NaIO₄-activated oligosaccharide, using squaric acid diester (1,2-diethoxycyclobutene-3,4-dione) as a coupling reagent, coupling via a peptide linker wherein the polysaccharide has a reducing terminal and is free of carboxyl groups (U.S. Pat. No. 5,342,770), coupling with a synthetic peptide carrier derived from human heat shock protein hsp65 (U.S. Pat. No. 5,736,146), and using the methods of U.S. Pat. No. 4,639,512. Methods for conjugating proteins to proteins include coupling with a synthetic peptide carrier derived from human heat shock protein hsp65 (U.S. Pat. No. 5,736,146), the methods used to conjugate peptides to antibodies (U.S. Pat. Nos. 5,194,254; 4,950,480), the methods used to conjugate peptides to insulin fragments (U.S. Pat. No. 5,442,043), the methods of U.S. Pat. No. 4,639,512, and the method of conjugating the cyclic decapeptide polymyxin B antibiotic to and IgG carrier using EDAC (1-ethyl-3-(3-dimethylaminopropyl)carbodiimide)-mediated amide formation. (See e.g., Drabick et al., Antimicrob. Agents Chemother., 42:583-588 (1998)). Approaches to conjugate nucleic acids to proteins are also known in the art, such as those described in U.S. Pat. Nos. 5,574,142; 6,117,631; and 6,110,687; each of is incorporated in its entirety by reference. Methods for conjugating lipids to peptides have been described in the art including, but not limited to, the use of reductive amination and an ether linkage which contains a secondary or tertiary amine (U.S. Pat. No. 6,071,532), the methods of U.S. Pat. No. 4,639,512, the methods used for covalently coupling peptides to unilamellar liposomes (Friede et al., Vaccine, 12:791-797 (1994)), of coupling human serum albumin to liposomes using the hetero-bifunctional reagent N-succinimidyl-S-acetylthioacetate (SATA) (Kamps et al., Biochim. Biophys. Acta, 1278:183-190 (1996)), of coupling antibody Fab′ fragments to liposomes using a phospholipid-poly(ethylene glycol)-maleimide anchor (Shahinian et al., Biochim. Biophys. Acta, 1239:157-167 (1995)), and of coupling Plasmodium CTL epitope to palmitic acid via cysteine-serine spacer amino acids (Verheul et al., J. Immunol. Methods, 182:219-226 (1995)).

A specifically bound agent can be detected in an individual using an in vivo imaging method, such as a radionuclide imaging, positron emission tomography, computerized axial tomography, X-ray or magnetic resonance imaging method, or can be detected using an ex vivo method, wherein, following administration, a sample of the tissue is obtained from the individual, and specific binding of the agent in the sample is detected (e.g., by immunohistochemical analysis).

An agent that is specifically bound to an integrin of interest in a sample can be detected directly by detecting the agent, or indirectly by detecting the presence of a moiety such as by detecting radioactivity emitted by a radionuclide moiety. Specifically bound agent also can be detected indirectly by further contacting it with a reagent that specifically interacts with the agent, or with a moiety linked to the agent, and detecting interaction of the reagent with the agent or label. For example, the moiety can be detected by contacting it with an antibody that specifically binds the moiety, particularly when the moiety is linked to the agent. The moiety also can be, for example, a substrate, which is contacted by an enzyme that interacts with and changes the moiety such that its presence can be detected. Such indirect detection systems, which include the use of enzymes such as alkaline phosphatase, horseradish peroxidase, beta-galactosidase and the like, are well known in the art and commercially available, as are the methods for incorporating or, linking the particular moiety to a particular type of agent.

II. Inducing Apoptosis

The present invention provides methods for inhibiting cell survival i.e., inducing apoptosis. In one embodiment, endothelial cells (HUVECs) were cultured on poly-L-lysine (which mediates nonspecific cell attachment to the substratum) and fibronectin substrates. Cells were analyzed for the binding of Annexin V, a Ca²⁺ dependent phospholipid-binding protein that binds to apoptotic cells with exposed phosphatidyl serine as discussed in P. Raynal and H. B Pollard. (P. Raynal and H. B Pollard, Biochemica et Biophysica Acta., 1197:63-93 (1994)). The present invention shows that HUVECs attached to fibronectin bind little Annexin V, while over 85% of cells on poly-L-lysine coated plates bound Annexin V. Additionally, lysates from HUVECs cultured in suspension or on poly-L-lysine and fibronectin were immunoblotted for poly (ADP ribose) polymerase (PARP). PARP is an enzyme involved in DNA repair that is cleaved by caspase 3 during the early stages of apoptosis to produce 85 kD and 25 kD fragments, resulting in loss of normal PARP function. (See e.g., D. W. Nicholson et al., Nature, 376: 37-43 (1995); and T. Partel et al., FASEB J. 10:587-597 (1995)). Cells in suspension or attached to poly-L-lysine displayed significant PARP cleavage, while cells attached to fibronectin showed little PARP cleavage. Similarly, attachment to fibronectin, but not poly-L-lysine, protects HUVECs from DNA fragmentation associated with apoptosis. These studies indicate that in certain embodiments of the present invention, fibronectin attachment promotes the survival of endothelial cells.

To determine if integrin α5β1 is the fibronectin receptor supporting HUVEC survival in vitro, HUVECs were cultured on surfaces coated with αvβ3 antibodies or control antibodies. The present invention contemplates that immobilized anti-integrin antibodies cluster integrins, thereby acting as agonists. (See e.g., S. Stromblad et al., J. Clin. Invest., 98:426-433 (1996); and D. I. Leaveley et al., J. Cell Biol., 121:163-170 (1993)). Cells attached to surfaces coated with antibodies directed against α5β1 bound little Annexin, and thus remained viable. In contrast, more than 75% of cells attached to control antibodies were Annexin positive. Cells attached to α5β1 antibody-coated surfaces also showed significantly less PARP cleavage and DNA fragmentation than cells attached to control antibodies. These results indicate that, in some embodiments of the present invention, α5β1 promotes endothelial cell survival.

Antagonists of integrin α5β1 block angiogenesis in vivo but have no effect on unstimulated blood vessels. (S. Kim et al., Am. J. Path., 156:1345-1362 (2000)). In one embodiment, to determine if the integrin antagonists induce endothelial cell apoptosis during angiogenesis in vivo, CAMs stimulated by bFGF were treated with saline and α5β1 function-blocking and isotype-matched control antibodies. Twenty-four hrs later, chicken embryos were injected intravenously with Annexin V-FITC. Anti-α5β1 antibodies, but not saline or control antibodies, induced Annexin V staining of endothelial cells in living CAMs, showing that α5β1 regulates survival in vivo of proliferating endothelial cells. Vessels in anti-α5β1 treated CAMs bound eight times more Annexin V than control treated CAMs. As α5β1 is only expressed at significant levels on proliferating endothelial cells, α5β1 function-blocking antibodies target these cells. Furthermore, as peptide and small molecule antagonists of α5β1 also inhibit angiogenesis and induce apoptosis, antibody-mediated complement activation is not likely to play a significant role in this apoptosis induction. Thus, in preferred embodiments, integrin α5β1 antagonists induce endothelial cell apoptosis in vivo.

To confirm that α5β1 antagonists cause apoptosis in vivo, sections of CAMs were analyzed for the expression of cleaved caspase 3 in blood vessels. Cleavage of caspase 3 into 17 and 12 kDa fragments is an indication of caspase 3 activation; the amount of cleaved caspase 3 is a quantitative index of apoptosis induction. Anti-mammalian caspase 3 antibodies cross-react with avian caspase 3, as avian caspases exhibit 65% overall sequence identity and 100% activation domain identity with mammalian caspases. (A. L. Johnson and J. T. Bridgham, Biology of Reproduction, 62:589-598 (2000)). Treatment with either α5β1 or αvβ3 antagonists induces caspase 3 cleavage (green) in blood vessels (red) in growth factor stimulated CAMs. Furthermore, isolated cells from CAMs treated with α5β1 and αvβ3 function-blocking antibodies bound significantly more FITC-Annexin V than in cells from control CAMs. Additionally, α5β1 antagonists induced a significant increase in DNA fragmentation in vivo. These studies show that integrin (e.g., α5β1) antagonists induce endothelial cell apoptosis during angiogenesis. Accordingly, preferred embodiments of the present invention provide methods and compositions for administering integrin antagonists to a subject to inhibit undesirable angiogenesis.

In still other embodiments, the present invention provides studies and examples of how integrin (e.g., α5β1) antagonists interfere with cell (e.g., normal cell, such as endothelial cells) survival during angiogenesis. For example, quiescent (serum-starved) and proliferating (cultured in complete medium) HUVECs were plated on poly-L-lysine, fibronectin, vitronectin, or collagen coated culture plates in the presence of antibody antagonists of integrins α5β1, αvβ3, or α2β1 for 24 hrs. Cells were then stained with FITC-Annexin V. While fibronectin and vitronectin promote the survival of serum starved as well as proliferating endothelial cells, collagen promotes only the survival of quiescent endothelial cells. In some embodiments, integrin antagonists that block cell attachment to the substratum induce apoptosis in endothelial cells. For example, antagonists of α5β1 induce apoptosis on fibronectin, while antagonists of αvβ3 induce apoptosis on vitronectin and antagonists of α2β1 induce apoptosis on collagen. However, α5β1 antagonists also induce apoptosis of cells on vitronectin without affecting attachment to vitronectin. In contrast, α5β1 antagonists do not affect cell survival on collagen. Anti-α5β1, but not control antibodies also induce PARP cleavage in HUVECs plated on vitronectin substrates. Thus, the present invention shows that unligated α5β1 integrin inhibits endothelial cell survival on provisional matrix proteins such as fibronectin and vitronectin, but not on other matrix proteins such as collagen. Interestingly, α5β1 and αvβ3 antagonists induce apoptosis in proliferating cells more effectively than in quiescent cells, thus these two integrins provide important survival signals to proliferating endothelial cells such as those participating in angiogenesis. These studies further show that integrin α5β1 can directly and indirectly regulate the survival of proliferating cells (e.g., endothelial cells).

Additional embodiments of the present invention are directed to determining the nature of the cell death pathway induced by integrin (e.g., α5β1) antagonists contemplated for in the methods and compositions of the present invention. In one such embodiment, endothelial cells were plated on ECM protein coated culture plates in the presence of integrin antagonists and caspase inhibitors or vehicle control (0.33% DMSO). As α5β1 antagonists do not induce cell death on collagen, these studies were restricted to examination of effects of antagonists on cells attached to fibronectin and vitronectin. Cell death induced by anti-α5β1 antibodies was blocked by caspase 3 inhibitors whether the cells were attached to fibronectin or vitronectin. Cell attachment to poly-L-lysine rapidly activated caspases 3 and 8 while attachment to vitronectin did not. α5β1 antibodies, but not control antibodies also activated caspases 3 and 8 (P=0.0001 and P=0.002, respectively) in cells attached to vitronectin and fibronectin. Caspase 3 cleavage was also readily detected in cells treated with anti-α5β1 antibodies, but not control antibodies. In contrast, caspase 9 cleavage was not detected in cells treated with α5β1 antagonists. These studies show that, in some preferred embodiments, integrin α5β1 antagonists induce a pro-apoptotic pathway in proliferating endothelial cells that results from activation of initiator caspases (8) rather than stress caspase (9) pathways. These results further show, that in some additional embodiments, that blocking integrin (e.g., α5β1) ligation induces caspase 3-mediated death even when cells are still attached to provisional matrix ligands through other integrins.

Still further embodiments of the present invention show that integrin-mediated survival depends on suppression of caspase 3 and 8 activation in vitro. As α5β1 and αvβ3 antagonists block angiogenesis in vivo, these antagonists are contemplated to induce caspase 3 and 8 activation in vivo. In one embodiment, CAMs stimulated with bFGF were treated with saline, vehicle control (DMSO), caspase 3 or 8 inhibitors, and anti-integrin antibodies in the presence or absence of caspase inhibitors. Angiogenesis was inhibited by anti-α5β1 antibodies. In some of these embodiments, inhibition was partially reversed by cell permeable caspase 3 inhibitors and fully reversed by caspase 8 inhibitors. Similarly, in some other embodiments, angiogenesis was inhibited by anti-αvβ3 and was partially reversed with caspase 3 and caspase 8 inhibitors. However, caspase 9 inhibitors had little effect on angiogenesis. And in still other embodiments, caspase inhibitors alone had no effect on angiogenesis or on unstimulated CAMs. These results indicate that integrin (e.g., α5β1 and αvβ3) antagonists activate caspases 8 and 3 in vivo, thereby inhibiting angiogenesis.

In preferred embodiments, the present invention shows that integrin ligation suppresses PKA activation. Particularly preferred embodiments show that cell death induced by integrin antagonists is PKA dependent. For example, antagonists of α5β1 activate PKA even if endothelial cells are attached to vitronectin. Therefore, the present invention investigated whether PKA plays a role in integrin antagonist induced apoptosis in vitro and in vivo. Pharmacological inhibitors of PKA substantially suppressed apoptosis induced by anti-αvβ3 (P=0.05) or anti-α5β1 (P=0.05) in cells attached to vitronectin. Furthermore, PKA inhibitors blocked caspase 3 cleavage induced by anti-α5β1 in cells attached to vitronectin.

The present invention shows that integrin antagonists induce cell death by activating PKA. Thus, in still further embodiments, the role of PKA in integrin antagonist induced cell death was examined. Briefly, in one embodiment, endothelial cells were plated on fibronectin, vitronectin, or collagen coated plates in the presence of anti-α5β1, anti-αvβ3, or anti-α2β1 antibodies and transfected with a mutationally inactive form of PKA (dnPKA). The mutationally inactive PKA, but not a control transgene (GFP), prevented cell death (P=0.004 or less) induced by integrin antagonists even whether these antagonists inhibit cell attachment or not. Therefore, certain embodiments of the present invention show that both direct (anoikis) and indirect integrin antagonist-mediated cell death is PKA dependent.

Other embodiments of the present invention are directed to determining whether activation of PKA directly induces endothelial cell death. To that end, in one embodiment, endothelial cells were treated with dibutyryl cAMP or were transiently transfected with the catalytic subunit of PKA to activate PKA. Both cAMP and expression of the catalytic subunit of PKA significantly induce apoptosis (P=0.009 and P=0.003, respectively) in endothelial cells attached to vitronectin or to other ECM substrates. Accordingly, some preferred embodiments of the present invention show that PKA directly induces apoptosis in endothelial cells.

To determine whether PKA plays a role in the negative regulation of angiogenesis, bFGF stimulated CAMs were transfected with GFP and mutationally inactive PKA (dnPKA) expression plasmids in the presence and absence of α5β1 antibodies. While integrin (e.g., α5β1) antagonists significantly block angiogenesis, the mutationally inactive PKA partially reverses this inhibition. In other embodiments, similar results have been observed with pharmacological PKA inhibitors. (See e.g., S. Kim et al., J. Biol. Chem., 275: 33920-33928 (2000)). Since PKA plays a role in the negative regulation of angiogenesis by integrin antagonists, the present invention investigated whether direct activation of PKA also inhibits angiogenesis. Expression of the PKA catalytic subunit during angiogenesis in vivo completely suppresses angiogenesis (P=0.0005), as does exposure of CAMs to cAMP. VEGF stimulated angiogenesis is also inhibited by activation of PKA. This inhibition results from apoptosis induction, as it is accompanied by cleavage of caspase 3 in vivo. Thus, in some embodiments, activation of PKA by integrin antagonists, by cAMP or by overexpression of the catalytic subunit of PKA induces endothelial cell apoptosis and ultimately inhibits angiogenesis.

III. Screening Compounds

The present invention further provides methods for identifying compounds which are capable of inhibiting angiogenesis, inhibiting endothelial cells adhesion, and/or inhibiting endothelial cell migration. A screening assay of the invention can be performed by contacting cells with a test compound, and detecting activation of PKA in the cells, and/or detecting inhibition of angiogenesis, cells adhesion, cell migration and/or cell survival thereby identifying the compound. A tissue can be contacted with the agent in vivo or ex vivo (See e.g., U.S. Pat. No. 5,622,699, incorporated by reference). Where a screening method of the invention is performed using an in vitro format, it can be adapted to automated procedure, thus allowing high throughput screening assays for examining libraries of molecules.

Methods for preparing libraries of molecules, which can be screened using a method of the invention to identify antagonists of integrins of interest that inhibit angiogenesis, endothelial cells adhesion and/or endothelial cell migration processes which are associated with expression of an integrin of interest, are known in the art. These are exemplified by methods for preparing oligonucleotide libraries (See e.g., Gold et al., U.S. Pat. No. 5,270,163, incorporated by reference); peptide libraries (See e.g., Koivunen et al. J. Cell Bio, 124: 373-380 (1994)); peptidomimetic libraries (See e.g., Blondelle et al., Trends Anal. Chem. 14:83-92 (1995)) oligosaccharide libraries (See e.g., York et al., Carb. Res. 285:99-128 (1996); Liang et al., Science 274:1520-1522 (1996); and Ding et al., Adv. Expt. Med. Biol. 376:261-269 (1995)); lipoprotein libraries (See e.g., de Kruif et al., FEBS Lett., 399:232-236 (1996)); glycoprotein or glycolipid libraries (See e.g., Karaoglu et al., J. Cell Biol. 130:567-577 (1995)); or chemical libraries containing, for example, drugs or other pharmaceutical agents (See e.g., Gordon et al., J. Med. Chem. 37:1385-1401 (1994); Ecker and Crook, Bio/Technology 13:351-360 (1995); and U.S. Pat. No. 5,760,029, incorporated by reference). Libraries of diverse molecules also can be obtained from commercial sources. Libraries of diverse molecules also can be obtained from commercial sources.

IV. Exemplary Agents

A wide range of agents find use in the methods of the present invention (e.g., diagnostic, therapeutic, screening, etc.). The terms “test compound,” “compound,” “agent,” “test agent,” “molecule,” and “test molecule,” as used herein refer to any type of molecule (for example, a peptide, nucleic acid, carbohydrate, lipid, organic molecule, and inorganic molecule, etc.) obtained from any source (for example, plant, animal, and environmental source, etc.), or prepared by any method (for example, purification of naturally occurring molecules, chemical synthesis, and genetic engineering methods, etc.). In one embodiment, the term “test compound” refers to any chemical entity, pharmaceutical, drug, and the like that can be used to treat or prevent a disease, illness, sickness, or disorder of bodily function. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

In one embodiment, any compound that modulates the activity of PKA in normal cells (e.g., endothelial, smooth muscles, fibroblasts, myocytes, lymphocytes, monocytes, macrophages, megakaryocytes, eosinophils, neurites, and the like), and tumor cells (e.g., lymphoma, leukemia, carcinomas, including, but not limited to, ovarian, uterine, testicular, breast, prostate, gastric, head and neck, squamous, and the like, sarcomas, osteosarcomas, melanomas, and the like) is a candidate compound for use in the methods of the present invention. In some of these embodiments, compounds that modulate the activity of PKA inhibit integrin ligation. In this regard, any agent/moiety that inhibits integrin ligation finds use in the present invention (e.g., mutationally inactive integrin subunits, anti-integrin antibodies, cell permeable forms of cAMP, drugs, small molecules, and the like).

In some other embodiments, the present invention provides methods and compositions that modulate undesirable apoptosis in a cell or tissue. Examples of undesirable apoptosis, include, but are not limited, hyperproliferative diseases, and cancers.

In still some other embodiments, the present invention provides compositions and methods that modulate undesirable angiogenesis, cell migration, cell adhesion, and/or cell survival.

In preferred embodiments, the present invention provides agents that activate PKA by blocking integrin function. For example, the present invention provides, but is not limited to, small molecule and peptide antagonists of integrins integrin function, for example, the present invention provides, but is not limited to, small molecule and peptide antagonists of integrins αvβ1, αvβ3, αvβ5, αvβ6, αvβ8, α1β1, α2β1, α4β1, α5β1, α6β1, α7β1, α8β1, α9β1, α10β1, α6β4, α4β7, αMβ2, αLβ2, αXβ2, and αIIββ2, and the like.

In still other preferred embodiments, the present invention provides agents that inhibit the migration of cells (e.g., normal cells and tumor cells) that are downstream of PKA, including, but not limited to, dominant positive Rho (e.g., RhoV14), dominant negative Src, and active Csk, and the like.

Other preferred embodiments of the present invention provide methods and compositions for modulating (e.g., inhibiting) cell migration and/or survival by elevating PKA levels in a cell of tissue by providing an agent, including, but not limited to, PKA catalytic subunits, pertussis toxin, cholera toxin, PTHrP and/or PTH (and derivatives thereof), Gαi minigene, dominant negative Gαi, dominant negative Gα 12/13, constitutively active Gαs, and anti-CD-47 antibodies, and the like.

The term “pertussis toxin” refers to a cell permeable bacterial enzyme that ADP ribosylates the heterotrimeric Gai subunit. Exemplary pertussis toxin is commercially available from Calbiochem and other commercial sources. The inventors' data shows that pertussis toxin inhibits PKA activation.

The term “cholera toxin” refers to a cell permeable bacterial toxin that ADP ribosylates and activates Gas. Exemplary cholera toxin is commercially available from Calbiochem. The inventors' data shows that cholera toxin activates PKA.

The term “Galpha i minigene” refers to a gene sequence representing the small fragment of Gai that binds to its receptors, thus reducing and/or preventing activation (Gilchrist A et al., Methods Enzymol. (2002) 344:58-69) It is also commercially available from cue BIOtech, 303 E. Chicago Avenue, Ward Building, Suite 17-171 Chicago, Ill. 60611.

The term “dominant negative Gai” refers to a Gai protein that has a point mutation (Q to L and D to N) that inhibits its ability to block adenyl cyclase activation, thereby causing PKA activation. Dominant negative Gai is available from Guthrie cDNA Resource Center, Guthrie Research Institute, One Guthrie Square, Sayre, Pa. 18840.

The term “dominant negative Ga12” refers to to a Ga12 protein that has a point mutation (Q to L and D to N) that inhibits its ability to block adenyl cyclase activation, thereby causing PKA activation. Dominant negative Ga12 is available from Guthrie cDNA Resource Center, PA.

The term “dominant negative Ga13” refers to a Ga13 protein that has a point mutation (Q to L and D to N) that inhibits its ability to block adenyl cyclase activation, thereby causing PKA activation.

The term “constitutively active G alpha s” refers to a G alpha s protein that has a point mutation (Q to L) that activates its ability to stimulate adenyl cyclase activation, thereby causing PKA activation. Constitutively active G alpha s is available from Guthrie cDNA Resource Center, PA.

The term “anti-CD47” refers to an antibody that binds CD47. In one embodiment, anti-CD47 inhibits the function of CD47, an integrin associated Glpha i linked receptor. It is described in Gao et al. J Cell Biol. (1996 Oct) 135(2):533-44. Thrombospondin modulates alpha v beta 3 function through integrin-associated protein.

The terms “dominant positive Rho” and “Rho V14” refer to constitutively active Rho (due to point mutation) that causes increased focal adhesion formation and/or decreased cell migration. Rho V14 is available from Guthrie cDNA Resource Center, PA

The terms “dominant negative Src” and “Src 251” refer to Src protein that is truncated after amino acid 251. It has no kinase activity as it lacks the kinase domain. It inhibits native Src activity and causes inhibition of cell migration (Eliceiri et al. (1999) Mol Cell. 4:915-24). Exemplary Src nucleotide sequences, proteins, and/or at least portions thereof are known in the art such as GenBank accession numbers: NM_{—}031977.1, AF157016.1, AF157016, NM_{—}009271.1, M17031.1, AF130457.1, BC039953.1, BC051270.1, NM_{—}005417.2, BC011566.1, BC011566, J00844.1, V00402.1, X51863.1, M21526.1, L21974.1, AK024281.1, V01169.1, L29199.1, X84074.1, X13745.1, M33292.1, S37068.1, M11753.1, D10652.1, M84475.1, X84073.1, X52822.1, AF033808.1, AF033808, V01197.1, X15345.1, J02342.1, X14718.1, X51861.1, X68524.1, AF052428.1, AF052428, BC045134.1, M23422.1, M24704.2, AL672259.9, V01167.1, AL133293.28, K03218.1, AK091756.1, X04000.1, AF077754.1, U01149.1, AF440202.1, U01148.1, X54971.1, J02350.1, AK017629.1, K03214.1, X03996.1, M16237.1, and AB025550.1

The terms “active C-terminal Src Kinase” and “Csk” refer to an enzyme that phosphorylates Src on Tyr 527 and inhibits its activity (Eliceiri et al. (1999) Mol Cell. 4:915-24). The inventors' data shows it causes inhibition of cell migration. Exemplary Exemplary Csk nucleotide sequences, proteins, and/or at least portions thereof are known in the art such as GenBank accession numbers: NM_{—}007783.2, BC052006.1, C018394.1, U05247.1, X58631.1, XM_{—}236290.1, AK013057.1, NM_{—}004383.1, X60114.1, X59932.1, AY007162.1, M85039.1, X57242.1, AC091230.23, X74765.1, AF052430.1, and NM_{—}004383.

In yet another embodiment, the invention provides a method for reducing at least one of cell migration, cell survival, cell adhesion, and angiogenesis, comprising: a) providing: i) at least one cell; and ii) at least one Src inhibitor; and b) treating the at least one cell with the at least one Src inhibitor such that at least one of cell migration, cell survival, adhesion by the cell, and angiogenesis by the cell is reduced. In one embodiment, the method further comprises step c) detecting a reduction in at least one of migration of the cell, survival of the cell, adhesion by the cell, and angiogenesis by the cell.

The term “Src inhibitor” refers to an agent that reduces kinase activity of one or more members of the Src family (such as pp60 Src, p56 Lck, p59 Fyn, and Hck) to phosphorylate on tyrosine a Src family substrate (such as the exemplary substrates polyGluTyr, and Ac-IleTyrGlyGluPhe-NH2). Cellular substrates of Src include focal adhesion kinase, paxillin and pp190RhoGAP and others. Methods for determining the level of Src kinase activity are known in the art. For example, protein sample containing Src family members and 50 mM Tris, pH 7.5, 10 mM MgCl2, 0.25 mg/ml BSA, 50 μM Src substrate (e.g., polyGluTyr), 3000 Ci/mmol [g32 P] ATP and 100 μM ATP are combined for 5 minutes at 30° C. Twenty μl samples of reaction mixtures are spotted onto phosphocellulose discs and washed repeatedly with 1% (v/v) phosphoric acid in water. Incorporated radioactivity may be determined by scintillation counting. The specific activity of the sample is calculated as μCi 32P incorporated per μg protein per min. Alternatively, a protein sample containing Src family members and 50 mM Tris, pH 7.5, 10 mM MgCl2, 0.25 mg/ml BSA, 50 μM Src protein substrate (e.g., Focal adhesion kinase or fusion protein fragment), 3000 Ci/mmol [g32 P] ATP and 100 μM ATP are combined for 5 minutes at 30° C. Samples of reaction mixtures are electrophoresed on SDS polyacrylamide gels. Gels are dried and autoradiographed. Src activity is determined by incorporation of radioactivity into the specific substrate band on the gel. Alternatively, gels may be blotted onto nitrocellulose prior to autoradiography.

In one embodiment, exemplary Src inhibitors are chosen from 4-Amino-1-tert-butyl-3-(1′-naphthyl)pyrazolo[3,4-d]pyrimidine (PP1), 4-Amino-5-(4-chlorophenyl)-7-(t-butyl)pyrazolo[3,4-d]pyrimidine (PP2), (Z)-3-[4-(Dimethylamino)benzylidenyl]indolin-2-one, a-Cyano-(3,4-dihydroxy)cinnamoyl-(3′,4′-dihydroxyphenyl)ketone, 5-Amino-[(N-2,5-dihydroxybenzyl)-N′-2-hydroxybenzyl]salicylic Acid, 2′,4′,3,4-Tetrahydroxychalcone, 3-Hydroxy-1-methoxyanthraquinone-2-aldehyde, 5-Amino-[(N-2,5-dihydroxybenzyl)-N′-2-hydroxybenzyl]salicylic Acid, (Z)-5-Bromo-3-(4,5,6,7-tetrahydro-1H-indol-2-ylmethylene)-1,3-dihydroindol-2-one, 2′,4′3,4-Tetrahydoxychalcone, 3-Hydroxy-1-methoxyanthraquinone-2-aldehyde, 4-(4′-Phenoxyanilino)-6,7-dimethoxyquinazoline, Herbimycin A, Streptomyces sp., Lavendustin A, Radicicol, Diheterospora chlamydosporia, and p60v-src 137-157 Inhibitor Peptide (VAPSDSIQAEEWYFGKITRRE) (SEQ ID NO:116). These exemplary Src inhibitors are commercially available (such as from Calbiochem, San Diego, Calif.). In a preferred embodiment, the invention uses the exemplary Src inhibitors PP1 and/or PP2 (Calbiochem, San Diego, Calif.). PP1 and/or PP2 are pseudo substrates of the enzymes; they bind and are not released. In one embodiment, PP1 and/or PP2 reduce cell migration.

Accordingly, in some embodiments, the present invention contemplates methods and compositions for expression of an exogenous gene (or polynucleotide) sequences of interest into a cell or tissue. Likewise, in some other embodiments, the present invention provides compositions and methods for increasing the expression of endogenous genes (or polynucleotides) of interest in a cell or tissue.

The methods of the present invention are useful for treating a number of diseases and conditions characterized by undesirable cell migration and/or angiogenesis, cell migration, cell adhesion, and/or cell survival, including, but not limited to: endothelial or vascular smooth muscle angiogenesis; restenosis (e.g., vascular smooth muscle); arteriosclerosis (e.g., vascular smooth muscle, monocytes, and/or macrophages, etc.); cancer (e.g., begin and/or malignant tumor cells); tumor metastasis (e.g., malignant tumor metastasis); fibrosis (e.g., fibroblasts in heart, lung, liver, etc.); hemangiomas (e.g., endothelial cells, etc.); lymphomas (e.g., immune cells, etc.); leukemia (e.g., immune cells, etc.); psoriasis (endothelial cells, etc.); arthritis (e.g., endothelial cells, synoviocytes, and/or fibroblasts, etc.); other auto-immune disease (e.g., Lupus, Crohn's disease, multiple sclerosis, diabetes, etc.); ALS (e.g., immune cells, etc.); graft rejection (e.g., immune cells, etc.); retinopathies (e.g., macular degeneration, and retinal tearing, etc.), and the like. Particularly useful are agents that have undergone extensive testing and are readily available.

The present invention also provides compositions and methods for screening test compounds either alone or in association with one or more compounds known to activate PKA in a particular cell or tissue. The present invention is not limited, however, to the above mentioned screening methods. Those skilled in the art will readily appreciate, in view of the disclosure of the present invention, other screening and treatment methods are contemplated by the present invention.

Some embodiments of the present invention provide a method of administering to a subject an effective amount of an agent that activates PKA and/or inhibits integrin ligation (and enantiomers, derivatives, and pharmaceutically acceptable salts thereof) and at least one anticancer agent (e.g., a conventional anticancer agent, such as, chemotherapeutic drugs, and/or radiation therapy).

Anticancer agents suitable for use with the present invention include, but are not limited to, agents that induce apoptosis or nucleic acid damage, inhibit nucleic acid synthesis, affect microtubule formation, and affect protein synthesis or stability.

Classes of anticancer agents suitable for use the methods of the present invention include, but are not limited to: 1) alkaloids, including, microtubule inhibitors (e.g., Vincristine, Vinblastine, and Vindesine, etc.), microtubule stabilizers (e.g., Paclitaxel (Taxol), and Docetaxel, etc.), and chromatin function inhibitors, including, topoisomerase inhibitors, such as, epipodophyllotoxins (e.g., Etoposide (VP-16), and Teniposide (VM-26), etc.), and agents that target topoisomerase I (e.g., Camptothecin and Isirinotecan (CPT-11), etc.); 2) covalent DNA-binding agents (alkylating agents), including, nitrogen mustards (e.g., Mechlorethamine, Chlorambucil, Cyclophosphamide, Ifosphamide, and Busulfan (Myleran), etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); 3) noncovalent DNA-binding agents (antitumor antibiotics), including, nucleic acid inhibitors (e.g., Dactinomycin (Actinomycin D), etc.), anthracyclines (e.g. Daunorubicin (Daunomycin, and Cerubidine), Doxorubicin (Adriamycin), and Idarubicin (Idamycin), etc.), anthracenediones (e.g., anthracycline analogues, such as, (Mitoxantrone), etc.), bleomycins (Blenoxane), etc., and plicamycin (Mithramycin), etc.; 4) antimetabolites, including, antifolates (e.g., Methotrexate, Folex, and Mexate, etc.), purine antimetabolites (e.g., 6-Mercaptopurine (6-MP, Purinethol), 6-Thioguanine (6-TG), Azathioprine, Acyclovir, Ganciclovir, Chlorodeoxyadenosine, 2-Chlorodeoxyadenosine (CdA), and 2′-Deoxycoformycin (Pentostatin), etc.), pyrimidine antagonists (e.g.,-fluoropyrimidines (e.g., 5-fluorouracil (Adrucil), 5-fluorodeoxyuridine (FdUrd) (Floxuridine)) etc.), and cytosine arabinosides (e.g., Cytosar (ara-C) and Fludarabine, etc.); 5) enzymes, including, L-asparaginase, and hydroxyurea, etc.; 6) hormones, including, glucocorticoids, such as, antiestrogens (e.g., Tamoxifen, etc.), nonsteroidal antiandrogens (e.g., Flutamide, etc.), and aromatase inhibitors (e.g., anastrozole (Arimidex), etc.); 7) platinum compounds (e.g., Cisplatin and Carboplatin, etc.); 8) monoclonal antibodies conjugated with anticancer drugs, toxins, and/or radionuclides, etc.; 9) biological response modifiers (e.g., interferons (e.g., IFN-α, etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., Batimistat, etc.); and 17) inhibitors of angiogenesis. Any pharmaceutical that is routinely used in a cancer therapy context finds use in the present invention. Conventional anticancer agents that are suitable for administration with the disclosed gossypol compounds include, but are mot limited to, adriamycin, 5-fluorouracil, etoposide, camptothecin, actinomycin-D, mitomycin C, or more preferably, cisplatin. These agents may be prepared and used as a combined therapeutic composition, or kit, by combining it with an immunotherapeutic agent, as described herein.

In some embodiments of the present invention, the therapeutic compound treatments further comprise one or more agents that directly cross-link nucleic acids (e.g. DNA) to facilitate DNA damage leading to a synergistic, antineoplastic agents of the present invention. The compositions of the present invention may be delivered via any suitable method, including, but not limited to, injection intravenously, subcutaneously, intratumorally, intraperitoneally, or topically (e.g., to mucosal surfaces).

Agents that damage DNA also include compounds that interfere with DNA replication, mitosis, and chromosomal segregation. Such chemotherapeutic compounds include, but are not limited to, adriamycin, also known as doxorubicin, etoposide, verapamil, podophyllotoxin, and the like. These compounds are widely used in clinical settings for the treatment of neoplasms, and are administered through bolus injections intravenously at doses ranging from 25-75 Mg/M² at 21 day intervals for adriamycin, to 35-50 Mg/M² for etoposide intravenously or double the intravenous dose orally.

Agents that disrupt the synthesis and fidelity of nucleic acid precursors and subunits also lead to DNA damage and find use as chemotherapeutic agents in the present invention. A number of nucleic acid precursors have been developed. As such, agents such as 5-fluorouracil (5-FU) are preferentially used by neoplastic tissue, making this agent particularly useful for targeting to neoplastic cells. The doses delivered may range from 3 to 15 mg/kg/day, although other doses may vary considerably according to various factors including stage of disease, amenability of the cells to the therapy, amount of resistance to the agents and the like.

In preferred embodiments, the anticancer agents used in the present invention are those that are amenable co-administration with the disclosed therapeutic compounds or are otherwise associated with the disclosed therapeutic compounds such that they can be delivered into a subject, tissue, or cell without loss of fidelity of anticancer effect. For a more detailed description of cancer therapeutic agents such as a platinum complex, verapamil, podophyllotoxin, carboplatin, procarbazine, mechlorethamine, cyclophosphamide, camptothecin, ifosfamide, melphalan, chlorambucil, bisulfan, nitrosurea, adriamycin, dactinomycin, daunorubicin, doxorubicin, bleomycin, plicomycin, mitomycin, etoposide (VP16), tamoxifen, taxol, transplatinum, 5-fluorouracil, vincristin, vinblastin and methotrexate and other similar anti-cancer agents, those of skill in the art are referred to any number of instructive manuals including, but not limited to, the Physician's Desk reference and to Goodman and Gilman's “Pharmaceutical Basis of Therapeutics” ninth edition, Eds. Hardman et al., 1996.

In still further embodiments of the present invention, yet even more specific examples of potential therapeutic anti-integrin and PKA activating compounds contemplated for use in the present invention are described below.

A. Antibodies

In some preferred embodiments, the agent employed to inhibit angiogenesis, cell migration, cell adhesion and/or cell survival is an antibody. In one preferred embodiment, an antibody is selected which is specific for an integrin such as, but not limited to, αvβ1; αvβ3; αvβ5; αvβ6; αvβ8; α1β1; α2β1; α4β1; α5β1; α6β1; α7β1; α8β1; α9β1; α10β1; α6β4; α4β7; αMβ2; αLβ2; αXβ2; and αIIββ2) with its corresponding ligand(s). In preferred embodiments, the terms “antibody” and “immunoglobulin” are used interchangeably to refer to a glycoprotein or a portion thereof (including single chain antibodies), which is evoked in an animal by an immunogen and which demonstrates specificity to the immunogen, or, more specifically, to one or more epitopes contained in the immunogen. In preferred embodiments, antibodies includes the antigen binding fragments of such antibodies, including, for example, Fab, F(ab′)₂, Fd or Ev fragments. In additional preferred embodiments, the antibodies of the present invention further include chimeric and humanized antibodies. Antibodies may be polyclonal or monoclonal. The term “polyclonal antibody” refers to an immunoglobulin produced from more than a single clone of plasma cells.

Antibodies contemplated to be within the scope of the invention include naturally occurring antibodies as well as non-naturally occurring antibodies, including, for example, single chain antibodies, chimeric, bifunctional and humanized antibodies, as well as antigen-binding fragments thereof. Naturally occuring antibodies may be generated in any species including murine, rat, rabbit, hamster, human, and simian species using methods known in the art. Non-naturally occurring antibodies can be constructed using solid phase peptide synthesis, can be produced recombinantly or can be obtained, for example, by screening combinatorial libraries of variable heavy chains and variable light chains as previously described (Huse et al., Science 246:1275-1281 (1989)). These and other methods of making, for example, chimeric, humanized, CDR-grafted, single chain, and bifunctional antibodies are well known to those skilled in the art (Winter and Harris, Immunol. Today 14:243-246 (1993); Ward et al., Nature 341:544-546 (1989); Hilyard et al., Protein Engineering: A practical approach (IRL Press 1992); Borrabeck, Antibody Engineering, 2d ed. (Oxford University Press 1995).

In one embodiment, an anti-integrin antibody, or antigen binding fragment thereof, is characterized by having specific binding activity for its corresponding integrin binding partner of at least about 1×10⁵M⁻¹, more preferably at least about 1×10⁶M⁻¹, and yet more preferably at least about 1×10⁷M⁻¹.

Those skilled in the art know how to make polyclonal and monoclonal antibodies which are specific for a desirable polypeptide. For example, monoclonal antibodies may be generated by immunizing an animal (e.g., mouse, rabbit, etc.) with a desired antigen and the spleen cells from the immunized animal are immortalized, commonly by fusion with a myeloma cell.

Immunization with antigen may be accomplished in the presence or absence of an adjuvant, e.g., Freund's adjuvant. Typically, for a mouse, 10 μg antigen in 50-200 μl adjuvant or aqueous solution is administered per mouse by subcutaneous, intraperitoneal or intramuscular routes. Booster immunization may be given at intervals, e.g., 2-8 weeks. The final boost is given approximately 2-4 days prior to fusion and is generally given in aqueous form rather than in adjuvant.

Spleen cells from the immunized animals may be prepared by teasing the spleen through a sterile sieve into culture medium at room temperature, or by gently releasing the spleen cells into medium by pressure between the frosted ends of two sterile glass microscope slides. The cells are harvested by centrifugation (400×g for 5 min.), washed and counted.

Spleen cells are fused with myeloma cells to generate hybridoma cell lines. Several mouse myeloma cell lines which have been selected for sensitivity to hypoxanthine-aminopterin-thymidine (HAT) are commercially available and may be grown in, for example, Dulbecco's modified Eagle's medium (DMEM) (Gibco BRL) containing 10-15% fetal calf serum. Fusion of myeloma cells and spleen cells may be accomplished using polyethylene glycol (PEG) or by electrofusion using protocols which are routine in the art. Fused cells are distributed into 96-well plates followed by selection of fused cells by culture for 1-2 weeks in 0.1 ml DMEM containing 10-15% fetal calf serum and HAT. The supernatants are screened for antibody production using methods well known in the art. Hybridoma clones from wells containing cells which produce antibody are obtained, e.g., by limiting dilution. Cloned hybridoma cells (4-5×10⁶) are implanted intraperitoneally in recipient mice, preferably of a BALB/c genetic background. Sera and ascites fluids are collected from mice after 10-14 days.

The invention also contemplates humanized antibodies which are specific for at least a portion of an integrin (e.g., α5β1) of interest or its ligands. Humanized antibodies may be generated using methods known in the art, such as those described in U.S. Pat. Nos. 5,545,806; 5,569,825 and 5,625,126, the entire contents of which are incorporated by reference. Such methods include, for example, generation of transgenic non-human animals which contain human immunoglobulin chain genes and which are capable of expressing these genes to produce a repertoire of antibodies of various isotypes encoded by the human immunoglobulin genes.

In a preferred embodiment, the anti-integrin antibodies are specific for their target integrins or to portions thereof. In more preferred embodiments, anti-integrin antibodies bind to their target integrins with at least 2 times greater, preferably at least 5 times greater, more preferably at least 10 times greater, and yet more preferably at least 100 times greater, affinity than it binds another integrin. Anti-integrin antibodies include, without limitation, mouse anti-human integrin antibodies. Also included within the scope of this invention are humanized anti-human-anti-integrin antibodies. In yet another preferred embodiment, the antibody is specific for fibronectin or a portion thereof.

B. Peptides and Peptide Memitics

In other alternative embodiments, the agent employed to inhibit angiogenesis, cell migration, cell adhesion and/or cell survival is one which inhibits the specific binding of an integrin of interest (including, but not limited to, integrins: αvβ1; αvβ3; αvβ5; αvβ6; αvβ8; α1β1; α2β1; α4β1; α5β1; α6β1; α7β1; α8β1; α9β1; α10β1; α6β4; α4β7; αMβ2; αLβ2; αXβ2; and αIIββ2) with its corresponding ligand(s). The term “peptide,” as used herein, is used broadly to refer to at least two amino acids or amino acid analogs which are covalently linked by a peptide bond or an analog of a peptide bond. The term peptide includes oligomers and polymers of amino acids or amino acid analogs. The term peptide also includes molecules which are commonly referred to as peptides, which generally contain from about two to about twenty amino acids. The term peptide also includes molecules which are commonly referred to as polypeptides, which generally contain from about twenty to about fifty amino acids. The term peptide also includes molecules which are commonly referred to as proteins, which generally contain from about fifty to about 3000 amino acids. The amino acids of the peptide antagonists may be L-amino acids or D-amino acids.

The peptide agent may be a derivative peptide. The terms “derivative” or “modified” when used in reference to a peptide mean that the peptide contains at least one derivative amino acid. A “derivative” of an amino acid and a “modified” amino acid is a chemically modified amino acid. Derivative amino acids can be “biological” or “non-biological” amino acids. Chemical derivatives of one or more amino acid members may be achieved by reaction with a functional side group. Illustrative derivatized molecules include for example those molecules in which free amino groups have been derivatized to form amine hydrochlorides, p-toluene sulfonyl groups, carboxybenzoxy groups, t-butyloxycarbonyl groups, chloroacetyl groups or formyl groups. Free carboxyl groups may be derivatized to form salts, methyl and ethyl esters or other types of esters and hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides which contain naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine, and ornithine for lysine. Other included modifications are amino terminal acylation (e.g., acetylation or thioglycolic acid amidation), terminal carboxylamidation (e.g., with ammonia or methylamine), and similar terminal modifications. Terminal modifications are useful, as is well known, to reduce susceptibility by proteinase digestion and therefore to prolong the half-life of the peptides in solutions, particularly in biological fluids where proteases may be present. Exemplary modified amino acids include, without limitation, 2-Aminoadipic acid, 3-Aminoadipic acid, beta-Alanine, beta-Aminopropionic acid, 2-Aminobutyric acid, 4-Aminobutyric acid, piperidinic acid, 6-Aminocaproic acid, 2-Aminoheptanoic acid, 2-Aminoisobutyric acid, 3-Aminoisobutyric acid, 2-Aminopimelic acid, 2,4-Diaminobutyric acid, Desmosine, 2,2′-Diaminopimelic acid, 2,3-Diaminopropionic acid, N-Ethylgilycine, N-Ethylasparagine, Hydroxylysine, allo-Hydroxylysine, 3-Hydroxyproline, 4-Hydroxyproline, Isodesmosine, allo-Isoleucine, N-Methylglycine, sarcosine, N-Methylisoleucine, N-Methylavaline, Norvaline, Norleucine, and Ornithine. Derivatives also include peptides containing one or more additions or deletions as long as the requisite activity is maintained.

The amino acids of the peptides are contemplated to include biological amino acids as well as non-biological amino acids. Accordingly, as used herein, the term “biological amino acid” refers to any one of the known 20 coded amino acids that a cell is capable of introducing into a polypeptide translated from an mRNA. The term “non-biological amino acid,” as used herein, refers to an amino acid that is not a biological amino acid. Non-biological amino acids are useful, for example, because of their stereochemistry or their chemical properties. The non-biological amino acid norleucine, for example, has a side chain similar in shape to that of methionine. However, because it lacks a side chain sulfur atom, norleucine is less susceptible to oxidation than methionine. Other examples of non-biological amino acids include aminobutyric acids, norvaline and allo-isoleucine, that contain hydrophobic side chains with different steric properties as compared to biological amino acids.

Peptides which are useful in the instant invention may be synthesized by several methods, including chemical synthesis and recombinant DNA techniques. Synthetic chemistry techniques, such as solid phase Merrifield synthesis are preferred for reasons of purity, freedom from undesired side products, ease of production, etc. A summary of the techniques available are found in several articles, including Steward et al., Solid Phase Peptide Synthesis, W. H. Freeman, Co., San Francisco (1969); Bodanszky, et al., Peptide Synthesis, John Wiley and Sons, Second Edition (1976); J. Meienhofer, Hormonal Proteins and Peptides, 2:46, Academic Press (1983); Merrifield, Adv. Enzymol. 32:221-96 (1969); Fields, et al., Intl. Peptide Protein Res., 35:161-214 (1990), and U.S. Pat. No. 4,244,946 for solid phase peptide synthesis; and Schroder et al., The Peptides, Vol 1, Academic Press (New York) (1965) for classical solution synthesis. Protecting groups usable in synthesis are described as well in Protective Groups in Organic Chemistry, Plenum Press, New York (1973). Solid phase synthesis methods consist of the sequential addition of one or more amino acid residues or suitably protected amino acid residues to a growing peptide chain. Either the amino or carboxyl group of the first amino acid residue is protected by a suitable selectively removable protecting group. A different, selectively removable protecting group is utilized for amino acids containing a reactive side group such as lysine.

The resultant linear peptides may then be reacted to form their corresponding cyclic peptides. A method for cyclizing peptides is described in Zimmer, et al., Peptides, 393-394 (1992), ESCOM Science Publishers, B.V., 1993. To cyclize peptides containing two or more cysteines through the formation of disulfide bonds, the methods described by Tam et al, J. Am. Chem. Soc., 113:6657-6662 (1991); Plaue, Int. J. Peptide Protein Res., 35:510-517 (1990); Atherton, J. Chem. Soc. Trans. 1:2065 (1985); B. Kamber, et al., Helv. Chim. Acta 63:899 (1980) are useful in some embodiments. Polypeptide cyclization is a useful modification to generate modified peptides (e.g., peptidomimetics) because of the stable structures formed by cyclization and in view of the biological activities observed for cyclic peptides.

Alternatively, selected peptides which are useful in the present invention are produced by expression of recombinant DNA constructs prepared in accordance with well-known methods once the peptides are known. Such production can be desirable to provide large quantities or alternative embodiments of such compounds. Production by recombinant means may be more desirable than standard solid phase peptide synthesis for peptides of at least 8 amino acid residues. The DNA encoding the desired peptide sequence is preferably prepared using commercially available nucleic acid synthesis methods. Following these nucleic acid synthesis methods, DNA is isolated in a purified form which encodes the peptides. Methods to construct expression systems for production of peptides in recombinant hosts are also generally known in the art. Preferred recombinant expression systems, when transformed into compatible hosts, are capable of expressing the DNA encoding the peptides. Other preferred methods used to produce peptides comprise culturing the recombinant host under conditions that are effective to bring about expression of the encoding DNA to produce the peptide of the invention and ultimately to recover the peptide from the culture.

Expression can be effected in either procaryotic or eukaryotic hosts. Prokaryotes most frequently are represented by various strains of E. coli. However, other microbial strains may also be used, such as bacilli, for example Bacillus subtilis, various species of Pseudomonas, or other bacterial strains. In such procaryotic systems, plasmid vectors which contain replication sites and control sequences derived from a species compatible with the host are used. For example, a workhorse vector for E. coli is pBR322 and its derivatives. Commonly used procaryotic control sequences, which contain promoters for transcription initiation, optionally with an operator, along with ribosome binding-site sequences, include such commonly used promoters as the beta-lactamase (penicillinase) and lactose (lac) promoter systems, the tryptophan (trp) promoter system, and the lambda-derived P_{L} promoter and N-gene ribosome binding site. However, any available promoter system compatible with procaryote expression can be used.

Expression systems useful in eukaryotic hosts comprise promoters derived from appropriate eukaryotic genes. A class of promoters useful in yeast, for example, includes promoters for synthesis of glycolytic enzymes, e.g., those for 3-phosphoglycerate kinase. Other yeast promoters include those from the enolase gene or the Leu2 gene obtained from YEp13. Suitable mammalian promoters include the early and late promoters from SV40 or other viral promoters such as those derived from polyoma, adenovirus II, bovine papilloma virus or avian sarcoma viruses. Suitable viral and mammalian enhancers may also be used. In the event plant cells are used as an expression system, the nopaline synthesis promoter, for example, is appropriate.

Once the expression systems are constructed using well-known restriction and ligation techniques, transformation of appropriate host cells is done using standard techniques appropriate to such cells. The cells containing the expression systems are cultured under conditions appropriate for production of the peptides, and the peptides are then recovered and purified.

In a preferred embodiment, the peptide agent that specifically binds an integrin of interest (e.g., α5β1) is used in a method of the invention where the peptide binds to integrin with at least about a two-fold greater, more preferably at least about five-fold greater, even more preferably at least about ten-fold greater, and most preferably at least about one hundred-fold greater, specificity for the integrin of interest than for another integrins.

In still further embodiments, modified peptides which inhibit the specific binding of an integrin of interest to one or more of its ligands include a peptidomimetic, i.e., an organic molecules that mimics the structure of a peptide; or a peptoid such as a vinylogous peptoid. Methods for generating libraries of mimetics and for evaluating the library of mimetics for inhibiting the binding of receptors to their ligands are known in the art (See e.g., Souers et al, Bioorg. Med. Chem. Lett., 8:2297-2302 (1998)). Other peptides useful as integrin antagonists that reduce or inhibit angiogenesis can be purchased from commercial sources, and can be identified by screening libraries of peptides, which can be prepared using well known methods of chemical synthesis. (See e.g., Koivunen et al., J. Cell Bio., 124:373-380 (1994)). For example, peptide agonists of integrins of interest may be identified using methods known in the art, such as by panning phage-display peptide libraries as described in U.S. Pat. No. 5,780,426 to Palladino et al., the entire contents of which are herein incorporated by reference. For example, phage-display peptide libraries are panned with the receptor of the integrin of interest attached to a solid support, such as a small diameter (1 μm) polystyrene latex beads. Phage selected by this method can then be tested for specific binding to the integrin of interest via ELISA or other immunologically-based assays. Individual peptide sequences are then determined via sequencing of phage DNA. Further analysis of the minimal peptide sequence required for binding can be assessed via deletion and site-directed mutagenesis, followed by testing of the phage for binding to the integrin of interest via ELISA. Since the identified peptide candidates are fused to the major phage coat protein, soluble peptides are then chemically synthesized and the activity of these free peptides are tested in various in vitro and in vivo assays for the ability to act as antagonists of the receptor of the integrin of interest.

C. Nucleic Acid Sequences

In still other alternative embodiments, the agent employed to inhibit angiogenesis, cell migration, cell adhesion and/or cell survival is a nucleic acid which inhibits the specific binding of an integrin of interest (including, but not limited to, integrins: αvβ1; αvβ3; αvβ5; αvβ6; αvβ8; α1β1; α2β1; α4β1; α5β1; α6β1; α7β1; α8β1; α9β1; α10β1; α6β4; α4β7; αMβ2; αLβ2; αXβ2; and αIIββ2) with its corresponding ligand(s). For example, the present invention contemplates that oligonucleotides, polynucleotides, and fragments or portions thereof, DNA or RNA of genomic or synthetic origin which may be single- or double-stranded, and represent the sense or antisense strand are useful in some embodiments of the present invention. The present invention further contemplates that nucleic acid sequences which are particularly useful in the instant invention include, without limitation, antisense sequences and ribozymes. The nucleic acid sequences are contemplated to bind to genomic DNA sequences or RNA sequences which encode the integrin of interest or one or more of its ligands, thereby inhibiting the binding of the integrin of interest with one or more of its ligands. Antisense and ribozyme sequences may be delivered to cells by transfecting the cell with a vector that expresses the antisense nucleic acid or the ribozyme as an mRNA molecule. Alternatively, delivery may be accomplished by entrapping ribozymes and antisense sequences in liposomes.

1. Antisense Sequences

As used herein, the term “antisense” is used in reference to DNA or RNA sequences that are complementary to a specific DNA or RNA sequence (e.g., mRNA). Included within this definition are antisense RNA (“asRNA”) molecules involved in gene regulation by bacteria. Antisense sequences have been successfully used to inhibit the expression of several genes (See e.g., Markus-Sekura, Anal. Biochem. 172:289-295 (1988); Hambor et al., J. Exp. Med., 168:1237-1245 (1988); and patent EP 140 308), including the gene encoding VCAM1, one of the integrin α4β1 ligands (U.S. Pat. No. 6,252,043, incorporated in its entirety by reference). A “sense strand” of a DNA duplex refers to a strand in a DNA duplex which is transcribed by a cell in its natural state into a “sense mRNA.” Sense mRNA generally is ultimately translated into a polypeptide. Thus, in some embodiments, an antisense DNA sequence is a sequence which has the same sequence as the non-coding strand in a DNA duplex, and which encodes an antisense RNA, i.e., a ribonucleotide sequence whose sequence is complementary to a sense mRNA sequence. The designation (−) (i.e., “negative”) is sometimes used in reference to the antisense strand, with the designation (+) sometimes used in reference to the sense (i.e., “positive”) strand. Antisense RNA may be produced by any method, including synthesis by splicing an antisense DNA sequence to a promoter which permits the synthesis of antisense RNA. The transcribed antisense RNA strand combines with natural mRNA produced by the cell to form duplexes. These duplexes then block either the further transcription of the mRNA or its translation, or promote its degradation.

Any antisense sequence is contemplated to be within the scope of this invention if it is capable of reducing the level of expression of the integrin of interest and/or one or more of its ligands (e.g., VCAM and fibronectin) to a quantity which is less than the quantity of the integrin of interest or the ligand of the integrin of interest in a corresponding control tissue which is (a) not treated with the antisense integrin or integrin ligand sequence, (b) treated with a corresponding sense integrin or integrin ligand sequence, or (c) treated with a nonsense sequence.

Antisense integrin sequences and antisense integrin ligand sequences within the scope of this invention may be designed using approaches known in the art. In a preferred embodiment, the antisense integrin sequences and antisense integrin ligand sequences are designed to be hybridizable to mRNA of the integrin of interest or to the mRNA of the lignad of the integrin of interest. Alternatively, in some other embodiments, antisense integrin or integrin ligand sequences may be designed to reduce transcription by hybridizing to upstream nontranslated sequences, thereby preventing promoter binding to transcription factors.

In a preferred embodiment, the antisense oligonucleotide sequences of the invention range in size from about 8 to about 100 nucleotide residues. In yet a more preferred embodiment, the oligonucleotide sequences range in size from about 8 to about 30 nucleotide residues. In a most preferred embodiment, the antisense sequences have 20 nucleotide residues.

However, the invention is not intended to be limited to the number of nucleotide residues in the oligonucleotide sequence disclosed herein. Any oligonucleotide sequence which is capable of reducing expression of the integrin of interest or of the ligand of the integrin of interest is contemplated to be within the scope of this invention. For example, oligonucleotide sequences may range in size from about 3 nucleotide residues to the entire integrin or integrin ligand cDNA sequence. The art skilled know that the degree of sequence uniqueness decreases with decreasing length, thereby reducing the specificity of the oligonucleotide for mRNA of the integrin of interest, or to the mRNA of the ligand of the integrin of interest.

The antisense oligonucleotide sequences which are useful in the methods of the instant invention may comprise naturally occurring nucleotide residues as well as nucleotide analogs. Nucleotide analogs may include, for example, nucleotide residues which contain altered sugar moieties, altered inter-sugar linkages (e.g., substitution of the phosphodiester bonds of the oligonucleotide with sulfur-containing bonds, phosphorothioate bonds, alkyl phosphorothioate bonds, N-alkyl phosphoramidates, phosphorodithioates, alkyl phosphonates and short chain alkyl or cycloalkyl structures), or altered base units. Oligonucleotide analogs are desirable, for example, to increase the stability of the antisense oligonucleotide compositions under biologic conditions since natural phosphodiester bonds are not resistant to nuclease hydrolysis. Oligonucleotide analogs may also be desirable to improve incorporation efficiency of the oligonucleotides into liposomes, to enhance the ability of the compositions to penetrate into the cells where the nucleic acid sequence whose activity is to be modulated is located, in order to reduce the amount of antisense oligonucleotide needed for a therapeutic effect thereby also reducing the cost and possible side effects of treatment.

Antisense oligonucleotide sequences may be synthesized using any of a number of methods known in the art, as well as using commercially available services (e.g. Genta, Inc.). Synthesis of antisense oligonucleotides may be performed, for example, using a solid support and commercially available DNA synthesizers. Alternatively, antisense oligonucleotides may also be synthesized using standard phosphoramidate chemistry techniques. For example, it is known in the art that for the generation of phosphodiester linkages, the oxidation is mediated via iodine, while for the synthesis of phosphorothioates, the oxidation is mediated with ³H-1,2-benzodithiole-3-one,1,-dioxide in acetonitrile for the step-wise thioation of the phosphite linkages. The thioation step is followed by a capping step, cleavage from the solid support, and purification on HPLC, e.g., on a PRP-1 column and gradient of acetonitrile in triethylammonium acetate, pH 7.0.

In another embodiment, the antisense DNA sequence is a “vascular cell adhesion molecule antisense DNA sequence,” i.e., and antisense DNA sequence which is designed to bind with at least a portion of the genomic sequence or with mRNA of an integrin of interest.

2. Ribozymes

In still other alternative embodiments, the agent which inhibits the specific binding of integrin of interest to its ligand is a ribozyme. Ribozyme sequences have been successfully used to inhibit the expression of several genes including the gene encoding VCAM1, which is one of the integrin α4β1 ligands (U.S. Pat. No. 6,252,043, incorporated by reference in its entirety).

The term “ribozyme” refers to an RNA sequence that hybridizes to a complementary sequence in a substrate RNA and cleaves the substrate RNA in a sequence specific manner at a substrate cleavage site. Typically, a ribozyme contains a “catalytic region” flanked by two “binding regions.” The ribozyme binding regions hybridize to the substrate RNA, while the catalytic region cleaves the substrate RNA at a “substrate cleavage site” to yield a “cleaved RNA product.” The nucleotide sequence of the ribozyme binding regions may be completely complementary or partially complementary to the substrate RNA sequence with which the ribozyme binding regions hybridize. Complete complementarity is preferred in order to increase the specificity, as well as the turnover rate (i.e., the rate of release of the ribozyme from the cleaved RNA product), of the ribozyme. Partial complementarity, while less preferred, may be used to design a ribozyme binding region containing more than about 10 nucleotides. While contemplated to be within the scope of the claimed invention, partial complementarity is generally less preferred than complete complementarity since a binding region having partial complementarity to a substrate RNA exhibits reduced specificity and turnover rate of the ribozyme when compared to the specificity and turnover rate of a ribozyme which contains a binding region having complete complementarity to the substrate RNA. A ribozyme may hybridize to a partially or completely complemetary DNA sequence but cannot cleave the hybridized DNA sequence since ribozyme cleavage requires a 2′-OH on the target molecule, which is not available on DNA sequences.

The ability of a ribozyme to cleave at a substrate cleavage site may readily be determined using methods known in the art. These methods include, but are not limited to, the detection (e.g., by Northern blot analysis as described herein, reverse-transcription polymerase chain reaction (RT-PCR), in situ hybridization and the like) of reduced in vitro or in vivo levels of RNA which contains a ribozyme substrate cleavage site for which the ribozyme is specific, compared to the level of RNA in controls (e.g., in the absence of ribozyme, or in the presence of a ribozyme sequence which contains a mutation in one or both unpaired nucleotide sequences which renders the ribozyme incapable of cleaving a substrate RNA).

Ribozymes contemplated to be within the scope of this invention include, but are not restricted to, hammerhead ribozymes (See, e.g., Reddy et al., U.S. Pat. No. 5,246,921; Taira et al., U.S. Pat. No. 5,500,357; Goldberg et al., U.S. Pat. No. 5,225,347 the contents of each of which are herein incorporated by reference), Group I intron ribozyme (See e.g., Kruger et al., Cell, 31:147-157 (1982)), ribonuclease P (See e.g., Guerrier-Takada et al., Cell, 35:849-857 (1983)), hairpin ribozyme (See e.g., Hampel et al., U.S. Pat. No. 5,527,895 incorporated by reference), and hepatitis delta virus ribozyme (See e.g., Wu et al. Science, 243:652-655 (1989)).

A ribozyme may be designed to cleave at a substrate cleavage site in any substrate RNA so long as the substrate RNA contains one or more substrate cleavage sequences, and the sequences flanking the substrate cleavage site are known. Expression in vivo of such ribozymes and the resulting cleavage of RNA transcripts of a gene of interest would in effect reduce or ablate expression of the corresponding gene.

For example, where the ribozyme is a hammerhead ribozyme, the basic principle of a hammerhead ribozyme design involves selection of a region in the substrate RNA which contains a substrate cleavage sequence, creation of two stretches of antisense oligonucleotides (i.e., the binding regions) which hybridize to sequences flanking the substrate cleavage sequence, and placing a sequence which forms a hammerhead catalytic region between the two binding regions.

In order to select a region in the substrate RNA which contains candidate substrate cleavage sites, the sequence of the substrate RNA needs to be determined. The sequence of RNA encoded by a genomic sequence of interest is readily determined using methods known in the art. For example, the sequence of an RNA transcript may be arrived at either manually, or using available computer programs (e.g., GENEWORKS, from IntelliGenetic Inc., or RNADRAW available from the internet at ole@mango.mef.ki.se), by changing the T in the DNA sequence encoding the RNA transcript to a U.

Substrate cleavage sequences in the target RNA may by located by searching the RNA sequence using available computer programs. For example, where the ribozyme is a hammerhead ribozyme, it is known in the art that the catalytic region of the hammerhead ribozyme cleaves only at a substrate cleavage site which contains a NUH, where N is any nucleotide, U is a uridine, and H is a cytosine (C), uridine (U), or adenine (A) but not a guanine (G). The U-H doublet in the NUH cleavage site does not include a U-G doublet since a G would pair with the adjacent C in the ribozyme and prevent ribozyme cleavage. Typically, N is a G and H is a C. Consequently, GUC has been found to be the most efficient substrate cleavage site for hammerhead ribozymes, although ribozyme cleavage at CUC is also efficient.

In a preferred embodiment, the substrate cleavage sequence is located in a loop structure or in an unpaired region of the substrate RNA. Computer programs for the prediction of RNA secondary structure formation are known in the art and include, for example, “RNADRAW” (ole@mango.mefki.se); “RNAFOLD;” Hofacker et al., Monatshefte F. Chemie, 125:167-188 (1994); McCaskill, Biopolymers, 29:1105-1119 (1990); and “DNASIS” (Hitachi), and The Vienna Package (ftp://nrcbsa.bio.nrc.ca/pub and ftp://ftp.itc.univie.ac.at).

In addition to the desirability of selecting substrate cleavage sequences which are located in a loop structure or an unpaired region of the substrate RNA, it is also desirable, though not required, that the substrate cleavage sequence be located downstream (i.e., at the 3′-end) of the translation start codon (AUG or GUG) such that the translated truncated polypeptide is not biologically functional.

It is known in the art that the specificity of ribozyme cleavage for a substrate RNA molecule is determined by the sequence of nucleotides which flank the substrate cleavage site and which hybridize with the ribozyme binding regions. Thus, ribozymes can be designed to cleave at different locations within a substrate RNA molecule by altering the sequence of the binding regions that surround the ribozyme catalytic region of the ribozyme such that the binding regions hybridize with any known sequence on the substrate RNA.

In addition to varying the sequence of the binding regions to effect binding to different locations on the RNA substrate, the number of nucleotides in each of the ribozyme binding regions may also be altered in order to change the specificity of the ribozyme for a given location on the RNA substrate. The number of nucleotides in a binding region is preferably between about 5 and about 25 nucleotides, more preferably between about 11 and about 15 nucleotides, yet more preferably between about 7 nucleotides and about 10 nucleotides.

One of skill in the art appreciates that it is not necessary that the two binding regions which flank the ribozyme catalytic region be of equal length. Binding regions which contain any number of nucleotides are contemplated to be within the scope of this invention so long as the desirable specificity of the ribozyme for the RNA substrate and the desirable cleavage rate of the RNA substrate are achieved. One of skill in the art knows that binding regions of longer nucleotide sequence, while increasing the specificity for a particular substrate RNA sequence, may reduce the ability of the ribozyme to dissociate from the substrate RNA following cleavage to bind with another substrate RNA molecule, thus reducing the rate of cleavage. On the other hand, though binding regions with shorter nucleotide sequences may have a higher rate of dissociation and cleavage, specificity for a substrate cleavage site may be compromised.

It is well within the skill of the art to determine an optimal length for the binding regions of a ribozyme such that a desirable specificity and rate of cleavage are achieved. Both the specificity of a ribozyme for a substrate RNA and the rate of cleavage of a substrate RNA by a ribozyme may be determined by, for example, kinetic studies in combination with Northern blot analysis or nuclease protection assays.

In a preferred embodiment, the complementarity between the ribozyme binding regions and the substrate RNA is complete. However, the invention is not limited to ribozyme sequences in which the binding regions show complete complementarity with the substrate RNA. Complementarity may be partial so long as the desired specificity of the ribozyme for a substrate cleavage site, and the rate of cleavage of the substrate RNA are achieved. Thus, base changes may be made in one or both of the ribozyme binding regions as long as substantial base pairing with the substrate RNA in the regions flanking the substrate cleavage sequence is maintained and base pairing with the substrate cleavage sequence is minimized. The term “substantial base pairing” means that greater than about 65%, more preferably greater than about 75%, and yet more preferably greater than about 90% of the bases of the hybridized sequences are base-paired.

It may be desirable to increase the intracellular stability of ribozymes expressed by an expression vector. This is achieved by designing the expressed ribozyme such that it contains a secondary structure (e.g., stem-loop structures) within the ribozyme molecule. Secondary structures which are suitable for stabilizing ribozymes include, but are not limited to, stem-loop structures formed by intra-strand base pairs. An alternative to the use of a stem-loop structure to protect ribozymes against ribonuclease degradation is by the insertion of a stem loop at each end of the ribozyme sequence. (See e.g., Sioud and Drlica, Proc. Natl. Acad. Sci. U.S.A., 88:7303-7307 (1991)). Other secondary structures which are useful in reducing the susceptibility of a ribozyme to ribonuclease degradation include hairpin, bulge loop, interior loop, multibranched loop, and pseudoknot structure as described in “Molecular and Cellular Biology,” Stephen L. Wolfe (Ed.), Wadsworth Publishing Company (1993) p. 575. Additionally, circularization of the ribozyme molecule protects against ribonuclease degradation since exonuclease degradation is initiated at either the 5′-end or 3′-end of the RNA. Methods of expressing a circularized RNA are known in the art. (See, e.g., Puttaraju et al., Nucl. Acids Res., 21:4253-4258 (1993)).

Once a ribozyme with desirable binding regions, a catalytic region and nuclease stability has been designed, the ribozyme may be produced by any known means including chemical synthesis. Chemically synthesized ribozymes may be introduced into a cell by, for example, microinjection electroporation, lipofection, etc. In a preferred embodiment, ribozymes are produced by expression from an expression vector which contains a gene encoding the designed ribozyme sequence.

D. Other Agents

While the specific embodiments of the present invention are illustrated using antibodies, peptides, and nucleic acid sequences that inhibit the specific binding of integrin of interest to one or more of ligands, the invention expressly contemplates within its scope that other agents (e.g., organic molecules, inorganic molecules, and more specifically, drugs etc.) are useful in the methods of the invention, so long as the selected agent is capable of inhibiting the specific binding of an integrin of interest to one or more of its ligands, thereby reducing PKA catalytic activity. Such agents may be identified by screening libraries of test compounds using, for example, a competitive binding assay or a cell adhesion assay. In a competitive binding assay, for example, an integrin of interest (e.g., α5β1) is coated on plastic microtiter plates and contacted with a labeled known integrin ligand (e.g., α5β1 ligand). The test compounds are tested for their ability to inhibit binding of the labeled ligand to the integrin of interest. Compounds which inhibit such binding are identified as agents which are capable of inhibiting the specific binding of the integrin of interest to its ligand.

Alternatively, in a cell adhesion assay, a labeled known integrin ligand is coated on culture plates, and cells which express the integrin of interest are allowed to adhere to the ligand for 20-30 min in the presence of libraries of test compounds. Compound which inhibit the binding of the integrin expressing cells to the coating of integrin ligand are identified as agents which inhibit the specific binding of integrin of interest to its ligand(s).

V. Pharmaceutical Formulations, Administration Routes, and Dosing Considerations

The present invention provides pharmaceutical compositions comprising at least one PKA activating and/or integrin ligation antagonistic compound administered in any sterile, biocompatible pharmaceutical carrier, including, but not limited to, saline, buffered saline, dextrose, and water.

In some embodiments, the pharmaceutical compositions of the present invention may contain one agent (e.g., a PKA activating agent). In other embodiments, the pharmaceutical compositions may contain a mixture of at least two agents (e.g., a PKA activating agent(s) and integrin ligation inhibiting agent(s)) of similar or dissimilar type co-administered to a subject. In still further embodiments, the pharmaceutical compositions of the present invention contain at least two agents (e.g., a PKA activating agent(s) and integrin ligation inhibiting agent(s)) that are administered to a patient under one or more of the following conditions: at different periodicities, different durations, different concentrations, different administration routes, etc.

The compositions and methods of the present invention find use in treating diseases or altering physiological states characterized by undesirable cell migration, angiogenesis, or apoptotic control.

The invention contemplates administering therapeutic compounds and, in some embodiments in accordance with acceptable pharmaceutical delivery methods and preparation techniques. For example, some therapeutic compounds of the present invention can be administered to a subject intravenously in a pharmaceutically acceptable carrier such as physiological saline. Standard methods for intracellular delivery of pharmaceutical agents can be used (e.g., delivery via liposome). Such methods are well known to those of ordinary skill in the art.

In some other embodiments, the formulations of the present invention are useful for parenteral administration, such as intravenous, subcutaneous, intramuscular, and intraperitoneal. Therapeutic co-administration of some contemplated anticancer agents (e.g., therapeutic polypeptides) can also be accomplished using gene therapy as described above.

As is well known in the medical arts, dosages for any one patient depends upon many factors, including the patient's size, body surface area, age, the particular compound to be administered, sex, time and route of administration, general health, and interaction with other drugs being concurrently administered. Routine methods are available for optimization of dosages.

In some embodiments of the present invention, therapeutic compounds are administered to a patient alone, or in combination with one or more other drugs or therapies (e.g., conventional anticancer agents, including, but not limited to, nucleotide sequences, drugs, hormones, etc.) or in pharmaceutical compositions where it is mixed with excipient(s) or other pharmaceutically acceptable carriers. In one embodiment of the present invention, the pharmaceutically acceptable carrier is pharmaceutically inert.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

For injection, the pharmaceutical compositions of the invention may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In other embodiments, the pharmaceutical compositions of the present invention can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated. In some preferred embodiments, the therapeutic compounds are administered orally to a patient orally.

Pharmaceutical compositions suitable for use in the present invention include compositions wherein the active ingredients are contained in an effective amount to achieve the intended purpose. For example, an effective amount of therapeutic compound(s) may be that amount that inhibits angiogenesis, cell migration, cell adhesion, and/or cell survival in a cell as compared to control cells. Determination of effective amounts is well within the capability of those skilled in the art, especially in light of the disclosure provided herein.

In addition to the active ingredients, preferred pharmaceutical compositions may contain suitable pharmaceutically acceptable carriers comprising excipients and auxiliaries that facilitate processing of the active compounds into preparations which can be used pharmaceutically. The preparations formulated for oral administration may be in the form of tablets, dragees, capsules, or solutions.

The pharmaceutical compositions of the present invention may be manufactured in a manner that is itself known (e.g., by means of conventional mixing, dissolving, granulating, dragee-making, levigating, emulsifying, encapsulating, entrapping or lyophilizing processes).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Pharmaceutical preparations for oral use can be obtained by combining the active compounds (e.g., PKA activator compounds and/or integrin ligation antagonists) with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc.; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

Ingestible formulations of the present compositions may further include any material approved by the United States Department of Agriculture for inclusion in foodstuffs and substances that are generally recognized as safe (GRAS), such as, food additives, flavorings, colorings, vitamins, minerals, and phytonutrients. The term phytonutrients as used herein, refers to organic compounds isolated from plants that have a biological effect, and includes, but is not limited to, compounds of the following classes: isoflavonoids, oligomeric proanthcyanidins, indol-3-carbinol, sulforaphone, fibrous ligands, plant phytosterols, ferulic acid, anthocyanocides, triterpenes, omega 3/6 fatty acids, polyacetylene, quinones, terpenes, cathechins, gallates, and quercitin.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, (i.e., dosage).

Pharmaceutical preparations that can be used orally include push-fit capsules made of gelatin, as well as soft, sealed capsules made of gelatin and a coating such as glycerol or sorbitol. The push-fit capsules can contain the active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Compositions comprising a compound of the invention formulated in a pharmaceutical acceptable carrier may be prepared, placed in an appropriate container, and labeled for treatment of an indicated condition. For therapeutic compounds, conditions indicated on the label may include treatment of conditions related to faulty regulation of apoptosis, hyperproliferative diseases, cancers, angiogenesis, fibrosis, psoriases, leukemia, lymphomas, arthritis, graft rejection, degenerative conditions, and vascular diseases and the like. The pharmaceutical compositions may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

For any compound used in the method of the invention, the therapeutically effective dose can be estimated initially from cell culture assays. Then, preferably, dosage can be formulated in animal models (particularly murine models) to achieve a desirable circulating concentration range that results in increased PKA activity in cells/tissues characterized by undesirable cell migration, angiogenesis, cell migration, cell adhesion, and/or cell survival. A therapeutically effective dose refers to that amount of therapeutic compound(s) that ameliorate symptoms of the disease state (e.g., unregulated angiogenesis, cell migration, and/or apoptosis). Toxicity and therapeutic efficacy of such compounds can be determined by standard pharmaceutical procedures in cell cultures or experimental animals, e.g. for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from cell culture assays and additional animal studies can be used in formulating a range of dosage, for example, mammalian use (e.g., humans, Equus caballus, Felis catus, and Canis familiaris, etc.). The dosage of such compounds lies preferably, however the present invention is not limited to this range, within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation. Other pharmaceutical compositions may be administered daily or several times a day.

Normal dosage amounts may vary from 0.1 to 100,000 micrograms, up to a total dose of about 1 g, depending upon the route of administration. Guidance as to particular dosages and methods of delivery is provided in the literature (See, U.S. Pat. Nos. 4,657,760; 5,206,344; or 5,225,212, all of which are herein incorporated by reference). Administration of some agents to a patient's bone marrow may necessitate delivery in a manner different from intravenous injections.

Preferred embodiments of the present invention provide pharmaceutical compositions and methods for administering an effective amount of therapeutic compound/agent to a patient to inhibit angiogenesis, cell migration, cell adhesion, and/or cell survival In preferred embodiments, the subject has a disease characterized by the undesirable angiogenesis, cell migration, cell adhesion, and/or cell survival. For example, in some preferred embodiments, the subject has a cancer including, but not limited to, breast cancer, prostate cancer, lymphoma, skin cancer, pancreatic cancer, colon cancer, melanoma, ovarian cancer, brain cancer, liver cancer, bladder cancer, non-small lung cancer, cervical carcinoma, myeloma, adrenal carcinoma, leukemia, neuroblastoma, and glioblastoma. However, the present invention is not intended to be limited to treating any particular type of cancer.

In some embodiments, diseases suspected of being characterized by having undesirable angiogenesis, cell migration, cell adhesion, and/or cell survival suitable for treatment by the present invention are selected by obtaining a sample of interest (e.g. cells, tissues, fluids, etc.) and by measuring one or more markers (e.g., protein levels) of these undesirable states using one or more well established immunohistochemical techniques (e.g., ELISA and Western blots, etc.), and comparing the sample's indicators against the markers measured in relevant reference nonpathological cells and/or tissues. In still other embodiments, diseases suspected of being ameliorated by the therapeutic compounds and methods of the present invention are selected by comparing levels of one or more markers (e.g., polynucleotides, polypeptides, lipids, etc.) in a sample (e.g. cells, tissues, fluids, etc.) that directly or indirectly indicate undesirable cellular angiogenesis, cell migration, cell adhesion, and/or cell survival in the sample as compared to levels of these markers in relevant nonpathological samples.

In some preferred embodiments, therapeutic compounds of the present invention are administered to a patient at a dosage range of about 1 to 200 mg/day, from about 5 to 50 mg/day, and most preferably from about 10 to 40 mg/day. In particularly preferred embodiments, therapeutic compounds are administered to a patient (e.g., orally) in a tolerable daily dose (e.g., 30 to 40 mg/day) shown to have some biologic activity.

In preferred embodiments, following treatment, the diseased cells and tissues are subjected to assays for angiogenesis, cell migration, cell adhesion, and/or cell viability, such as, morphological changes, DNA integrity, mitochondria pathways, alterations of expression of Bcl-2 family proteins, and caspase activation as well as upstream and downstream effectors of caspases and caspase inhibitors. Those skilled in the art will be able to readily design and execute assays to test these and an number of other cellular and biochemical parameters in the treated cells and tissues.

### EXAMPLES

The following examples are provided to demonstrate and further illustrate certain preferred embodiments of the present invention and are not to be construed as limiting the scope of the present invention.

### Example 1

### Reagents

Anti-α5β1 and anti-α2β1 antibodies were from Chemicon. Anti-αvβ3 and anti-αvβ5 antibodies were from David Cheresh, Scripps Research Institute. Anti-V5 and GFP antibodies were from Invitrogen. (Invitrogen, Corp., Carlsbad, Calif.). Anti-Rho, Rac, HA and Myc antibodies were from Santa Cruz. (Santa Cruz Biotechonology, Inc., Santa Cruz, Calif.). Tissue culture plastic including transwells were from Costar. (Corning Costar, Corp., Midland, Mich.). Fibronectin and collagen I were from Collaborative Biomedical Products (Bedford, Mass.). Vitronectin was purified from outdated human plasma by denaturing heparin sepharose chromatography. Poly-L-lysine was from Sigma (St. Louis, Mo.). HUVECs, EBM (minimal medium), EGM-2 (bFGF and VEGF, but no FBS) and EGM (2% FBS, bFGF and VEGF) were from Clonetics (San Diego, Calif.). cDNAs were subcloned into topoTA-pcDNA 3.1 V5/His according to manufacturer's directions (Invitrogen, Carlsbad, Calif.). HUVECs were cultured in EGM. All statistical analyses were performed using Student's t-test.

Anti-caspase antibodies were from New England Biolabs (Beverly, Mass.). Anti-PARP and FITC-Annexin V were from Pharmingen (San Diego, Calif.). HA1004 was obtained from Biomol. Caspase inhibitors and activity assays were from Calbiochem (La Jolla, Calif.). Ten-day old chicken embryos were from McIntyre Poultry (Ramona, Calif.). bFGF was from Genzyme, Inc. (Cambridge, Mass.).

N1-GFP (green fluorescent protein) reporter and GFP expressing adenovirus vectors were from Dr. David Cheresh. Murine PKA catalytic subunit and dnPKA (RImut) cDNAs (from Dr. Susan Taylor and Dr. Stanley McKnight) were subcloned into topoTA-pcDNA 3.1 V5/His by PCR-based TA cloning according to manufacturer's directions (Invitrogen, Carlsbad, Calif.). Myc tagged V12 Rac was from Dr. Martin Schwartz. The sequence and orientation of constructs were verified by DNA sequencing. Transfections were performed by electroporating 5×10⁶ cells in 300 μl of EBM with a total of 30 μg of DNA (20 μg expression vectors, 2 μg of N1-GFP and 8 μg of pBluescript as carrier DNA) at 300 V, 450 μF. Expression levels of the transfected PKA subunits were measured by Western blotting of cell lysates with anti-V5 antibodies (and with anti-GFP antibodies). Cells were cultured for 48 hours prior to use in experiments.

### Example 2

### Expression Constructs and Transfections

N1-GFP (green fluorescent protein) reporter vector was a gift from Dr. David Cheresh, The Scripps Research Institute. dnPKA (RImut) cDNA was a gift from Dr. Stanley McKnight, University of Washington. Murine PKA catalytic subunit (PKAcat) and mutationally inactive PKA (dnPKA; 41) cDNAs (gifts from Dr. Susan Taylor, University of California, San Diego and Dr. Renate Pilz, University of California, San Diego, respectively) were subcloned into topoTA-pcDNA 3.1 V5/His by PCR-based TA cloning according to manufacturer's directions (Invitrogen, Carlsbad, Calif.). Myc tagged V12 Rac was a gift from Dr. Martin Schwartz, The Scripps Research Institute. HA-tagged V14Rho, N19 Rho, V12Rac and N17Rac as well as Myc-tagged dnPAK and dpPAK were gifts from Dr. Martin Schwartz, The Scripps Research Institute. The sequence and orientation of all constructs were verified by DNA sequencing. Transfections were performed by electroporating 5×10⁶ cells in 300 μl of EBM with a total of 30 μg of DNA (20 μg expression vectors, 2 μg of N1-GFP and 8 μg of pBluescript as carrier DNA) at 300 V, 450 μF. Expression levels of the transfected genes were measured by Western blotting cell lysates with anti-V5, anti-HA, anti-Myc antibodies and/or anti-GFP antibodies. Seventy to eighty percent of cells routinely expressed the transgenes, as determined by counting GFP expressing cell. Cells were cultured for 48 hrs prior to use in experiments.

### Example 3

### Chorioallantoic Membrane Angiogenesis Assays

The CAMs of 10 day-old chicken embryos (McIntyre Poultry, Ramona, Calif.) were stimulated with 30 ng bFGF (Genzyme, Cambridge, Mass.) or saline as described in S Kim et al. (S. Kim et al., Amer. J. Biol. Chem., 275:33920-33928 (2000); and S. Kim et al., Amer. J. Path., 156:1345-1362 (2000)). bFGF stimulated CAMs were transfected by direct application of 4 μg N1-GFP, dnPKA or PKA catalytic subunit plasmid DNA or were treated with 250 μM cAMP. Ten embryos were used per group. CAMs were fixed with 3.7% paraformaldehyde prior to excision. Representative CAMs were photographed at 10× magnification. Blood vessel branch points were counted in each CAM at 30× and graphed as average branchpoints above background+/−S.E.M. per treatment group. Statistical analyses were performed using paired Student's t-test. Experiments were performed 3 times and selected representative experiments are presented. Transgene expression was verified by immunoblotting lysates from CAMs and by immunohistochemistry of CAM cryosections.

In regard to embodiments of the present invention directed to administration of PTH and/or PTHrP, chorioallantoic membrane angiogenesis assays were conducted as follows: CAMs of 10 day-old chicken embryos (McIntyre Poultry, Ramona, Calif.) were stimulated with 30 ng bFGF, VEGF, IL-8, TNFa (Genzyme, Cambridge, Mass.) or saline as described in kim et al., infra. Twenty μl of 0.001-10 μM PTHrP (1-173, 1-141, 1-86, 1-34, 15-34, 1-10 or scrambled 1-10 (H L Q A H S V E D L) peptide fragments), calcitonin, calcitonin-gene related peptide (CGRP) or saline were applied to CAMs 24 hrs after stimulation. In some experiments, PTHrP peptides were applied together with 10 μg function-blocking antibody directed against PTHrP (8B12) or 480 nM H89, a selective PKA inhibitor. Embryos stimulated with bFGF or VEGF were also injected intravenously with 10⁷ pfu of adenoviruses expressing GFP or PTHrP 1-173. Adenovirus expressing GFP was obtained from Dr. David Cheresh (Scripps Research Institute). Adenovirus expressing PTHrP 1-173 was prepared as described in R. Terkeltaub et al., infra. Additionally, bFGF stimulated CAMs were transfected by direct application of 4 μg N1-GFP, dnPKA or PKA catalytic subunit plasmid DNA. In some embodiments, 50 mg fragments of DU145 prostate carcinoma cell tumors were placed on CAMs. 107 pfu of adenoviruses expressing GFP or PTHrP were injected intravenously into embryos and tumors were excised after 7 days. Tumor weights were measured. Five hundred μl of 3.7% paraformaldehyde were applied to CAMs prior to excision. Ten embryos were used per group. Representative CAMs were photographed at 10× magnification. Blood vessel branch points were counted in each CAM at 30× and graphed as average branchpoints above background+/−S.E.M. per treatment group. Statistical analyses were performed using paired Student's t-test. Unfixed CAMs were flash frozen, sectioned, and stained with anti-PTHrP (9H7) anti-αvβ3, anti-smooth muscle actin and anti-vWF antibodies. Experiments were performed 5-10 times and representative experiments are presented.

### Example 4

### Murine Angiogenesis and Tumor Assays

In some embodiments murine angiogenesis and tumor assays were conducted as follows: matrigel induced angiogenesis assays were performed as described in Eliceiri et al., incorporating saline, or 1 μg/ml bFGF in growth factor-depleted matrigel. (See, B. P. Eliceiri et al., Mol. Cell, 4:915-924 (1999)). PTHrP peptides were incorporated in the matrigel at 10 μM. PTHrP adenoviruses were also incorporated in the matrigel at 10⁷ pfu per 400 μl matrigel. Ten animals were used per group. Statistical analyses were performed using paired t-test. Nude mice were inoculated with one million PTHrP negative tumor cells. Two weeks later, when tumors averaged 30 mm3 in volume, mice were treated for ten days with daily injections of saline, PTHrP or a scrambled control PTHrP peptide (1 μM final concentration). Tumor volume was measured again at 6 days and at 10 days of treatment. After 10 days of treatment, tumors were excised, snap frozen in OCT and cryosectioned. Tumor sections were immunostained for expression of CD31, a marker of blood vessels. Ten animals were used per group. Statistical analyses were performed using paired t-test.

### Example 5

### Cell Culture, Migration, and Adhesion Assays

Human umbilical vein endothelial cells (HUVEC) were grown in Endothelial Growth Medium (containing 2% fetal bovine serum, bFGF and VEGF) (Clonetics, San Diego, Calif.). To render HUVECs quiescent for Rac and/or Rho assays, monolayers of endothelial cells were incubated for 18 hrs in Endothelial Basal Medium supplemented with 0.1% serum (Clonetics). Cell migration and adhesion assays on ECM substrates were performed as previously described in Kim et al. (S. Kim et al., Amer. J. Biol. Chem., 275:33920-33928 (2000); and S. Kim et al., Amer. J. Path., 156:1345-1362 (2000)).

### Example 6

### PKA Assays

PKA activity was measured in HUVECs essentially as previously described in Kim et al (S. Kim et al., Amer. J. Biol. Chem., 275:33920-33928 (02000)). HUVECs were placed in suspension, allowed to attach to ECM protein coated plates in the absence or presence of anti-integrin antibodies or were treated with 10 μM PTHrP and/or 250 μM cAMP. PKA activity was measured at various times from 0-4 hrs (e.g., 2-30 min).

In one embodiment, Culture plates were coated overnight at 4° C. with 10 μg/ml fibronectin, vitronectin or collagen or with 3% bovine serum albumin for suspension cultures. Plates were washed and blocked in 3% bovine serum albumin for 1 hour at 37° C. HUVECS were trypsinized and resuspended in adhesion buffer (Hanks balanced salt solution supplemented with 10 mM HEPES, pH 7.4, 2 mM MgCl₂, 2 mM CaCl₂, 0.2 mM MnCl₂, 1% BSA. Cells were plated on matrix protein coated plates in culture medium or in the presence of 25 μg/ml anti-α5β1, anti-αvβ3 or anti-α2β1 antibodies for various times from 0 minutes to 4 hours. Plates were washed with phosphate buffered saline and cells were extracted with cold extraction buffer (50 mM Tris, pH 7.5, 5 mM EDTA, 25 μg/ml aprotinin and 25 μg/ml leupeptin). Protein Kinase A activity was assessed using a kit from Gibco BRL. Briefly, equal volumes of lysate and reaction mixture containing final concentrations of 50 mM Tris, pH 7.5, 10 mM MgCl₂, 0.25 mg/ml BSA, 50 μM Kemptide (a protein kinase A substrate), 3000 Ci/mmol (γ³² P) ATP and 100 μM ATP were combined for 5 minutes at 30° C. Total PKA in each sample was measured by performing the assay in the presence of 10 μM cAMP. Background PKA activity in test and total PKA samples were determined by performing the assay in the presence of a PKA inhibitor peptide, PKI (6-22) amide. Twenty μl samples of reaction mixtures were spotted onto phosphocellulose discs and washed repeatedly with 1% (v/v) phosphoric acid in water. Incorporated radioactivity was determined by scintillation counting. Activated PKA was calculated from the ratio of pmol/min test sample PKA to pmol/min total PKA. PKA activity in triplicate samples was measured and experiments were repeated at least three times.

In another embodiment, PKA assays were performed with a kit from Calbiochem (San Diego, Calif.). Cell lysates were incubated in ELISA plates coated with a PKA peptide substrate, then incubated with anti-phosphoserine antibodies. Bound antibody was detected after incubation with anti-rabbit IgG-horse radish peroxidase. Statistical analyses were performed using Student's t-test.

### Example 7

### Rho and Rac Assays

Rho activity was determined by measuring the amount of Rho affinity bound to GST-Rhotekin binding domain (RBD). Levels of active Rho were determined by Western blotting affinity precipitated protein with anti-Rho antibodies while total Rho levels were measured by immunoblotting total cell lysates with anti-Rho. Rac activity was determined by measuring the amount of Rac affinity precipitated by the GST-p21-activated protein binding domain, PBD (Upstate Biotechnology, Syracuse, N.Y.). Levels of active Rac were determined by immunoblotting affinity precipitated protein with anti-Rac antibodies. In some embodiments, endothelial cells were maintained in suspension for 2 hrs prior to plating on vitronectin coated plates for 30 min in the presence/ansence of 10 μM PTHrP or cAMP. Total Rac levels were measured by immunoblotting total cell lysates with anti-Rac.

### Example 8

### Annexin V-FITC Staining

Culture plates were coated with 10 mg/ml of fibronectin, vitronectin, collagen, or poly-L-lysine at 4° C. for 16 hrs and blocked with denatured BSA. Plates were also coated with 25 mg/ml goat anti-mouse IgG (Southern Biotechnology Associates, Inc., Birmingham, Ala.) at 37° C. for 2 h, rinsed, blocked with denatured BSA, and then incubated with 10 mg/ml anti-a5b1 or control IgG (anti-MHC) at 4° C. for 16 hrs. HUVECs were incubated on plates in the presence or absence of 10 mg/ml anti-α5β1, -αvβ3, -α2β1, or -MHC for 0-24 hrs. In some experiments, HUVECs were incubated with antibodies and 100 mM caspase-3, -8 or -9 inhibitors or vehicle control (0.5% DMSO). Cells were then incubated with Annexin V-FITC for 15 min at room temperature in the dark. Washed cells were fixed in 1% paraformaldehyde, then incubated in DAPI. The percentage of Annexin positive cells in 5 microscopic fields was determined at 200× magnification by fluorescence microscopy.

### Example 9

### Apoptosis Assays

In some embodiments, HUVECs were incubated on ECM or antibody coated plates for 0-24 hrs. DNA fragmentation was detected by agarose gel electrophoresis as described in M. Herrmann et al. (See, M. Herrmann et al., Nucleic Acids Res., 22:5506-5507 (1994)). Alternatively, HUVECS on plates were lyzed with RIPA buffer (See, S. Kim et al., J. Biol. Chem., 275:33920-33928 (2000)) on ice. Lysates were electrophoresed on 8% SDS-PAGE gels and immunoblotted with anti-PARP, -caspase-3, -caspase-9, -cleaved caspase 3, or -cleaved caspase-9 antibodies. Immunoreactive bands were identified by chemiluminescence and quantified by densitometry. Caspase 3 and 8 activities were determined in cell lysates according to manufacturer's instructions (Calbiochem, La Jolla, Calif.).

### Example 10

### Immunohistochemical Analysis of Focal Contacts

Round 2.5 mm thick glass coverslips were coated with 5 μg/ml vitronectin for 1 hour at 37° C., then blocked with 3% bovine serum albumin for 2 hours at 37° C. HUVECS were removed from culture dishes by trypsizination, washed in serum-free culture medium, and resuspended in endothelial basal culture medium. Cells were then incubated for one hour at 37° C. on coated coverslips. Cells were incubated in the presence of 25 μg/ml anti-α5β1, anti-α2β1 or no antibodies for 60 minutes. Alternatively, cells in endothelial growth medium containing VEGF and bFGF but no serum were plated on coverslips for 48 hours and then treated with cAMP or medium. Coverslips were then washed to remove unbound cells, fixed for 5 minutes in 3.7% paraformaldehyde and permeabilized in 0.3% TX-100 for 3 minutes. Coverslips were incubated with 0.2 μg/ml anti-vinculin antibodies or rhodamine phalloidin (20 ng/ml) in 20 mg/ml bovine serum albumin in phosphate buffered saline for one hour at room temperature. Coverslips were well washed in PBS and incubated in 10 μg/ml goat anti-mouse-Alexa 568 (for anti-vinculin) for 1 hour at room temperature. Coverslips were mounted using mounting medium prior to fluorescent microsopy.

### Example 11

### Cell Adhesion Assays

Cell adhesion assays were performed as described (Kim, et. al., 2000). In brief, the wells of 48 well non-tissue culture treated culture dishes (Costar, Inc.) were coated with 5 μg/ml vitronectin, fibronectin, Del-1 or collagen for one hour at 37° C. and blocked with 2% heat denatured bovine serum albumin in phosphate buffered saline for one hour. Fifty thousand cells in a final concentration of 25 μg/ml of an anti-α5β1 function blocking antibody (JBS5), 25 μg/ml of an anti-αvβ3 function blocking antibody (LM609), 25 μg/ml of an anti-α2β1 function blocking antibody, or 25 μg/ml of an anti-αvβ5 function blocking antibody (P1F6) in adhesion buffer (Hepes buffered Hanks balanced salt solution, HBSS, containing 1% bovine serum albumin, 2 mM MgCl₂, 2 mM CaCl₂ and 0.2 mM MnCl₂) were allowed to adhere to dishes for twenty minutes at 37° C. In some experiments, dibutyryl cAMP was used at 250 μM. Each experiment was performed in triplicate, with triplicate samples per condition. The data are presented as percent of adhesion exhibited by the positive control (adhesion medium alone)+/−standard error of the mean. Similar assays were performed using transfected cells.

### Example 12

### Migration Assays

Migration assays were performed essentially as described (Kim, et. al., 2000). The lower side of 8 μm pore transwell inserts (Costar, Inc.) were coated with 5 μg/ml of fibronectin, vitronectin, Del-1, collagen or no protein for one hour and were blocked with 2% bovine serum albumin in phosphate buffered saline for one hour. HUVECS (25,000) were added to the upper chamber of inserts in migration buffer (Hepes buffered M199 medium containing 1% BSA, 2 mM MgCl₂, 2 mM CaCl₂ and 0.2 mM MnCl₂) containing 25 μg/ml of various antibodies. In some experiments, 250 μM dibutyryl cAMP was placed in the upper and lower chambers of the transwell. Cells were allowed to migrate from the upper to the lower chamber for four hours at 37° C. The number of cells which had migrated were counted in three representative high power (200×) fields per insert. The data are presented as number of cells migrating+/−standard error of the mean. Migration assays were similarly performed on transfected cells. In this case, migrated transfected cells were identified by fluorescent microscopy and counted.

### Example 13

### Videomicroscopy and Data Analysis

2.5-cm-diameter glass coverslips were coated with 5 μg/ml vitronectin for 1 hour at 37° C., then blocked with 3% bovine serum albumin for 2 hours. HUVECs were removed from culture dishes by trypsizination, and plated on coverslips for 48 hours. Coverslips were then placed on the microscope stage (Zeiss, Oberkochen, Germany) connected to a Charged Coupled Device (CCD) camera and viewed at a 200× magnification in bright field. Cells on the coverslips were kept at 37° C. with 5% CO₂, bathed in culture media with 20 mM HEPES (pH7.4). Images were recorded using IP Lab software (Webster, N.Y.) at 1-minute intervals for 60 minutes. Dibutyryl-cAMP was then directly added into the culture medium at concentration of 500 μM. Images of cells were then recorded for an additional 60 minutes at 1-minute intervals.

Cell shape and migration distance changes were quantified using IMAQ Vision Builder software (National Instruments Corporation, Austin, Tex.). Cell shape and nuclear positions were measured at 10-minute time points. Ellipse Ratio was used as a parameter for polarity. As cell shape approaches a circle, the Ellipse Ratio approaches the number one. Distance migrated was determined by measuring the distance nuclei moved in ten minute intervals.

### Example 14

### Migration Online Supplementary Materials

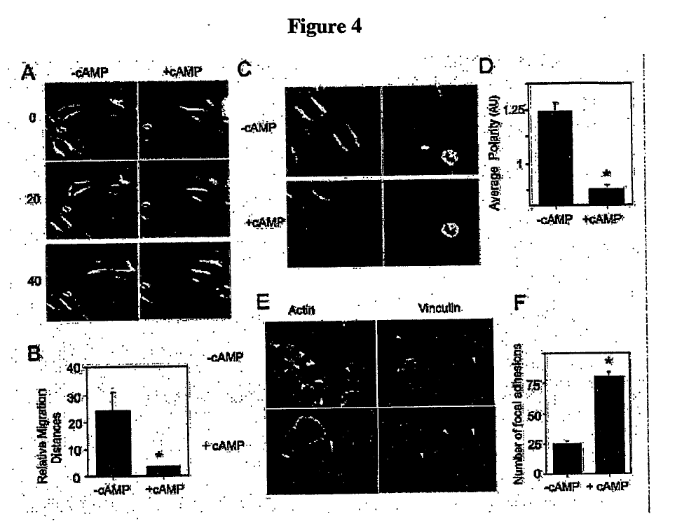

Time lapse videos: 2.5-cm-diameter glass coverslips were coated with 5 μg/ml vitronectin for 1 hour at 37° C., then blocked with 3% bovine serum albumin for 2 hours. HUVECs were removed from culture dishes by trypsizination, and plated on coverslips for 48 hours. Coverslips were then placed on the microscope stage (Zeiss, Oberkochen, Germany) connected to a Charged Coupled Device (CCD) camera at a 200× magnification in bright field. Cells on the coverslips were kept at 37° C. with 5% CO₂, bathed in culture media with 20 mM HEPES (pH7.4). Images were recorded using IP Lab Spectrum software (Webster, N.Y.) at 1-minute intervals for 60 minutes. Movies play at 10 frames/sec. FIG. 4.Mov.1 depicts normal endothelial cells migrating in culture medium. FIG. 4.Mov.2 depicts endothelial cells after addition of 500 μM dibutyryl-cAMP.

### Example 15

### Role of PKA in Cell Survival

HUVECs were plated for 24 h on ECM protein-coated plates in the presence of anti-αvβ3 or anti-α5β1, 1 μM HA1004 (a PKA inhibitor), and anti-αvβ3 or anti-α5β1 with 1 μM HA1004. Cells were stained with FITC-Annexin V or immunoblotted with anti-cleaved caspase 3 antibodies as described above. 5×10⁶ HUVECs were electroporated in 300 μl of EGM with 30 μg of DNA (20 μg dnPKA or PKAcat plasmids, 2 μg N1-GFP and 8 μg pBluescript) at 300 V, 450 μF as previously described (Dormond et al. (2001) Nature Med. 7,1041-7). Expression of transgenes was determined by Western blotting of cell lysates with anti-V5 and anti-GFP antibodies. Transfection efficiency was quantified by determining the percent of cells that were co-transfected with GFP-expression vectors. Eighty-percent transfection efficiency was generally achieved using this electroporation method.

### Example 16

### Chick Chorioallantoic Membrane (CAM) Angiogenesis Assay

CAM assays were performed as described (Kim et al. (2000) Am. J. Path. 156:1345-1362). Saline or bFGF stimulated CAMs were treated for 24 h with 20 μg anti-α5β1, anti-αvβ3 or control antibodies and then injected intravenously with 50 μl Annexin V-FITC. CAMs were harvested after 2 h, then analyzed by confocal microscopy. Alternatively, CAMs treated as above were excised, minced on ice, and treated with 0.1% dispase/0.1% collagenase for 1 h at 37° C. Single cell suspensions were incubated with FITC-Annexin V and analyzed as described above. bFGF stimulated CAMs were also treated with anti-α5β1 or anti-αvβ3 in the presence or absence of 500 μM caspase-3, -8 or -9 inhibitors. bFGF stimulated CAMs were also transfected with 4 μg N1-GFP, dnPKA or PKAcat expression plasmids. Freshly excised CAMs were homogenized in ice cold RIPA buffer containing protease inhibitors and immunoblotted to detect intact and cleaved caspase 3 or 8. For all treatment groups, n=10. CAMs were fixed with 3.7% paraformaldehyde prior to excision. The average number of blood vessel branch points per treatment group+/−SEM is presented. Some excised CAMs were cryopreserved, cut into 5 μm sections, immunostained with anti-cleaved caspase 3, anti-cleaved caspase 8 and anti-VFW antibodies (to detect blood vessels). CAMs were also stained by the TUNEL method to detect fragmented DNA.

### Example 17

### Unligated Integrins Disrupt Cell Migration

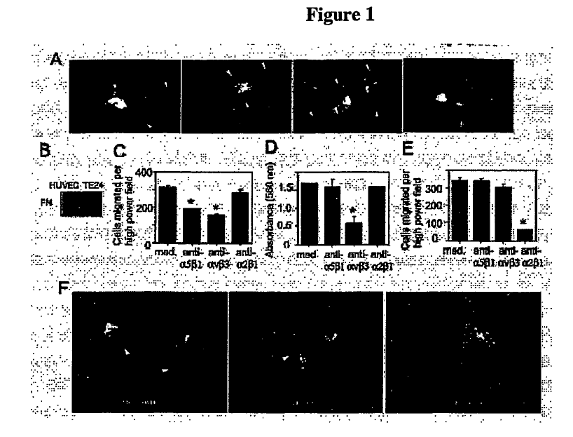

In order to study the role of unligated integrins in the regulation of motility, we examined the effect of unligated integrins on the migration of primary endothelial cells. In culture, endothelial cells plated on vitronectin secrete fibronectin (FIG. 1A-B), creating a mixed matrix that supports the attachment and localization to focal adhesions of integrins α5β1 and αvβ3 (FIG. 1A-B). Using this system, we were able to inhibit the ligation of integrin α5β1 by fibronectin without disrupting the ligation of integrin αvβ3 by vitronectin. Antagonists of α5β1 which block endothelial cell attachment to fibronectin (Kim et al., Amer. J. Path. 156:1345-1362 (2000); Kim et al., J. Biol. Chem., 275:33920-33928 (2000)) inhibit endothelial cell migration on vitronectin without inhibiting cellular attachment to vitronectin (FIG. 1C-D). In contrast, integrin αvβ3 antagonists block migration and adhesion to vitronectin, while antibodies against integrin α2β1 have no effect on cell attachment or migration on vitronectin. Antagonists of α5β1 also blocked cell migration but not adhesion on other provisional matrix protein ligands for integrin αvβ3 such as Del-1 (Penta et al. (1999) J. Biol. Chem. 274, 11101-11109) or proteolyzed collagen. In contrast, integrin α5β1 antagonists do not block endothelial cell migration on non-provisional matrix proteins such as intact collagen (FIG. 1E) or laminin, even though fibronectin is secreted while cells are plated on these matrices as well (Kim et al., Amer. J. Path. 156:1345-1362 (2000); Kim et al., J. Biol. Chem., 275:33920-33928 (2000)). As α5β1 antagonists do not block collagen-mediated migration, these results indicate that α5β1 antagonists specifically inhibit αv but not α2β1 mediated cell migration. Importantly, the ability of α5β1 to regulate cell migration on provisional matrix proteins is not restricted to endothelial cells as similar results were obtained for fibroblasts, breast carcinoma, prostate carcinoma, melanoma and other tumor cells.

As cell migration requires remodeling of the actin cytoskeleton to facilitate cell shape change and forward motility, we examined the effect of selective integrin inhibition on the cytoskeleton in endothelial cells. Endothelial cells attached to vitronectin in culture medium or in the presence of anti-integrin α2β1 antibody antagonists rapidly spread, change shape, and form lamellipodia and thick actin filaments that traverse the cell (FIG. 1F). In contrast, cells treated with α5β1 antagonists exhibit a pancake-like morphology (FIG. 1F). These cells lack the filamentous actin network normally associated with the migratory phenotype and exhibit thin, disorganized actin bundles surrounding the center of the cell. These results suggest that ligation of integrin α5β1 by cell-secreted fibronectin indirectly regulates the ability of αv integrins, such as αvβ3, to promote the actin assembly events that are required for cell migration.

### Example 18

### Unligated Integrins Activate PKA

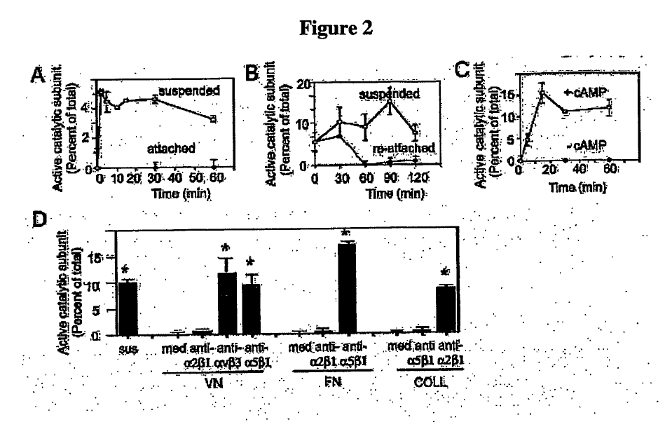

As α5β1 antagonists indirectly block αv integrin mediated cell migration, we examined the role of signal transduction in this process. Our previous studies indicated that ligation of α5β1 suppresses activation of the enzyme protein kinase A (Kim, et. al., 2000). Here we show that detachment of cells from the ECM rapidly activates PKA (FIG. 2A). Peak PKA activity was observed as little as 2 minutes after cell detachment; this activity remained elevated for at least six hours in suspension. In contrast, cell re-attachment to vitronectin (and other matrix proteins) rapidly suppresses PKA activity, with little PKA activity detectable after one hour of attachment (FIG. 2B). Exposure of endothelial cells to cell permeable cAMP similarly activates PKA in endothelial cells attached to ECM proteins (FIG. 1C). Notably, inhibition of cell attachment to the extracellular matrix with specific anti-integrin antibodies also activates PKA (FIG. 2D). For example, inhibition of cell attachment to vitronectin by integrin αvβ3 antagonists, to fibronectin by integrin α5β1 antagonists, or to collagen by integrin α2β1 antagonists activates PKA (FIG. 2D). These results indicate that cell attachment is required to suppress PKA activity. Notably, inhibition of α5β1 in cells attached to vitronectin also activates PKA (FIG. 2D). As endothelial cells plated on vitronectin secrete fibronectin which ligates α5β1 (FIG. 1A-B), these results suggest that ligation of integrin α5β1 by fibronectin suppresses PKA activation in cells attached to vitronectin. Similar results were observed for other cell types including tumor cells. Together, these results indicate that integrin ligation suppresses PKA activation.

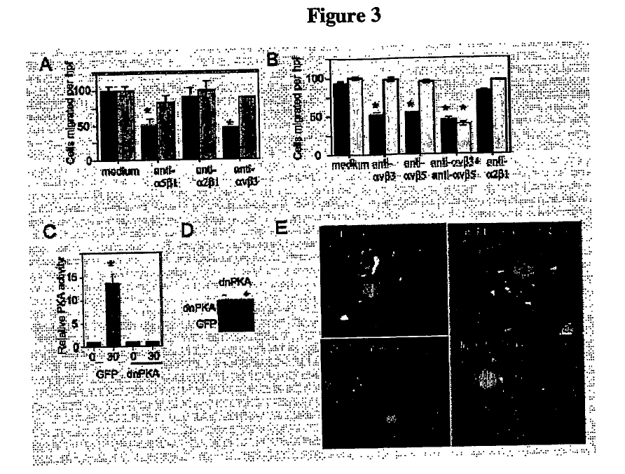

Since integrin α5β1 antagonists activate PKA and inhibit cell migration on vitronectin, we examined the role of PKA in this process. Expression of a dominant negative form of PKA (dnPKA), a mutated PKA regulatory subunit that blocks activation of the catalytic subunit (Clegg et al. (1987) J. Biol. Chem. 262:13111-13119), promotes normal cell migration in the presence of α5β1 antagonists. Thus, inhibition of PKA activity overcomes the anti-migratory effect of α5β1 antagonists in endothelial cells FIG. 3A) and tumor cells. Similar effects were seen for migration on proteolyzed collagen, another provisional matrix protein. Therefore, selective inhibition of α5β1 ligation activates PKA and inhibits cell migration on vitronectin.

In fact, selective inhibition of other integrins can also activate PKA and suppress cell migration. For example, integrin αvβ3 and αvβ5 antagonists can each partially inhibit cell migration on vitronectin. Each of these inhibitions can be reversed by expression of dnPKA (FIG. 3A-B). As integrins αvβ3 and αvβ5 both contribute to cell migration on vitronectin, these results suggest that inhibition of either integrin activates PKA and partially suppresses cell migration. However, expression of dnPKA can not suppress the combined inhibitory effects of αvβ3 and αvβ5 antibodies on cell migration, as endothelial cells cannot attach to the substratum in the presence of inhibitors of both αv integrins. Similarly, inhibition of cell migration on collagen by integrin α2β1 antagonists can not be reversed by expression of dnPKA, as endothelial cells cannot attach to collagen in the presence of α2β1 inhibitors. PKA activity assays verify that dnPKA expression does suppress PKA activation in endothelial cells (FIG. 3C-D). These observations indicate that unligated or antagonized integrins activate PKA and that activated PKA inhibits cell migration. These results also indicate that PKA activation by integrin antagonists suppresses cell migration even in ECM attached cells.

In addition to restoring normal cell migration to endothelial cells treated with α5β1 antagonists, expression of dnPKA restores a normal migratory phenotype to ECs treated with α5β1 antagonists (FIG. 3E). These studies indicate that activation of PKA by integrin antagonists suppresses the organization of the actin cytoskeleton that is associated with the migratory phenotype. Taken together, these studies suggest that integrin antagonists can inhibit cell migration by inhibiting actin remodeling and cell polarization in a PKA-dependent manner.

### Example 19

### PKA Activation Inhibits Cell Migration

Our studies indicate that inhibition of integrin ligation activates PKA and blocks cell migration. Furthermore, inhibition of PKA activity permits cell migration in the presence of integrin antagonists. These results suggest that direct activation of PKA might inhibit cell migration. In fact, time lapse videomicroscopy of migrating endothelial cells treated with cAMP indicates that cAMP rapidly inhibits endothelial cell migration (FIG. 4A-B) and rapidly induces a flattening of the cell shape (FIG. 4C-D)(see also online supplementary movies, FIG. 4. Mov.1 and FIG. 4. Mov.2). Cell nuclei do not significantly move after treatment with cAMP; in contrast, untreated nuclei move significant distances (FIG. 4A-B). Additionally, cAMP treated cells rapidly flatten out and remain flattened for up to one hour after addition of cAMP (FIG. 4C-D). This morphology is similar to that of PKA RI_{α}-/-fibroblasts (Amieux, et. al., 2002). These studies indicate that endothelial cells cease all movement possibly because of a decreased ability to detach from the substrate within minutes after addition of an activator of PKA, dibutyryl cAMP.

To determine whether the cAMP induced flattening results from disruption in stress fiber and focal adhesion formation, we examined the pattern of actin filaments and vinculin containing focal adhesion structures in cells treated with or without cAMP. While normal endothelial cells exhibit long, spindle shapes as well as stress fibers and cortical actin in the leading edge of cells, cAMP treated cells remain round and flat and exhibit neither stress fibers nor cortical actin; instead these cells exhibit a subcortical basket-like network of thin actin filaments or no filaments at all (FIG. 4E). Interestingly, normal ECs form focal adhesions at the leading edges of migrating cells (FIG. 4E). However, cAMP treated cells exhibit a 3 fold increase in the number of focal adhesions (FIG. 4E-F). These focal adhesions also exhibit an altered distribution so that the adhesions are distributed throughout the entire cell rather than at the leading and trailing edges as in normal cells (FIG. 4E-F).

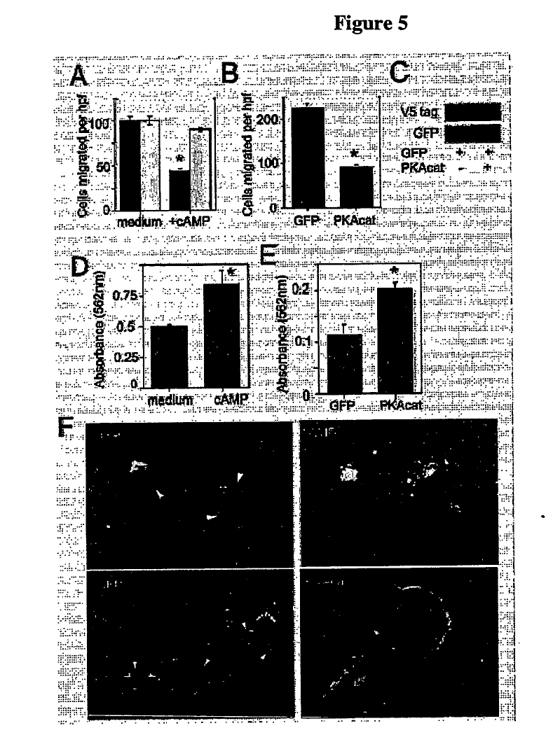

Treatment of endothelial cells with cAMP also quantitatively inhibits endothelial cell migration in manner that can be inhibited by expression of dnPKA in a transwell model of migration (FIG. 5A). In addition, expression of the active catalytic subunit of PKA in endothelial cells (FIG. 5B-C) significantly blocked cell migration on vitronectin (FIG. 5B), fibronectin and collagen. Taken together with the results of FIG. 4, these results indicate that direct activation of PKA negatively regulates cell migration. As cAMP treatment induces cell flattening and an increase in focal adhesion number, it is possible that PKA inhibits migration in part by enhancing the strength of cell attachment to the substratum. Notably, cAMP, as well as expression of the PKA catalytic subunit, enhanced cell attachment to vitronectin (FIG. 5D-E). Activation of PKA by cAMP or by expression of the PKA catalytic subunit in endothelial cells also inhibited actin remodeling and induced a flattened morphology (FIG. 5F). These studies indicate that activation of PKA suppresses remodeling of the actin cytoskeleton by enhancing focal adhesion formation and increasing cell attachment. Taken together, these studies indicate that PKA activation inhibits cell migration by regulating molecules that control remodeling of the cytoskeleton.

### Example 20

### Integrin Antagonists Stimulate Rho

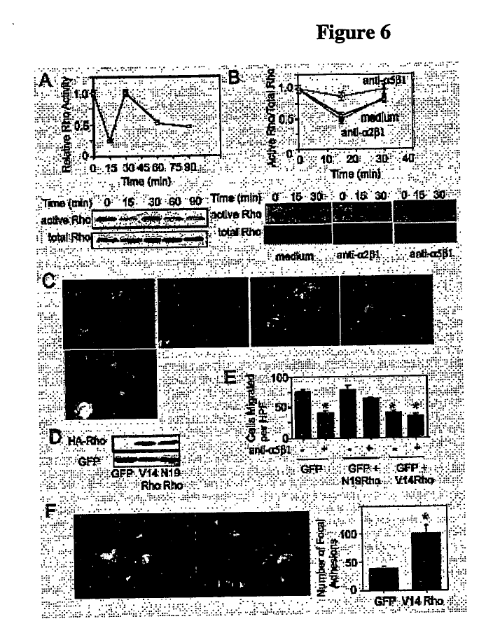

Rearrangements in the actin cytoskeleton are regulated by the Rho family of small GTPases (Nobes CD and Hall (1999) J. Cell Biol. 144:1235-1244; Ridley et al. (1992) Cell 70:389-99; Nobes CD and Hall (1995) Cell 81:53-62). As PKA inhibits cytoskeletal rearrangements associated with cell migration, we examined the regulation of Rho family proteins by integrin antagonists and by PKA activation. It has been previously shown that detachment of fibroblasts from the ECM stimulates Rho activity (GTP-loading) while adhesion to the ECM initially suppresses this activity (Ren et al. (1999) EMBO J. 18, 578-585). This suppression in Rho activity is required for the remodeling of the actin cytoskeleton and for motility (Arthur et al. (2001) Mol. Biol. Cell. 12, 2711-2720; Ren et al. (2000) J Cell Sci. 113, 3673-3855). We found that endothelial cell attachment to vitronectin similarly suppresses Rho activity, with maximal suppression occurring after 15 minutes of cell attachment (FIG. 6A). Interestingly, inhibitors of α5β1 prevent this suppression of Rho activity (FIG. 6B). In fact, Rho activity in cells treated with α5β1 inhibitors is similar to that of unattached endothelial cells.

This stimulation of Rho activity plays a critical role in the inhibition of cell shape change and migration on vitronectin mediated by α5β1 antagonists. Expression of mutationally inactive Rho (N19Rho) restores normal shape and actin filament formation to cells treated with integrin α5β1 antagonists (FIG. 6C). Additionally, expression of mutationally inactive Rho (N19Rho) in endothelial cells prevented α5β1 antagonist-induced inhibition of cell migration (FIG. 6D-E). Similar results were observed in other cell types, including several tumor cell types. In contrast, expression of constitutively active Rho (V14Rho) suppresses migration (FIG. 6C), flattens cell shape (FIG. 6C), and increases the number and distribution of focal adhesions formed in cells (FIG. 6F). As these results are similar to those induced by PKA activation, they suggest that inhibition of integrin ligation activates PKA which then activates Rho and suppresses cell migration. Importantly, recent studies similarly show that high levels of active Rho suppress actin remodeling and cell polarization in undifferentiated embryonic mesenchymal cells (Beqaj et al. (2002) J. Cell Biol. 156, 893-903) and cause neurite retraction in vitro and in vivo (Brouns et al. (2001) Nat. Cell Biol. 3, 361-367; Billuart et al. (2001) Cell 107, 195-207). These indicate that inappropriate activation of Rho inhibits cell shape change and cell migration.

### Example 21

### Activation of PKA Stimulates Rho GTP Loading, Thereby Inhibiting Migration

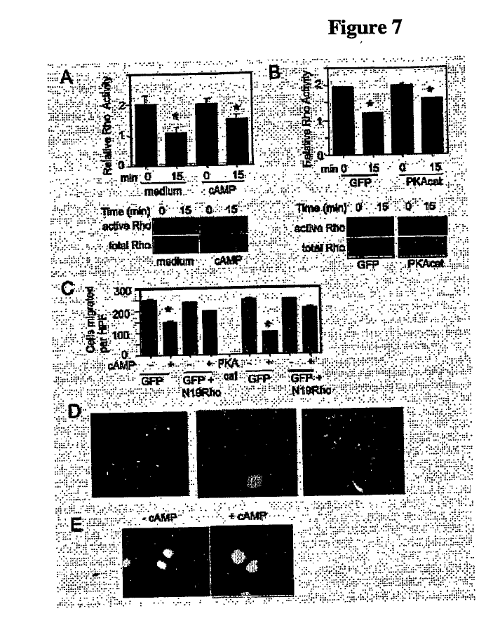

As integrin antagonists activate PKA as well as Rho, we determined whether direct activation of PKA activates Rho. Therefore, we performed Rho activity assays on cell treated with cAMP and on cells expressing the catalytic subunit of PKA. Activation of PKA by cAMP (FIG. 7A) or by expression of the PKA catalytic subunit (FIG. 7B) stimulates Rho activity (GTP-loading) in cells attached to vitronectin. In fact, expression of mutationally inactive Rho (N19Rho) restores cell migration (FIG. 7C) as well as stress fiber formation and the polarized cell shape change associated with the migratory phenotype (FIG. 7D) in the presence of cAMP treatment and PKA catalytic subunit expression. Similar observations were made for other cell types. These results indicate that PKA suppresses cell migration by activating Rho. In fact, activation of PKA by cAMP stimulates the translocation of Rho A to the plasma membrane (FIG. 7E). As Rho prenylation and translocation to the membrane is required for its function (Newman et al. (1993) Biochim. Biophys. Acta 1155, 79-96), these studies indicate that PKA activates Rho, resulting in increased translocation to the membrane and increased cell adhesion due to increased focal adhesion formation.

### Example 22

### Integrin Antagonists Inhibit Rac

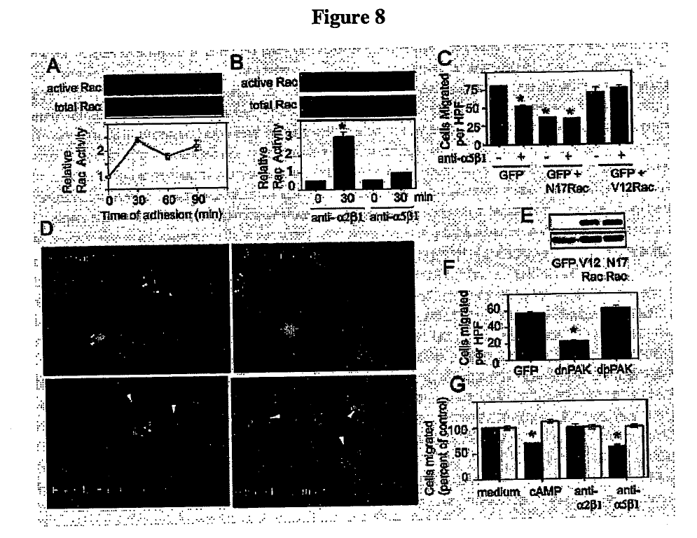

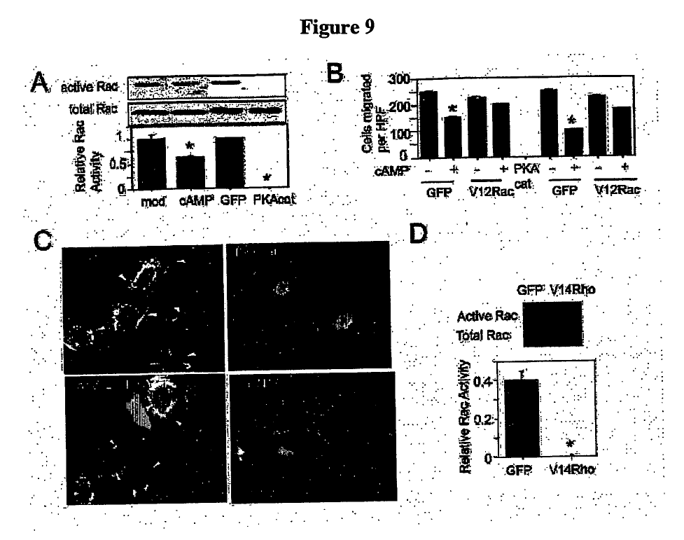

As cell motility is also regulated by the small GTPase Rac (Kiosses et al. (2001) Nat. Cell Biol. 3, 316-320; Kjoller et al. (2001) J. Cell Biol. 152,1145-1157), we examined the effect of integrin α5β1 antagonists on Rac activity. Rac is rapidly activated upon cell attachment to vitronectin (FIG. 8A). In contrast, in the presence of α5β1 antagonists, Rac activation is inhibited (FIG. 8B). Accordingly, the inhibition of motility by α5β1 antagonists in endothelial cells was reversed by expression of constitutively active Rac (V12Rac, FIG. 8C). In contrast, mutationally inactive Rac (N17Rac) blocks migration of endothelial cells (FIG. 8C). Similar observations were made for other cell types. In fact, expression of mutationally active Rac restores normal actin filaments and shape to cells treated with integrin α5β1 antagonists, while inactive Rac inhibits the normal morphology of endothelial cells (see FIG. 9C). These findings indicate that integrin α5β1 antagonists inhibit Rac, thereby blocking cell migration and cell polarization.

p21 activated kinase (PAK) is a substrate of Rac that regulates endothelial cell migration (Kiosses et al. (1999) J. Cell Biol. 147, 831-843). In fact, mutationally inactive PAK suppresses endothelial cell migration (FIG. 8F), while constitutively active PAK reverses the inhibition of cell migration induced by either α5β1 antagonists or cAMP (FIG. 8G). Taken together, these results indicate that integrin antagonists inactivate Rae and its substrate PAK, thereby preventing cell migration.

### Example 23

### PKA Activation Inhibits Rac Activity

As integrin antagonists activate PKA, we examined the effect of direct PKA activation on Rac activity. Exposure of endothelial cells to cell permeable cAMP or expression of the catalytic subunit of PKA inhibits Rac activation (FIG. 9A). This PKA catalytic subunit and cAMP-induced inhibition of motility can be prevented by expression of constitutively active Rac (V12Rac; FIG. 9B). Accordingly, expression of mutationally active Rac (V12Rac) restores normal morphology to cells expressing the PKA catalytic subunit (FIG. 9C) and to those treated with cAMP. In contrast, expression of dominant negative Rac (N17Rac) inhibits cell shape and actin filament changes FIG. 9C). These results indicate that activate PKA inhibits Rac activation, thereby suppressing cell migration.

As PKA activates Rho and inhibits Rac, we examine the effect of active Rho on Rac activity. In fact, we found that active Rho directly suppressed Rac activity (FIG. 9D). In contrast, active Rac does not inhibit Rho activity in these cells. These results indicate that PKA activates Rho, which then inhibits Rac. Since integrin antagonists and PKA activate Rho at a time when it is normally suppressed and when Rac becomes activated, these results indicate that unligated integrins and PKA suppress cell migration by disrupting the normal cycling of small GTPase activity that is required for cell migration.

Taken together, these results indicate that unligated integrins activate PKA, thereby activating the small GTPase Rho and subsequently inhibiting Rac. Increased Rho activity in the context of decreased Rac activity enhances the formation of focal adhesions and increases the strength of cell attachment to the ECM, thereby preventing cell migration and cell polarization. These studies demonstrate that the ligation state of integrin receptors for matrix proteins can regulate cell migration and that PKA activation plays a significant role in this process. Together with previous studies indicating that activated PKA negatively regulates angiogenesis (Bakre et al. (2002) Nature Med. 8, 995-1003; Kim et al. (2002) J. Clin. Invest, 110, 933-941), these studies show that PKA plays an important role in the negative regulation of cell migration in vitro and in vivo.

### Example 24

### Integrin α5β1 Promotes Endothelial Cell Survival In Vitro and In Vivo

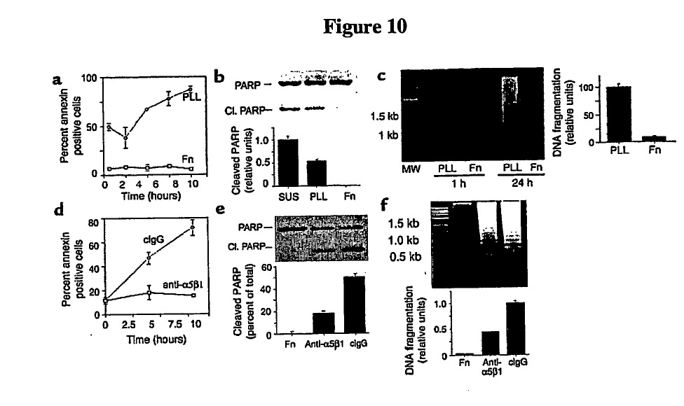

Antagonists of α5β1 and fibronectin inhibit growth factor and tumor-induced angiogenesis (Kim et al. (2000) Am. J. Path. 156:1345-1362; Kim et al. (2000) J. Biol. Chem. 275: 33920-33928). As integrins have been shown to promote cell survival, we examined the roles of α5β1 and fibronectin in endothelial cell survival. Endothelial cells (HUVECs) cultured on poly-L-lysine (which mediates nonspecific cell attachment to the substratum) and fibronectin substrates were analyzed for the binding of Annexin V, a Ca²⁺ dependent phospholipid-binding protein that binds to apoptotic cells with exposed phosphatidyl serine (Raynal et al. (1994) Biochemica et Biophysica Acta. 1197: 63-93). HUVECs attached to fibronectin bind little annexin V, while over 85% of cells on poly-L-lysine coated plates bound annexin V (FIG. 10A, P=0.01). Additionally, lysates from HUVECs cultured in suspension or on poly-L-lysine and fibronectin were immunoblotted for poly (ADP ribose) polymerase (PARP), an enzyme involved in DNA repair that is cleaved by caspase 3 during the early stages of apoptosis to produce 85 kD and 25 kD fragments, resulting in loss of normal PARP function (Nicholson et al. (1995) Nature 376: 37-43; Partel et al. (1996) FASEB J. 10: 587-597). Cells in suspension or attached to poly-L-lysine displayed significant PARP cleavage, while cells attached to fibronectin showed little PARP cleavage (FIG. 10B, P=0.006). Similarly, attachment to fibronectin, but not poly-L-lysine, protects HUVECs from DNA fragmentation associated with apoptosis (FIG. 10C, P=0.0001). These studies indicate that fibronectin attachment promotes the survival of proliferating endothelial cells.

To determine if integrin α5β1 is the fibronectin receptor supporting HUVEC survival in vitro, HUVECs were cultured on surfaces coated with anti-α5β1 or control antibodies. Immobilized anti-integrin antibodies cluster integrins, thereby acting as agonists (Stromblad et al. (1996) J. Clin. Invest. 98: 426-433; Leaveley et al. (1993) J. Cell Biol. 121: 163-170). Cells attached to surfaces coated with antibodies directed against α5β1 bound little annexin (FIG. 10D, P=0.001), and thus remained viable. In contrast, more than 75% of cells attached to control antibodies were annexin positive. Cells attached to α5β1 antibody-coated surfaces also showed significantly less PARP cleavage (FIG. 10E, P=0.007) and DNA fragmentation (FIG. 10F, P=0.003) than cells attached to control antibodies. These results indicate that α5β1 promotes endothelial cell survival.

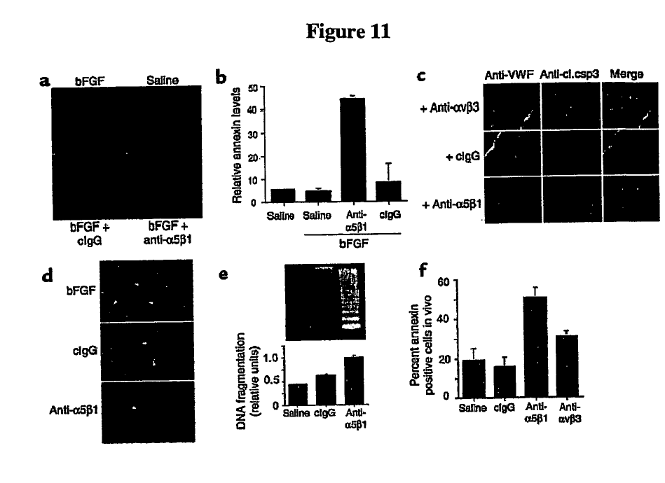

Antagonists of integrin α5β1 block angiogenesis in vivo but have no effect on unstimulated blood vessels (Kim et al. (2000) Am. J. Path. 156:1345-1362). To determine if these antagonists induce endothelial cell apoptosis during angiogenesis in vivo, CAMs were stimulated with bFGF, treated with saline, function-blocking anti-α5β1 antibodies and isotype-matched control antibodies and then analyzed for markers of apoptosis. Intravenous injection of fluorescently labeled annexin V demonstrated that α5β1 antagonists induce endothelial cell apoptosis in vivo. Anti-α5β1 antibodies, but not saline or control antibodies, induced annexin V staining of blood vessels in living CAMs, indicating that α5β1 regulates survival in vivo of proliferating endothelial cells (FIG. 11A). In fact, vessels in anti-α5β1 treated CAMs bound eight times more annexin V than control treated CAMs (FIG. 11B, P=0.015). These anti-α5β1 antibodies were previously shown to react with α5β1 on chicken endothelial cells and to block its function in vitro and in vivo (Kim et al. (2000) Am. J. Path. 156:1345-1362). As α5β1 is only expressed at significant levels on proliferating endothelial cells (17), α5β1 function-blocking antibodies target proliferating endothelial cells. As peptide and small molecule antagonists of α5β1 also inhibit angiogenesis (Kim et al. (2000) Am. J. Path. 156:1345-1362) and induce apoptosis (not shown), antibody-mediated complement activation is not likely to play a significant role in this apoptosis induction. Thus, these results suggest that integrin α5β1 antagonists induce endothelial cell apoptosis in vivo.

To determine further whether α5β1 antagonists cause apoptosis in vivo, sections of CAMs were analyzed for the expression of cleaved caspase 3 in blood vessels (FIG. 11C, P=0.01). Cleavage of caspase 3 into 17 and 12 kDa fragments is an indication of caspase 3 activation; the amount of cleaved caspase 3 is a quantitative index of apoptosis induction. Antibodies directed against mammalian caspases cross-react with avian caspases, as avian caspases exhibit 65% overall sequence identity and 100% activation domain sequence identity with mammalian caspases (Johnson et al. (2000) Biology of Reproduction 62: 589-598). Treatment with either α5β1 or αvβ3 antagonists induces caspase 3 cleavage (green) in blood vessels (red) in growth factor stimulated CAMs. Antagonists of a α5β1 but not control antibodies also induce DNA fragmentation in bFGF stimulated endothelial cells, as evaluated by TUNEL staining (FIG. 11D) and by analysis of DNA fragmentation of agarose gels (FIG. 11E, P=0.003). Furthermore, isolated cells from CAMs treated with α5β1 and αvβ3 function-blocking antibodies bound significantly more FITC-annexin V than in cells from control CAMs (FIG. 11F, P=0.0002 and 0.003, respectively). These studies definitively show that α5β1 antagonists induce endothelial cell apoptosis during angiogenesis.

### Example 25

### Unligated Integrin α5β1 Induces Apoptosis in Adherent Endothelial Cells

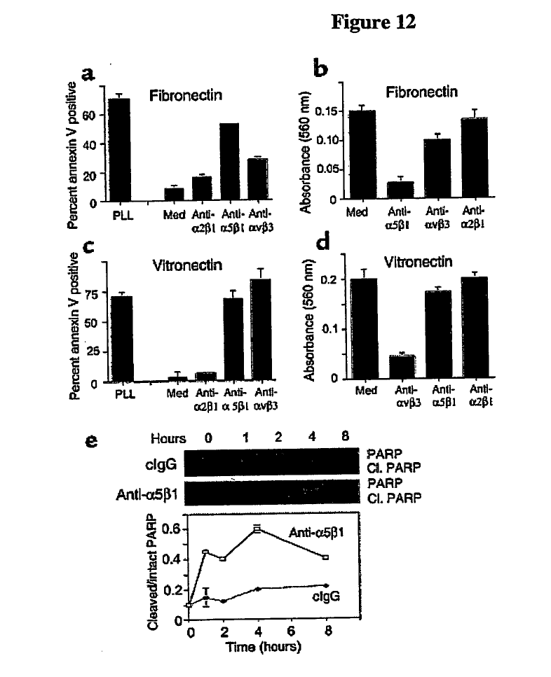

To determine how α5β1 antagonists interfere with cell survival during angiogenesis, proliferating HUVECs in complete culture medium were plated on poly-L-lysine, fibronectin, vitronectin, or collagen coated culture plates in the presence of antibody antagonists of integrins α5β1, αvβ3, or α2β1 for 24 h. Cells were then stained with FITC-annexin V. While fibronectin and vitronectin promote the survival of proliferating endothelial cells (FIG. 12A, 3C), collagen does not (Montgomery et al. (1994) Proc. Natl. Acad. Sci. USA 91: 8856-8860). Integrin antagonists that block cell attachment to the substratum (FIG. 12B,D) induce apoptosis, or anoikis, in endothelial cells (FIG. 12A,C). For example, antagonists of α5β1 induce apoptosis on fibronectin (FIG. 12A, P=0.001), while antagonists of αvβ3 induce apoptosis on vitronectin (FIG. 12C, P=0.02) by blocking cell attachment to the substratum.

In vivo, however, apoptosis (anoikis) induced by loss of attachment to the ECM is unlikely to occur as endothelial cells are attached to multiple matrix proteins through multiple integrins at any one time. Therefore, we also examined the ability of integrin antagonists to induce apoptosis in cells that remain attached through other integrins. In fact, α5β1 antagonists induce apoptosis of cells on vitronectin (FIG. 12C, P=0.05) without affecting their attachment to vitronectin (FIG. 12D). Anti-α5β1, but not control antibodies, also induce PARP cleavage in HUVECs plated on vitronectin substrates (FIG. 12E, P=0.003). Thus, unligated α5β1 inhibits endothelial cell survival on provisional matrix proteins such as fibronectin and vitronectin. These studies indicate that integrin α5β1 provides critical survival signals to proliferating endothelial cells such as those participating in angiogenesis. These studies indicate that integrin α5β1 can directly and indirectly regulate survival of proliferating endothelial cells.

### Example 26

### Unligated Integrins α5β1 Activates a Caspase 8 Mediated Apoptosis Pathway In Vitro and In Vivo

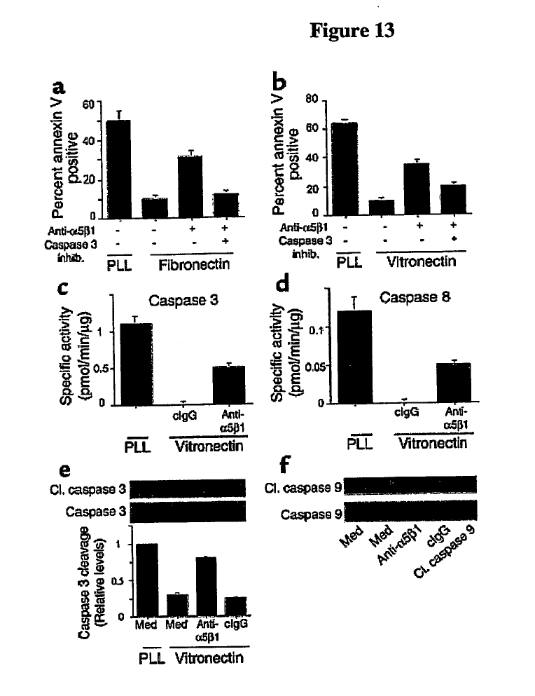

To determine the nature of the cell death pathway induced by α5β1 antagonists, endothelial cells were plated on ECM protein coated culture plates in the presence of integrin antagonists and caspase inhibitors or vehicle control (0.33% DMSO). Cell death induced by anti-α5β1 antibodies was blocked by caspase 3 inhibitors whether the cells were attached to fibronectin (P=0.0005, FIG. 13A) or vitronectin (P=0.02, FIG. 13B). Cell attachment to poly-L-lysine rapidly activated caspases 3 and 8 while attachment to vitronectin did not (FIG. 13C-D). Anti-α5β1 antibodies but not control antibodies also activated caspases 3 and 8 (P=0.0001 and P=0.002, respectively) in cells attached to vitronectin (FIG. 13C-D) and fibronectin (not shown). Caspase 3 cleavage was also readily detected in cells treated with anti-α5β1 but not control antibodies (P=0.01, FIG. 13E). In contrast, caspase 9 cleavage was not detected in cells treated with α5β1 antagonists (FIG. 13F). These studies show that integrin α5β1 antagonists induce a pro-apoptotic pathway in proliferating endothelial cells that results from activation of initiator caspases (8) rather than stress caspase (9) pathways. These results also indicate that blocking α5β1 ligation induces caspase 8- and 3-mediated death even when cells are still attached to provisional matrix ligands through other integrins.

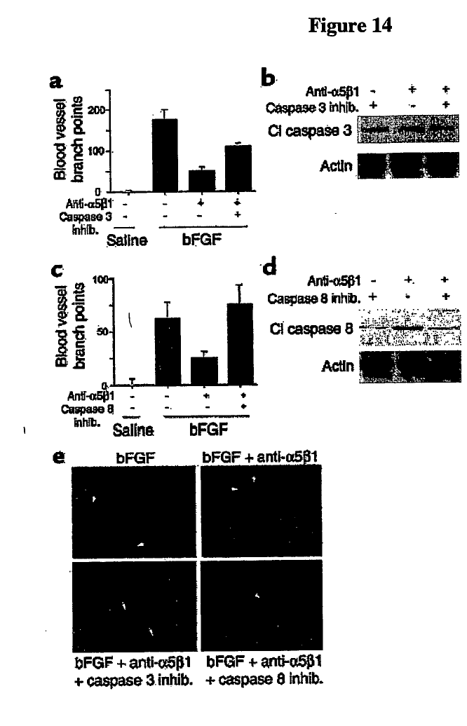

Our studies show that integrin-mediated survival depends on suppression of caspase 3 and 8 activity in vitro. As α5β1 antagonists block angiogenesis in vivo, these antagonists may induce caspase 3 and 8 activation in vivo. CAMs stimulated with bFGF were treated with saline, vehicle control (DMSO), caspase 3 or 8 inhibitors, and anti-integrin antibodies in the presence or absence of caspase inhibitors. Angiogenesis was inhibited by anti-α5β1 antibodies (FIG. 14A-D); this inhibition was partially reversed by cell permeable caspase 3 inhibitors (P=0.04, FIG. 14A) and fully reversed by caspase 8 inhibitors (P=0.05, FIG. 14C). Caspase 3 inhibitors partially blocked caspase 3 activity in vivo (FIG. 14B), while caspase 8 inhibitors completely blocked its activity in vivo (FIG. 14D). Furthermore, caspase 3 and 8 inhibitors prevented in vivo endothelial cell DNA fragmentation induced by α5β1 inhibition (FIG. 14E). Caspase 9 inhibitors had little effect on angiogenesis (not shown). Caspase inhibitors alone had no effect on angiogenesis or on unstimulated CAMs. These results indicate that α5β1 antagonists activate caspases 8 and 3 in vivo, thereby inhibiting angiogenesis.

### Example 27

### Unligated Integrins Induce PKA-Dependent Apoptosis

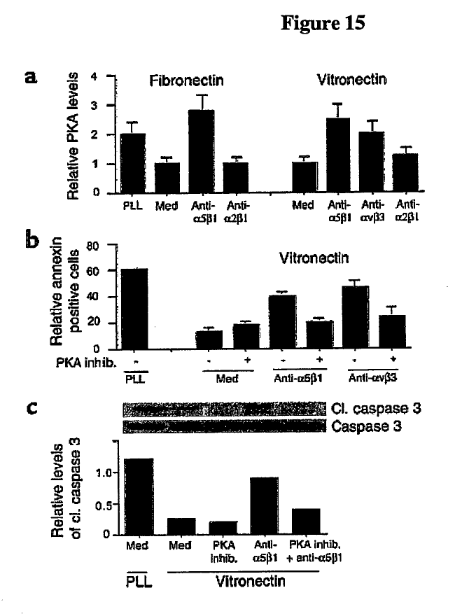

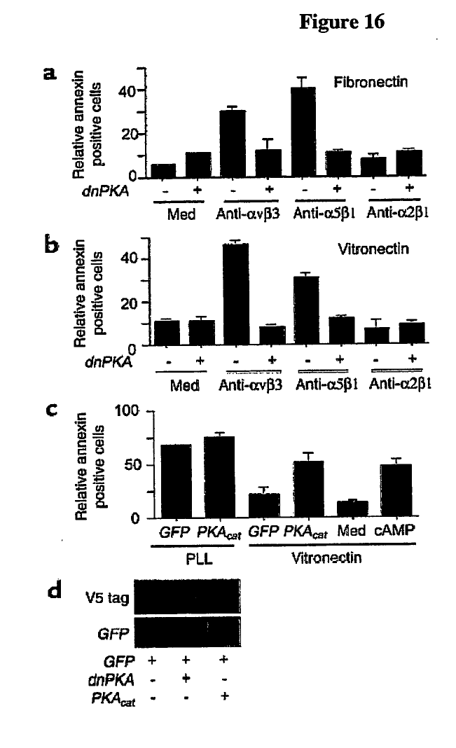

Integrin ligation activates signaling pathways that promote cell migration, proliferation and survival. Typically, integrin-mediated signaling is characterized by increases in tyrosine phosphorylation of signaling intermediates such as focal adhesion kinase, src, and ERK family members. However, integrin ligation also suppresses the activation of at least one kinase, protein kinase A (PKA, Kim et al. (2000) J. Biol. Chem. 275: 33920-33928). Importantly, we found that integrin ligation suppresses PKA activation, while antagonists of integrins activate this enzyme (FIG. 15A). Notably, antagonists of integrin α5β1 activate endothelial cell PKA whether endothelial cells are plated on fibronectin or on vitronectin (FIG. 15A). Therefore, we investigated the contribution of PKA to integrin-mediated cell death. We found that a pharmacological inhibitor of PKA, HA1004, substantially suppressed the apoptosis induced by integrin antagonists anti-αvβ3 (P=0.05) or anti-α5β1 (P=0.05) in cells attached to vitronectin, as detected by annexin V binding to intact cells (FIG. 15B). PKA inhibitors also blocked caspase 3 cleavage induced by anti-α5β1 (P=0.002, FIG. 15C). Expression of mutationally inactive PKA (dnPKA), but not a control transgene (GFP), also prevented integrin-antagonist induced cell death (P=0.004; FIG. 16A-B). More than 80% of cells expressed the transgene, which was also detected by Western blotting (FIG. 16D). These studies indicate that both direct (anoikis) and indirect integrin antagonist-mediated cell death is PKA dependent.

### Example 28

### PKA Activation Induces Endothelial Cell Apoptosis In Vitro and In Vivo

Our studies show that inhibition of PKA suppresses integrin antagonist-induced cell death. To determine whether activation of PKA directly induces endothelial cell death, endothelial cells were treated with dibutyryl cAMP or were transiently transfected with the active, catalytic subunit of PKA. Both cAMP and expression of the catalytic subunit of PKA significantly induce apoptosis (P=0.009 and P=0.003, respectively) in endothelial cells in vitro (FIG. 16C). Thus, PKA directly induces apoptosis in endothelial cells.

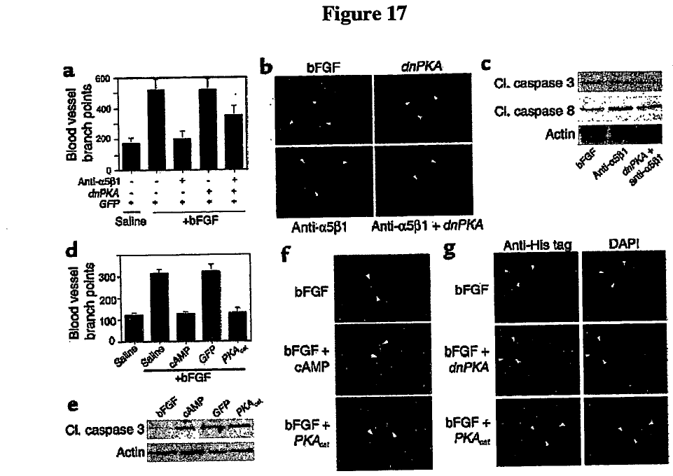

To determine whether PKA plays a role in the negative regulation of angiogenesis in vivo, bFGF stimulated CAMs were transfected with expression plasmids encoding GFP and mutationally inactive PKA (dnPKA) and treated with α5β1 antibodies. While α5β1 antagonists block angiogenesis (FIG. 17A, P=0.004), dnPKA reverses this inhibition (FIG. 17A, P=0.02). Expression of dnPKA also prevents α5β1 antagonist induced DNA fragmentation in vivo as detected by TUNEL staining (FIG. 17B) and inhibits activation of caspases 3 and 8 in vivo (FIG. 17C). Since integrin antagonists induce PKA-dependent apoptosis in vivo, direct activation of PKA may also inhibit angiogenesis. Expression of the PKA catalytic subunit during angiogenesis in vivo completely suppresses angiogenesis (P=0.0005), as does exposure to cAMP (P=0.001, FIG. 17D). VEGF stimulated angiogenesis is also inhibited by activation of PKA. This inhibition results from apoptosis induction, as it is accompanied by DNA fragmentation detected by TUNEL staining (FIG. 17E) and caspase 3 cleavage in vivo (FIG. 17F). The expression of transgenes could be demonstrated in endothelial cells in vivo by immunohistochemical staining for the presence of His tagged proteins (FIG. 17G). Thus, activation of PKA by integrin antagonists, by cAMP or by expression of the catalytic subunit of PKA induces endothelial cell apoptosis and inhibits angiogenesis.

These studies demonstrate that the unligated integrin α5β1 induces endothelial cell apoptosis in vivo in a PKA-dependent manner, thereby inhibiting angiogenesis. These studies demonstrate that activation of PKA by unligated integrins is an essential step in the induction of apoptosis. Importantly, our studies demonstrate that direct activation of PKA in vivo induces endothelial cell apoptosis and inhibition of angiogenesis.

### Example 29

### PTHrP Suppresses Angiogenesis In Vivo

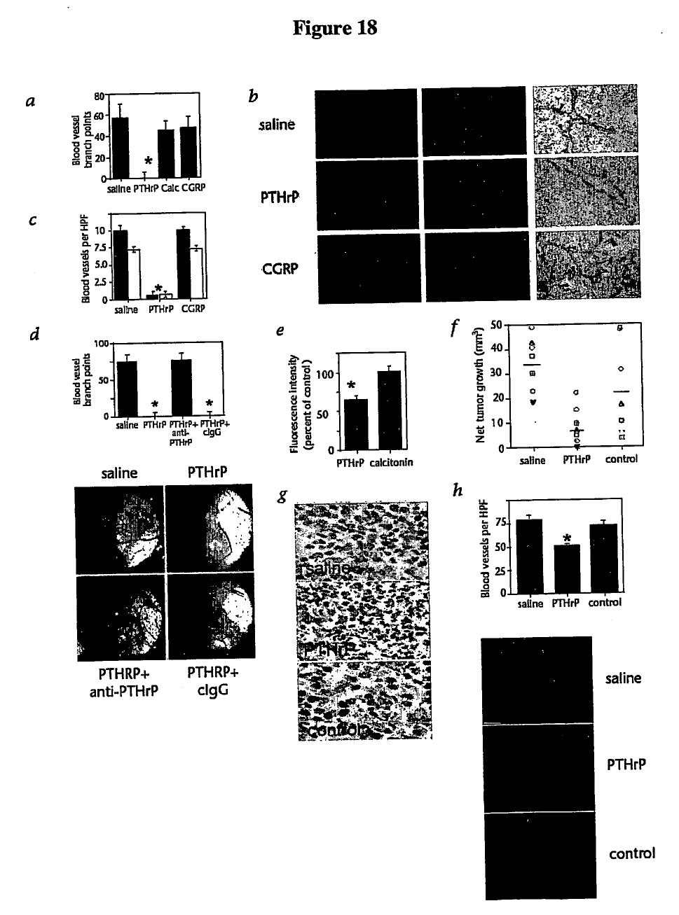

To evaluate the role of PTHrP in blood vessel development, we tested the effects of PTHrP on angiogenesis in the chick chorioallantoic membrane (CAM). CAMs from 10 day old chick embryos were stimulated with basic fibroblast growth factor (bFGF) in the presence or absence of PTHrP (1-173) or two other peptide hormones, calcitonin (a calcium regulating peptide hormone (Bukoski et al. (1995) Semin. Nephrol. 15, 536-549)) and calcitonin gene related peptide (CGRP, a vasodilatory peptide hormone (Bukoski et al. (1995) Semin. Nephrol. 15, 536-549)). Only PTHrP inhibited angiogenesis whether analyzed by enumerating blood vessel branchpoints (P<0.01, FIG. 18a) or density of vessels positive for integrin αvβ3 (P<0.0001, FIG. 18b) or von Willebrand factor, VWF (P<0.001, FIG. 18b). An analysis of blood vessel morphology also indicates that PTHrP inhibits bFGF-induced vascular branching (FIG. 18b). PTHrP had no effect on the number, size or composition of pre-existing vessels, as determined by macroscopic evaluation of vessels in saline-stimulated CAMs or by examining the number and nature of αvβ3 and VWF positive vessels in saline-stimulated CAMs. These studies indicate that PTHrP selectively prevents the formation of new microvessels. The half-maximal inhibitory dose of PTHrP was 0.01 μM. This inhibition could be blocked by addition of an anti-PTHrP function blocking antibody (8B12), directed against amino acids 1-34 (Terkeltaub et al. (1998) Arthritis and Rheumatism 41, 2152-64) (FIG. 18c). PTHrP also blocked bFGF-stimulated angiogenesis in the mouse (P<0.001, FIG. 18d). These studies indicate that PTHrP functions as an angiogenesis inhibitor.

PTHrP also inhibits tumor angiogenesis and growth. We treated nude mice bearing DU145 prostate carcinoma tumors (that did not express PTHrP) with daily intravenous injections of PTHrP (final serum concentration 1 μM). PTHrP suppressed tumor growth (FIG. 18e; P<0.005), induced tumor necrosis (FIG. 18f) and inhibited tumor angiogenesis (FIG. 18g; P<0.0001). While saline treated tumors doubled in size during the ten-day study, PTHrP treated tumors increased in size by only 30%. These studies indicate that PTHrP inhibits tumor angiogenesis as well as growth factor-induced angiogenesis.

### Example 30

### Inhibition of Angiogenesis and Tumor Growth by PTHrP Gene Delivery

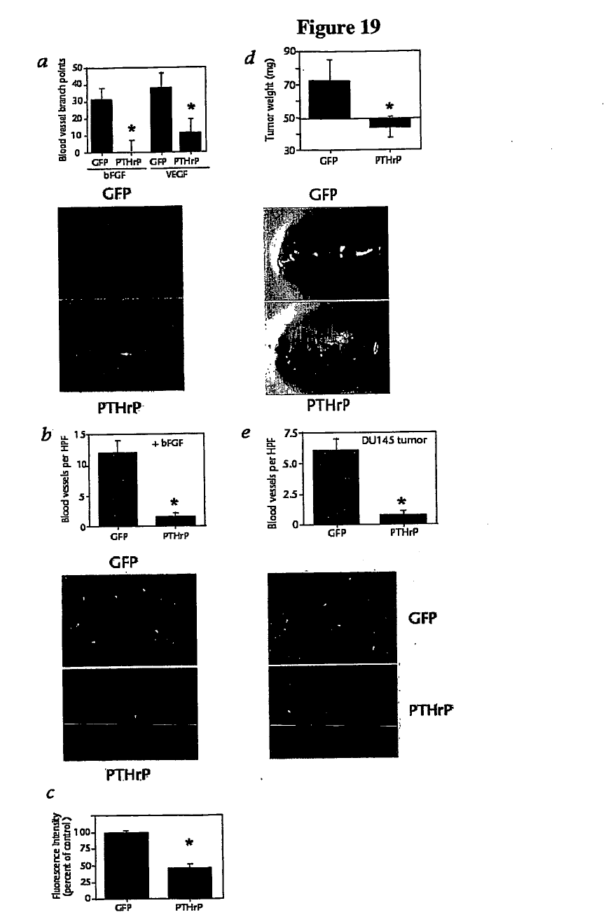

To determine if gene delivery of PTHrP might be a useful strategy for the therapeutic inhibition of angiogenesis in vivo, we stimulated angiogenesis in chick CAMs with bFGF or vascular endothelial growth factor (VEGF). We then transduced CAMs by injecting adenoviruses expressing Green Fluorescent Protein (GFP) or full length PTHrP (1-173) (Terkeltaub et al. (1998) Arthritis and Rheumatism 41, 2152-64) into the embryonic circulation. Virally expressed PTHrP, but not GFP, inhibited angiogenesis stimulated by bFGF (P<0.001) or VEGF (P=0.01) as shown in by quantifying blood vessel branchpoints (FIG. 19a, left panel) or integrin αvβ3 immunoreactive vessels (P<0.0001, FIG. 19b). PTHrP expression was detected in blood vessels (arrows) in CAMs transduced with PTHrP using an antibody directed against PTHrP C-terminal amino acids 109-141 (Abdeen et al. (1995) Am. J. Gastroenterology 90,1864-1867) (FIG. 19a, right panel). Virally expressed PTHrP also inhibited angiogenesis in the adult mouse. Viral delivery of PTHrP inhibited bFGF stimulated murine angiogenesis (P<0.05, FIG. 19c). Virally induced expression of PTHrP also inhibited tumor growth on the CAM (P<0.03, FIG. 19d), suggesting that locally delivered PTHrP can suppress tumor angiogenesis. PTHrP treated tumors are smaller, obviously necrotic and associated with fewer integrin αvβ3 positive blood vessels than GFP treated tumors (FIG. 19e, P<0.0001). Thus, virally expressed PTHrP can inhibit angiogenesis and may serve as a useful therapeutic angiogenesis inhibitor.

### Example 31

### PTHrP Inhibits Endothelial Cell Migration

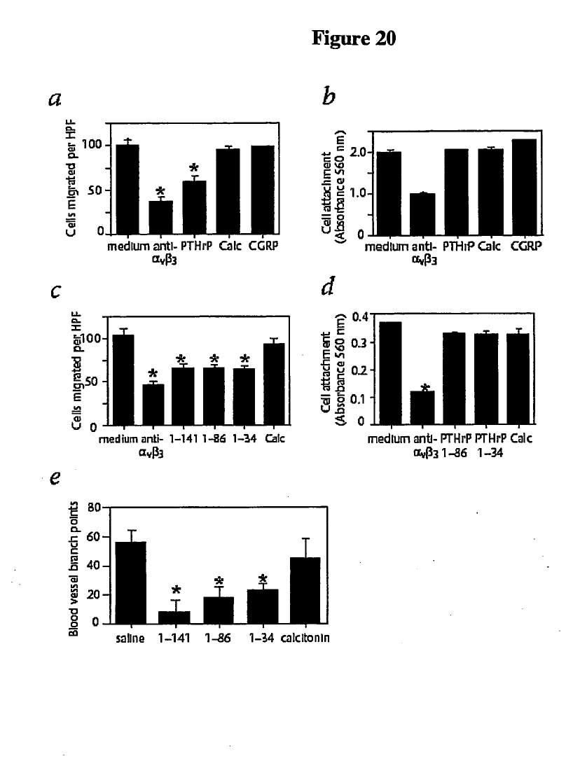

To evaluate the role of PTHrP in endothelial functions in vitro, we tested the effects of PTHrP (1-173) and other peptide hormones on endothelial cell migration on extracellular matrix substrates such as vitronectin, collagen and fibronectin. PTHrP significantly inhibited cell migration on vitronectin (FIG. 20a), collagen and fibronectin, yet had no effect on attachment to these proteins (FIG. 20b). Fifty percent inhibition of cell migration was achieved with PTHrP in the range of 1-10 μM. These studies demonstrate the PTHrP inhibits endothelial cell migration, but is not an inhibitor of integrin ligation.

### Example 32

### Mapping of the Anti-Migratory and Anti-Angiogenic Active Sites of PTHrP

PTHrP is composed of several domains with distinct physiological properties (Jin et al. (2000) J. Biol. Chem. 275: 27238-27244). To identify the domain(s) of PTHrP responsible for the anti-migratory and anti-angiogenic effects, we evaluated the effects of various fragments of the peptide hormone in migration and angiogenesis assays. Like PTHrP (1-173), fragments containing the N-terminus (1-141, 1-86 and 1-34) inhibited migration (FIG. 20c), but not attachment (FIG. 20d), on vitronectin. Fragments lacking the N-terminus, such PTHrP 107-138, were not able to inhibit cell migration. In vivo, PTHrP 1-141, 1-86 and 1-34 potently inhibited angiogenesis (FIG. 20e). As the first 34 amino acids contain the angiogenesis inhibition properties, further studies were performed to identify the critical residues responsible for angiogenesis inhibition.

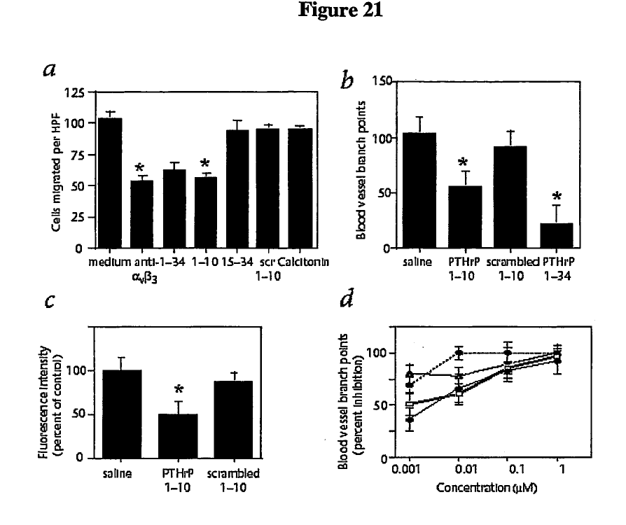

Studies have shown that the first six amino acids of PTH are required for activation of PTH/PTHrP receptor signaling while the last fifteen are required for high affinity binding to the receptor (Jin et al. (2000) J. Biol. Chem. 275: 27238-27244). Five out of the first ten amino acids in PTH and PTHrP are identical and structure predictions indicate these regions have similar conformations³⁰. Therefore, to further delineate the active angiogenesis and cell migration inhibition sites on PTHrP, we compared the activities of fragments of PTHrP extending from amino acids 1-10, 1-34, and 15-34 to a scrambled 1-10 peptide. While amino acids 1-34 and 1-10 were similarly effective in inhibiting cell migration on vitronectin, amino acids 15-34 and a scrambled version of 1-10 were unable to inhibit cell migration (FIG. 21a). The N-terminal PTHrP fragment 1-10 inhibited cell migration in a dose-dependent manner, with a maximum of 50% inhibition at a concentration of 10 μM. PTHrP 1-10 and 1-34 were also effective in inhibiting angiogenesis in the CAM assay, while scrambled 1-10 was not (FIG. 21b). PTHrP 1-10 is slightly less effective at inhibiting angiogenesis than 1-34, however. PTHrP 1-10 was also effective in inhibiting angiogenesis in a mouse model of angiogenesis while scrambled PTHrP 1-10 peptide was not (P<0.01, FIG. 21c). These studies indicate that the first ten PTHrP residues are critical for activation of endothelial cell PTHrP receptor in vitro and in vivo.

Angiogenesis can be induced by many growth factors. To determine if PTHrP is a general inhibitor of angiogenesis, we evaluated the effect of PTHrP 1-34 on bFGF, VEGF, IL-8 and TNFα stimulated angiogenesis (FIG. 21d). Angiogenesis induced by each growth factor was inhibited by PTHrP (1-34), with complete inhibition occurring at 1 μM. The IC₅₀ for each growth factor was 0.001 μM or lower. Thus, PTHrP is a potent general angiogenesis inhibitor.

### Example 33

### Mechanism of PTHrP Anti-Angiogenic Effects

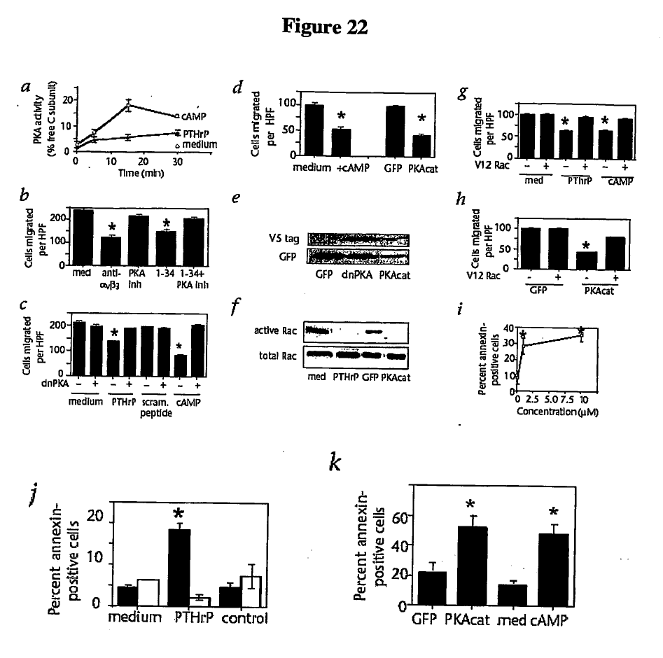

While an understanding of the mechanism of the invention is not required, and without intending to limit the invention to any particular mechanism, we set out to establish the mechanism by which PTHrP inhibits cell migration in vitro and angiogenesis in vivo. We evaluated the effects of PTHrP on endothelial cell signal transduction. Like PTH, PTHrP interacts with the PTH1 receptor, a G protein coupled receptor that activates PKA (Abou-Samra et al. (1992) Proc. Natl. Acad. Sci. USA 89: 2732-2736; Hoare et al. (2001) J. Biol. Chem. 276, 7741-7753) expressed on endothelial cells (Jiang et al. (1998) J. Cardiovascular Pharmacol. 1998, S142-1444). We found that PTHrP 1-34, as well as a cell permeable cAMP (dibutyryl cAMP), rapidly stimulate PKA activity in endothelial cells (FIG. 22a). PKA activation is detected in as little as 2 minutes, with maximal activity 15 minutes after stimulation with both cAMP and PTHrP. To determine if the anti-migratory properties of PTHrP result from signals transduced through PKA, we evaluated the effects of PTHrP 1-34 on cell migration in the presence and absence of the PKA inhibitor, N-(2-(p-bromocinnamylamino)ethyl)-5-isoquinoline sulfonamide (H89, FIG. 22b). This PKA inhibitor blocked the anti-migratory properties of PTHrP (P<0.01). Furthermore, expression of a mutationally inactive form of PKA (dnPKA), which blocks PKA activation (Howe et al. (2000) Nature Cell Biol. 2, 593-600) suppresses the PTHrP- and cAMP-mediated inhibition of endothelial cell migration (P<0.0001, FIG. 22c). In fact, direct activation of PKA by either cAMP (P<0.0001) or by transient transfection with the PKA catalytic subunit (P<0.0001) also inhibits endothelial cell migration (FIG. 22d). Expression of both transgenes could be detected by Western blotting of lysates of transfected cells (FIG. 22e). These results indicate that PTHrP inhibition of migration is PKA dependent and that activation of PKA blocks endothelial cell migration.

The small GTPase Rac plays an essential role in regulating cell motility by influencing actin assembly and lamellipodia extension (Ridley et al. (1992) Cell 70,401-10). Cell adhesion as well as growth factor stimulation upregulate Rac activity in endothelial cells (Price et al. (1998) Mol. Biol. Cell 9, 1863-71). We found that activation of Rac is blocked by PTHrP, by expression of activated PKA (FIG. 22f) or by cAMP (not shown). Overexpression of mutationally active Rac (V12 Rac) overcomes the PTHrP (P<0.007), cAMP (P<0.003, FIG. 22g), or PKA catalytic subunit (FIG. 22h, P<0.006) mediated inhibition of cell motility. These studies indicate that PTHrP activation of PKA inhibits cell migration by inhibiting Rac activation.

Signal transduction pathways that promote cell migration often also promote cell survival (Cho et al. (2000) J Cell Biol. 149:223-36). Since PTHrP inhibits endothelial cell migration, it is possible that it inhibits endothelial cell survival. In fact, PTHrP induces apoptosis of endothelial cells in a dose-dependent manner (FIG. 22i). Like its effects on cell migration, PTHrP-induced apoptosis is PKA-dependent, as expression of dominant negative PKA suppresses this induction of cell death (FIG. 22J). These studies suggest that direct activation of PKA may induce apoptosis in endothelial cells. We found that endothelial cell expression of the catalytic subunit of PKA or exposure to dibutyryl cAMP also induced apoptosis in endothelial cells (FIG. 22k). These studies indicate that PTHrP not only inhibits endothelial cell migration but also induces endothelial cell apoptosis in a PKA-dependent manner.

### Example 34

### PTHrP Inhibition of Angiogenesis is Protein Kinase A Dependent

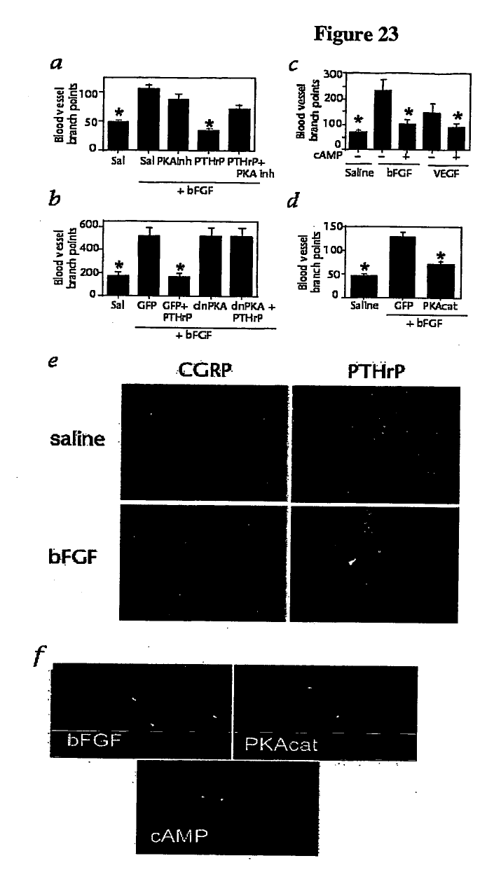

Our studies suggest that activation of PKA in vivo may inhibit angiogenesis. We therefore evaluated the role of PKA in the inhibition of angiogenesis by PTHrP. We found that either pharmacological or genetic inhibition of PKA reverses PTHrP inhibition of angiogenesis. A selective PKA inhibitor (H89) blocks the suppression of angiogenesis induced by PTHrP 1-34 (P<0.0002, FIG. 23a). Expression of mutationally inactive PKA (dnPKA) also reverses the inhibitory effects of PTHrP (P<0.0003, FIG. 23b). Furthermore, activation of PKA by cAMP (P<0.0003, FIG. 23c) or by expression of the catalytic subunit of PKA in the CAM (P=0.0005, FIG. 23d) potently inhibits angiogenesis. These studies demonstrate that PTHrP inhibits angiogenesis by activating PKA. Recent studies demonstrated that Rac activity is required for angiogenesis (Dormond et al. (2001) Nature Med. 7,1041-7). Taken together, these studies suggest that activation of PKA in endothelial cells in vivo induces Rac inactivation and likely inhibition of cell migration in vivo. In addition, PTHrP or PKA may inhibit angiogenesis by inducing apoptosis of proliferating endothelial cells in vivo. In fact, TUNEL staining of PTHrP-treated CAMs reveals that this hormone induces apoptosis in bFGF- but not saline-stimulated endothelial cells in vivo (FIG. 23e). Expression of the PKA catalytic subunit in bFGF-stimulated CAMs or exposure of CAMs to cAMP also induces endothelial cell apoptosis in vivo (FIG. 23f). These studies suggest that PTHrP inhibits angiogenesis by directly and specifically by inducing PKA dependent endothelial cell apoptosis. These studies further show that activation of PKA by hormonal, pharmacological or genetic means is a potent means to inhibit angiogenesis.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiment, it should be understood that the invention as claimed should not be unduly limited to such specific embodiment. Indeed, various modifications of the described modes for carrying out the invention which are obvious to those skilled in the art and in fields related thereto are intended to be within the scope of the following claims.

We claim:

1. A method for reducing angiogenesis in a subject, comprising:
a) providing:
i) a subject comprising tissue that comprises endothelial cells; and
ii) at least one nucleotide sequence encoding a protein comprising a protein kinase A catalytic subunit; and
b) expressing said nucleotide sequence in said endothelial cells such that angiogenesis by said endothelial cells is reduced.

2. The method of claim 1, further comprising step c) detecting a reduction in angiogenesis by said endothelial cells.

3. The method of claim 1, wherein said subject is human.

4. The method of claim 1, wherein said tissue comprises at least one of ocular tissue, skin tissue, bone tissue, and synovial tissue.

5. The method of claim 1, wherein said tissue comprises a tumor.

6. The method of claim 5, wherein said tumor is malignant.

7. The method of claim 6, wherein said malignant tumor is metastatic.

8. The method of claim 1, wherein said subject has a pathological condition associated with angiogenesis in said tissue.

9. A method for increasing cell apoptosis in a subject, comprising:
a) providing:
i) a subject comprising tissue that comprises cells; and
ii) at least one nucleotide sequence encoding a protein comprising a protein kinase A catalytic subunit; and
b) expressing said nucleotide sequence in said cells such that apoptosis of said cells is increased.

10. The method of claim 9, wherein said subject is human.

11. The method of claim 9, wherein said cell is chosen from endothelial cell, vascular smooth muscle cell, monocyte cell, macrophage cell, benign tumor cell, malignant tumor cell, fibroblast cell, B cell, T cell, myocyte cell, megakaryocyte cell, eosinophil cell, neurite cell, and synoviocyte cell.

12. The method of claim 9, wherein said subject has a pathological condition chosen from angiogenesis, restenosis, atherosclerosis, cancer, tumor metastasis, fibrosis, hemangioma, lymphoma, leukemia, psoriasis, arthritis, autoimmune disease, diabetes, amyotrophic lateral sclerosis, graft rejection, retinopathy, macular degeneration, and retinal tearing.

13. The method of claim 12, wherein said pathological condition is fibrosis and said tissue is chosen from heart, lung, and liver.

14. The method of claim 12, wherein said pathological condition is an autoimmune disease chosen from Lupus, Crohn's disease, and multiple sclerosis.

15. A method for reducing angiogenesis in a subject, comprising:
a) providing:
i) a subject comprising tissue that comprises endothelial cells; and
ii) at least one polypeptide sequence comprising a sequence chosen from at least one of AVSEHQLLHS/D (SEQ ID NO:114) and SVSEIQLMNL (SEQ ID NO:115); and
b) treating said endothelial cells with said polypeptide sequence such that angiogenesis by said endothelial cells is reduced.

16. The method of claim 15 further comprising step c) detecting a reduction in angiogenesis by said endothelial cells.

17. A method for increasing cell apoptosis in a subject, comprising:
a) providing:
i) a subject comprising tissue that comprises cells; and
ii) at least one polypeptide sequence comprising a sequence chosen from at least one of AVSEHQLLHS/D (SEQ ID NO:114) and SVSEIQLMNL (SEQ ID NO:115); and
b) treating said cells with said polypeptide sequence such that apoptosis of said cells is increased.

18. The method of claim 17 further comprising step c) detecting an increase in apoptosis of said cells.

19. A method for reducing angiogenesis in a subject, comprising:
a) providing:
i) a subject comprising a tissue that comprises endothelial cells; and
ii) at least one agent chosen from pertussis toxin, cholera toxin, G alpha i minigene, dominant negative G alpha i, dominant negative G alpha 12/13, constitutively active G alpha s, anti-CD47 antibody, dominant positive Rho (RhoV14), dominant negative Src, and active Csk; and
b) treating said endothelial cells with said at least one agent such that angiogenesis by said endothelial cells is reduced.

20. The method of claim 19, further comprising step c) detecting a reduction in angiogenesis by said endothelial cells.

21. A method for increasing cell apoptosis in a subject, comprising:
a) providing:
i) a subject comprising a tissue that comprises cells; and
ii) at least one agent chosen from pertussis toxin, cholera toxin, G alpha i minigene, dominant negative G alpha i, dominant negative G alpha 12/13, constitutively active G alpha s, anti-CD47 antibody, dominant positive Rho (RhoV14), dominant negative Src, and active Csk; and
b) treating said cells with said at least one agent such that apoptosis of said cells is increased.

22. The method of claim 21, further comprising step c) detecting an increase in apoptosis of said cells.

23. A method for reducing angiogenesis in a subject, comprising:
a) providing:
i) a subject comprising a tissue that comprises endothelial cells; and
ii) at least one Src inhibitor; and
b) treating said endothelial cells with said at least one Src inhibitor such that angiogenesis by said endothelial cells is reduced.

24. A method for increasing cell apoptosis in a subject, comprising:
a) providing:
i) a subject comprising a tissue that comprises cells; and
ii) at least one Src inhibitor; and
b) treating said cells with said at least one Src inhibitor such that apoptosis of said cells is increased.
